(19) United States
(12) Patent Application Publication
(10) Pub. No.: US 2024/0269189 A1
(43) Pub. Date: Aug. 15, 2024

(54) EXTRACELLULAR VESICLE-ENRICHED SECRETOME COMPOSITION DERIVED FROM INDUCED PLURIPOTENT STEM CELL DERIVED-MICROGLIA AND METHODS OF USE THEREOF

(71) Applicant: FUJIFILM Holdings America Corporation, Valhalla, NY (US)

(72) Inventors: Nisa RENAULT, Madison, WI (US); Michele HAMRICK, Madison, WI (US); Jacquelyn WONG, Madison, WI (US); Dana NELSON, Madison, WI (US); Kiranmayee BAKSHY, Madison, WI (US); Steve SMITH, Madison, WI (US); Coby CARLSON, Madison, WI (US); Megan K. LIVINGSTON, Madison, WI (US); Kimihiko TOMOTOSHI, Madison, WI (US); Lisa HARMS, Madison, WI (US)

(73) Assignee: FUJIFILM Holdings America Corporation, Valhalla, NY (US)

(21) Appl. No.: 18/545,837

(22) Filed: Dec. 19, 2023

Related U.S. Application Data
(60) Provisional application No. 63/476,102, filed on Dec. 19, 2022.

(51) Int. Cl. A61K 35/30; A61K 9/50; C12N 5/079; C12N 15/113

(52) U.S. Cl. CPC A61K 35/30; A61K 9/5068; C12N 5/0622; C12N 15/113; C12N 2310/141; C12N 2506/45

(57) ABSTRACT

The present disclosure provides extracellular vesicle-enriched compositions derived from iPSC-derived microglia. Further provided herein are methods for using the extracellular vesicle-enriched compositions from iPSC-derived microglia in screening assays to discover properties, including therapeutic properties.

### PRIORITY CLAIM

This application claims benefit of priority to U.S. Provisional Application Ser. No. 63/476,102 filed Dec. 19, 2022, the entire contents of which are hereby incorporated by reference.

### BACKGROUND

### 1. Field

The present invention relates generally to the field of molecular biology and medicine. More particularly, it concerns compositions of extracellular vesicles isolated from microglia (MGL) differentiated from induced pluripotent stem cells and methods of use thereof.

### 2. Description of Related Art

Cells, including those in in vitro or ex vivo culture, secrete a large variety of molecules and biological factors (collectively known as a secretome) into the extracellular space (Vlassov et al., 2012). As part of the secretome, various bioactive molecules are secreted from cells within membrane-bound extracellular vesicles, such as exosomes. Extracellular vesicles are capable of altering the biology of other cells through signaling, or by the delivery of their cargo (including, for example, proteins, lipids, and nucleic acids). The cargo of extracellular vesicles is encased in a membrane which, amongst others, allows for specific targeting (e.g., to target cells) via specific markers on the membrane; and increased stability during transport in biological fluids, such as through the bloodstream or across the blood-brain-barrier (BBB).

Exosomes exert a broad array of important physiological functions, e.g., by acting as molecular messengers that traffic information between different cell types. For example, exosomes deliver proteins, lipids and soluble factors including RNA and microRNAs which, depending on their source, participate in signaling pathways that can influence apoptosis, metastasis, angiogenesis, tumor progression, thrombosis, immunity by directing T cells towards immune activation, immune suppression, growth, division, survival, differentiation, stress responses, and the like (Vlassov et al., 2012). Extracellular vesicles may contain a combination of molecules that may act in concert to exert particular biological effects. Exosomes incorporate a wide range of cytosolic and membrane components that reflect the properties of the parent cell. Therefore, the terminology applied to the originating cell can in some instances be used as a simple reference for the secreted exosomes.

Progenitor cells have proliferative capacity and can differentiate into mature cells, making progenitor cells attractive for therapeutic applications such as regenerative medicine. It has been reported that extracellular vesicles secreted by stem cell-derived progenitor cells produce similar therapeutic effects to their secreting cells, suggesting that a significant mechanism of action of transplanted progenitor cells is in the release of biological factors following transplantation. This raises the possibility of effective, cell-free therapies with benefits such as improved convenience, stability, and operator handling (El Harane et al., 2018).

Microglia are innate immune cells of the central nervous system that perform critical roles in brain development, homeostasis, and immune regulation. They are hard to acquire from human fetal and primary tissues. Microglial exosomes have been implicated in the progression of neurodegeneration and neuronal functioning, including neurite outgrowth. However, there is an unmet need for compositions of extracellular vesicles derived from induced pluripotent stem cell (iPSC)-derived microglia.

### SUMMARY

In certain embodiments, the present disclosure provides a composition comprising a microglia-derived extracellular vesicle-enriched secretome. In some aspects, the microglia are induced pluripotent stem cell (iPSC)-derived microglia. In some embodiments, the present disclosure provides a composition comprising an extracellular vesicle-enriched secretome produced by induced pluripotent stem cell (iPSC)-derived microglia. A further embodiment provides a method of making a composition comprising an extracellular vesicle-enriched secretome, the method comprising culturing induced pluripotent stem cell (iPSC)-derived microglia to produce a conditioned media and isolating the extracellular vesicle-enriched secretome therefrom.

In some aspects, the microglia were not cryopreserved after differentiation from iPSCs. In certain aspects, the extracellular vesicle-enriched secretome was obtained from conditioned media (or spent media) of microglia or progenitors thereof prior to cell harvest after differentiation from iPSCs. In particular aspects, the microglia were not stimulated with lipopolysaccharide (LPS), phosphatidylserine positive (PS+) neurons, TNF-alpha, IFN-gamma, IL-4, or IL-10. In some aspects, the conditioned media was produced from a spent media which was previously diluted 1:1 with a buffer, such as phosphate-buffered saline (PBS). In some aspects, the extracellular vesicle-enriched secretome was isolated from the conditioned media (or spent media) by ultracentrifugation, tangential flow filtration (TFF) and size exclusion chromatography (TFF-SEC), or phosphatidyl-serine (PS) affinity capture.

In certain aspects, the extracellular vesicle-enriched secretome comprises hsa-miR-4669 and/or hsa-miR-4777-3p. In some aspects, the extracellular vesicle-enriched secretome comprises hsa-miR-16-5p, hsa-miR-223-3p, hsa-miR-93-5p, hsa-miR-146a-5p, hsa-miR-142-3p, hsa-miR-191-5p, hsa-miR-142-5p, hsa-miR-21-5p, hsa-miR-103a-3p/107, hsa-miR-26b-5p, hsa-miR-122-5p, hsa-miR-125b-5p, hsa-miR-25-3p, hsa-miR-146b-5p, hsa-miR-101-3p, hsa-miR-29a-3p, hsa-miR-30e-5p, hsa-let-7a-5p/7c-5p, hsa-miR-342-3p, hsa-miR-148b-3p, hsa-miR-27a-3p/27b-3p, hsa-miR-224-5p, hsa-let-7f-5p, hsa-miR-125a-5p, and/or hsa-miR-26a-5p.

In some aspects, the microglia were positive for TREM2, P2RY12, TMEM119, IBA-1, and/or CX3CR1. In certain aspects, the microglia were derived from donors expressing disease-associated SNPs. In some aspects, the microglia were generated from disease associated iPSC donors with TREM2, APOE, CD33, BIN, ABCA7, SNPS or genotypes associated with neurodegeneration. In some aspects, the microglia comprise a disruption in TREM2, Methyl-CpG Binding Protein 2 (MeCP2), and/or Alpha-synuclein (SCNA). In some aspects, the iPSCs are human.

In certain aspects, the extracellular vesicle-enriched secretome is positive for CD9, CD63, or CD81. In some aspects, the extracellular vesicle-enriched secretome is positive for CD9, CD63, CD81, beta-actin, and/or Flottilin-1. The extracellular vesicle-enriched secretome may be positive for GBA, LRRK2, and/or phosphor-LRRK2. In some aspects, the iPSC-derived microglia and/or extracellular vesicle-enriched secretome comprise a GBA protein larger than 80 kDa, such as a GBA protein of 84-88 kDa. The extracellular vesicle-enriched secretome and/or iPSC-derived microglia may comprise a TREM2 C-terminal cleavage fragment. In particular aspects, the extracellular vesicle-enriched secretome has no or essentially no HLA-ABC, CD86, and/or CD142.

In some aspects, the extracellular vesicle-enriched secretome has higher protein levels of CD9 as compared to CD81. In certain aspects, the extracellular vesicle-enriched secretome has higher protein levels of CD81 as compared to CD63. In some aspects, the extracellular vesicle-enriched secretome has higher protein levels of CD9 as compared to CD63. In some aspects, the extracellular-enriched secretome has higher protein levels of CD9 as compared to protein levels of CD81 and higher protein levels of CD81 as compared to expression of CD63. In some aspects, the CD81⁺CD9+ double-positive clusters (17%) are about three times as abundant as the CD63+CD9+ double-positive clusters or the CD63+CD81 double-positive clusters.

In some aspects, the composition comprises a pharmaceutically acceptable excipient or carrier. In certain aspects, the composition is free of or essentially free of LPS and/or PS+. In certain aspects, the composition further comprises dPBS.

In certain aspects, the extracellular vesicles are singlets, doublets, concentric multi-vesicular bodies, and/or non-concentric multi-vesicular bodies. In some aspects, the extracellular vesicle-enriched secretome substantially comprises concentric, nearly spherical multi-vesicular bodies. For example, the composition may comprise over 50%, 55%, 60%, 65%, 70%, 75%, 80%, 85%, 90%, or 95% concentric, nearly spherical bodies.

In some aspects, the extracellular vesicle-enriched secretome results in increased endothelial cell migration as measured by HUVEC scratch wound healing assay as compared to MV controls. In certain aspects, the extracellular vesicle-enriched secretome influences electrical activity of neurons. In some aspects, the extracellular vesicle-enriched secretome results in increased dopaminergic neuron viability, decreased neurite outgrowth, increased clustering of neuron cells, decreased number of cell body clusters, and/or increased cell body cluster area when contacted with dopaminergic neurons as compared to untreated controls. In certain aspects, the extracellular vesicle-enriched secretome results in an increased number of network bursts in dopaminergic neurons with a Parkinson's disease mutation as compared to untreated controls. In some aspects, the Parkinson's disease mutation is LRRK2 or GBA.

In certain aspects, the composition comprises relative protein levels of about 35-60% CD9, less than 10% CD63, and/or about 30-60% CD81. In certain aspects, the composition comprises less than 5% CD63. In particular aspects, the composition comprises less than 10% (e.g., less than 9%, 8%, 7%, 6%, 5%, 4%, 3%, 2%, 1%, or 0.1%) CD63 and more than 30% (e.g., 35%, 40%, 45%, 50%, 55%, or 60%) CD9. In specific aspects, the composition comprises less than 10% (e.g., less than 9%, 8%, 7%, 6%, 5%, 4%, 3%, 2%, 1%, or 0.1%) CD63 and less than 60% (e.g., 55%, 50%, 45%, 40%, 35%, or 30%) CD81. In some aspects, the composition comprises less than 10% (e.g., less than 9%, 8%, 7%, 6%, 5%, 4%, 3%, 2%, 1%, or 0.1%) CD63 and greater than 30% (e.g., 35%, 40%, 45%, 50%, 55%, or 60%) CD9 and/or less than 60% (e.g., 55%, 50%, 45%, 40%, 35%, or 30%) CD81. In certain aspects, the composition comprises between 0.5 and 8% CD63, between 36 and 62% CD9, and between 34 and 60% CD81, wherein each individual sample's three values add up to 100%. In some aspects, the composition has a relative expression of CD9:CD63:CD81 of about 57:3:40. In some aspects, the composition has a relative protein level of about 50-70% CD9:1-10% CD63: 30-50% CD81. In certain aspects, the composition relative protein levels of about 50-60% CD9, 1-5% CD63, and/or about 35-45% CD81. In some aspects, the composition comprises at least 5% CD63+CD9⁺. In certain aspects, the composition comprises at least 5% +CD53+CD81⁺. In some aspects, the composition comprises at least 15% CD81⁺CD9⁺.

In some aspects, the extracellular vesicle-enriched secretome comprises a median D50 (e.g., median particle size, in nm, of a sample, wherein 50% of measured particles were that size or lower, as determined by the Nanosight) of 110-130 nm, such as about 110-120, 110-115, 115-120, 120-125, or 120-130 nm.

Another embodiment provides a method of obtaining neurons with increased clustering comprising contacting said neurons with an extracellular-enriched secretome composition of the present embodiments or aspects thereof. In some aspects, the neurons are dopaminergic neurons, glutamatergic neurons or GABAergic neurons. The neurons can comprise increased viability, decreased neurite outgrowth, decreased number of cell body clusters, and/or increased cell body cluster area as compared to untreated controls.

A further embodiment provides a method for screening an extracellular vesicle-enriched composition of the present embodiments or aspects thereof comprising (a) contacting an extracellular vesicle-enriched composition of the present embodiments or aspects thereof with a target cell and/or target cell under a stress condition; and (b) measuring the functional activity of the target cell and/or stress condition. In some aspects, the screening is to detect a function of the extracellular vesicle-enriched secretome. In some aspects, the screening is for measuring the effect of the extracellular vesicle-enriched composition of the present embodiments or aspects thereof on wound healing. In certain aspects, the screening is for measuring the efficacy of the extracellular vesicle-enriched composition of the present embodiments or aspects thereof on treating a neurodegenerative disease. In some aspects, the target cells are dopaminergic neurons, HUVEC or microglia. In certain aspects, the target cells are astrocytes, glutamatergic neurons, or GABAergic neurons. In certain aspects, an increase in functional activity of the cells indicates the extracellular vesicle-enriched composition of the present embodiments or aspects thereof is capable of treating a neurodegenerative disease. In certain aspects, measuring functional activity comprises measuring cell viability, neurosphere (e.g., astrocytes, glutamatergic neurons, and GABAergic neurons) electrical activity, and/or neurite outgrowth. In certain aspects, the neurodegenerative disease is Parkinson's disease, Alzheimer's disease or multiple sclerosis.

A further embodiment provides a method of treating a neurodegenerative disease by administering an effective amount of an extracellular vesicle-enriched composition of the present embodiments or aspects thereof to a subject. In some aspects, the neurodegenerative disease is Parkinson's disease, Alzheimer's disease or multiple sclerosis.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Representative particle size distribution histogram of a sample of microglia extracellular vesicles (MGL-EV) as determined by Nanosight (sample of MGL-EV.TFF.SEC.LY), utilizing nanoparticle tracking analysis (NTA). EV and MV were prepared by an ultracentrifugation method (UC), or by a tangential flow filtration (TFF) method with size exclusion chromatography (SEC) and lyophilization (LY).

FIG. 2: Representative particle size distribution histogram of a sample of microglia-EV as determined by Zetaview Analyzer (sample of MGL-EV.TFF.SEC.LY).

FIG. 3: Representative particle size distribution histogram of a sample of microglia-EV (“MGL-EV.UC”) and its mock-EV (MV) control isolated from virgin media (“MGL-MV.UC”) as determined by resistive pulse sensing (RPS) on the nCS1 instrument. The MGL-EV have a higher concentration of particles than the matched MV control, with MGL-EV.UC having a concentration of 7.27E+10 particles/mL and the MGL-MV.UC having a concentration of 3.05E+9 particles/mL.

FIGS. 4A-4D: (FIG. 4A) Six biological replicates of apparently healthy normal (AHN) MGL-EV.UC were analyzed by NTA using a Nanosight. The D50 (i.e., median particle size, in nm, of a sample, wherein 50% of measured particles were that size or lower) of the particle size distributions were plotted and analyzed. For 1 sample, the D50 was in the 100-110 nm range. For 3 samples, the D50 was in the 110-120 nm range. For 2 samples, the D50 was in the 120 to 130 nm range. The median D50 of the six biological replicates is 113.75 nm. Additional statistics describing the distribution of D50s for the six biological replicates are noted in the lower part of the figure. (FIG. 4B) Histogram showing the distribution of the D50 (median particle size, in nm, of a sample, wherein 50% of measured particles were that size or lower, as determined by the Nanosight) of each EV.UC sample in a set of iPSC-EV and iPSC-derived cell-type EV, including AHN MGL-EV.UC (light grey bars, N=77). Overlayed are the AHN MGL-EV.UC data alone (hashed bars), as depicted in FIG. 4A. For this analysis, mutant MGL-EV (MGL APO E4/E4-EV, MGL TREM2 HO-EV, MGL TREM2 HZ-EV, MGL-EV MGL TREM2 R47H-EV, and the EV from MGL genetically modified to express GFP) were not included in the library data or the MGL-EV data. The distribution of D50s in the MGL-EV samples are skewed to the right as compared to the overall library, showing that the D50 particle size of AHN MGL-EV.UC is larger than the expected D50 based on all the EV samples from iPSC and iPSC-derived cells analyzed. The median D50 of the library distribution is 102.4 nm. Five out of six of the AHN MGL-EV.UC samples are above this median, showing that the D50 of AHN MGL-EV.UC tends to be larger. The median D50 of the six biological AHN MGL-EV.UC (113.75 nm) is larger than the third quartile of the overall library distribution (112.9 nm). Additional statistics describing the distribution of D50s for the 77 samples in the library are noted in the lower part of the figure. (FIG. 4C) Six biological replicates of AHN MGL-EV.UC and seven additional MGL-EV samples from mutant and genetically modified cell lines (1 MGL APO E4/E4-EV, 2 MGL TREM2 HO-EV, 1 MGL TREM2 HZ-EV, 1 MGL TREM2 R47H-EV, and 2 replicates of EV from MGL genetically modified to express GFP) were analyzed by NTA using a Nanosight. The distribution of the D50 (median particle size, in nm, of a sample, wherein 50% of measured particles were that size or lower). A total of 13 samples were analyzed. For one sample the D50 was in the 80-90 nm range, for three samples the D50 was in the 100-110 nm range, for six samples the D50 was in the 110-120 nm range, for two samples the D50 was in the 120-130 nm range, and for one sample the D50 was in the 130-140 nm range. The median D50 of the N=13 samples, AHN, diseased, and corrected, is 112 nm. Additional statistics describing the distribution of D50s for the 13 MGL-EV.UC samples are noted in the lower part of the figure. (FIG. 4D) Histogram showing the distribution of the D50s (median particle size, in nm, of a sample, wherein 50% of measured particles were that size or lower, as determined by the Nanosight) of each EV.UC sample in a library of iPSC-EV and iPSC-derived cell-type EV (light grey bars, N=84). Included in these 84 samples were 6 AHN MGL-EV.UC, and mutant MGL-EV.UC samples (1 MGL APO E4/E4-EV, 2 MGL TREM2 HO-EV, 1 MGL TREM2 HZ-EV, 1 MGL TREM2 R47H-EV, and 2 replicates of EV from MGL genetically modified to express GFP). Overlayed are the 13 MGL-EV.UC samples (hashed bars), as depicted in FIG. 4C. The distribution of D50s in just the 13 MGL-EV samples are skewed to the right as compared to the overall library of N=84, showing that the D50 particle size of MGL-EV.UC is larger than the expected D50 based on all the EV samples from the N=84 library. The median D50 of the library distribution is 104.05 nm. The median D50 of the 13 MGL-EV.UC samples is 112 nm, which is nearly at the third quartile of the overall library distribution (112.9 nm). Additional statistics describing the distribution of D50s for the 84 samples in the library are noted in the lower part of the figure.

FIGS. 5A-5B: (FIG. 5A) Tetraspanin marker expression in a sample of MGL-EV.TFF.SEC.LY as determined by ELISA method 1. This ELISA confirms the presence of EV expressing canonical EV tetraspanin markers, CD9, CD63 or CD81. (FIG. 5B) Tetraspanin marker expression of an iPSC-derived neural cell type EV sample (which was isolated using the method described in Example 1 “TFF and SEC”) (“Neuron-EV.TFF.SEC.LY”) as determined by ELISA method 1. This ELISA confirms the presence of EV expressing canonical EV tetraspanin markers, CD9, CD63 or CD81. Note that the relative expression levels of CD9, CD63 and CD81 for this Neuron-EV.TFF.SEC.LY are different from the relative expression levels of CD9, CD63 and CD81 in a sample of MGL-EV.TFF.SEC.LY depicted in FIG. 5A.

FIG. 6: Tetraspanin marker expression in samples of MGL-EV isolated in three different ways (by ultracentrifugation, “.UC”; by PS-affinity, “.UFW”; or by the TFF and SEC method, “.TFF.SEC.LY”), as determined by ELISA method 2. Depending on the isolation method, varying levels of CD9, CD63 and CD81 were detected per input when looking at absolute absorbance/input values.

FIGS. 7A-7B: (FIG. 7A) Depicted are the tetraspanin signatures for apparently-healthy-normal (AHN) MGL-EV across different isolation methods. Relative expression levels of CD9, CD63 and CD81 are depicted as a percentage of the total absorbance measured for the three markers analyzed. In the figure, AHN is referred to as wild-type (“WT”). For all three isolation methods, the tetraspanin signature determined by ELISA method 2 has a relative expression of CD9≥CD81>>CD63. The values depicted are the averages for each isolation type. The number of samples analyzed per isolation type (“N”) is given below the graph. (FIG. 7B) Alternative representation of the data in FIG. 7A. Depicted are the tetraspanin signatures for apparently-healthy-normal (AHN) MGL-EV across different isolation methods. Relative expression levels of CD9, CD63 and CD81 are depicted as a percentage of the total absorbance measured for the three markers analyzed. For all three isolation methods, the tetraspanin signature determined by ELISA method 2 has a relative expression of CD9≥CD81>>CD63. The values are given as averages, +/− the standard deviation.

FIGS. 8A-8B: (FIG. 8A) Depicted are the tetraspanin signatures determined using ELISA method 2 for apparently-healthy-normal (AHN) MGL-EV and various MGL-mutant cells. In FIG. 8A, AHN is referred to simply as “Microglia”. Mutant cell EV included in this analysis are “Microglia APO E4/E4”, “Microglia TREM2 HO”, “Microglia TREM2 HZ”, and “Microglia TREM2 R47H”. Relative expression levels of CD9, CD63 and CD81 are depicted as a percentage of the total absorbance measured for the three markers analyzed. Values are averages. The number of samples analyzed per EV type (“N”) is given below the graph. For all EV types analyzed here, CD63<5% of the total absorbance, ranging from 1.4% to 4.3%. For all EV types analyzed here, CD9 is at least 40% of the total absorbance, ranging from 41.6% to 50.9%. For all EV types analyzed here, CD81 is at least 45% of the total absorbance, ranging from 45.5% to 55.8%. (FIG. 8B) Alternative representation of the data in FIG. 8A. Depicted are the tetraspanin signatures determined using ELISA method 2 for apparently-healthy-normal (AHN) MGL-EV and various MGL-mutant cells. In FIG. 8B, AHN is referred to simply as “Microglia”. Mutant cell EV included in this analysis are “Microglia APO E4/E4”, “Microglia TREM2 HO”, “Microglia TREM2 HZ”, and “Microglia TREM2 R47H”. Relative expression levels of CD9, CD63 and CD81 are depicted as a percentage of the total absorbance measured for the three markers analyzed. Values are averages with standard deviations shown as error bars. For all EV types analyzed here, CD63<5% of the total absorbance, ranging from 1.4% to 4.3%. For all EV types analyzed here, CD9 is at least 40% of the total absorbance, ranging from 41.6% to 50.9%. For all EV types analyzed here, CD81 is at least 45% of the total absorbance, ranging from 45.5% to 55.8%.

FIG. 9: The tetraspanin signature for each MGL-EV sample (including AHN and mutant cell EV types, and including .UC; .TFF.SEC.LY; .UFW preparations) was determined by ELISA method 2. The MGL-EV tetraspanin signatures were compared to the EV tetraspanin signatures determined for a set of 63 additional EV samples, from non-MGL cells types, all of which are differentiated iPSC-derived cell-types. These 63 EV samples were generated from 30 distinct cell types. They were isolated using the UC method. The 30 cell types have been categorized into 14 sub-sets (as depicted by 14 different symbols on the figure). Tetraspanin signatures for the other iPSC-derived cell-type EV were also determined using the ELISA method 2. The percentage of CD9, CD63 and CD81 in the tetraspanin signatures for each sample was graphed on a ternary plot. MGL-EV signatures are represented as solid circles in the figure. Regardless of genotype and isolation method, the 12 MGL-EV samples in the analysis distinctly cluster together and are separate from all other samples in the figure. All MGL-EV signatures analyzed fall within a space on the graph with the following parameters: CD81: between 30-60%; CD63: <10%; and CD9: between 35-60%. No other iPSC-derived differentiated cell-type-EV samples analyzed occupy this space on the graph.

FIG. 10: Representative images of an individual EV, or “cluster”, identified by ONi super resolution microscopy in a sample of MGL-EV.TFF.SEC.LY. The cluster depicted is triple positive for CD9, CD81, and CD63. Results of the three individual tetraspanins are shown individually and as an overlay in the figure to illustrate the triple-positive nature of this cluster.

FIG. 11: PCA plot analyzing the differences in miR content between AHN MGL-EV (n=3), iPSC-EV (n=7), and other iPSC-derived cell-type EV (n=26). MGL-EV (represented as solid circles in the figure) had a miR content distinct from other EV types analyzed.

FIGS. 12A-12B: (FIG. 12A) Wordcloud of categories associated with the miR content identified in MGL-EV. miR content was determined by sequencing (top 100 miR by p-value were considered). Terms show that the miR content identified in MGL-EV are associated with extracellular vesicles. Six of the 10 terms are synonyms or subsets of extracellular vesicles. (FIG. 12B) An over-representation analysis was run using the predicted target genes of the 15 most abundant miRNA in MGL-EV. The analysis identified pathways which are predicted to be affected by the MGL-EV miRNA. While this analysis can predict pathways that will be affected, it cannot predict if these pathways would be upregulated or downregulated. The top 15 pathways predicted to be affected by MGL-EV miR are depicted in FIG. 12B, including the False Discovery Rate (FDR). The pathways with the lowest FDR (smallest bars) are the pathways for which there is the most confidence of enrichment. The top three pathways are therefore cell cycle arrest, intrinsic apoptotic signaling pathway, and response to oxygen levels. The full list of predicted pathways are presented in Table 15.

FIG. 13: Scatterplot of P10 MGL-EV miR versus the P90 of the miR in other EV in an “EV-library” identifies two unique miR which are expressed in AHN MGL-EV (RNA samples 50evrna, 58evrna 59evrna, 60evrna and 95evrna; extracted from EV495.TFF.SEC.LY, EV439.UC, EV561.UC, EV581.UC, and EV648.UC, respectively) and not in other EV types analyzed. hsa-miR-4669 and hsa-miR-4777-3p are unique miR in MGL-EV.

FIGS. 14A-14G: Cryo-electron microscopy images. (FIGS. 14A-14G) Representative images of MGL-EV composed of lipid bilayer structures. These include single, double, and multi-vesicular bodies of various sizes and shapes, including concentric and non-concentric structures. Images were collected from a sample of MGL-EV.TFF.SEC.LY. Some images depict scale bars and others include magnification information under the image.

FIG. 15: A sample of MGL-EV.TFF.SEC.LY was analyzed using the Protein Simple Wes, probing for the presence of B-Actin. A peak was identified at the expected size indicating the presence of B-actin in the EV sample. For each identified peak, the estimated molecular weight of the protein, the peak height, the peak area, the peak width, and the signal to noise ratio (“S/N”) at the peak are given in Table 5.

FIG. 16: A sample of MGL-EV.UC was analyzed using the Protein Simple Wes, probing for the presence of B-Actin. A peak was identified at the expected size indicating the presence of B-actin in the EV sample. For each identified peak, the estimated molecular weight of the protein, the peak height, the peak area, the peak width, and the signal to noise ratio (“S/N”) at the peak are given in Table 5.

FIG. 17: A sample of MGL-EV.TFF.SEC.LY was analyzed using the Protein Simple Wes, probing for the presence of CD-9. A peak was identified at the expected size indicating the presence of CD-9 in the EV sample. For each identified peak, the estimated molecular weight of the protein, the peak height, the peak area, the peak width, and the signal to noise ratio (“S/N”) at the peak are given in Table 5.

FIG. 18: A sample of MGL-EV.TFF.SEC.LY was analyzed using the Protein Simple Wes, probing for the presence of Flotillin-1. A peak was identified at the expected size indicating the presence of Flotillin-1 in the EV sample. For each identified peak, the estimated molecular weight of the protein, the peak height, the peak area, the peak width, and the signal to noise ratio (“S/N”) at the peak are given in Table 5.

FIG. 19: A sample of MGL-EV.TFF.SEC.LY was analyzed using the Protein Simple Wes, probing for the presence of TREM2, utilizing an antibody recognizing the N-terminus of the protein. As a control, a lysate of microglia cells (“MGL-Cells”) was included in the experiment. These cells are AHN (also referred to as WT). A peak was identified at the expected size indicating the presence of full-length TREM2 in the MGL-Cells and in the MGL-EV.TFF.SEC.LY sample. For each identified peak, the estimated molecular weight of the protein, the peak height, the peak area, the peak width, and the signal to noise ratio (“S/N”) at the peak are given in Table 5. The result for the MGL-EV.TFF.SEC.LY is depicted in the figure as a grey trace. The result for the MGL-Cells is depicted as a black trace in the figure.

FIG. 20: A sample of MGL-EV.TFF.SEC.LY was analyzed using the Protein Simple Wes, probing for the presence of TREM2, utilizing an antibody recognizing the C-terminus of the protein. As a control, a lysate of microglia cells (“MGL-Cells”) was included in the experiment. These cells are AHN (also referred to as WT). A short peak was identified at the expected apparent weight of full-length TREM2 (˜48 kDa), indicating the presence of full-length TREM2 in the MGL-Cells and in the MGL-EV.TFF.SEC.LY sample. A peak at ˜8 kDa was also detected in both MGL-Cells and MGL-EV.TFF.SEC.LY. TREM2 protein undergoes multiple cleavage events resulting in numerous smaller peptides. The small product detected here matches the predicted size of one of the C-terminal cleavage products of TREM2. The 8 kDa peptide was clearly enriched in the MGL-EV.TFF.SEC.LY as compared to the MGL-Cells. In the MGL-EV.TFF.SEC.LY, the area under the 8 kDa peak was 15.6 times greater than the area for the full-length protein in that sample. In the MGL-Cells, the area under the 8 kDa peak was less than the area of the full-length protein peak in that sample. For each identified peak, the estimated molecular weight of the protein, the peak height, the peak area, the peak width, and the signal to noise ratio (“S/N”) at the peak are given in Table 5. The result for the MGL-EV.TFF.SEC.LY is depicted in the figure as a grey trace. The result for the MGL-Cells is depicted as a black trace in the figure.

FIGS. 21A-21B: (FIG. 21A) Samples of MGL-EV.TFF.SEC.LY, MGL-EV.UC, and MGL-MV.UC control were analyzed using the Protein Simple Wes, probing for the presence of GBA. As a control, a lysate of microglia cells (“MGL-Cells”) was included in the experiment. These cells are AHN (also referred to as WT). A major peak in the 84-88 kDa size range was identified in the MGL-EV.TFF.SEC.LY, MGL-EV.UC, and MGL-cells. This peak is not present in the MGL-MV control. This is larger than the expected 77 kDa size which is typically seen for GBA by automated western blot, according to the manufacturer of the antibody. This is also larger than the GBA detected in another EV type, produced by an iPSC-derived neuron cell type as depicted in FIG. 22A and FIG. 22B, which was analyzed in parallel. For each identified peak, the estimated molecular weight of the protein, the peak height, the peak area, the peak width, and the signal to noise ratio (“S/N”) at the peak are given in Table 5. The result for the MGL-EV.TFF.SEC.LY is depicted as a dark grey trace on the figure. The result for the MGL-EV.UC is depicted as a black trace in the figure. The result for the MGL-MV.UC control sample is depicted as the lightest shade of grey in the figure; no label for this trace is present on this figure however a label is present in FIG. 21B. The result for the MGL-cells is depicted as a medium shade of grey in the figure. (FIG. 21B) Close up of the data depicted in FIG. 21A for MGL-EV.TFF.SEC.LY, MGL-EV.UC, and MGL-MV.UC control samples that were analyzed using the Protein Simple Wes, probing for the presence of GBA. For each identified peak, the estimated molecular weight of the protein, the peak height, the peak area, the peak width, and the signal to noise ratio (“S/N”) at the peak are given in Table 5. The results for the MGL-EV.TFF.SEC.LY are shown in a dark grey trace on this figure. The results for the MGL-EV.UC are shown in a black trace on this figure. The result for the MGL-MV.UC control samples are depicted in the figure as a light shade of grey.

FIGS. 22A-22B: (FIG. 22A) An automated Western Blot, on the Protein Simple Wes, was performed on EV produced by an iPSC-derived neuron cell-type and isolated by the TFF and SEC method (Neuron-EV.TFF.SEC.LY) or the ultracentrifugation method (Neuron-EV.UC), probing for the presence of GBA. As a control, a lysate of the iPSC-derived neuron cells was included in the experiment (Neuron-Cells). A major peak in the 64-69 kDa size range was identified in the Neuron-EV.TFF.SEC.LY and Neuron-EV.UC, and Neuron-Cell samples. This is similar to the 77 kDa expected size for GBA on the Wes, as predicted by the manufacturer. This is markedly smaller than the size of the GBA peak for the MGL-cells, MGL-EV.TFF.SEC.LY and MGL-EV.UC samples illustrated in FIG. 21A, and FIG. 21B. For each identified peak, the estimated molecular weight of the protein, the peak height, the peak area, the peak width, and the signal to noise ratio (“S/N”) at the peak are given in Table 5. The result for the Neuron-EV.TFF.SEC.LY is depicted as a dark grey trace in the figure. The result for the Neuron-EV.UC is depicted as a black trace in the figure. The result for the Neuron-Cells is depicted as a light grey trace in the figure. (FIG. 22B) Close up of the data depicted in FIG. 22A for Neuron-EV.TFF.SEC.LY, and Neuron-EV.UC that were analyzed using the Protein Simple Wes, probing for the presence of GBA. For each identified peak, the estimated molecular weight of the protein, the peak height, the peak area, the peak width, and the signal to noise ratio (“S/N”) at the peak are given in Table 5. The results for the Neuron-EV.TFF.SEC.LY are shown in a dark grey trace on this figure. The results for the Neuron-EV.UC are shown in a black trace on this figure.

FIG. 23: An HTRF (Homogeneous Time Resolved Fluorescence) assay was performed to detect the presence of LRRK2 protein in microglia in cell lysates and in vesicles. Results are shown as a ratio of fluorescence intensity. A higher ratio indicates a greater amount of target. The higher ratio indicates the presence of LRRK2 in MGL-EV.TFF.SEC.LY and MGL-EV.UC compared to negative controls (Lysis Buffer and MGL-MV.UC). The fluorescence ratio indicates the presence of LRRK2 in the microglia cell lysate (“MGL RIPA Buffer Lysate”).

FIG. 24: An HTRF (Homogeneous Time Resolved Fluorescence) assay was performed to detect the presence of Phospho-LRRK2 protein in microglia vesicles. Results are shown as a ratio of fluorescence intensity. The higher fluorescence ratio indicates the presence of Phospho-LRRK2 in MGL-EV.TFF.SEC.LY and MGL-EV.UC compared to negative controls (Lysis Buffer and MGL-MV.UC).

FIGS. 25A-25B: (FIG. 25A) AHN MGL-EV.UC and MGL-MV.UC control were incubated with the MACSPlex EV Kit and analyzed on a MACSQuant Analyzer 10 flow cytometer. Results are shown as the Median Fluorescence Intensity of the APC channel for each bead. Results show the presence of various proteins on the surface of MGL-EV.UC (lower panel of the figure). As expected, no significant signal was detected in the MGL-MV.UC control for any of the markers (upper panel of the figure). Results are sorted from highest to lowest MFI for the MGL-EV.UC. A line is added to the figure at 15 APC MFI. Sixteen surface markers were detected at levels above 15. The highest APC MFI were for the CD81, CD9, HLA-DRDPDQ and CD49e markers. Note that bead-bound EV (bound to the bead by binding of the antibody to the EV-surface ligand) are detected with a cocktail of tetraspanins (CD81, CD9 and CD63). Therefore, both the number of marker-positive EV bound to the bead and the number of tetraspanins on the bead-bound EV will influence the overall signal from each bead detected in this assay. (FIG. 25B) mRNA levels of the genes that code for the proteins assayed in the MACSplex kit were assessed by bulk RNA sequencing of AHN microglia cells. Results are shown as log 2 FPKM (Fragments Per Kilobase of transcript per Million mapped reads) with the median expression level of biological replicates listed below the plot. Genes are ordered to match the sequence of markers in FIG. 25A. The corresponding marker name, if different from the gene name, is indicated after the gene name in parentheses. Note that HLA-ABC is related to three separate genes, and HLA-DRDPDQ is related to two separate genes and SSEA-4 does not have a corresponding gene. The horizontal line at 2 log 2 FPKM separates high RNA expression genes from low RNA expression genes. Genes to the left of the vertical line correspond to surface markers with APC MFI>15 in FIG. 25A. Genes to the right of the vertical line correspond to surface markers with APC MFI<15 in FIG. 25A. For all of the markers with an APC MFI above 15 in FIG. 25A (high marker detection), median RNA expression levels for AHN MGL-Cells depicted in FIG. 25B were at or above 2.8 log 2 FPKM (high RNA expression). For all of the markers with an APC MFI less than 15 in FIG. 25A (low or no marker detection), median RNA expression levels for AHN MGL-cells depicted in FIG. 25B were at or below 0.9305 (low or no RNA expression), with three exceptions. For these exceptions, the APC MFI for the EV were in the low or no marker detection category, whereas the gene expression results from the cells were in the high RNA expression category, suggesting that the proteins may be selectively excluded from the EV. These exceptions are the HLA-ABC, CD86, and CD142.

FIGS. 26A-26C: (FIG. 26A) AHN MGL-EV.TFF.SEC.LY in vitro function as measured by HUVEC scratch wound healing assay. A MGL-MV.UC control sample was included. All three doses of the MGL-EV.TFF.SEC.LY improved scratch wound healing beyond the Poor media control. All three doses of the MGL-MV.UC resulted in less wound healing than the Poor media control alone. The 24-hour time point results for all of the samples depicted in FIG. 26A are tabulated in Table 6. (FIG. 26B) AHN MGL-EV.UC in vitro function as measured by HUVEC plating assay. A MGL-MV.UC control sample was included. Both doses of the MGL-EV.UC improved HUVEC plating and confluence (as measured by the Incucyte) ≥25% beyond the Poor media control. Both doses of the MGL-MV.UC resulted in <15% improvement in plating and confluence over Poor media control. (FIG. 26C) AHN MGL-EV.UC in vitro function as measured by HUVEC plating assay. A MGL-MV.UC control sample was included. Both doses of the MGL-EV.UC improved HUVEC cell viability (as measured by the CellTiter-Glo assay) ≥8% beyond the Poor media control. Both doses of the MGL-MV.UC resulted in ≤5% improvement in cell viability over Poor media control alone.

FIGS. 27A-27C (FIG. 27A) Assessment of the effects of MGL-EV.UC on neurite length for iPSC-derived dopaminergic neurons with typical viability. MGL-EV.UC decrease neurite length in a dose-dependent manner (an increase in EV treatment further reduces neurite length). MGL-MV.UC control had little to no effect on neurite length. (FIG. 27B) Assessment of the effects of MGL-EV.UC on the number of cell body clusters for iPSC-derived dopaminergic neurons with typical viability. MGL-EV.UC decrease the number of cell body clusters in a dose-dependent manner (an increase in EV treatment further reduces the number of cell body clusters). MGL-MV.UC control had little to no effect on the number of cell body clusters. (FIG. 27C) Assessment of the effects of MGL-EV on cell body cluster area for iPSC-derived dopaminergic neurons with typical viability. MGL-EV.UC increase the cell body cluster area in a dose-dependent manner (an increase in EV treatment further increases cell body cluster area). MGL-MV.UC control had little to no effect on cell body cluster area.

FIGS. 28A-28C: (FIG. 28A) Assessment of the effects of MGL-EV.UC on neurite length for iPSC-derived dopaminergic neurons with low viability. MGL-EV.UC decrease neurite length in a dose-dependent manner (an increase in EV treatment further reduces neurite length). MGL-MV.UC control had little to no effect on neurite length. (FIG. 28B) Assessment of the effects of MGL-EV.UC on the number of cell body clusters for iPSC-derived dopaminergic neurons with low viability. MGL-EV.UC decrease the number of cell body clusters in a dose-dependent manner (an increase in EV treatment further reduces the number of cell body clusters). MGL-MV.UC control had little to no effect on the number of cell body clusters. (FIG. 28C) Assessment of the effects of MGL-EV on cell body cluster area for iPSC-derived dopaminergic neurons with low viability. MGL-EV.UC increase the cell body cluster area in a dose-dependent manner (an increase in EV treatment further increases cell body cluster area). MGL-MV.UC control had little to no effect on cell body cluster area.

FIG. 29: Representative traces of neurosphere electrical activity with and without AHN MGL-EV.TFF.SEC.LY treatment. The trace with the higher amplitude and thinner line is for the untreated neurosphere. The trace with the lower amplitude and thicker line is for one of the MGL-EV treatment conditions. When incubated with MGL-EV.TFF.SEC.LY, the electrical traces are altered in several ways. 3D neural spheroids composed of glutamatergic and GABAergic neurons plus astrocytes were cultured in ultra-low attachment (ULA) plates for 21 days. Cells were exposed to different amounts of MGL-EV.TFF.SEC.LY for 24 hours and then assayed for spontaneous neuronal activity. The higher amplitude trace represents baseline calcium oscillations from untreated control wells while the lower amplitude trace depicts signal from MGL-EV treated wells. Exposure to MGL-EV resulted in a decrease in Peak Amplitude and an altered rising slope (from the bottom to the top of the peak), indicating an effect on the calcium handling activity in the neural spheroids. Ca²⁺ is a universal secondary messenger and is of critical importance to neurons, as it participates in the transmission of the depolarizing signal and contributes to synaptic activity.

FIGS. 30A-30G: Effect of MGL-EV.TFF.SEC.LY treatment on indicated parameters. Calcium data from the assay performed on the FDSS/uCell (Hamamatsu) was analyzed with Wavechecker software. Six different treatments of MGL-EV are plotted in dose-response format. Graphs addressing various metrics are presented, including (FIG. 30A) “Peak Counts” (the number of calcium peaks) which did not change; (FIG. 30B) “P-P time_std” (the standard deviation for peak-to-peak timing) which increased, meaning the irregularity of the oscillations increased; (FIG. 30C) “RMP” (the peak bottom intensity, which is a measure of intracellular [Ca²⁺,] concentration) which increased; (FIG. 30D)“(AMP+RMP)/RMP” (the ratio of peak amplitude to peak bottom) which decreased. Many of these metrics are mathematically related to each other. (FIG. 30E) “Peak Amplitude” (the difference in signal from top of the peak to the bottom) decreases with increased number of EV used. (FIG. 30F) “Area Under Curve” (the total peak area) also decreases with increased number of EV used. (FIG. 30G) Finally, the “Rising Slope” (also known as the upstroke slope with respect to depolarization) is decreased/less sharp with increased number of EV used.

FIG. 31: Assessment of the impact of AHN MGL-EV.UC on the electrophysiology of AHN and iPSC-dopaminergic neurons with Parkinson's Disease mutations as assessed by MEA.

FIG. 32: Effect of AHN MGL-EV.UC on the electrophysiology of AHN and iPSC-dopaminergic neurons with Parkinson's Disease mutations when normalized to baseline (pre-EV) values for each data set, as measured by MEA.

FIG. 33: MGL-EV.UC increase the number of spikes per burst relative to baseline, in LRRK2 mutants, compared to untreated LRKK2 controls. Multi-electrode array (MEA) was used to assess the effect on neuronal activity from human iPSC-derived dopaminergic neurons exposed to MGL-EV.UC. The MEA assay detects electrophysiological activity from cultured neurons. A panel of iPSC-derived dopaminergic neurons with Parkinson's Disease-relevant mutations (LRRK2-G2019S, SNCA-A53T, GBA-N370S) and the WT control (also referred to as AHN) were tested. After 4 days of culture with MGL-EV, the number of spikes per network burst increased 200% relative to baseline in the cells harboring the LRRK2 mutation. No change was observed for the control cells or other two mutations.

### DESCRIPTION OF ILLUSTRATIVE EMBODIMENTS

Microglia are innate immune cells of the central nervous system that perform critical roles in brain development, homeostasis, and immune regulation. They are hard to acquire from human fetal and primary tissues. Thus, certain embodiments of the present disclosure provide methods for the isolating of extracellular vesicles from human iPSC-derived microglia (iMGL). Cryopreserved iMGL retain purity, secrete immunomodulatory cytokines and phagocytose pHrodo Red labelled bacterial BioParticles and Amyloid βeta aggregates. The ability to produce essentially limitless quantities of EVs from iMGLs holds great promise for accelerating human neuroscience search into the role of microglia in normal and diseased states.

In further embodiments, the extracellular vesicles produced by the present methods may be used for disease modeling, drug discovery, and regenerative medicine.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains ≤30%, ≤20%, ≤15%, more preferably ≤10%, even more preferably ≤5%, or most preferably ≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Feeder-free” or “feeder-independent” is used herein to refer to a culture supplemented with cytokines and growth factors (e.g., TGFβ, bFGF, LIF, analogs or mimetics thereof) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct ¾), Sox2, c-Myc, Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

As used herein, the term “progenitor cell” refers to a descendant of a stem cell which is capable of further differentiation into one or more kinds of specialized cells, but which cannot divide and reproduce indefinitely. That is, unlike stem cells (which possess an unlimited capacity for self-renewal), progenitor cells possess only a limited capacity for self-renewal. Progenitor cells may be multipotent, oligopotent, or unipotent, and are typically classified according to the types of specialized cells they can differentiate into.

As used herein, “expand” or “proliferate” may refer to a process by which the number of cells in a cell culture is increased due to cell division.

“Multipotent” implies that a cell is capable, through its progeny, of giving rise to several different cell types found in an adult animal.

“Pluripotent” implies that a cell is capable, through its progeny, of giving rise to all the cell types that comprise the adult animal, including the germ cells. Embryonic stem cells, induced pluripotent stem cells, and embryonic germ cells are pluripotent cells under this definition.

The term “autologous cells” as used herein refers to donor cells that are genetically identical with the recipient.

As used herein, the term “allogeneic cells” refers to cells derived from a different, genetically non-identical, individual of the same species.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps.

As used herein, the term “extracellular vesicles or EVs” collectively refers to biological nanoparticles derived from cells, and examples thereof include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and apoptotic bodies, but are not limited thereto.

Extracellular vesicles can be categorized, for example, according to size. For instance, as used herein, the term “small extracellular vesicle” refers to extracellular vesicles having a diameter of between about 50-200 nm. In contrast, extracellular vesicles having a diameter of more than about 200 nm, but less than 400 nm, may be referred to as “medium extracellular vesicles,” and extracellular vesicles having a diameter of more than about 400 nm may be referred to as “large extracellular vesicles.” As used herein, the term “small extracellular vesicle fraction” (“sEV”) refers to a part, extract, or fraction, of secretome or conditioned medium, that is concentrated and/or enriched for small extracellular vesicles having a diameter of between about 50-200 nm. Such concentration and/or enrichment may be obtained using one or more of the purification, isolation, concentration, and/or enrichment, techniques disclosed herein. In some alternative embodiments herein, enrichment may not be performed, may not be achieved, or may not be possible.

The term “exosome” as used herein refers to an extracellular vesicle that is released from a cell upon fusion of the multivesicular body (MVB) (an intermediate endocytic compartment) with the plasma membrane.

“Exosome-like vesicles,” which have a common origin with exosomes, are typically described as having size and sedimentation properties that distinguish them from exosomes and, particularly, as lacking lipid raft microdomains. “Ectosomes,” as used herein, are typically neutrophil- or monocyte-derived microvesicles.

“Microparticles” as used herein are typically about 100-1000 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, z.e., diseased, unwanted and/or aberrant cells.

Within the class of extracellular vesicles, important components are “exosomes” themselves, which may be between about 20 to 50 nm and about 200 nm in diameter and being membranous vesicles, z.e., vesicles surrounded by a phospholipid bilayer, of endocytic origin, which result from exocytic fusion, or “exocytosis” of multivesicular bodies (MVBs). In some cases, exosomes can be between about 20 to 50 nm up to about 200 nm in diameter, such as being from 60 nm to 180 nm.

As used herein, the terms “secretome” and “secretome composition” interchangeably refer to one or more molecules and/or biological factors that are secreted by cells into the extracellular space (such as into a culture medium). A secretome or secretome composition may include, without limitation, extracellular vesicles (e.g., exosomes, microparticles, etc.), proteins, nucleic acids, cytokines, and/or other molecules secreted by cells into the extracellular space (such as into a culture medium). A secretome or secretome composition may be left unpurified or further processed (for example, components of a secretome or secretome composition may be present within culture medium, such as in a conditioned medium; or alternatively, components of a secretome or secretome composition may be purified, isolated, and/or enriched, from a culture medium or extract, part, or fraction thereof). A secretome or secretome composition may further comprise one or more substances that are not secreted from a cell (e.g., culture media, additives, nutrients, etc.). Alternatively, a secretome or secretome composition does not comprise one or more substances (or comprises only trace amounts thereof) that are not secreted from a cell (e.g., culture media, additives, nutrients, etc.).

An “extracellular vesicle (EV)-enriched secretome” as used herein refers to a secretome enriched for concentration, i.e. EV counts relative to volume, or to EV counts/markers relative to another component. The extent of separation or concentration can be assessed by characterization. The terms “EV-enriched secretome” and “EV(s)” are used interchangeably herein.

As used herein, the term “conditioned media” refers to a culture media (or extract, part, or fraction thereof) in which one or more cells of interest have been cultured. Preferably, conditioned medium is separated from the cultured cells before use and/or further processing. Removal of cells before freezing liquid can avoid exploding cells at −80° C. and releasing their contents. The culturing of cells in culture medium may result in the secretion and/or accumulation of one or more molecules and/or biological factors (which may include, without limitation, extracellular vesicles (e.g., exosomes, microparticles, etc.), proteins, nucleic acids, cytokines, and/or other molecules secreted by cells into the extracellular space); the medium containing the one or more molecules and/or biological factors is a conditioned medium. Examples of methods of preparing conditioned media have been described in, for example, U.S. Pat. No. 6,372,494, which is incorporated by reference herein in its entirety.

As used herein, “spent media” refers to a crude culture medium in which one or more cells of interest have been cultured which has not had the cultured cells fractioned out. Specifically, the spent media is a mixture of culture media components, cells and/or cell debris, cellular metabolites, and the secretome released by the cells. This spent media is clarified to remove the cells and large debris, resulting in the conditioned media. The conditioned media contains the metabolites, secretome and the remaining culture media components. The conditioned media is processed by methods such as ultracentrifugation, ultrafiltration, tangential flow filtration, size exclusion chromatography and/or affinity capture, etc. to generate the extracellular vesicle-enriched secretome.

As used herein, the term “cell culture” refers to cells grown under controlled condition(s) outside the natural environment of the cells. For instance, cells can be propagated completely outside of their natural environment (in vitro), or can be removed from their natural environment and the cultured (ex vivo). During cell culture, cells may survive in a non-replicative state, or may replicate and grow in number, depending on, for example, the specific culture media, the culture conditions, and the type of cells. An in vitro environment can be any medium known in the art that is suitable for maintaining cells in vitro, such as suitable liquid media or agar, for example.

As used herein, a “cell line” is an established cell culture derived from one cell or set of cells of the same type that will proliferate indefinitely under certain conditions. The cells of the cell line may comprise a uniform genetic makeup.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel that has been supplemented with medium suitable for further cell proliferation.

As used herein, the terms “culture medium,” “growth medium” or “medium” are used interchangeably and refer to a composition that is intended to support the growth and survival of organisms. While culture media is often in liquid form, other physical forms may be used, such as, for example, a solid, semi-solid, gel, suspension, and the like.

As used herein, the term “serum-free,” in the context of a culture medium or growth medium, refers to a culture or growth medium in which serum is absent. Serum typically refers to the liquid component of clotted blood, after the clotting factors (e.g., fibrinogen and prothrombin) have been removed by clot formation. Serum, such as fetal bovine serum, is routinely used in the art as a component of cell culture media, as the various proteins and growth factors therein are particularly useful for the survival, growth, and division of cells.

As used herein, the term “basal medium” refers to an unsupplemented synthetic medium that may contain buffers, one or more carbon sources, amino acids, and salts. Depending on the application, basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic (bFGF), also known as fibroblast growth factor 2 (FGF-2)).

As used herein, the terms “wild-type,” “naturally occurring,” “apparently healthy normal (AHN),” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

The term “extracellular matrix protein” refers to a molecule which provides structural and biochemical support to the surrounding cells. The extracellular matrix protein can be recombinant and also refers to fragments or peptides thereof. Examples include collagen and heparin sulfate.

A “three-dimensional (3-D) culture” refers to an artificially-created environment in which biological cells are permitted to grow or interact with their surroundings in all three dimensions. The 3-D culture can be grown in various cell culture containers such as bioreactors, small capsules in which cells can grow into spheroids, or non-adherent culture plates. In particular aspects, the 3-D culture is scaffold-free. In contrast, a “two-dimensional (2-D)” culture refers to a cell culture such as a monolayer on an adherent surface.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

II. iPSC Differentiation Methods

### A. HPCs

The iPSCs can be differentiated into HPCs by methods known in the art such as described in U.S. Pat. No. 8,372,642, which is incorporated by reference herein. In one method, combinations of BMP4, VEGF, Flt3 ligand, IL-3, and GM-CSF may be used to promote hematopoietic differentiation. In certain embodiments, the sequential exposure of cell cultures to a first media to prepare iPSCs for differentiation, a second media that includes BMP4, VEGF, and FGF, followed by culture in a third media that includes Flt3 ligand, SCF, TPO, IL-3, and IL-6 can differentiate pluripotent cells into HPCs and hematopoietic cells. The second defined media can also comprise heparin. Further, inclusion of FGF-2 (50 ng/ml) in the media containing BMP4 and VEGF can enhance the efficiency of the generation of hematopoietic precursor cells from pluripotent cells. In addition, inclusion of a Glycogen synthase kinase 3 (GSK3) inhibitor (e.g., CHIR99021, BIO, and SB-216763) in the first defined media can further enhance the production of HPCs.

Generally, differentiation of pluripotent cells into hematopoietic precursor cells may be performed using defined or undefined conditions. It will be appreciated that defined conditions are generally preferable in embodiments where the resulting cells are intended to be administered to a human subject. Hematopoietic stem cells may be derived from pluripotent stem cells under defined conditions (e.g., using a TeSR media), and hematopoietic cells may be generated from embryoid bodies derived from pluripotent cells. In other embodiments, pluripotent cells may be co-cultured on OP9 cells or mouse embryonic fibroblast cells and subsequently differentiated.

Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins. Three-dimensional EBs can thus be used to produce some fraction of hematopoietic cells and endothelial cells.

To promote aggregate formation, the cells may be transferred to low-attachment plates for an overnight incubation in serum-free differentiation (SFD) medium, consisting of 75% IMDM (Gibco), 25% Ham's Modified F12 (Cellgro) supplemented with 0.05% N2 and 1% B-27 without RA supplements, 200 mM 1-glutamine, 0.05 mg/ml Ascorbic Acid-2-phosphate Magnesium Salt (Asc 2-P) (WAKO), and 4.5×10⁻⁴ MTG. The next day the cells may be collected from each well and centrifuged. The cells may then be resuspended in “EB differentiation media,” which consists of SFD basal media supplemented with about 50 ng/ml bone morphogenetic factor (BMP4), about 50 ng/ml vascular endothelial growth factor (VEGF), and 50 ng/ml zb FGF for the first four days of differentiation. The cells are half fed every 48 hrs. On the fifth day of differentiation the media is replaced with a second media comprised of SFD media supplemented with 50 ng/ml stem cell factor (SCF), about 50 ng/ml Flt-3 ligand (Flt-3L), 50 ng/ml interleukin-6 (IL-6), 50 ng/ml interleukin-3 (IL-3), 50 ng/ml thrombopoieitin (TPO). The cells are half fed every 48 hrs with fresh differentiation media. The media changes are performed by spinning down the differentiation cultures at 300 g for 5 minutes and aspirating half the volume from the differentiating cultures and replenishing it with fresh media. In certain embodiments, the EB differentiation media may include about BMP4 (e.g., about 50 ng/ml), VEGF (e.g., about 50 ng/ml), and optionally FGF-2 (e.g., about 25-75 ng/ml or about 50 ng/ml). The supernatant may be aspirated and replaced with fresh differentiation medium. Alternately the cells may be half fed every two days with fresh media. The cells may be harvested at different time points during the differentiation process.

HPCs may be cultured from pluripotent stem cells using a defined medium. Methods for the differentiation of pluripotent cells into hematopoietic CD34⁺ stem cells using a defined media are described, e.g., in U.S. application Ser. No. 12/715,136 which is incorporated by reference in its entirety. It is anticipated that these methods may be used with the present disclosure.

For example, a defined medium may be used to induce hematopoietic CD34⁺ differentiation. The defined medium may contain the growth factors BMP4, VEGF, Flt3 ligand, IL-3 and/or GMCSF. Pluripotent cells may be cultured in a first defined media comprising BMP4, VEGF, and optionally FGF-2, followed by culture in a second media comprising either (Flt3 ligand, IL-3, and GMCSF) or (Flt3 ligand, IL-3, IL-6, and TPO). The first and second media may also comprise one or more of SCF, IL-6, G-CSF, EPO, FGF-2, and/or TPO. Substantially hypoxic conditions (e.g., less than 20% 02) may further promote hematopoietic or endothelial differentiation.

Cells may be substantially individualized via mechanical or enzymatic means (e.g., using a trypsin or TrypLE™). A ROCK inhibitor (e.g., H1152 or Y-27632) may also be included in the media. It is anticipated that these approaches may be automated using, e.g., robotic automation.

In certain embodiments, substantially hypoxic conditions may be used to promote differentiation of pluripotent cells into hematopoietic progenitor cells. As would be recognized by one of skill in the art, an atmospheric oxygen content of less than about 20.8% would be considered hypoxic. Human cells in culture can grow in atmospheric conditions having reduced oxygen content as compared to ambient air. This relative hypoxia may be achieved by decreasing the atmospheric oxygen exposed to the culture media. Embryonic cells typically develop in vivo under reduced oxygen conditions, generally between about 1% and about 6% atmospheric oxygen, with carbon dioxide at ambient levels. Without wishing to be bound by theory, it is anticipated that hypoxic conditions may mimic an aspect of certain embryonic developmental conditions. As shown in the below examples, hypoxic conditions can be used in certain embodiments to promote additional differentiation of induced pluripotent cells into a more differentiated cell type, such as HPCs.

The following hypoxic conditions may be used to promote differentiation of pluripotent cells into hematopoietic progenitor cells. In certain embodiments, an atmospheric oxygen content of less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, about 5%, about 4%, about 3%, about 2%, or about 1% may be used to promote differentiation into hematopoietic precursor cells. In certain embodiments, the hypoxic atmosphere comprises about 5% oxygen gas.

Regardless of the specific medium being used in any given hematopoietic progenitor cell expansion, the medium used is preferably supplemented with at least one cytokine at a concentration from about 0.1 ng/mL to about 500 ng mL, more usually 10 ng/mL to 100 ng/mL. Suitable cytokines include but are not limited to, c-kit ligand (KL) (also called steel factor (StI), mast cell growth factor (MGF), and stem cell factor (SCF)), IL-6, G-CSF, IL-3, GM-CSF, IL-la, IL-11 MIP-1α, LIF, c-mpl ligand/TPO, and flk2/flk3 ligand (Flt2L or Flt3L). Particularly, the culture will include at least one of SCF, Flt3L and TPO. More particularly, the culture will include SCF, Flt3L and TPO.

In one embodiment, the cytokines are contained in the media and replenished by media perfusion. Alternatively, when using a bioreactor system, the cytokines may be added separately, without media perfusion, as a concentrated solution through separate inlet ports. When cytokines are added without perfusion, they will typically be added as a 10× to 100× solution in an amount equal to one-tenth to 1/100 of the volume in the bioreactors with fresh cytokines being added approximately every 2 to 4 days. Further, fresh concentrated cytokines also can be added separately in addition, to cytokines in the perfused media.

### Exemplary HPC Differentiation Method

2D HPC differentiation: iPSCs may be maintained on MATRIGEL™ or Vitronectin in the presence of E8 and adapted to hypoxia for at least 5-10 passages. Cells are split from sub confluent iPSCs and plated at a density of 0.25 million cells/well onto Amine culture dishes in the presence Serum Free Defined (SFD) media supplemented with 5 uM blebbistatin. 24 hrs post plating SFD media supplemented with 50 ng/ml of BMP4, VEGF and FGF2 is added to the culture. The following day, fresh media is exchanged to remove blebbistatin. On the fifth day of the differentiation process, the cells are placed in media containing 50 ng/ml Flt-3 Ligand, SCF, TPO, IL3 and IL6 with 5U/ml of heparin. The cells are fed every 48 hrs throughout the differentiation process. The entire process is performed under hypoxic conditions and on charged amine plates. HPCs are quantified by the presence of CD43/CD34 cells and CFU.

3D HPC Differentiation: Cells were split from sub confluent iPSCs and plated at a density of 0.25-0.5 million cells per ml into a spinner flask in the presence of Serum Free Defined (SFD) media supplemented with 5 μM blebbistatin or 1 μM H1152. 24 hrs post plating SFD media supplemented with 50 ng/ml of BMP4, VEGF and FGF2 was exchanged. On the fifth day of the differentiation process the cells were placed in media containing 50 ng/ml Flt-3 Ligand, SCF, TPO, IL3 and IL6 with 5-10 U/ml of heparin. The cells were fed every 48 hrs throughout the differentiation process. The entire process was performed under hypoxic conditions. HPCs quantified by presence of CD43/CD34. HPCs are MACS sorted using CD34 beads.

### B. Gene Disruption

In certain aspects, TREM2, MeCP2, and/or SCNA gene expression, activity or function is disrupted in cells, such as PSCs (e.g., ESCs or iPSCs). In some embodiments, the gene disruption is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the disruption can be effected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the disruption of the expression, activity, and/or function of the gene is carried out by disrupting the gene. In some aspects, the gene is disrupted so that its expression is reduced by at least at or about 20, 30, or 40%, generally at least at or about 50, 60, 70, 80, 90, or 95% as compared to the expression in the absence of the gene disruption or in the absence of the components introduced to effect the disruption.

In some embodiments, the disruption is transient or reversible, such that expression of the gene is restored at a later time. In other embodiments, the disruption is not reversible or transient, e.g., is permanent.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene disruption is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct. In particular aspects, the siRNA suppresses both wild-type and mutant protein translation from endogenous mRNA.

In some embodiments, the disruption is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 2005/0064474; 2006/0188987 and 2007/0218528, incorporated by reference in their entireties herein, for details regarding fusions of DNA-binding domains and nuclease cleavage domains. In some aspects, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

### 1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include zinc-finger nucleases (ZFNs), transcription activator-like effectors (TALEs), and transcription activator-like effector nucleases (TALENs).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some aspects, disruption of MeCP2 is carried out by contacting a first target site in the gene with a first ZFP, thereby disrupting the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains.

In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

In some embodiments, ZFNs target a gene present in the engineered cell. In some aspects, the ZFNs efficiently generate a double strand break (DSB), for example at a predetermined site in the coding region of the gene. Typical regions targeted include exons, regions encoding N terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

### 2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, N1 to A, NN binds to G or A, and NO binds to T and non-canonical (atypical) RVDs are also known. See, U.S. Patent Publication No. 2011/0301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, 1998) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. A library of TALENs targeting 18,740 human protein-coding genes has been constructed. Custom-designed TALE arrays are commercially available through Cellectis Bioresearch (Paris, France), Transposagen Biopharmaceuticals (Lexington, KY, USA), and Life Technologies (Grand Island, NY, USA).

In some embodiments the TALENs are introduced as trans genes encoded by one or more plasmid vectors. In some aspects, the plasmid vector can contain a selection marker which provides for identification and/or selection of cells which received said vector.

3. RGENs (CRISPR/Cas systems)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

### C. Differentiation of HPCs to Microglia

Microglia are innate immune cells of the central nervous system that perform critical roles in brain development, homeostasis, and immune regulation. They are hard to acquire from human fetal and primary tissues. In certain embodiments, the present methods describe the generation, characterization and cryopreservation of human iPSC-derived microglia (iMGL) from episomally reprogrammed HPCs under defined conditions. Cryopreserved iMGL retain purity, secrete immunomodulatory cytokines and phagocytose pHrodo Red labelled bacterial BioParticles and Amyloid βeta aggregates. The ability to produce essentially limitless quantities of iMGLs holds great promise for accelerating human neuroscience research into the role of microglia in normal and diseased states.

In certain embodiments, the present EV-enriched secretome may be generated by iPSC-derived microglia from various differentiation methods, including those described in US20200239844 and WO2022/235911, both incorporated herein by reference in their entirety.

In an exemplary method, fresh or cryopreserved HPCs are thawed and plated in Microglia Differentiation Media comprising FLT-3 ligand and IL-3. The cells may be plated at a density of 10-50 K/cm², such as 20-35 K/cm². The Microglia Differentiation Medium may comprise microglia basal medium with IL-34 (e.g., 25 ng/mL), TGFβ1 (e.g., 50 ng/mL), and M-CSF (100 ng/mL) (i.e., MDM) or the respective analogs or mimetics thereof. The culturing may be performed on MATRIGEL™ coated plate or a charged surface such as a Primaria plate or Ultra low attachment plate or a tissue culture plate (TC) or a non-tissue culture plate (Non-TC) and may be high-throughput, such as a 96 well plate (e.g., 200 μl Microglia Differentiation Medium per well). The cells may be half fed every 48 hrs with 50 μl media per well of 2× Microglia Differentiation media (MDM) the next 23 days of differentiation. In specific aspects, the differentiation is performed in the absence of ECM proteins, such as MATRIGEL®. The cells are harvested with cold PBS on day 23 and the total viable cell number is quantified using an automated cell counter. The cells are stained for surface expression of CD11b, CD11c, CD45, CD33, TREM-2 and intracellular expression of TREM-2, IBA, CX3CR1, P2RY12, PU1, and TMEM119.

In one specific method, iPSCs maintained on MATRIGEL™ or Vitronectin in the presence of E8 were adapted to hypoxia for at least 5-10 passages. Cells were split from sub confluent iPSCs and plated at a density of 0.25-0.5 million cells per ml into a spinner flask in the presence of Serum Free Defined (SFD) media supplemented with 5 uM blebbistatin or 1 uM H1152. 24 hours post plating SFD media supplemented with 50 ng/ml of BMP4, VEGF and FGF2 was exchanged. On the fifth day of the differentiation process the cells were placed in media containing 50 ng/ml Flt-3 Ligand, SCF, TPO, IL3 and IL6 with 5U/ml of heparin. The cells were fed every 48 hours throughout the differentiation process. The entire process was performed under hypoxic conditions. HPCs were quantified by presence of CD43/CD34. HPCs were placed in microglia differentiation media MDM OR 2×-MDM.

In another method, embryoid bodies may be generated from iPSC using IL-3, MCSF and β-mercaptoethanol. Myeloid progenitors may be selected and further differentiated using IL-34, MCSF and TGF-β for 2 weeks, in addition to CX3CL1 and CD200 for the final 3 days. The generated microglia may be characterized in terms of their microglial signature gene expression, and display typical microglial functions, such as phagocytosis of particles, intracellular signaling and responses to inflammatory stimuli.

Microglia can be stimulated to a pro-inflammatory state by treating them with lipopolysaccharides (LPS), heat-shocked phosphatidyl-serine-positive neurons, IFN-gamma, or TNF-alpha. Microglia can be stimulated to an anti-inflammatory state by treating with resolving cytokines, such as IL-4, or IL-10. In particular aspects, the present microglia have not been stimulated to a pro-inflammatory or anti-inflammatory phenotype. In particular aspects, the microglia been cultured in a differentiation media, including the differentiation factors TGF-beta, M-CSF, IL-34, and sodium bicarbonate on a Matrigel extracellular matrix.

### D. Differentiation Media

Cells can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully-defined and feeder-free media.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

### E. Cryopreservation

The cells produced by the methods disclosed herein can be cryopreserved, see for example, PCT Publication No. 2012/149484 A2, which is incorporated by reference herein, at any stage of the process, such as Stage I, Stage II, or Stage III. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C. and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 6, 7, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8%. to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C./minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

III. Production of Extracellular Vesicles from iPSC-Derived Microglia

In certain embodiments, the present disclosure provides methods for the culturing of microglia for extracellular vesicle production, such as under GMP-ready and/or GMP-compatible conditions.

The one or more microglia can be, for example, microglia that have recently been isolated or differentiated from iPSCs. Alternatively, in some embodiments, microglia that have previously been refrigerated, frozen, and/or cryopreserved, may be used in the culturing methods of the present disclosure. In some embodiments, microglia are thawed from a cryopreserved state (e.g., −80° C. or colder) before use. In some embodiments thereof, the cells are thawed in a thawing medium.

The recovered, conditioned medium may in some embodiments be subjected to one or more further processing steps. The culture medium used during the vesiculation period may be removed, analyzed, recovered, concentrated, enriched, isolated, purified, refrigerated, frozen, cryopreserved, lyophilized, sterilized, etc.

In some embodiments, the recovered, conditioned medium may be pre-cleared or clarified to remove particulates of greater than a certain size. For instance, the recovered, conditioned medium may be pre-cleared or clarified by one or more centrifugation and/or filtration techniques.

In some embodiments, the recovered, conditioned medium is further processed to obtain a particular extract or fraction of the recovered, conditioned medium. For instance, the recovered, conditioned medium may be further processed to separate a small extracellular vesicle-enriched fraction (sEV) therefrom. An sEV fraction may be separated from the recovered, conditioned medium (or from a previously processed extract or fraction thereof) by one or more techniques such as centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

In some embodiments, conditioned medium is subjected to clarification by one or more filtration steps. In some embodiments thereof, one or more of the filtration steps utilizes a filter membrane having a particular pore size. In some embodiments thereof, a filter is used having a pore size of between 0.2 μm and 500 μm, or between 0.5 μm and 200 μm; or having a pore size less than or equal to 500 μm, 400 μm, 300 μm, 200 μm, 100 μm, 50 μm, 40 pm, 30 μm, 20 μm, 15 μm, 10 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, or 0.2 μm.

In some embodiments, the clarification comprises at least 1, at least 2, at least 3, at least 4, at least 5, at least 6, or at least 7, filtration steps. In some embodiments, the clarification comprises 4 filtration steps. In some embodiments, successive filtration steps utilize filters having increasingly smaller pores.

In some embodiments thereof, a first filtration step comprises use of an approximately 100-300 μm filter; a second filtration step comprises use of an approximately 5-25 μm filter; a third filtration step comprises use of an approximately 0.2-0.5 μm filter; and a fourth filtration step comprises use of an approximately 0.2-0.5 μm filter.

In some embodiments, conditioned medium may be subjected to clarification by one or more centrifugation steps. In some embodiments, conditioned medium may be subjected to clarification by a combination of centrifugation and filtration step(s).

In some embodiments, one or more additives are added to the conditioned medium, such as before clarification, and/or after clarification. In some embodiments, an additive is added that reduces aggregation. In some embodiments thereof, the additive is one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E).

In some embodiments, conditioned medium or EV may be subjected to isolation, enrichment, and/or concentration step(s) using tangential flow filtration (TFF). In some embodiments, the conditioned medium or EVs is subjected to TFF after clarification that employed one or more clarification steps (e.g., such as after one or more filtration and/or centrifugation steps). TFF is a rapid and efficient method for separating, enriching and purifying biomolecules. In some embodiments, TFF can be used, e.g., for concentrating (e.g., concentrating small extracellular vesicles from conditioned media); for diafiltration; and for concentrating and diafiltration. Diafiltration is a type of ultrafiltration process in which the retentate (the fraction that does not pass through the membrane) is diluted with buffer and re-ultrafiltered, to reduce the concentration of soluble permeate components and increase further the concentration of retained components.

In some embodiments, TFF is used for enriching, concentrating and diafiltration of conditioned medium or EVs (e.g., for concentration and diafiltration of the spent medium or the conditioned medium, resulting in an EV-enriched secretome EV secretome). In some embodiments, TFF is first used to concentrate conditioned medium or EVs, and is subsequently used for diafiltration. In some embodiments, a TFF process may comprise a further step of concentrating after diafiltration. In some embodiments, TFF is used for diafiltration but not concentrating. In some embodiments, TFF is used for concentrating but not diafiltration.

In some embodiments, the TFF membrane has a cut-off value of or less than 10 kDa, of or less than 20 kDa, of or less than 30 kDa, of or less than 40 kDa, of or less than 50 kDa, of or less than 60 kDa, of or less than 70 kDa, of or less than 80 kDa, of or less than 90 kDa, of or less than 100 kDa, or of or less than 150 kDa. In some embodiments, the TFF membrane has a cut-off value of about 10 kDa, about 30 kDa, about 100 kDa, or about 500 kDa. In some embodiments, the TFF membrane has a cut-off value of 30 kDa or about 30 kDa. In certain embodiments, the TFF membrane has a cut-off value of or less than 5 nm, of or less than 10 nm, 20 nm, 30 nm, 40 nm, 50 nm, 60 nm, 70 nm, 80 nm, 90 nm, 100 nm, or 150 nm.

In some embodiments, the TFF membrane comprises cellulose. In some embodiments, the TFF membrane comprises regenerated cellulose. In some embodiments, the TFF membrane comprises a polyethersulfone (PES) membrane. In some embodiments, the TFF membrane comprises a polysulfone hollow fiber membrane.

In some embodiments, conditioned media or EVs subjected to TFF can be further purified, isolated, and/or enriched (after TFF) using one or more purification, isolation, and/or enrichment, techniques. For instance, the resulting product from TFF can be subjected to a chromatography step, such as an ion exchange chromatography step, a steric exclusion chromatography step, or size exclusion chromatography step, to even further purify small extracellular vesicles. In some embodiments, conditioned media subjected to TFF, with or without further purification, isolation, and/or enrichment, may be further concentrated, such as by ultracentrifugation. In some aspects, the method may comprise further processing after TFF, such as size exclusion chromatography and a second round of TFF.

Any of the above-described processing techniques can be performed on recovered, conditioned medium (or a previously processed extract or fraction thereof) that is fresh, or has previously been frozen and/or refrigerated, for example.

In some embodiments, the EV compositions produced by the methods herein may have added thereto at least one additive to prevent aggregation. The additive may be one or more selected from trehalose, histidine (e.g., L-histidine), arginine (e.g., L-arginine), citrate-dextrose solution, a Dnase (e.g., Dnase I), ferric citrate, or Anti-Clumping Agent (Gibco/Life technologies, Ref: 01-0057; Lonza, Ref: BE02-058E).

In some embodiments, the EVs fraction is CD63⁺, CD81⁺, and/or CD9⁺. The EVs fraction may contain one or more extracellular vesicle types, such as, for example, one or more of exosomes, microparticles, and extracellular vesicles. The EVs fraction may also contain secreted proteins (enveloped and/or unenveloped). Extracellular vesicles within conditioned media or EVs fractions of the present disclosure may contain, for example, one or more components selected from tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSP70 and HSP90), exosome biogenesis proteins (ALIX, Tsg101), LT, prostaglandins, and SI 00 proteins.

In some embodiments, the presence of desired extracellular vesicle types in a fraction can be determined, for example, by nanoparticle tracking analysis (to determine the sizes of particles in the fraction); and/or by confirming the presence of one or more markers associated with a desired extracellular vesicle types. For instance, a fraction of recovered, conditioned media can be analyzed for the presence of desired extracellular vesicle types by detecting the presence of one or more markers in the fraction, such as, for example, CD9, CD63 and/or CD81.

In some embodiments, the present EV formulation or composition is positive for CD9, CD63 and CD81 (canonical EV markers). In certain aspects, the EV composition if positive for microglia-related markers.

In some embodiments, the EVs formulation or composition is at least one of the following: an EVs formulation or composition that has been enriched for extracellular vesicles having a diameter of between about 50-200 nm or between 50-200 nm; an EVs formulation or composition that has been enriched for extracellular vesicles having a diameter of between about 50-150 nm or between 50-150 nm; an EVs formulation or composition that is substantially free or free of whole cells; and an EVs formulation or composition that is substantially free of one or more culture medium components (e.g., phenol-red).

In some embodiments, such as, for example, some GMP-compatible processes, testing panels are conducted to analyze and/or determine one or more properties of the processes, products thereof, or intermediate products, etc.

For instance, during the vesiculation stage (including, e.g., thawing, plating, culturing and/or harvesting steps), one or more properties of the cells may be examined (including, for example: the number of viable cells, the percentage viability of the cells; morphologies of the cells; identity of the cells; karyotype of the cells; and/or transcriptome of the cells).

Additionally, or alternatively, one or more properties of a secretome and/or extracellular vesicle-containing fraction, extract, or composition can be analyzed using one or more tests (including, e.g., particle concentration and/or particle size distribution; protein concentration; protein profile concentration; RNA profile; potency; marker identity; host cell protein assessment; residual DNA quantification and/or characterization; sterility; mycoplasma; endotoxin; appearance; pH; osmolarity; extractable volume; hemolytic activity; complement activation; platelet activation; and/or genotoxicity), to determine one or more properties of the secretome/extracellular vesicles. For instance, one or more of these properties can be assessed on conditioned media before clarification; on conditioned media after clarification; on isolated and/or concentrated secretome/extracellular vesicles; and/or on final formulations. In some embodiments, final formulations may be tested immediately after production and/or 1-week, 2-weeks, 1-month, 2-months, 3-months, 6-months, 1-year or several years, after being formulated.

### IV. Methods of Use

In certain embodiments, the present disclosure provides EV compositions derived from iPSC-derived microglia. The EV compositions can be used for a number of important research, development, and commercial purposes. These include, but are not limited to, transplantation or implantation in vivo and screening cells in vitro to discover new properties.

The present disclosure contemplates EV compositions useful as therapeutic agents. In some embodiments, the methods of the present disclosure comprise administering an effective amount of an EV composition to a subject in need thereof.

The tissue to be treated may be damaged or fully or partly non-functional due to an injury, age-related degeneration, acute or chronic disease, neurodegenerative diseases, including Parkinson's Disease, Parkinson Like Syndrome, Alzheimer's Disease, Dementia, Stroke, Seizures, epilepsy, psychiatric conditions such as depression, anxiety, schizophrenia, Huntington, other neuromuscular diseases, rare conditions, other conditions especially involving neuron or glia pathologies, or brain damage including congenital or traumatic brain damage. Such tissues may be treated, for example, by intravenous administration of an EV composition.

In some embodiments, the administration comprises administration at a tissue or organ site that is the same as the target tissue. In some embodiments, the administration comprises administration at a tissue or organ site that is different from the target tissue. Such administration may include, for example, intravenous administration.

An EV composition may contain, or be administered with, a pharmaceutically-acceptable diluent, carrier, or excipient. Such a composition may also contain, in some embodiments, pharmaceutically acceptable concentrations of one or more of a salt, buffering agent, preservative, or other therapeutic agent. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations. For instance, in some embodiments, an EV composition, may be formulated with a biomaterial, such as an injectable biomaterial. Exemplary injectable biomaterials are described, for example, in WO 2018/046870, incorporated by reference herein in its entirety.

The EV compositions of the present disclosure may be administered in effective amounts, such as therapeutically effective amounts, depending on the purpose. An effective amount will depend upon a variety of factors, including the material selected for administration, whether the administration is in single or multiple doses, and individual patient parameters including age, physical condition, size, weight, and the stage of disease. These factors are well known to those of ordinary skill in the art.

Any appropriate route of administration may be employed, for example, administration may be parenteral, intravenous, intra-arterial, subcutaneous, intratumoral, intramuscular, intracranial, intraorbital, ophthalmic, intraventricular, intrahepatic, intracapsular, intrathecal, intracisternal, intraperitoneal, intranasal, intramyocardial, intracoronary, aerosol, suppository, epicardial patch, oral administration, or by perfusion. For instance, therapeutic compositions for parenteral administration may be in the form of liquid solutions or suspensions; for oral administration, formulations may be in the form of tablets or capsules; and for intranasal formulations, in the form of powders, nasal drops, or aerosols.

In some embodiments, a single dose of an EV composition may be administered. In other embodiments, multiple doses, spanning one or more doses per day, week, or month, are administered to the subject. In some embodiments, single or repeated administration of an EV composition, including two, three, four, five or more administrations, may be made. In some embodiments, the EV composition may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks), depending on the nature and/or severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ. As an example, if symptoms worsen, or do not improve, the EV composition, may be administered more frequently. Contrarily, if symptoms stabilize or diminish, the EV composition may be administered less frequently.

In some embodiments, an EV composition is administered in several doses, for example three, on or about several days, weeks, or months apart, for example two weeks apart, by intravenous administration. In some embodiments thereof, the composition may be diluted with, formulated with, and/or administered together with, a carrier, diluent, or suitable material (e.g., saline).

The present disclosure also encompasses methods for analyzing the activity, functionality, and/or potency, of conditioned media; or of an EV composition. The activity, functionality, and/or potency, of conditioned media; or of an EV composition, can be assessed by various techniques. For instance, the activity, functionality, and/or potency, of conditioned media; or of an EV composition, can be assessed by administering the conditioned media, extracellular vesicle-containing composition, to target cells in vitro, ex vivo, or in vivo. One or more properties of the target cells can then be analyzed, such as, for example, cell viability, hypertrophy, cell health, cell adhesion, cell physiology, cell electrophysiology, ATP content, cell metabolism, cell number, cell morphology, and neurite length and structure, to determine the activity, functionality, and/or potency, of conditioned media; or of an EV composition.

In some embodiments, assays known in the art may be used to determine the activity, functionality, and/or potency, of conditioned media; or of an EV composition. Cell viability (in cell viability assays) may be measured using, for example, a DNA-labeling dye or a nuclear-staining dye. The dye may be used with live cell imaging. An activity, functionality, and/or potency, of conditioned media; or of an EV composition, may also be determined with reference to one or more control samples. For instance, control cells may be one or more of: serum-deprived control cells which are not administered the conditioned media or the EV composition; control cells which are not serum-deprived; or serum-deprived control cells which are administered a mock conditioned media or mock EV composition. In some aspects, the dye is Yo-Yo3 iodide dye or calcein AM dye, such as for dopaminergic neuron viability and neurite outgrowth.

In some methods of the present disclosure, an activity, functionality, and/or potency, of conditioned media; or of an EV composition, can be assessed by a method comprising administering the conditioned media or the EV composition, to target cells cultured under at least one stress-inducing condition, and analyzing at least one property of the cells. The one or more properties of the target cells that may be analyzed can be selected from, for instance, cell migration, cell survival, cell viability, hypertrophy, cell health, cell adhesion, cell physiology, cell electrophysiology ATP content, cell number, cell morphology, and neurite length and structure. In some embodiments, the at least one property measured is electrical activity in neurospheres.

In one method, target cells are cultured in a pre-treatment medium under at least one stress-inducing condition, including but not limited to depravation of media component such as serum or growth factor or combination thereof, followed by administering a conditioned medium or EV composition, to the cell culture. The target cells are then cultured in the presence of the conditioned medium or the extracellular vesicle-containing composition, and at least one property of the cultured cells is measured one or more times during the culturing. In some embodiments, the at least one property is measured multiple times during the culturing in the presence of the conditioned medium or the extracellular vesicle-containing composition (such as, for example, 5 minutes to 10 hours apart from each other; 10 minutes to 4 hours apart from each other; or 30 minutes to 2 hours apart from each other). In some aspects, the stress conditions are the mutant cells themselves.

The methods may comprise culturing negative control cells in parallel, wherein the negative control cells are not administered the conditioned medium or the extracellular vesicle composition. In some embodiments, the negative control cells comprise negative control cells subjected to the same steps as the target cells, except that they are not administered the EV composition.

In certain embodiments, the negative control cells comprise negative control cells cultured in the pre-treatment medium under the at least one stress-inducing condition. The at least one property measured in the target cells may also then be measured in the negative control cells, either during or after they are cultured in the pre-treatment medium under the at least one stress-inducing condition.

In some embodiments, the negative control cells comprise negative control cells to which a mock conditioned medium or a mock EV composition is added. In specific embodiments thereof, the mock conditioned medium or the mock EV composition is produced by omitting cells from the process of producing a conditioned medium or EV composition, such as a process of the present disclosure.

The use of such a negative control(s) allows an activity, functionality and/or potency, of a conditioned medium or a extracellular vesicle composition, to be evaluated. For instance, where the at least one property measured is viability of the cultured cells, a conditioned medium or EV composition, may be determined to have an activity, functionality, potency (and/or exhibit a therapeutic effect), when the viability of the target cells is higher than the viability of the negative control cells.

Any one or more samples, and/or any one or more positive and/or negative controls, may be performed in replicate, such as, for example, in duplicate, in triplicate, etc. In some embodiments thereof in which cell viability is measured, and where replicate cultures are performed, the number of positive control cells in the replicate cultures may be averaged to produce an average maximum cell number (and the number of target cells in each replicate test culture may be normalized to the average maximum cell number, to calculate cell viability).

To more accurately compare an activity, functionality, and/or potency, between different conditioned media or extracellular vesicle compositions, it may be beneficial to determine the amount of the conditioned medium or the extracellular vesicle composition, added to target cells. This can be determined, for example, based on one or more of: the amount of secreting cells that produced the EV composition; the protein content of said EV composition; the RNA content of said EV composition; the exosome amount of said EV composition; and particle number.

In some embodiments, one or more specific cells may be tested to determine if the EV composition has effects, such as that may be beneficial for the treatment of a disease. Based on the effects of the EV composition on the functional activity, one may then be able to determine if the EV composition may be useful for the treatment of a disease. In some embodiments, the cells are derived from iPS cells from a subject that has a disease (e.g., a genetic disease or a disease with a genetic component or risk factor) such as a neurological or neurodegenerative disease (e.g., autism, epilepsy, ADHD, schizophrenia, bipolar disorder, etc.).

The assays to determine functional activity of the cells may comprise survival assays, microglia phagocytosis assays, calcium assays, MEA assays, synaptic pruning by microscopy assays, signal transduction chasing phosphorylated intermediates of various pathways, analysis of analytes released in the media in mono-, bi- or tri-culture with normal and disease specific cell types. For example, for disease modeling applications where isogenically engineered or patient-specific cells are compared to AHN controls, the treatment or exposure to neurogenerative proteins like amyloid beta, myelin, synaptosomes or Tau would result in decreased calcium signaling and electrical activity as well as increased neuroinflammatory cytokines. In some aspects, measuring functional activity comprises measuring dendrite area (e.g., MAP2), synapse count (e.g., Synapsin ½), cell count (e.g., CUX2), or axon area (e.g., beta III tubulin). For example, an increase (e.g., more than 30%, 40%, 50%, 60%, 70%, 80%, or 90%) in any of these functional activity measurements may indicate a candidate agent.

The assays may be performed in a high-throughput manner. For example, the cell cultures can be positioned or placed on a culture dish, flask, roller bottle or plate (e.g., a single multi-well dish or dish such as 8, 16, 32, 64, 96, 384 and 1536 multi-well plate or dish. The screening platform may be automated, such as robotic automation. The culturing platform may comprise an automated cell washer and high content imager.

The term “neurodegenerative disease or disorder” and “neurological disorders” encompass a disease or disorder in which the peripheral nervous system or the central nervous system is principally involved. The compounds, compositions, and methods provided herein may be used in the treatment of neurological or neurodegenerative diseases and disorders. As used herein, the terms “neurodegenerative disease”, “neurodegenerative disorder”, “neurological disease”, and “neurological disorder” are used interchangeably.

Examples of neurological disorders or diseases include, but are not limited to chronic neurological diseases such as diabetic peripheral neuropathy (including third nerve palsy, mononeuropathy, mononeuropathy multiplex, diabetic amyotrophy, autonomic neuropathy and thoracoabdominal neuropathy), Alzheimer's disease, age-related memory loss, senility, age-related dementia, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical basal degeneration, ALS-Parkinson's-Dementia complex of Guam, subacute sclerosing panencephalitis, Huntington's disease, Parkinson's disease, multiple sclerosis (“MS”), synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-Korsakoffs related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Other conditions also included within the methods of the present disclosure include age-related dementia and other dementias, and conditions with memory loss including vascular dementia, diffuse white matter disease (Binswanger's disease), dementia of endocrine or metabolic origin, dementia of head trauma and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including, but not limited to, epidural, subdural, subarachnoid, and intracerebral), and intracranial and intravertebral lesions (including, but not limited to, contusion, penetration, shear, compression, and laceration). Thus, the term also encompasses acute neurodegenerative disorders such as those involving stroke, traumatic brain injury, schizophrenia, peripheral nerve damage, hypoglycemia, spinal cord injury, epilepsy, and anoxia and hypoxia.

### A. Pharmaceutical Compositions

Also provided herein are pharmaceutical compositions and formulations comprising the present EV compositions and a pharmaceutically acceptable carrier.

EV compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen.

Pharmaceutical compositions and formulations as described herein can be prepared by mixing the active ingredients (such as EV compositions) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn—protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

### B. Distribution for Commercial, Therapeutic, and Research Purposes

In some embodiments, a reagent system is provided that includes extracellular vesicles that exists at any time during manufacture, distribution or use. The kits may comprise any combination of the EV composition described in the present disclosure in combination with undifferentiated pluripotent stem cells or other differentiated cell types, often sharing the same genome. Each EV compositions may be packaged together, or in separate containers in the same facility, or at different locations, at the same or different times, under control of the same entity or different entities sharing a business relationship. Pharmaceutical compositions may optionally be packaged in a suitable container with written instructions for a desired purpose, such as the mechanistic toxicology.

In some embodiments, a kit that can include, for example, one or more media and components is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits of the present disclosure also will typically include a means for containing the kit component(s) in close confinement for commercial sale. Such containers may include injection or blow molded plastic containers into which the desired vials are retained. The kit can also include instructions for use, such as in printed or electronic format, such as digital format.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation of iPSC-Derived Microglia EV-Enriched Secretome (MGL-EV) Composition

iPSC-derived microglia cultures were fed using 1×-MDM every 48 hours in MATRIGEL™ coated flasks through Day 10. On Day 12 the cells were replated into new MATRIGEL™ coated flasks and fed using addition feeding (spent media is not removed) of 2×-MDM every 48 hours until cell harvest, and cryopreservation of microglia on Day 23. The cells are harvested with cold dPBS (−/−) on day 23. The cells suspended in spent media and dPBS (−/−) are centrifuged to separate the cells from the diluted spent media. The microglia yield was determined by hemacytometer. The purity of microglial cultures on day 23 of differentiation were assessed by measuring cell surface expression of CD45, CD33, TREM2, and CD11b, as well as intracellular expression of PU.1, IBA, P2RY12, TREM2 and TMEM119 by flow cytometry. Microglia from iPSC lines derived from apparently healthy, normal human donors, with no known disease mutations, are considered apparently healthy, normal (AHN) or wild-type (WT) microglia (MGL). Microglia from iPSC lines derived from donors with known microglia mutations or from iPSC lines which have been engineered to contain microglia-relevant mutations are referred to as mutant microglia. These include MGL APOE E4/E4, MGL TREM2 HO, MGL TREM2 HZ, and MGL TREM2 R47H.

Conditioned Media: The diluted spent media (diluted with dPBS as described above) contains the secretome from microglia from Day 12 to Day 23. The diluted spent media was clarified by differential centrifugation to remove any remaining cells or large debris (400 g×10 min; 2000 g×30 min at 4° C.). Clarified Conditioned Media (MC) were aliquoted and frozen at −80° C. The total volume of diluted spent media collected was divided by the total number of microglia harvested to give the number of mother cells per mL diluted spent media.

Virgin Media Controls: Culture vessels were coated with MATRIGEL™ and 2×-MDM was added to the vessel and incubated under same conditions as the microglia for 48 hours. Control vessels contained no cells. Incubated media were collected and clarified with differential centrifugation as above. In some examples, the clarified virgin media was diluted ˜1:1 with 1×dPBS. Clarified virgin media controls were aliquoted and frozen at −80° C.

EV-enriched secretomes (EV) and mock-EV controls (MV): EV-enriched secretomes were prepared from MC. Mock-EV controls were prepared from virgin media controls. EV and MV were prepared by an ultracentrifugation method (UC), a tangential flow filtration (TFF) with size exclusion chromatography (SEC) method, or a Phosphatidyl-Serine (PS) affinity capture method (UFW) as detailed below.

Ultracentrifugation: ˜45 mL of MC or Virgin Media controls were ultracentrifuged at 100,000 g for 16 hours at 4° C. Pellets were resuspended in 0.1 μm filtered dPBS, aliquoted, and frozen at −80° C. Resuspension volumes were adjusted so that the EV-enriched secretomes from 1,400,000 mother cells (as counted on harvest day) are resuspended per 45 μL dPBS. EV and MV isolated thus are designated with the suffix “.UC” or “.uc”.

TFF and SEC: ˜2 L of MC were thawed, further clarified by differential ultracentrifugation (300 g×10 min 4° C.; 1,200 g×20 min 4° C., 10,000 g×30 min 4° C.), concentrated using tangential flow filtration (TFF) (filter TFF-EASY, 5 nm pore size, HansaBioMed Life Sciences LTD [HBM-LS]), enriched using size exclusion chromatography (SEC) (HBM-LS columns), and then re-concentrated using TFF (TFF-EASY, 5 nm pore size, HBM-LS). Protein content was determined by BCA assay (Pierce™ BCA Protein Assay Kit). Particle concentration and size distribution were determined by NTA, using the Zetaview analyzer, Particle Metrix. The material was then aliquoted to ˜1E10 particles per vial. A portion was frozen at −80° C. (samples designated with the suffix “.TFF.SEC.FRZ” or “.tff.sec.frz”) and a portion of the vials was lyophilized in PBS 1× with 5% sucrose (samples designated with the suffix “.TFF.SEC.LY” or “tff.sec.ly”). Lyophilized material was stored at 4° C. Lyophilized materials were reconstituted with pure water (Water For Injection; Gibco part #A1287301) and aliquoted prior to use. Remaining aliquots of reconstituted material that were not used immediately were stored at −80° C.

PS affinity capture: ˜15 mL of MC or virgin media controls were thawed and 100× EV-Save Extracellular Vesicle Blocking Reagent was added at a 1:100 ratio (Wako, part #058-09261). The mixture was concentrated by ultrafiltration (UF) (Vivaspin20 Ultrafiltration unit, MW cut off 100 kDa; Viva products ref #VS2041; pre-sterilized with a 70% ethanol wash prior to use, following the manufacturer's directions). Concentrated retentates were diluted with 1× 0.1 μm filtered dPBS to a final volume of ˜1 mL. EV were isolated from these suspensions using the Wako EV Isolation kit (W) (MagCapture Exosome Isolation Kit PS Version 2; Wako ref #290-84103) according to the manufacturer's directions. EV and MV controls thus isolated are designated as “.UFW” or “.ufw” or “.ps”.

### MGL-EV Composition Evaluation.

Protein concentration, BCA: The protein content of EV and MV were determined by BCA analyses, using the Pierce BCA Protein Assay Kit (ThermoScientific ref: 23225), according to the manufacturer's directions.

Particles size distribution, Nanoparticle Tracking Analysis: Particle size distribution and overall particle concentration in samples was determined using Nanoparticle Tracking Analysis (NTA) using a NanoSight NS300 (Malvern), equipped with a blue laser (Blue488), and analyzed with Malvern software (NTA 3.2 Dev Build 3.2.16, or earlier versions). Samples were diluted in 0.1 μm filtered dPBS as necessary and loaded onto the instrument manually or using a syringe pump. The instrument temperature was set to 25° C., and 5×30-second videos were captured with a camera level set between 14-16. If a syringe pump was used, an appropriate speed (20-100) was set. For analyses, a threshold of 3-5 was used, and the appropriate dilution factor applied. The overall particle concentration for each sample was noted. The particle size distributions were noted. The D50 for particle size (D50; median particle size, in nm, of a sample, wherein 50% of measured particles were that size or lower, as determined by the Nanosight) of each sample was noted.

Particle size distribution by Resistive Pulse Sensing (RPS) using the nCS1: MGL-EV.UC and MGL-MV.UC control preparations were evaluated on an nCS1 (Spectradyne) to obtain particle size distributions and concentration information by means of resistive pulse sensing technology. This is a complementary method to the NTA method described above. MISEV 2018 guidelines recommend assessing particle size distribution and particle concentration using orthogonal methods since each technology has its limitations and advantages (Thery et al., Minimal information for studies of extracellular vesicles 2018 (MISEV2018): a position statement of the International Society for Extracellular Vesicles and update of the MISEV2014 guidelines. J Extracell Vesicles. 7(1):1535750, 2018). It is expected that the particle size distribution and the overall concentrations will be different when measured on two different platform types, such as on an NTA and an RPS instrument. For the nCS1 analysis, samples were first diluted in PBS buffer+1% polysorbate 20, filtered to 0.02 μm, as directed by the manufacturer. Samples were then loaded into their own cartridges and run on the instrument. Dilution factors were added post-acquisition and default peak filter settings were applied during analysis.

Tetraspanin marker expression, ELISA method 1: Lyophilized material was reconstituted with pure water and analyzed by ELISA double sandwich as follows. EV were immobilized on an anti-CD63 coated surface and detected using HBM-LS antibodies to all three common EV tetraspanin markers at 1:500 dilutions (ANTI-CD9-biotin conjugated; ANTI-CD63-biotin conjugated; ANTI-CD81-biotin conjugated). Antibodies are detected by HRP-STREPTAVIDIN (Biorad) at 1:5000. Note that this method will only detect those EV which are at least CD63 positive. Those particles which are CD9 single positive, CD81 single positive or CD9+/CD81⁺ double positive will not be captured or detected using this ELISA method.

Tetraspanin marker expression, ELISA method 2: PS Capture Exosome ELISA Kit (Streptavidin HRP; Wako, Part #298-80601) was used to assess bulk exosome marker expression in each sample type, according to the manufacturer's directions. EV were diluted with 0.1 μm filtered dPBS to working solutions having target protein concentrations depending on isolation type (100 ng/mL to 10,000 ng/mL). Different target protein concentrations were targeted since the different EV isolation methods are expected to result in different amounts of co-isolated soluble protein components, and therefore very different concentrations of total protein. Lyophilized samples were reconstituted in pure water first, prior to diluting with DPBS. MV controls were volume-matched to their EV test samples. Antibodies to three common EV tetraspanin markers (CD9, CD81, CD63) were diluted in Reaction Buffer to various working solution concentrations: 1:4800 dilutions (Control Biotinylated Antibody Anti CD9, Wako, Part #019-27953; Control Biotinylated Antibody Anti CD81, Wako, Part #011-28111) or 1:100 dilution (Control Biotinylated Antibody Anti CD63, Wako, Part #290-80661). Antibodies were detected by HRP-conjugated Streptavidin (Wako, part #297-80671) using a working solution diluted to 1:50. Note that this ELISA method will detect only PS positive EV that are positive for at least one of the three tetraspanins. PS negative EV are not expected to be detected. The results from ELISA method 1 and ELISA method 2 may be different due to the different EV capture methods.

Single particle tetraspanin marker expression profiles, ONi: The ONi Nanoimager is a d-STORM capable super-resolution microscope. EV were assayed using the EV Imaging Kit according to manufacturer's directions (ONi part #210917-01). The principle of this kit is that EV are immobilized onto a chip by PS-affinity capture and then are probed for tetraspanin marker expression. The ONi software identifies clusters of fluorescence (these are PS-positive, tetraspanin-positive EV). The tetraspanins present on the cluster, will define which sub-type is the cluster. The ONi software identifies all of the clusters and determines the percentage of each cluster sub-type. Two clusters belong to the same sub-type if they express the same combination of tetraspanin markers. For example, clusters defined as the CD9 single positive sub-type are positive for CD9, negative for CD63, and negative for CD81 (CD9+/CD63−/CD81−; the order in which the tetraspanins are noted in this application are interchangeable and are not meant to ascribe meaning or hierarchy). Clusters defined as the CD9+CD63+ double positive sub-type are positive for CD9 and CD63 and negative for CD81 (also referred to as CD9+/CD63+/CD81−, or any ordering of these tetraspanin states), and so on. EV tetraspanins were assayed using the EV Profiler Kit according to manufacturer's directions (ONi part #210917-01). Staining protocol overview: EV samples are incubated with blocking buffer. EV samples are stained with fluorophore-conjugated antibodies overnight (anti-CD63; anti-CD9; anti-CD81). Chip surface preparation overview: Surface solutions are applied to the chip lanes to prepare surface for EV capture. Capture supplement solution is added to the stained EV. Stained EV are applied to the chip lanes. Fixation solution is applied to fix EV. Imaging buffer is applied to the chip lanes. Chip imaged in ONi microscope using 488, 560, and 640 lasers. The instrument temperature was set to 30° C., an illumination angle of 520 was used, and 2500 frames were captured for each sample.

Cell RNA extraction and total RNA sequencing: Total RNA was extracted using the QIASymphony robot and QIASymphony RNA kit (QIAGEN 931636) according to manufacturer's protocol. Library preparation (Illumina) and sequencing (Illumina NovaSeq6000) were performed using standard methods in the field.

EV RNA extraction: RNA extraction was performed on 200 μL of EV preparation, using the Wako microRNA Extractor SP kit (Wako, Ref: 295-71701), according to the manufacturer's protocol. The extracted RNA was eluted with 50 μL of Elution Solution. 2 μL of this RNA preparation was then used to assess RNA concentration, using the Lunatic (Unchained Labs). Additionally, an aliquot was sent to the University of Wisconsin Gene Expression Center for QC testing and sequencing. For the QC testing, a 2100 Bioanalyzer was used with the Eukaryote Total RNA Pico Assay (Agilent Technologies).

Small RNA sequencing: Small RNA libraries were prepared using a QIAseq miRNA Library kit (Qiagen, USA), using 5 μL of input RNA. To each sample, 3′ and 5′ adaptors (at a 1:5 dilution) and reverse transcriptase initiator were added, and the adapter-ligated RNA was then reverse transcribed. The resulting cDNA was purified using QMN beads (Qiagen, USA), and the cDNA libraries were then amplified for 16-22 cycles and purified twice. The amplified libraries were resuspended in 19.5 μL of nuclease-free water, and 17 μL were recovered. The libraries were quantified with Qubit in singlet, using a 1:100 dilution, and QC tested using an Agilent Bioanalyzer HS DNA chip (Agilent Technologies, USA). Sequencing was performed using NovaSeq6000 (SP 2×50 bp lane) on the Illumina NGS Systems.

Bioinformatics analyses of small RNA sequencing data: For bioinformatic analysis of sequencing data, FastQC v0.11.9 was used to determine the quality of the raw reads. Trimmomatic v0.39 was used to trim the adaptors from the raw reads. The trimmed reads were used for read mapping, and quantification was performed using miRge 3.0 pipeline implemented in Python with default settings (miRge 3.0 uses Bowtie v1.3.0 and SAMtools v1.7 for read mapping and quantification).

miRbase 22 was used for miRNA annotations. The 100 most abundant miRNAs in MGL-EV were used for over-representation analysis to determine their localization (using RNALocate), using a web-based interface program (miEAA 2.0 at an FDR 5.0%, adjusting the p-values for each category independently). The same tool was also used to determine the target genes from miRTarBase, to which the top 15 miRs bind, at a 5% FDR cutoff. An over-representation analysis was run on WebGestalt (WEB-based Gene SeT AnaLysis Toolkit) using these target genes against the human genome as reference set with ˜14000 genes annotated to the functional categories to determine the biological processes that are affected by these top 15 miRs.

To generate PCA plots, miR data were analyzed using JMP (version 17), “Estimation Method: Full SVD”.

MGL-EV-specific miR signature was extracted by the following two similar methods: A) By calculating the gene-wise minimum of log 2 RPM values of AHN MGL-EV sample replicates and maximum of all the other samples in the study (mutant MGL-EV were excluded from this analysis). A scatterplot of these calculated min vs max then shows the miRs that are exclusively expressed in AHN MGL-EV on the corresponding axis. No miRs were observed to be exclusively expressed in MGL-EV samples using this method. B) By calculating the gene-wise 10^{th} percentile of log 2 RPM values of MGL-EV sample replicates and 90^{th} percentile of all the other samples in the study. A scatterplot of these calculated 10^{th} vs 90^{th} percentile columns then show the miRs that are exclusively expressed in MGL-EV on the corresponding axis.

### MGL-EV Structural Evaluation.

Cryo-Electron Microscopy: Cryo-EM was used to ascertain the structure of MGL-EV. Freshly reconstituted lyophilized EV samples (stock solutions) were immediately used to prepare electron microscopy grids (grids: Quantifoil R 1.2/1.3 Cu 200). Grids were glow discharged in the GloQube for 45 seconds at 10 mA. 2×3 μL or 3 μL sample was applied to each grid. Vitrobot conditions were used: temperature=4° C., humidity=95%, 0.5 second drain time. Images were collected on the Talos Arctica at 200 kV using a Gatan K3 direct electron detector in counting mode with energy filter at a 20 eV slit width. Images were captured at two magnifications for high-resolution imaging:
1. 24kx, spot size 7, C2 aperture 100, C2 lens power 38.3%, Objective aperture 100, pixel size 3.7 Å, dose 20 e-/Å2, exposure time 10 sec, defocus −2 μm.
2. 79kx, spot size 4, C2 aperture 70, C2 lens power 40.33%, Objective aperture 100, pixel size 1.1 Å, dose 48 e-/Å2, exposure time 4.2 sec, defocus −2 μm.

### MGL-EV Protein Analyses.

Automated Western blot: An automated capillary Western immunoassay was performed on a Wes system (Protein Simple) according to the manufacturer's instructions using a 12-230 kDa Separation Module and either the Anti-Rabbit Detection Module, the Anti-Mouse Detection Module, or the Anti-Goat Detection Module, depending on the primary antibody used. Reconstituted lyophilized MGL-EV (“MGL.TFF.SEC.LY;” Reconstituted 1:1 RIPA Lysis Buffer:WFI; RIPA Lysis Buffer contains 20 mM TRIS pH 7.4, 50 mM NaCl, 0.5% NP-40, 0.25% Na-Deoxycholate, 1 mM EDTA) and ultracentrifugation-isolated EV.UC and MV.UC control samples were mixed directly with Fluorescent Master Mix and heated at 60° C. for 10 min. The microglia cell lysate (MGL-Cell) and a neural cell lysate sample were first diluted with Sample Buffer (‘10× Sample Buffer 2’ diluted 1:100 in water) to a concentration of 0.625 mg protein/mL and then mixed with Fluorescent Master Mix to a final concentration of 0.500 mg protein/mL and heated at 60° C. for 10 min.

The denatured samples, blocking reagent (antibody diluent), primary antibodies (in antibody diluent), HRP-conjugated secondary antibodies and chemiluminescent substrate were pipetted into the plate (part of Separation Module) as directed. Instrument default settings were used: stacking and separation at 375 V for 25 min; blocking reagent for 5 min; primary and secondary antibody both for 30 min; Luminol/peroxide chemiluminescence detection for ˜15 min (exposures of 1-2-4-8-16-32-64-128-512s). The resulting electropherograms were inspected to check whether automatic peak detection required any manual corrections. The following criteria were used to discriminate low protein signals from background: A peak signal-to-noise ratio (S/N) threshold of ≥10 was selected to discriminate low protein signals from background.

HTRF Assay: A Homogeneous Time Resolved Fluorescence (HTRF) assay was performed to detect the presence of Total LRRK2 protein and Phospho-LRRK2 protein. The assays were performed according to the manufacturer's recommendations (Cisbio; Total LRRK2 6FNRKPEG and Phospho-LRRK2 6FLRKPEG). Each kit contains 2 antibodies (specific for either LRRK2 or phospho-LRRK2), one labelled with Eu³⁺-Cryptate (donor) and one labelled with d2 (acceptor). The donor can be excited by laser and, when both antibodies are bound to the same LRRK2 molecule, the acceptor can be excited by FRET. The excitation emission is read at 2 wavelengths (620 nm=donor emission and 665 nm=acceptor emission) and the fluorescence ratio of acceptor/donor represents the relative energy transfer from donor to acceptor. The EV released from 370,000 mother cells were analyzed for both the .UC and the .TFF.SEC.LY preparations. 22 μg of total protein was analyzed from the microglia cell RIPA buffer lysate sample (“MGL RIPA Buffer Lysate”). Plates were read on a CLARIOstar plate reader. Results are presented as a ratio of the fluorescence measured at 665 nm:the fluorescence measured at 620 nm times 1000.

MacsPlex surface protein analysis: A MACSPlex Exosome Kit, Human (Miltenyi) was used to detect 37 different EV surface epitopes on MGL-EV.UC and MGL-MV.UC control preparations. The kit contains a cocktail of various fluorescently labeled bead populations, each coated with a specific antibody binding the respective surface epitope. Sample preparations were incubated with the antibody-coated MACSPlex EV Capture beads according to the manufacturer's directions. Captured EV were then further labeled with MACSPlex EV detection reagents, a cocktail of fluorescently labeled tetraspanins (CD9, CD63, and CD81) antibodies, creating a sandwich complex between the MACSPlex EV Capture Bead, EV, and the detection cocktail. The sample complexes were then run on a MACSQuant Analyzer 10 and analyzed based on the fluorescence characteristics of both the MACSPlex EV Capture Beads and the detection reagents. Bead populations were identified in the PE and FITC channels, and a positive APC signal within these populations indicated the presence of tetraspanin-positive EV bound to the bead via the corresponding surface epitope.

### MGL-EV In Vitro Function.

HUVEC scratch wound healing assay: For the HUVEC scratch wound healing assay, a scratch wound healing assay (developed by Essen BioSciences, for the Incucyte) was employed, according to the manufacturer's directions. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete Media for two days. Cultures were maintained at 37° C. (atmospheric oxygen, 5% CO₂) throughout maintenance and assay process. Wells were scratched using a Wound Maker (EssenBio, Ref: 4493) according to the manufacturer's directions, and cells were then rinsed with Endothelial Cell Basal Media (PromoCell Ref: C-22210) and cultured overnight (either in HUVEC Complete Media alone, as a positive control (“Complete”); in Endothelial Cell Basal Media alone, as a negative control (“Poor”); or in Endothelial Cell Basal Media supplemented with EV or MV preparations; the EV isolated by UC from FBS was used as an additional control, “FBS”). Using an Incucyte with the Scratch Wound Healing Module, plates were imaged every three hours for up to 24 hours. Wound closure was determined using the manufacturer's software, and values were baseline (negative, Poor, control) subtracted, and normalized to the positive (Complete) control.

HUVEC plating assay: HUVEC cells are typically plated in HUVEC Complete Media. When they are plated in Endothelial Cell Basal Media alone (“Poor media”), they do not plate down, survive, and/or proliferate as well. For the HUVEC plating assay, 115 μL of positive control mastermix (prepared by combining 100 μL “Complete” media (HUVEC Complete Media) with 15 μL of 0.1 μm filtered dPBS per well) was added to the appropriate wells of a 96 well plate. Further, 115 μL of negative control mastermix (prepared by combining, per well, 100 μL of Endothelial Cell Basal Medium with 15 μL of 0.1 μm filtered dPBS) was added to the appropriate wells. Further still, test condition mastermixes (prepared by combining, per well: 100 μL of Endothelial Cell Basal Media; up to 15 μL of EV (“EV 648”; MGL-EV.UC) or mock-EV control (“MV 853”; MGL-MV.UC); and 0.1 μm filtered dPBS to a final volume of 115 μL) were added to the appropriate wells. dPBS was added to the remaining wells on the 96-well plates to reduce the evaporation of the experimental wells, and plates were incubated in a humidified incubator (37° C., 5% CO₂) for 30 minutes to adjust the pH of the culture media prior to plating. Following this incubation, thawed HUVEC cells (thawed in HUVEC Complete media to a final concentration of 10,000 cells per 24.2 μL) were added to the experimental wells (a total of 10,000 viable cells were added per well; viable cell concentration was determined using an automated cell counter). Seeded plates were then incubated for 48 hours in a humidified incubator (37° C., 5% CO₂). The cultured cells were then analyzed by assessing cell confluence on an Incucyte, and by measuring intracellular ATP, using a CellTitreGlo assay, according to the manufacturers' directions. Briefly, at the end of the 48-hour incubation described above, the 96-well plate was removed from the incubator, and 50 μL of spent culture medium was removed from each experimental well (reducing the total well volume to 89.2 μL). 65 μL of CellTiter-Glo® Reagent was added to each well, and the plate contents were then mixed for 2 minutes on an orbital shaker (to induce cell lysis). The ATP content therein was then quantified using the CellTiter-Glo® Luminescent Cell Viability Assay (Promega) according to the manufacturer's directions. The resulting signal was analyzed using a Tecan for Life Science® plate reader.

Dopaminergic (Dopa) Neuron Cell viability and neurite outgrowth: In order to test the effect of EV on Dopaminergic Neuron cell viability, viable cell number, and neurite outgrowth, MGL-EV and MV controls were tested in an iCell Dopa Neuron Viability assay. Two lots of iCell Dopa Neurons (one with acceptable/typical viability and one known to have low viability) were plated according to the manufacturer's instructions (FCDI DopaNeuron User Guide, Document ID: X1003, 2017). Briefly, 96-well plates were ECM coated per the User Guide. Prior to cell seeding, wells were filled with Complete Maintenance Medium for iCell Dopa Neurons, with or without MGL-EV or volume-matched MV control. A 1× dose of MGL-EV was equivalent to the EV-enriched secretome isolated from 64,000 mother cells. Cells were seeded into prepared wells to a total well volume of 200 μL and cultured in a 37° C. humidified incubator (atmospheric oxygen, 5% CO₂) overnight. Half-volume media exchanges were performed on all wells on days 1, 3, and 5 post-seeding, with iCell DopaNeuron Complete Maintenance Medium (MM) or with MM containing MGL-EV or volume-matched MV controls according to the plate map. Phase contrast images were collected after the first hour, at the 12^{th} hour and then every 12 hours up to Day 9 using an IncuCyte SX5 (Sartorius). The length of neurites, the number of cell body clusters, and the cell body cluster area were determined using the NeuroTrack analysis tool (Sartorius). On Day 7 a portion of the wells were imaged in the presence of YOYO3-iodide at a 1:1000 dilution (ThermoFisher part #Y3606) and Calcein AM at a 1:1000 dilution (ThermoFisher part #C3100). Plates were incubated at 37° C. for a minimum of 1 hour prior to imaging. The number of live cells (green) and dead cells (red) were determined using the IncuCyte software using the Basic Analyser tool. All remaining wells were fed on Day 7 (half volume media exchange) with Complete Maintenance Medium for iCell Dopa Neurons only. On Day 9, all remaining wells were dyed and imaged as above.

NeuroSphere electrical activity: iCell NeuroSpheres 3D cell culture was initiated using iCell Gluta Neurons, iCell GABA Neurons and iCell Astrocytes, with a total of 25,000 cells per well (90% neurons [70:30 iCell Gluta Neurons/iCell GABA Neurons] and 10% astrocytes). Two different Ultra Low Attachment (ULA) plates were utilized (Sbio and Corning). Cells were maintained at 37° C., in a humidified incubator (atmospheric 02, 5% CO₂), in Supplemented BrainPhys Neuronal Medium (according to the “Measuring Synchronous Neuronal Activity on the Maestro Multielectrode Array” Application Protocol) until day 20. Spheroids were incubated with various concentrations of reconstituted MGL-EV (reconstituted EV #495.TFF.SEC.LY) overnight. Approximately 24 hours after treatment, Ca²⁺ oscillations were measured on the FDSS/μCell (Hamamatsu), FLIPR Calcium 6 Assay Kit (Molecular Devices), according to the manufacturers' directions.

Assessment of the impact of MGL-EV on the Electrophysiology of Dopaminergic Neuron Cells: Apparently Healthy Normal (AHN, iCell DopaNeurons, 01279) and diseased Dopaminergic Neurons (iCell DopaNeurons, 11299 and iCell DopaNeurons, 11344) were plated on a PEI/laminin coated 48-well Axion CytoView Plate in MM at a concentration of 125,000 cells/well. Cells were cultured in Dopaminergic Neuron MM for 24 hours and media was changed to Supplemented BrainPhys Neuronal Medium (according to the “Measuring Synchronous Neuronal Activity on the Maestro Multielectrode Array” Application Protocol) and cultured for 15 days. On day 15, freshly fed cultures were assessed by MultiElectrode Array (MEA; MaestroPro, Axion) (“Pre EV Baseline” timepoint). EV were added to wells in volumes such that 1 target cell received the MGL-EV of 1 mother cell. Three days (“D3 Post EV” timepoint) and four days after EV treatment (“D4 Pre Feed” timepoint), MEA measurements were again taken on treated and control wells. After the MEA reading acquisition on Day 4, 200 μL BrainPhys media was added on top of the 300 μL of spent BrainPhys Neuronal Medium and two hours later, MEA measurements were taken (“D4 2 h Post Feed” timepoint). Cells were cultured for another 2 days prior to an additional MEA reading acquisition (“D6” timepoint).

Results: To evaluate the MGL-EV composition, protein concentration was determined by BCA. Depending on the isolation method, varying amounts of protein were detected in EV preparations and MV controls (Table 1). Values are expressed as μg protein per million mother cells as counted on harvest day (EV) or from an MV prepared from an equivalent media volume. The results show that the UC preparation contains a higher concentration of total protein, likely due to the co-isolation of soluble proteins along with the EV.

TABLE 1
Protein concentration as determined by BCA.

| Isolation | Protein | Protein concentration |
|---|---|---|
| method | concentration in EV | in MV control |
| TFF.SEC.LY | 3.5 μg/M cells | Not available |
| | and 130-178 μg/mL | |
| UC | 11.2 μg/M cells | 52.6 μg/volume |
| UFW | 0.9 μg/M cells | 0.95 μg/volume |

Depending on the isolation method, varying levels of particle concentration were detected in EV preparations and MV controls, as shown in Table 2. All samples were evaluated by NanoSight. The TFF.SEC,LY EV sample was also evaluated by Zetaview Analyser.

TABLE 2
Particle count as determined by NanoSight.

| Isolation | Particle | Particle concentration |
|---|---|---|
| method | concentration in EV | in MV control |
| TFF.SEC.LY | 7289 particles/cell | Not available |
| | (and 217e11 particles/mL | |
| | by Zetaview) | |
| UC | 2277 particles/cell | 1323 particles/volume |
| UFW | 6068 particles/cell | 158 particles/volume |

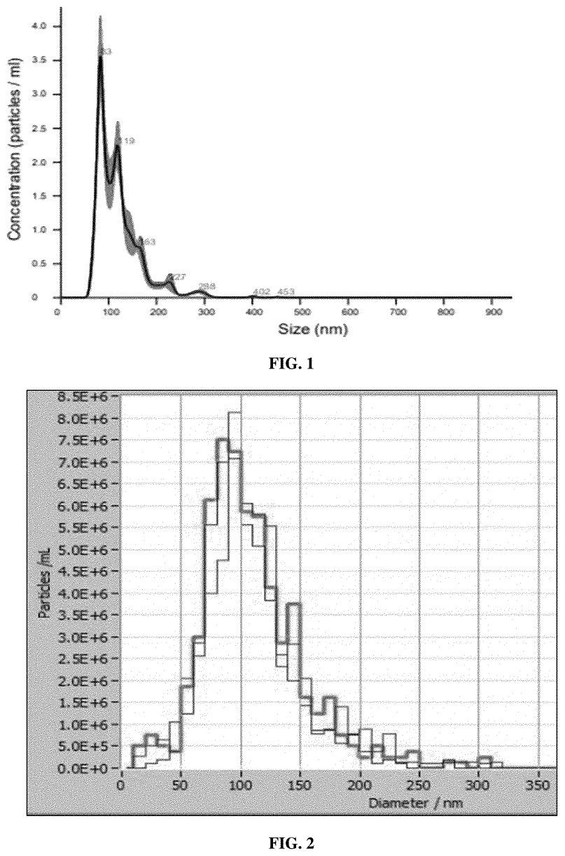

Representative particle size distribution histograms for a sample of MGL-EV.TFF.SEC.LY as determined by NanoSight and Zetaview Analyzer are given in FIG. 1 and FIG. 2, respectively. The majority of particles fall between 50 and 250 nm in diameter in both histograms, with a mode of approximately 80 to 100 nm.

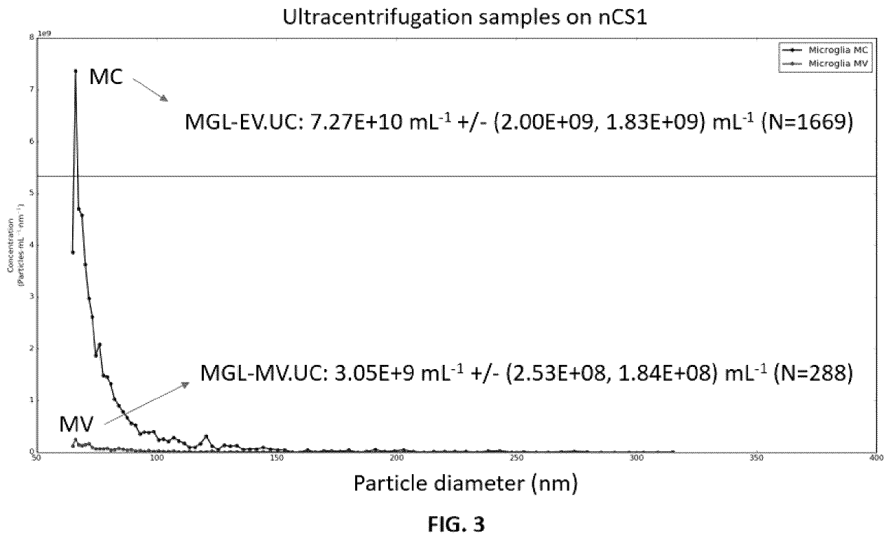

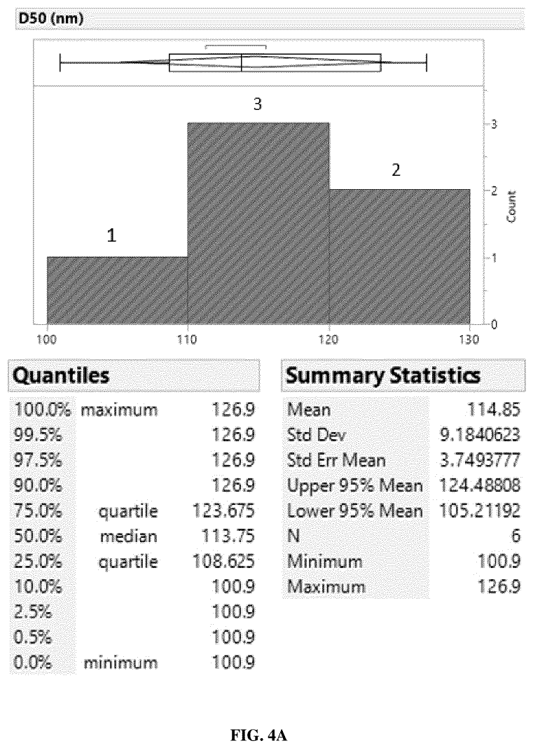

A sample of microglia-EV (“MGL-EV.UC”) and its MV control (“MGL-MV.UC”) were evaluated by nCS1. As depicted in FIG. 3, the MGL-EV have a higher concentration of particles than the matched MV control, with MGL-EV.UC having a concentration of 7.27E+10 particles/mL and the MGL-MV.UC having a concentration of 3.05E+9 particles/mL.

In order to evaluate if the size of MGL-EV were distinct from EV generated from other cell types, six biological replicates of apparently healthy normal (AHN) MGL-EV.UC were analyzed by NTA using a Nanosight. An additional 71 EV samples from iPSC and iPSC-derived cell types (non-MGL-EV) were evaluated by NTA. An additional 7 EV generated from mutant microglia (1 MGL APOE E4/E4-EV, 2 MGL TREM2 HO-EV, 1 MGL TREM2 HZ-EV, 1 MGL TREM2 R47H-EV and 2 EV samples from MGL genetically modified to express GFP) were evaluated by NTA. All of these EV samples were isolated by ultracentrifugation. The D50 (median particle size, in nm, of a sample, wherein 50% of measured particles were that size or lower, as determined by the Nanosight) was determined for each sample.

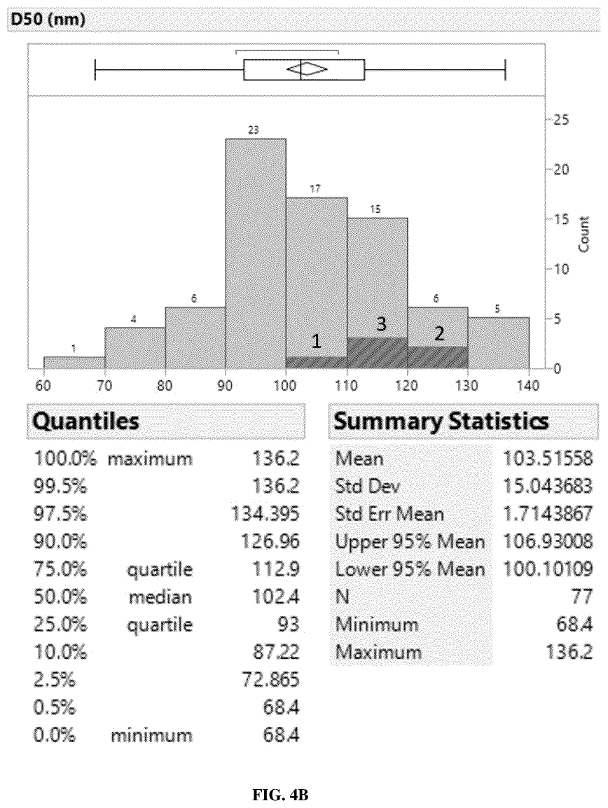

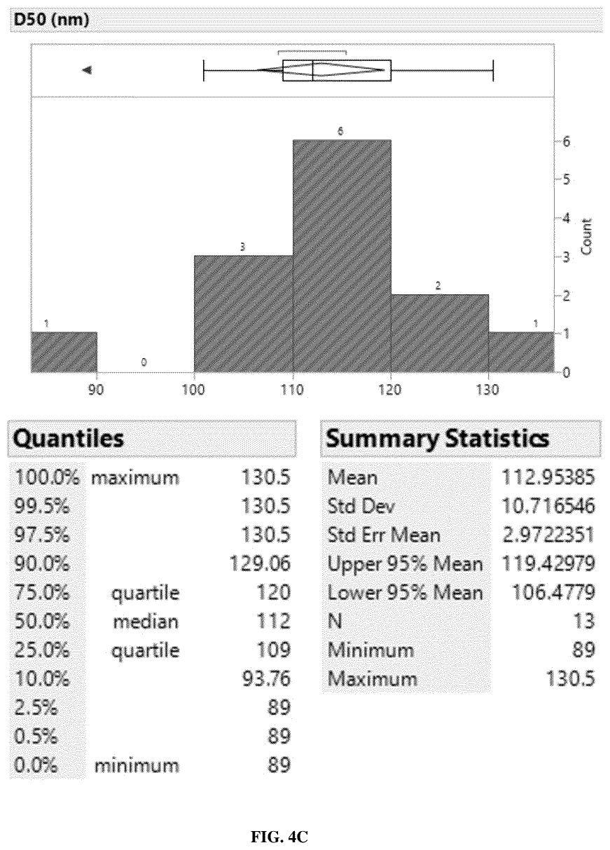

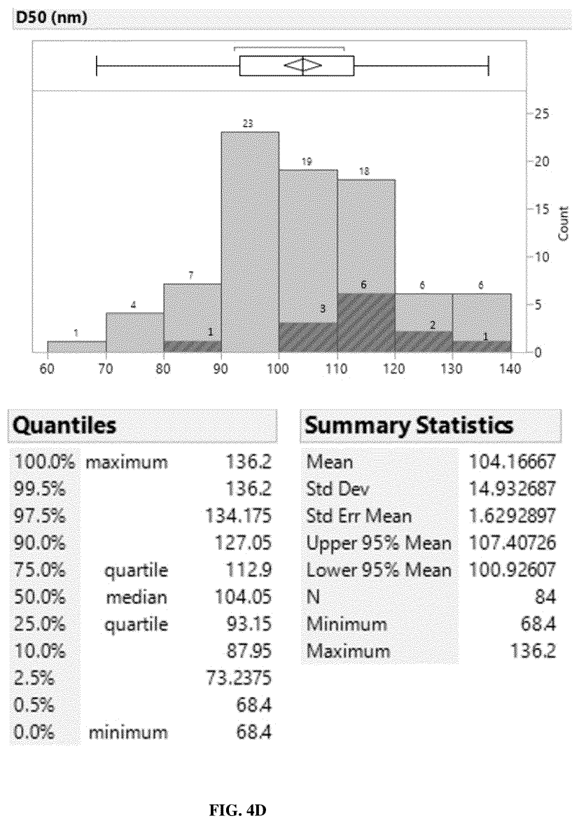

The D50s for the 6 AHN MGL-EV sample set are depicted in FIG. 4A. The median D50 of the six biological replicates is 113.75 nm. This is much larger than the median D50 for the 77-sample set (71 non-MGL-EV samples plus the 6 AHN MGL-EV samples) which is 102.4 nm (FIG. 4B). This is even larger than the third quartile of the 77-sample set, which is 112.9 nm.

The trend for MGL-EV to be larger than predicted by a mixed EV type sample set was also true when the mutant MGL-EV were included in the analysis. When all 13 MGL-EV samples were considered (6 AHN MGL-EV.UC, 1× MGL APOE E4/E4-EV,2 MGL TREM2 HO-EV, 1 MGL TREM2 HZ-EV, 1 MGL TREM2 R47H-EV, and 2 EV samples from MGL genetically modified to express GFP), the median D50 for MGL-EV is 112 nm (FIG. 4C). This is again much larger than the median D50 for the full 84-sample set (13 MGL-EV and the 71 non-MGL-EV), which is 104.05 nm, and is approximately equal to the third quartile of the 84-sample set, which is 112.9 nm (FIG. 4D). Taken together, these data show that MGL-EV are generally larger than other EV secreted by different cell types.

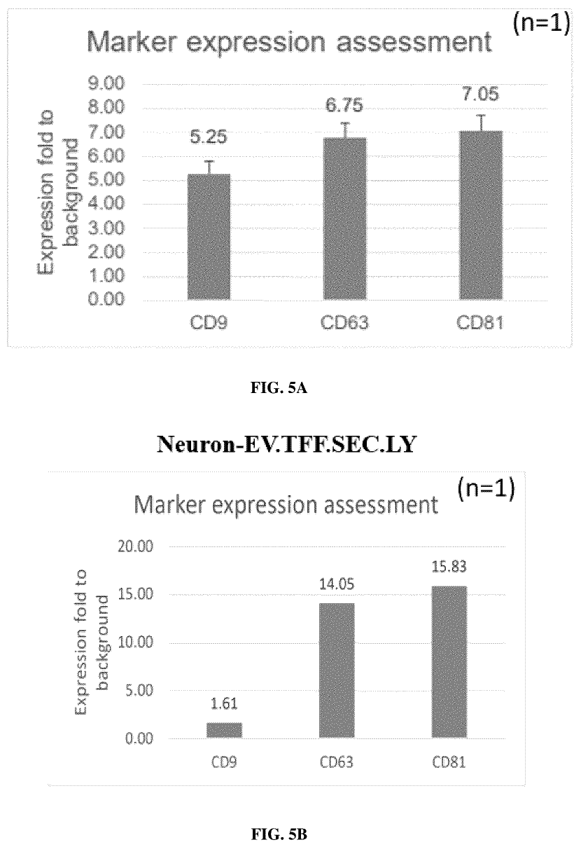

Tetraspanin marker expression was determined by ELISA method 1 (FIG. 5A). This ELISA confirmed the presence of EV expressing canonical EV tetraspanin markers, CD9, CD63, and CD81. Note that this ELISA method only detects EV that are at least CD63 positive. Any CD9 single positive, CD81 single positive or CD9/CD81 double positive EV would not be detected with this method. The marker expression profile for the MGL-EV was distinct from the marker profile detected in EV secreted by a different cell type, an iPSC-derived neuron cell type, as depicted in FIG. 5B.

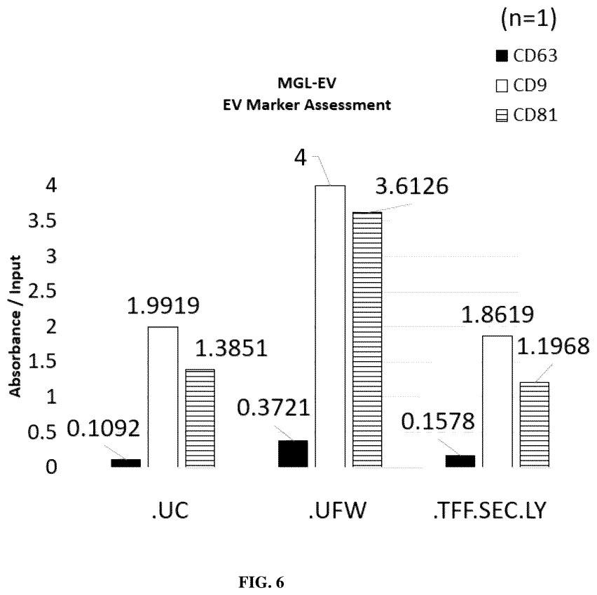

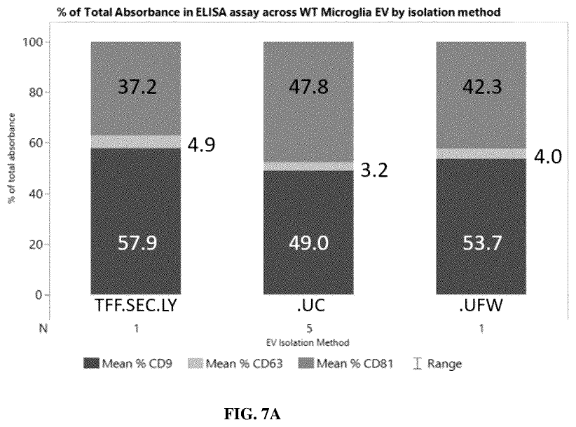

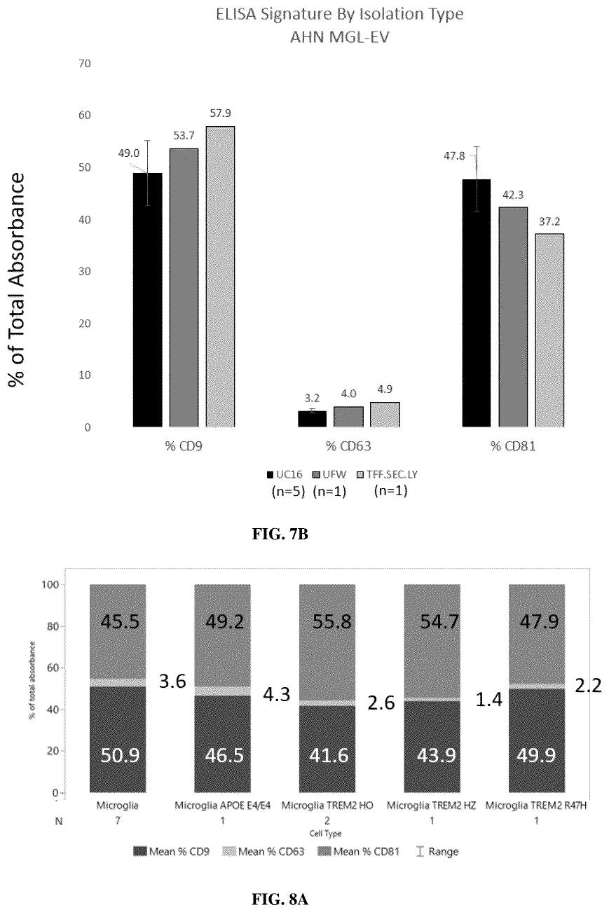

Tetraspanin marker expression was also determined by ELISA method 2 for AHN MGL-EV, isolated three different ways from the same lot of MC. Depending on the isolation method, varying levels of CD9, CD63 and CD81 were detected per input (FIG. 6). As expected, negligible signal was detected in MV controls. Note that different protein level inputs were used for the different MGL-EV samples, depending on the EV isolation method: UC input=100 ng protein; UFW input=10 ng protein; TFF.SEC.LY input=10 ng protein. However, the relative abundance of each (i.e., the tetraspanin signature) remained similar, regardless of isolation method (FIG. 7A, FIG. 7B). Note that the CD9 level for the UFW sample exceeded the instrument's maximum signal. Since the CD9 level for the UFW exceeded the instrument capabilities, the relative abundance of CD9 is not determined for this sample.

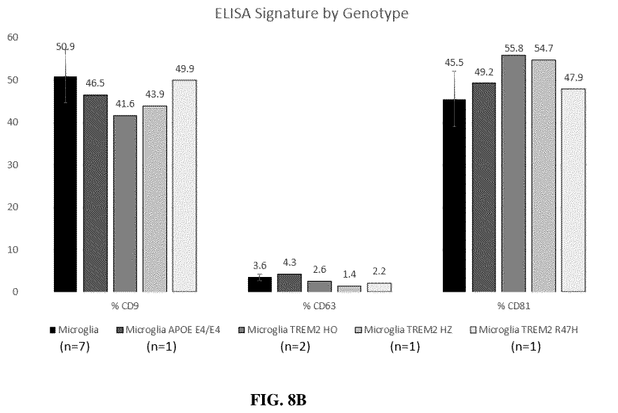

Additional MGL-EV were collected from the spent media produced by mutant microglia cells. EV were produced as described above, and isolated by ultracentrifugation as described above. The additional microglia cells were MGL TREM2 HO (a microglia cell line with mutations in the TREM2 gene on both alleles); MGL TREM2 HZ (a microglia cell line with a mutation in only one TREM2 gene allele); MGL APOE E4/E4 (a microglia cell line with a mutation in the APOE gene in both alleles); MGL TREM2 R47H (a microglia cell line with an R47H mutation). These EV were evaluated by ELISA method 2. As depicted in FIG. 8A and FIG. 8B, the tetraspanin signatures for MGL-EV from these different lines were highly similar to the AHN MGL-EV signature.

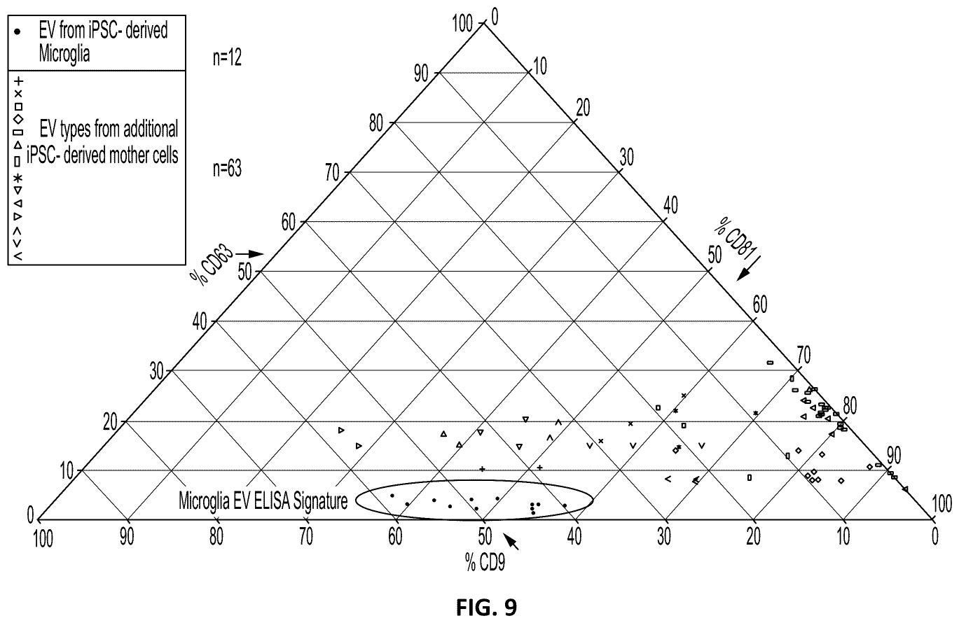

Importantly, the tetraspanin signature for all MGL-EV was remarkably different from the ELISA method 2 tetraspanin signatures determined for a set of 63 additional, non-MGL, differentiated iPSC-derived cell-types. These EV were generated from 30 distinct cell types using the UC method and have been categorized into 14 sub-sets (as depicted by 14 different symbols on the figure) as illustrated in FIG. 9. In this figure, the percentage of CD9, CD63 and CD81 in the tetraspanin signatures for each sample is graphed on a ternary plot. MGL-EV signatures are represented as solid circles in the figure. Regardless of whether the MGL are AHN or have a microglia-relevant mutation and regardless of isolation method (.UC, .UFW, and .TFF.SEC.LY), the 12 MGL-EV samples (1 AHN MGL-EV.TFF.SEC.LY, 5 AHN MGL-EV.UC, 1 AHN MGL-EV.UFW, 1 TREM2 R47H MGL-EV.UC, 1 APOE E4/E4 MGL-EV.UC, 1 TREM2 HZ MGL-EV.UC, and 2 TREM2 HO MGL-EV.UC) in the analysis cluster distinctly together and are separate from all other samples in the figure. All MGL-EV signatures analyzed fall within a space on the graph with the following parameters: CD81: between 30-60%; CD63: <10%; and CD9: between 35-60%. No other iPSC-derived differentiated cell-type-EV samples analyzed occupy this space on the graph.

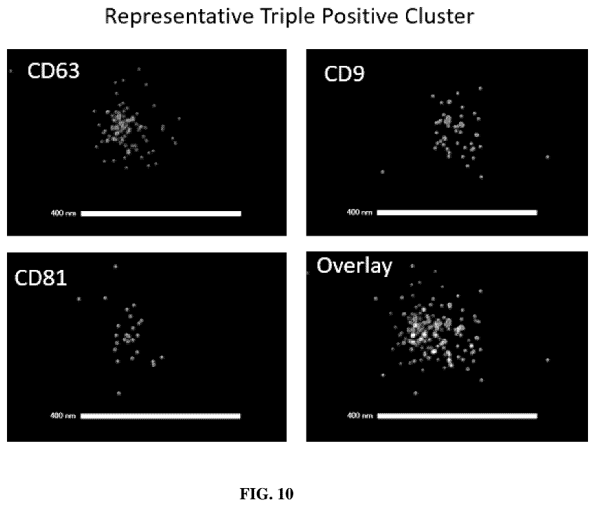

Both ELISA 1 and ELISA 2 methods measure overall tetraspanin protein levels, but do not inform on how those proteins are distributed amongst EV in a heterogeneous population. For example, it cannot be determined from an ELISA assay if there are single, double or triple tetraspanin positive EV sub-types, or their relative abundance. This can be accomplished using the ONi super resolution microscope. Individual clusters of tetraspanin signals are identified on the ONi chip. Each cluster is determined to be single, double or triple positive for CD9, CD81 or CD63. A representative triple positive cluster identified in a sample of AHN MGL-EV.TFF.SEC.LY is depicted in FIG. 10. Three panels show the individual marker signals, and a fourth panel shows the three tetraspanin signals overlayed. All clusters were identified on the chip and, the relative abundance of each cluster (i.e. EV) sub-population (referred to as a cluster sub-type) was determined by ONi and tabulated in Table 3. The cluster sub-type distribution was different in MGL-EV than in another EV type analyzed (Neural-EV.TFF.SEC.LY) using the same ONi method (Table 3). For the MGL-EV.TFF.SEC.LY, about two-thirds of clusters were single-positive (67%) with roughly even distribution of single-positive CD9 (26%), single-positive CD63 (20%), and single-positive CD81 (21%). Among the double-positive clusters, the CD81⁺/CD9+ double-positive clusters (17%) were almost three times as abundant as the CD63+/CD9+ double-positive clusters (6%) or the CD63+CD81⁺ double-positive clusters (6%). The triple-positive clusters were the least abundant at about 3%. Single-positive CD81, single-positive CD9, double-positive CD81⁺CD9+ proportions were all very different between MGL-EV and the EV of another iPSC-derived cell type EV examined.

TABLE 3
Relative abundance of cluster sub-types in two EV samples as imaged
and analyzed using the ONi super resolution microscope and software.

| | | MGL- | Neural- |
|---|---|---|---|
| | | EV.TFF.SEC.LY | EV.TFF.SEC.LY |
| Single Positive | CD9+/CD81−/CD63− | 25.7% | 58.0% |
| | CD9−/CD81+/CD63− | 21.3% | 3.1% |
| | CD9−/CD81−/CD63+ | 19.8% | 23.4% |
| Double Positive | CD9+/CD81+/CD63− | 17.2% | 0.6% |
| | CD9+/CD81−/CD63+ | 6.3% | 2.5% |
| Triple Positive | CD9−/CD81+/CD63+ | 6.4% | 10.9% |
| | CD9+/CD81+/CD63+ | 2.8% | 1.4% |

MGL-EV cargo were analyzed first by small RNA sequencing (results tabulated in Table 14). Analysis of the EV RNA cargo showed that MGL-EV had a miRNA content, distinct from other EV types analyzed. This is illustrated in a PCA plot depicted in FIG. 11, which includes 3 AHN MGL-EV samples and 33 other EV samples from iPSC and iPSC-derived other cell types. The mutant MGL-EV were not included in this analysis. The MGL-EV samples cluster with each other and are separate from the other EV types in the analysis, showing that the MGL-EV RNA content is distinct from other EV types. The 25 miR with the highest number of reads, along with the Mean log 2 RPM for the 3 biological replicates of AHN MGL-EV samples are given in Table 4 below. A full list of the miR identified is given in Table 14.

TABLE 4
The top 25 miR present in the MGL-EV with
average relative abundance are given.

| miRNA | Mean(log2 RPM) |
|---|---|
| hsa-miR-16-5p | 18.01848593 |
| hsa-miR-223-3p | 16.22093771 |
| hsa-miR-93-5p | 15.48290401 |
| hsa-miR-146a-5p | 15.34508981 |
| hsa-miR-142-3p | 15.17770068 |
| hsa-miR-191-5p | 14.853487 |
| hsa-miR-142-5p | 14.82514615 |
| hsa-miR-21-5p | 14.79274414 |
| hsa-miR-103a-3p/107 | 14.23487515 |
| hsa-miR-26b-5p | 14.12068795 |
| hsa-miR-122-5p | 13.97964014 |
| hsa-miR-125b-5p | 13.71921565 |
| hsa-miR-25-3p | 13.70605855 |
| hsa-miR-146b-5p | 13.65838066 |
| hsa-miR-101-3p | 13.4744615 |
| hsa-miR-29a-3p | 13.3460167 |
| hsa-miR-30e-5p | 13.27424721 |
| hsa-let-7a-5p/7c-5p | 13.17091411 |
| hsa-miR-342-3p | 13.10822515 |
| hsa-miR-148b-3p | 13.0695224 |
| hsa-miR-27a-3p/27b-3p | 13.0501307 |
| hsa-miR-224-5p | 12.93577274 |
| hsa-let-7f-5p | 12.9136024 |
| hsa-miR-125a-5p | 12.81587724 |
| hsa-miR-26a-5p | 12.78778667 |

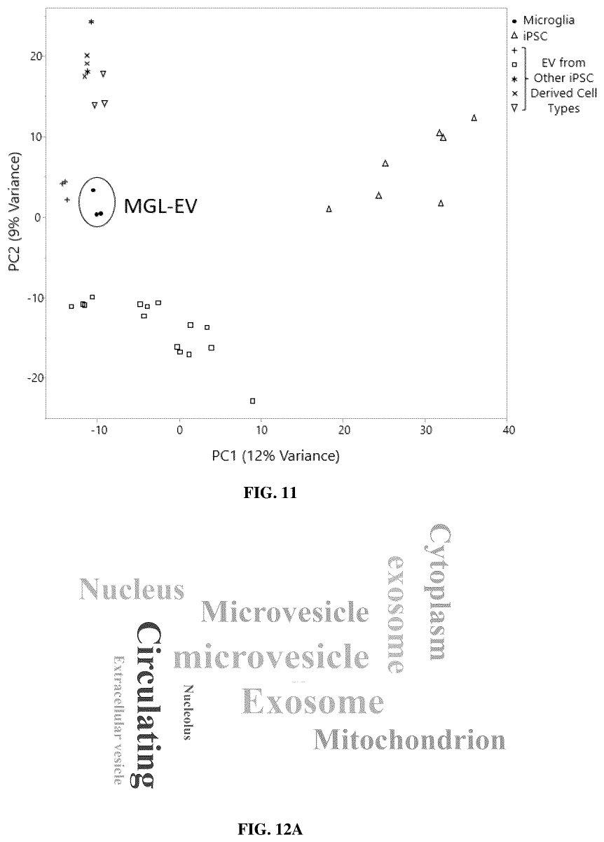

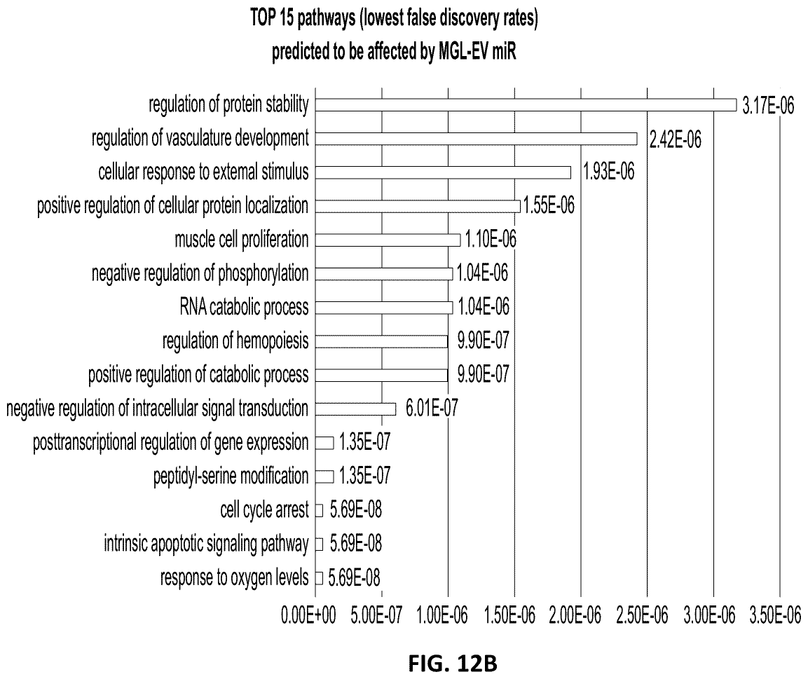

The top 100 highly abundant miRNA in the MGL-EV were used for over-representation analysis to determine their localization using RNALocate. The resulting wordcloud (FIG. 12A) supports that the RNA extracted from MGL-EV samples corresponds to miRNA associated with extracellular vesicles; six of the 10 terms are synonyms or subsets of extracellular vesicles.

An over-representation analysis was run using the predicted target genes of the 15 most abundant miRNA in MGL-EV. The analysis identified pathways which are predicted to be affected by the MGL-EV miRNA. While this analysis can predict pathways that will be affected, it cannot predict if these pathways would be upregulated or downregulated. The top 15 pathways predicted to be affected by MGL-EV miR are depicted in FIG. 12B, including the False Discovery Rate (FDR). The pathways with the lowest FDR (smallest bars) are the pathways for which there is the most confidence of enrichment. The most likely pathways affected by the MGL-EV-miR are therefore “response to oxygen levels”, “intrinsic apoptotic signaling pathway”, and “cell cycle arrest”. The full list of data are presented in Table 15.

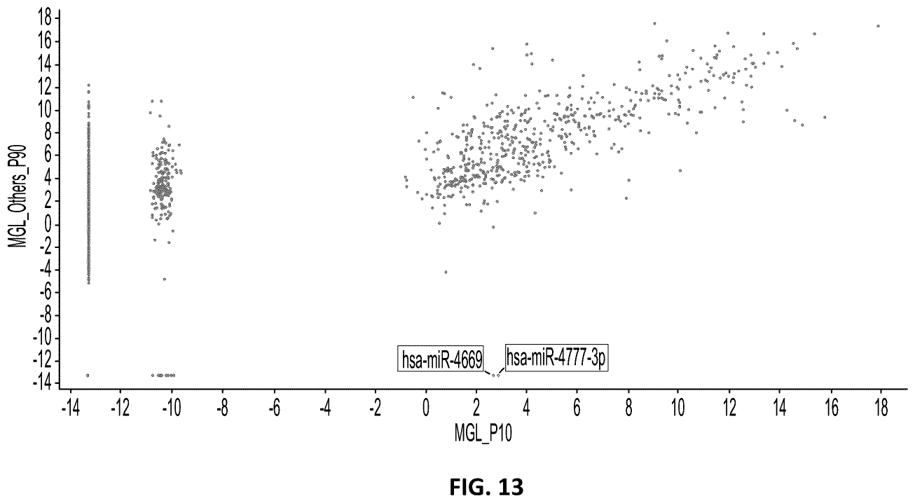

A scatterplot of the calculated 10^{th} vs 90^{th} percentile columns (second method for identifying miR signatures) showed that two miRNA are exclusively expressed in MGL-EV (FIG. 13). These miR are hsa-miR-4669 and hsa-miR-4777-3p.

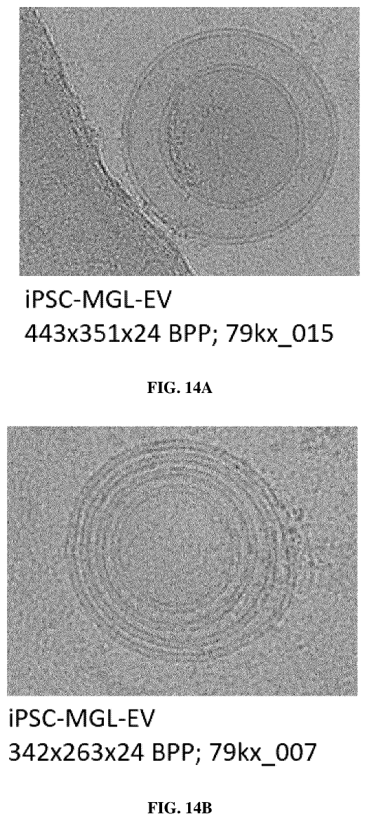

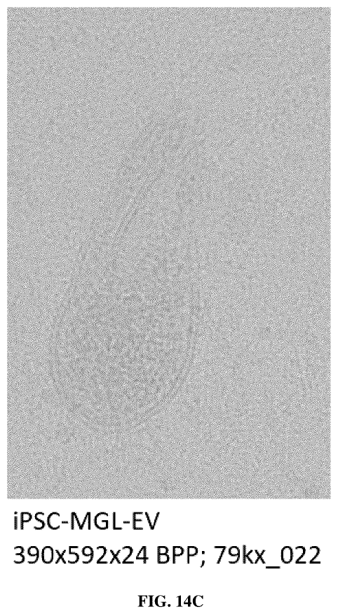

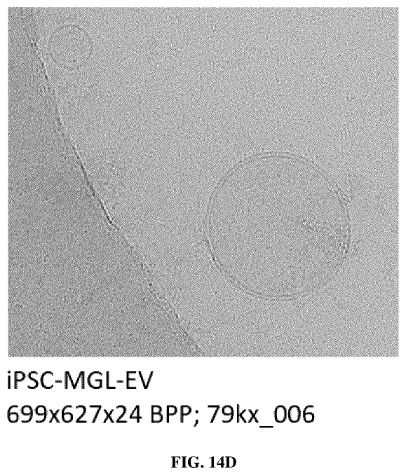

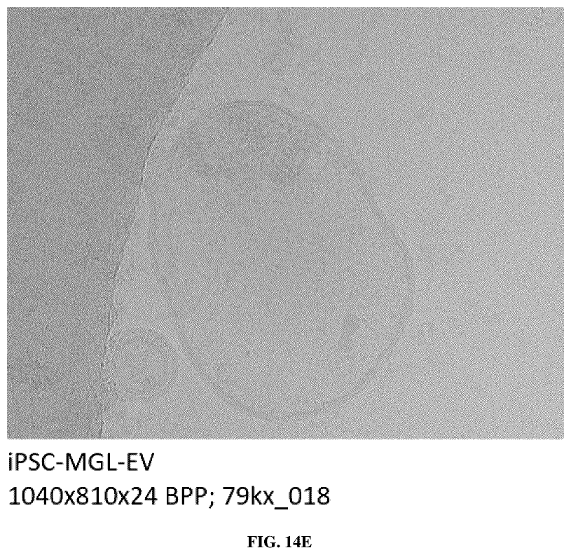

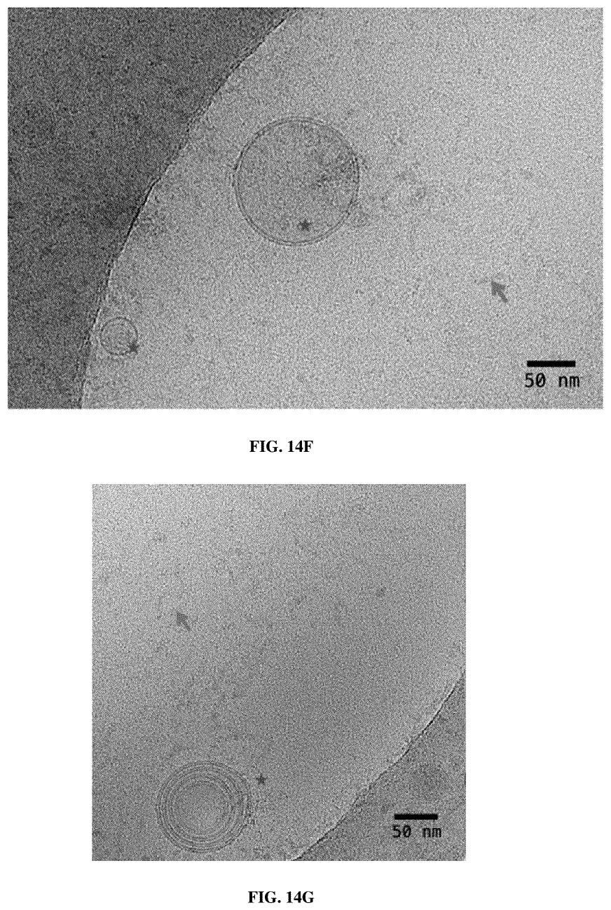

Representative images of MGL-EV composed of lipid bilayer structures are shown in FIGS. 14A-14G. The sample analyzed was a sample of AHN MGL-EV.TFF.SEC.LY. Identified EV structures include single, double, and multi-vesicular bodies of various sizes and shapes, including concentric and non-concentric organization. The presence of vesicle structures composed of at least one lipid bilayer confirms the presence of extracellular vesicles in the present EV preparations.

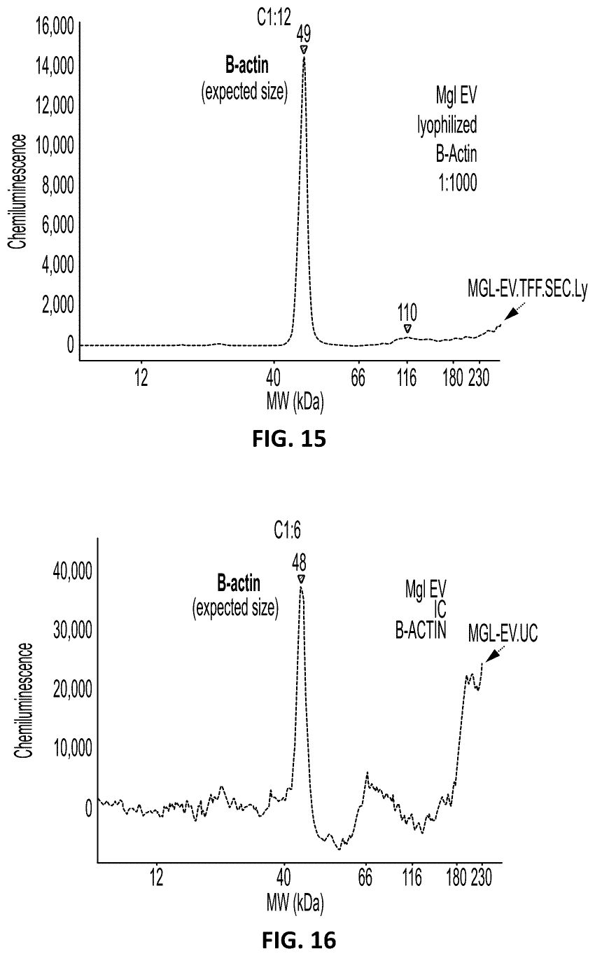

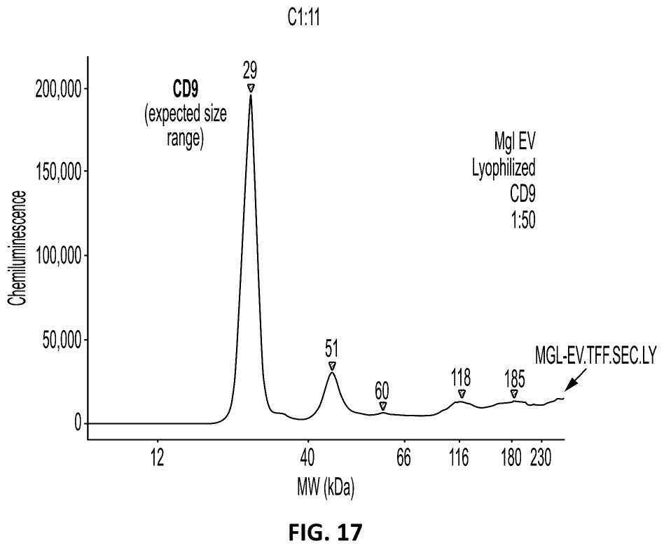

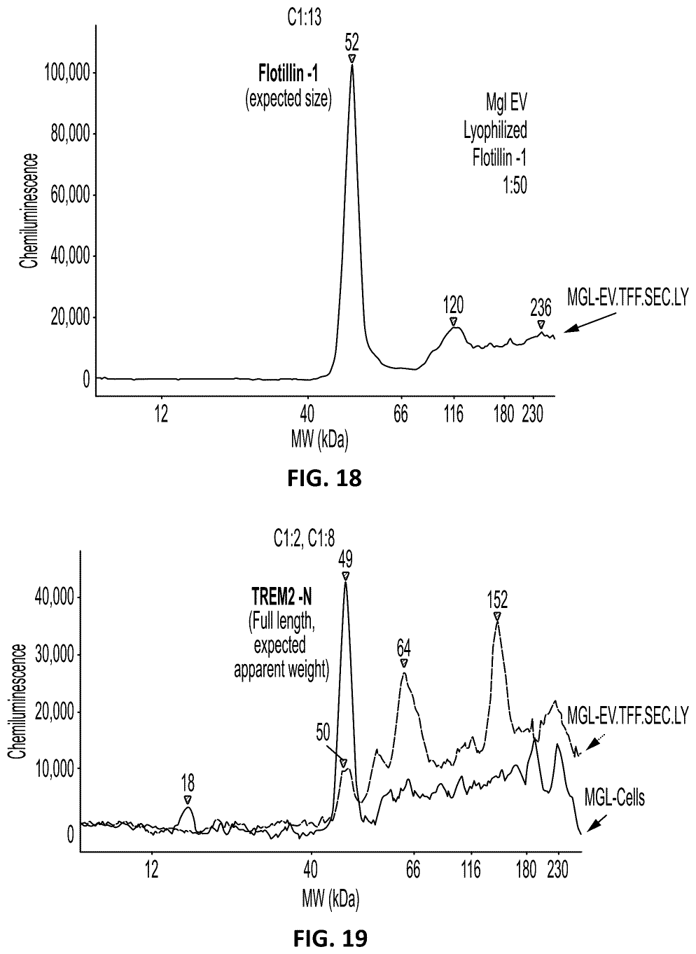

MGL-EV were analyzed by Western Blot to interrogate known EV markers as well as potential EV-type specific markers known to be present on microglia cells. FIGS. 15 and 16 confirm the presence in B-Actin in MGL-EV. FIG. 17 confirms the presence of CD9 in MGL-EV. FIG. 18 confirms the presence of Flotillin-1 in MGL-EV. The presence of these markers confirms the presence of extracellular vesicles in the present EV preparations. A summary of the identified peaks and their characteristics is given in Table 5.

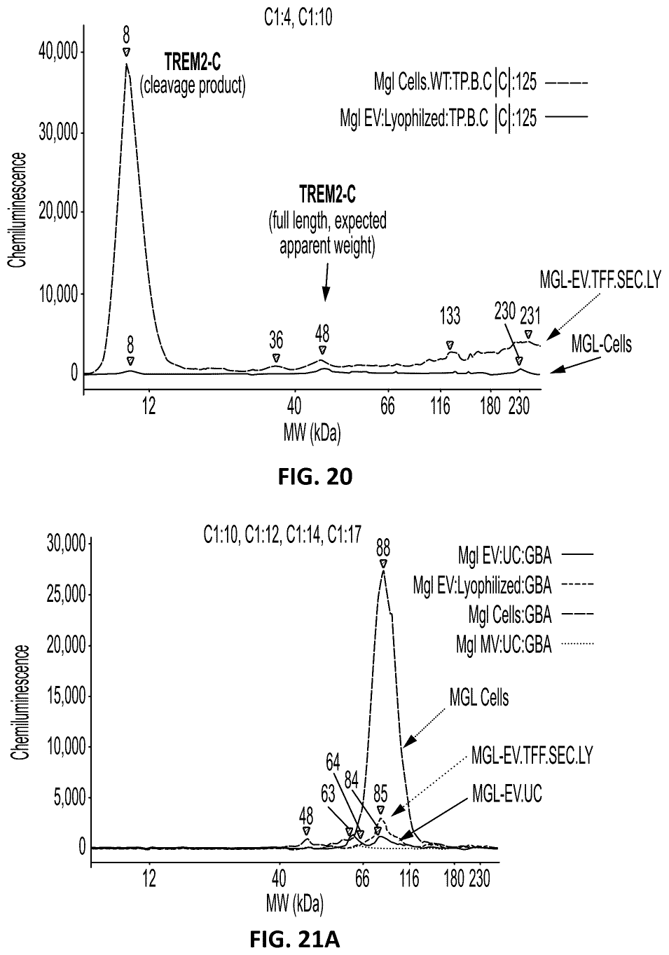

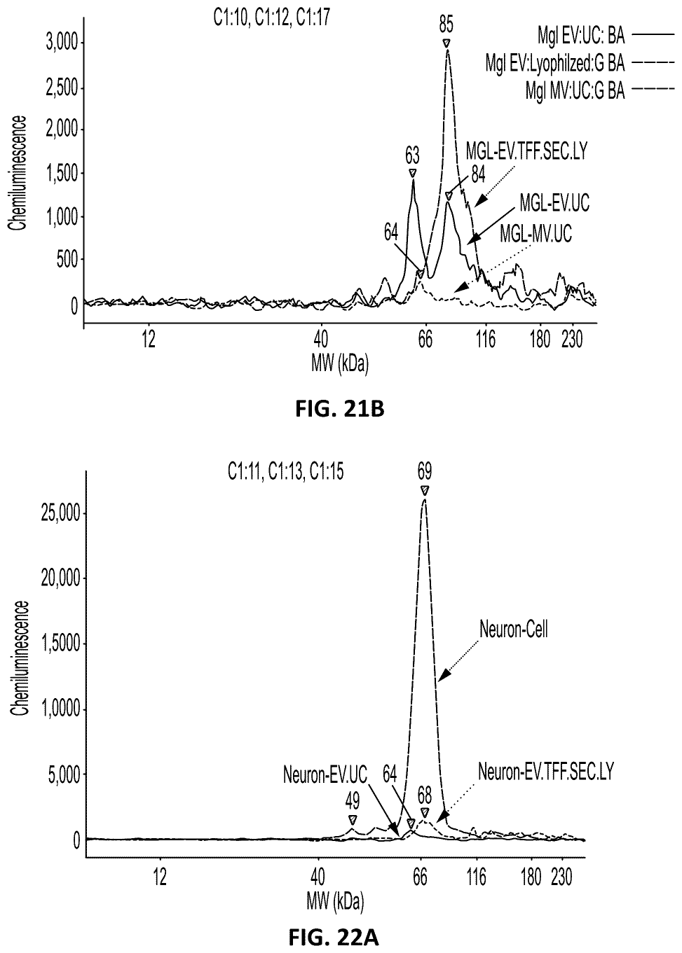

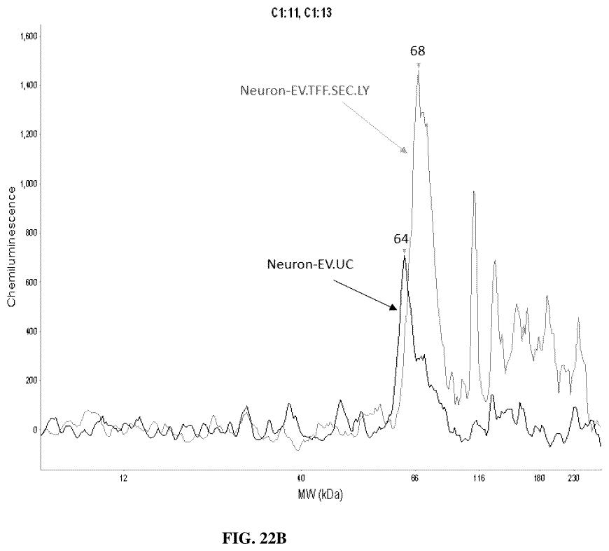

TABLE 5
Summary of automated western blot results for various samples and primary antibodies.

| Corresponding | | | | | MW | Major | | | | |
|---|---|---|---|---|---|---|---|---|---|---|
| figure | Sample | Attribute | Primary | Cap | (kDa) | Peak | Height | Area | Width | S/N |
| FIG. 15 | MGL-EV | Lyophilized | B-Actin | C1:12 | 49 | * | 14196.2 | 112006 | 7.4 | 2512.1 |
| | MGL-EV | Lyophilized | B-Actin | C1:12 | 110 | | 426.8 | 8773 | 19.3 | 31.3 |
| FIG. 16 | MGL-EV | UC | B-Actin | C1:6 | 48 | * | 38289 | 346208 | 8.5 | 84.5 |
| FIG. 17 | MGL-EV | Lyophilized | CD9 | C1:11 | 29 | * | 182018.1 | 2159506 | 11.1 | 3962.2 |
| | MGL-EV | Lyophilized | CD9 | C1:11 | 47 | | 27665.3 | 492734 | 16.7 | 498.2 |
| | MGL-EV | Lyophilized | CD9 | C1:11 | 60 | | 7301.7 | 192069 | 24.7 | 32 |
| | MGL-EV | Lyophilized | CD9 | C1:11 | 118 | | 13675.6 | 383702 | 26.4 | 79.2 |
| | MGL-EV | Lyophilized | CD9 | C1:11 | 185 | | 14824.1 | 423764 | 26.9 | 42.5 |
| FIG. 18 | MGL-EV | Lyophilized | Flotillin-1 | C1:13 | 52 | * | 97531.4 | 1199456 | 11.6 | 1262.7 |
| | MGL-EV | Lyophilized | Flotillin-1 | C1:13 | 120 | | 17324.5 | 603479 | 32.7 | 76.6 |
| | MGL-EV | Lyophilized | Flotillin-1 | C1:13 | 236 | | 16001.5 | 538910 | 31.6 | 24.7 |
| FIG. 19 | MGL-Cells | WT | TREM2 (N) | C1:2 | 18 | | 3576.4 | 24211 | 6.4 | 15.8 |
| | MGL-Cells | WT | TREM2 (N) | C1:2 | 49 | * | 42994.7 | 395717 | 8.6 | 141 |
| | MGL-EV | Lyophilized | TREM2 (N) | C1:8 | 50 | * | 6515.1 | 213295 | 30.8 | 33.2 |
| | MGL-EV | Lyophilized | TREM2 (N) | C1:8 | 64 | | 21947.5 | 666872 | 28.5 | 45 |
| | MGL-EV | Lyophilized | TREM2 (N) | C1:8 | 152 | | 25238.8 | 1601128 | 59.6 | 74.8 |
| FIG. 20 | MGL-Cells | WT | TREM2 (C) | C1:4 | 8 | | 396 | 5703 | 13.5 | 35.7 |
| | MGL-Cells | WT | TREM2 (C) | C1:4 | 48 | * | 690.9 | 10273 | 14 | 67.3 |
| | MGL-Cells | WT | TREM2 (C) | C1:4 | 234 | | 625.7 | 5747 | 8.6 | 70.9 |
| | MGL-EV | Lyophilized | TREM2 (C) | C1:10 | 8 | * | 35182.6 | 776241 | 20.7 | 1281.4 |
| | MGL-EV | Lyophilized | TREM2 (C) | C1:10 | 36 | | 938.8 | 25907 | 25.9 | 27.4 |
| | MGL-EV | Lyophilized | TREM2 (C) | C1:10 | 48 | | 1600.5 | 49677 | 29.2 | 46.9 |
| | MGL-EV | Lyophilized | TREM2 (C) | C1:10 | 133 | | 2825 | 100683 | 33.5 | 71.1 |
| | MGL-EV | Lyophilized | TREM2 (C) | C1:10 | 237 | | 4489.2 | 165799 | 34.7 | 34.9 |
| FIG. 21A | MGL-EV | UC | GBA | C1:10 | 63 | | 587.3 | 16179 | 25.9 | 40.7 |
| | MGL-EV | UC | GBA | C1:10 | 84 | * | 778.6 | 22019 | 26.6 | 27.1 |
| | MGL-EV | Lyophilized | GBA | C1:12 | 85 | * | 2699.3 | 48179 | 16.8 | 99.9 |
| | MGL-Cells | WT | GBA | C1:14 | 48 | | 679.2 | 14372 | 19.9 | 34.8 |
| | MGL-Cells | WT | GBA | C1:14 | 88 | * | 27331.3 | 668255 | 23 | 551.9 |
| | MGL-MV control | UC | GBA | C1:17 | 64 | | 267.8 | 1841 | 6.5 | 11.6 |
| FIG. 21B | MGL-EV | UC | GBA | C1:10 | 63 | | 587.3 | 16179 | 25.9 | 40.7 |
| | MGL-EV | UC | GBA | C1:10 | 84 | * | 778.6 | 22019 | 26.6 | 27.1 |
| | MGL-EV | Lyophilized | GBA | C1:12 | 85 | * | 2699.3 | 48179 | 16.8 | 99.9 |
| | MGL-MV control | UC | GBA | C1:17 | 64 | | 267.8 | 1841 | 6.5 | 11.6 |
| FIG. 22A | Neuron-EV | UC | GBA | C1:11 | 64 | * | 438.9 | 10903 | 23.3 | 36.1 |
| | Neuron-EV | Lyophilized | GBA | C1:13 | 68 | * | 1005.6 | 27731 | 25.9 | 60.3 |
| | Neuron-Cells | WT | GBA | C1:15 | 49 | | 653.3 | 14406 | 20.7 | 32.7 |
| | Neuron-Cells | WT | GBA | C1:15 | 69 | * | 25182.6 | 381729 | 14.2 | 1339.6 |
| FIG. 22B | Neuron-EV | UC | GBA | C1:11 | 64 | * | 438.9 | 10903 | 23.3 | 36.1 |
| | Neuron-EV | Lyophilized | GBA | C1:13 | 68 | * | 1005.6 | 27731 | 25.9 | 60.3 |

Table 5 Definitions: The attribute “Lyophilized” identifies the sample in question as being an “EV.TFF.SEC.LY” sample, or a sample that was lyophilized after being isolated using TFF and SEC. The attribute “WT” identifies the sample in question as being “AHN.” The attribute “UC” identifies the sample in question as being an “EV.UC” sample, or a sample that was isolated using ultracentrifugation. “Primary” refers to the primary antibody being used. “Cap” refers to the capillary position, or lane, that the sample was run in during the assay. “S/N” is the signal-to-noise ratio of the peak identified.

In addition to evaluating traditional EV markers, MGL-EV were evaluated for the presence of microglia-related markers GBA and TREM2 using the automated western blot method.

A sample of MGL-EV.TFF.SEC.LY was analyzed using the Protein Simple Wes, probing for the presence of TREM2, utilizing an antibody recognizing the N-terminus of the protein (FIG. 19). As a control, a lysate of microglia cells (“MGL-Cells”) was included in the experiment. These cells are AHN (also referred to as WT in FIG. 19 and in the corresponding sections of Table 5). A peak was identified at the expected size of TREM2, indicating the presence of full-length TREM2 in the MGL-Cells and in the MGL-EV.TFF.SEC.LY sample. For each identified peak, the estimated molecular weight of the protein, the peak height, the peak area, the peak width, and the signal to noise ratio (“S/N”) at the peak are given in Table 5. The result for the MGL-EV.TFF.SEC.LY is depicted in FIG. 19 as a grey trace. The result for the MGL-Cells is depicted as a black trace in the figure.

A sample of MGL-EV.TFF.SEC.LY was analyzed using the Protein Simple Wes, probing for the presence of TREM2, utilizing an antibody recognizing the C-terminus of the protein (FIG. 20). As a control, a lysate of microglia cells (“MGL-Cells”) was included in the experiment. These cells are AHN (also referred to as WT in FIG. 20 and in the corresponding sections of Table 5). A short peak was identified at the expected size of full-length TREM2 (˜48 kDa), indicating the presence of full-length TREM2 in the MGL-Cells and in the MGL-EV.TFF.SEC.LY sample. The small product detected here matches the predicted size of one of the C-terminal cleavage products of TREM2 (˜8 kDa) in both the MGL-Cells and the MGL-EV.TFF.SEC.LY. The 8 kDa peak was clearly enriched in the MGL-EV.TFF.SEC.LY as compared to the MGL-Cells. In the MGL-EV.TFF.SEC.LY, the area under the 8 kDa peak was 15.6 times greater than the area for the full-length protein in that sample. In the MGL-Cells, the area under the 8 kDa peak was less than the area of the full-length protein peak in that sample. For each identified peak, the estimated molecular weight of the protein, the peak height, the peak area, the peak width, and the signal to noise ratio (“S/N”) at the peak are given in Table 5. The result for the MGL-EV.TFF.SEC.LY is depicted in the figure as a grey trace. The result for the MGL-Cells is depicted as a black trace in the figure. Surprisingly and unexpectedly, the MGL-EV are highly enriched in the TREM2 C-terminal cleavage product, which could have therapeutic or diagnostic applications for neurodegenerative diseases, including Parkinson's Disease, Parkinson Like Syndrome, Alzheimer's Disease, Dementia, Seizures, epilepsy, psychiatric conditions such as depression, anxiety, schizophrenia, Huntington, other neuromuscular diseases, and rare conditions, especially involving neuron or glia pathologies.

Samples of MGL-EV.TFF.SEC.LY, MGL-EV.UC, and MGL-MV.UC control were analyzed using the Protein Simple Wes, probing for the presence of GBA (FIG. 21A and FIG. 21B). As a control, a lysate of microglia cells (“MGL-Cells”) was included in the experiment. These cells are AHN (also referred to as WT in FIG. 21A and corresponding sections of Table 5). A major peak in the 84-88 kDa size range was identified in the MGL-EV.TFF.SEC.LY, MGL-EV.UC, and MGL-Cells. This peak is not present in the MGL-MV control. This is larger than the expected 77 kDa size which is typically seen for GBA by automated western blot, according to the manufacturer of the antibody. This is also larger than the GBA detected in another EV type, that was secreted by an iPSC-derived neuron cell type that is depicted in FIG. 22A and FIG. 22B, which was analyzed in parallel. For each identified peak in FIGS. 21A, 21B, 22A and 22B, the estimated molecular weight of the protein, the peak height, the peak area, the peak width, and the signal to noise ratio (“S/N”) at the peak are given in the corresponding sections of Table 5. Surprisingly and unexpectedly, the MGL-EV comprise a form of GBA which has a larger apparent weight than expected, which may comprise a dimer or multimer with another protein or peptide, or a post-translational modification not present in neuronal cells, such structural or functional uniqueness may affect, without limiting, neurodegenerative diseases, psychiatric conditions, neuromuscular diseases, and rare conditions, especially involving neuron or glia pathologies.

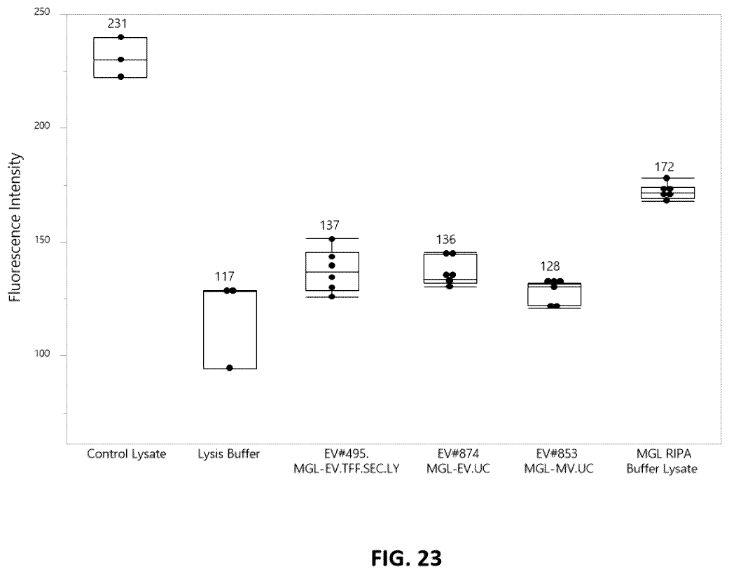

The presence of LRRK2 protein in MGL-EV was evaluated using an HTRF (Homogeneous Time Resolved Fluorescence) assay. This assay was performed to detect the presence of LRRK2 protein in microglia in cell lysates and in vesicles. Results depicted in FIG. 23 are given as a ratio of fluorescence intensities. A higher ratio indicates a greater amount of target. The higher ratio indicates the presence of LRRK2 in MGL-EV.TFF.SEC.LY and MGL-EV.UC compared to negative controls (Lysis Buffer and MGL-MV.UC). The fluorescence ratio indicates the presence of LRRK2 in the microglia cell lysate (“MGL RIPA Buffer Lysate”). Surprisingly and unexpectedly, results show the presence of LRKK2 in MGL-EV.

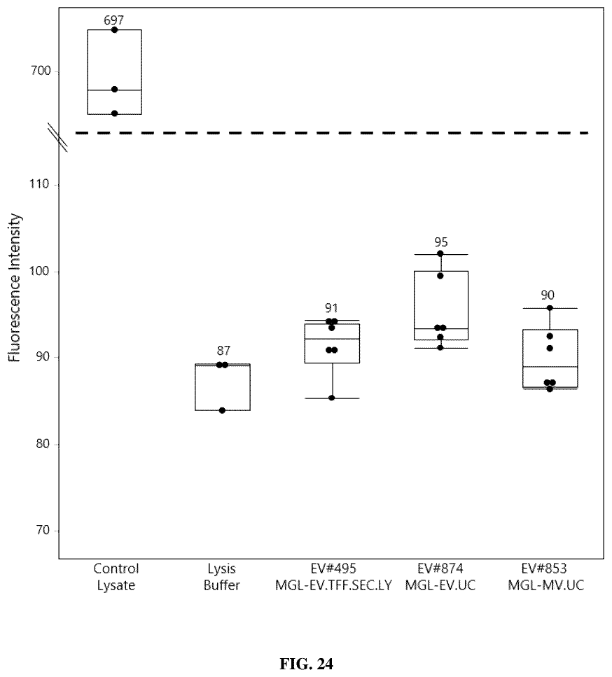

Similarly, an HTRF assay was performed to detect the presence of Phospho-LRRK2 protein in MGL-EV. Results depicted in FIG. 24 are given as a ratio of fluorescence intensities. The higher fluorescence ratio indicates the presence of Phospho-LRRK2 in MGL-EV.TFF.SEC.LY and MGL-EV.UC compared to negative controls (Lysis Buffer and MGL-MV.UC). Surprisingly and unexpectedly, results show the presence of phospho-LRKK2 in MGL-EV.

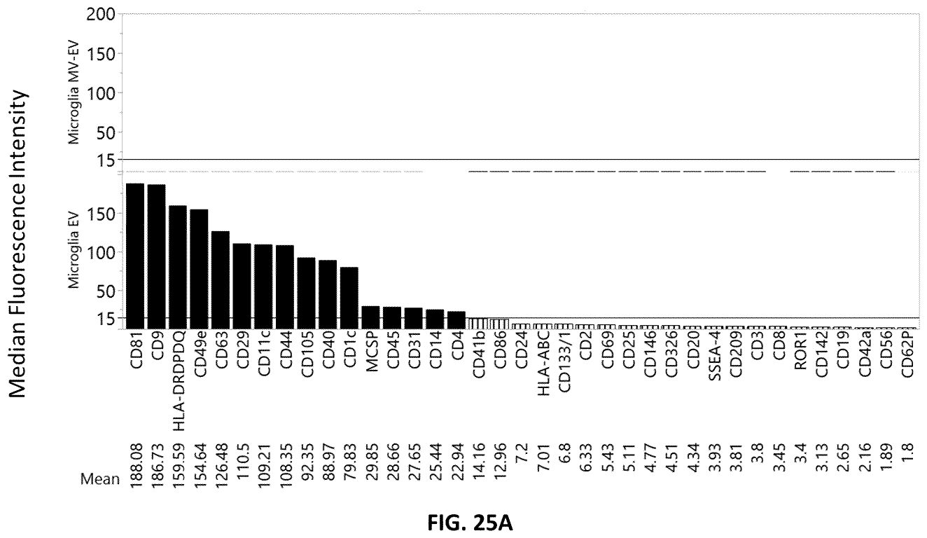

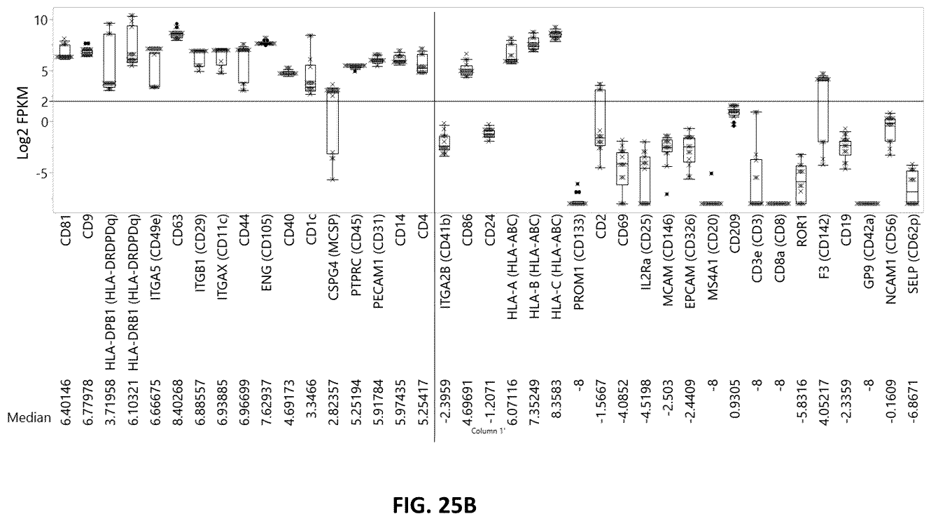

MGL-EV were evaluated using the MacsQuant Exosome kit, to investigate additional potential surface markers. AHN MGL-EV.UC and MGL-MV.UC control were incubated with the MACSPlex EV Kit and analyzed on a MACSQuant Analyzer 10 flow cytometer. Results depicted in FIG. 25A are given as the Median Fluorescence Intensity of the APC channel for each bead. Results show the presence of various proteins on the surface of MGL-EV.UC (lower panel of the FIG. 25A). As expected, no significant signal was detected in the MGL-MV.UC control for any of the markers (upper panel of FIG. 25A). Results are sorted from highest to lowest APC MFI for the MGL-EV.UC. A line is added to FIG. 25A at 15 APC MFI. Sixteen surface markers were detected at levels above 15. The highest APC MFI were for the CD81, CD9, HLA-DRDPDQ and CD49e markers. Note that bead-bound EV (bound to the bead by binding of the antibody to the EV-surface ligand) are being detected with a cocktail of tetraspanins (CD81, CD9 and CD63). Therefore, both the number of marker-positive EV bound to the bead and the number of tetraspanins on the bead-bound EV will influence the overall signal from each bead detected in this assay. For all of the markers with an APC MFI above 15 in FIG. 25A (high marker detection), median RNA expression levels for AHN MGL-Cells depicted in FIG. 25B were at or above 2.8 log 2 FPKM (high RNA expression). For all of the markers with an APC MFI less than 15 in FIG. 25A (low or no marker detection), median RNA expression levels for AHN MGL-Cells depicted in FIG. 25B were at or below 0.9305 (low or no RNA expression), with three exceptions. For these exceptions, the APC MFI for the EV were in the low or no marker detection category, whereas the gene expression results from the cells were in the high RNA expression category, suggesting that the proteins may be selectively excluded from the EV. These three exceptions are the HLA-ABC, CD86, and CD142 markers. The absence, or low to no expression of these protein markers in the MGL-EV represent a unique and surprising result which has implications for allogenic applications, such as, without limiting, reduced immunogenicity, immune evasion, reduced platelet activation and the like.

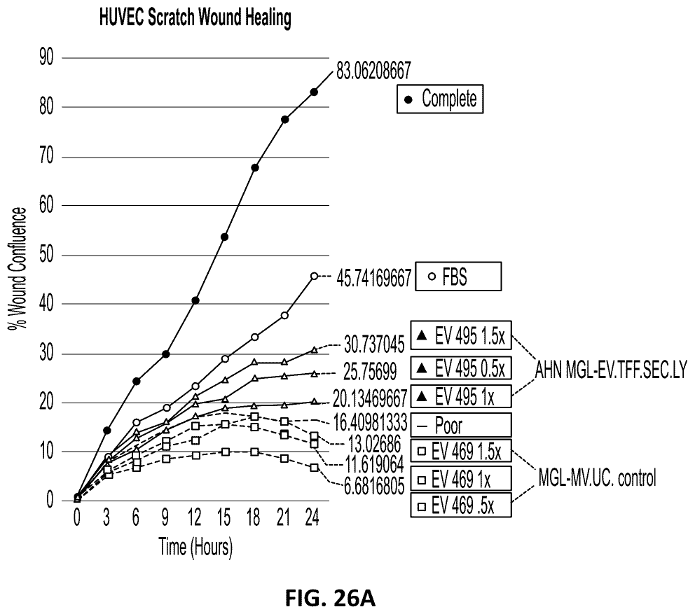

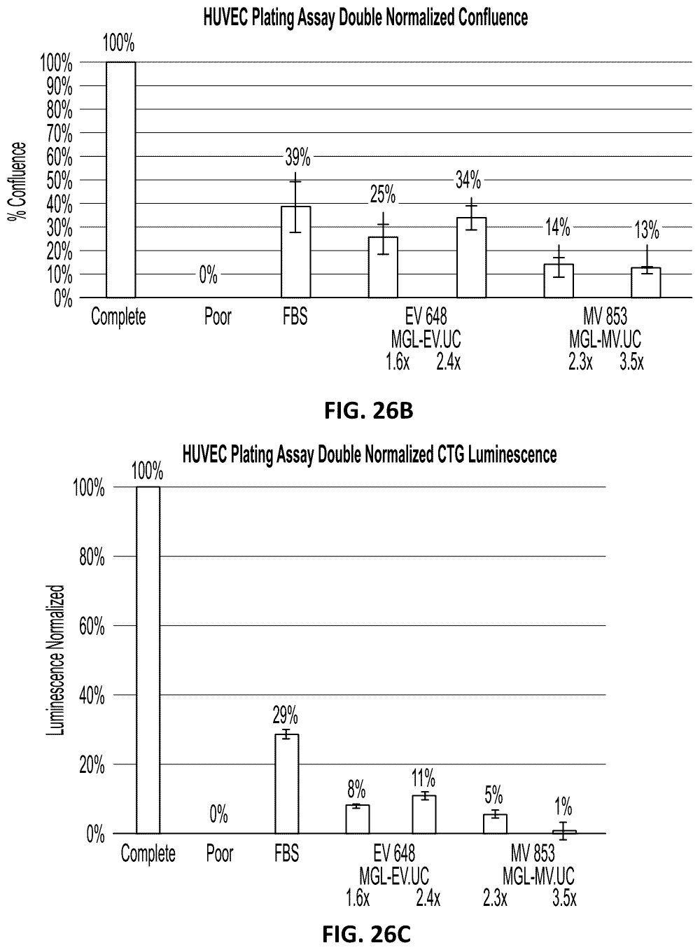

MGL-EV in vitro function was assayed by HUVEC scratch wound healing assay. AHN MGL-EV.TFF.SEC.LY in vitro function as measured by HUVEC scratch wound healing assay. A MGL-MV.UC control sample was included. All three doses of the MGL-EV.TFF.SEC.LY improved scratch wound healing beyond the Poor media control (FIG. 26A and Table 6). All three doses of the MGL-MV.UC resulted in less would healing than the Poor media control alone. The 24-hour time point results for all of the samples depicted in FIG. 26A are tabulated in Table 6. MGL-EV restored endothelial cell activation, proliferation and/or migration, resulting in partially restored scratch wound healing capabilities of stressed endothelial cells by up to 21.9% [baseline subtraction and normalization: 100%*(30.7-16.4)/(83.1-16.4)=21.9%].

TABLE 6
Description of the % wound confluence at 24-
hours for the analysis presented in FIG. 26A.

| | Additional | | % Wound |
|---|---|---|---|
| Sample Name | Labels | | Confluence |
| in FIG. 26 | in FIG. 26 | Dose | at 24-hours |
| Complete | | | 83.1 |
| FBS | | | 45.7 |
| Poor | | | 16.4 |
| EV 469 0.5x | MGL-MV.EV | 0.5x | 6.7 |
| | Control.UC | | |
| EV 469 1x | MGL-MV.EV | 1x | 11.6 |
| | Control.UC | | |
| EV 469 1.5x | MGL-MV.EV | 1.5x | 13 |
| | Control.UC | | |
| EV 495 0.5x | iPSC-MGL- | 0.5x | 25.8 |
| | EV.TFF.SEC.LY | | |
| EV 495 1x | iPSC-MGL- | 1x | 20.1 |
| | EV.TFF.SEC.LY | | |
| EV 495 2x | iPSC-MGL- | 1.5x | 30.7 |
| | EV.TFF.SEC.LY | | |

HUVEC cells are typically plated in HUVEC Complete Media. When they are plated in Endothelial Cell Basal Media alone (“Poor media”), they do not plate down, survive, and/or proliferate. To test if MGL-EV could promote HUVEC cell seeding/survival/proliferation, a HUVEC plating assay was employed. The results are depicted in FIG. 26B and FIG. 26C. FIG. 26B depicts the % confluence of each test condition, double normalized to Complete and Poor controls. FIG. 26C depicts the % luminescence of each test condition, after double normalization to Complete and Poor controls. Taken together, these two sets of analyses show that MGL-EV improve HUVEC cell survival/plating/proliferation under stressed conditions.

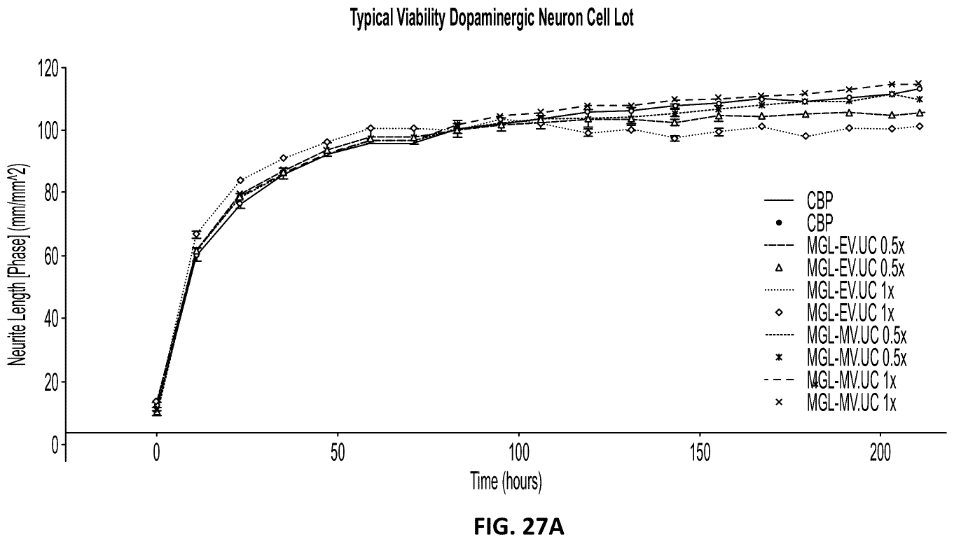

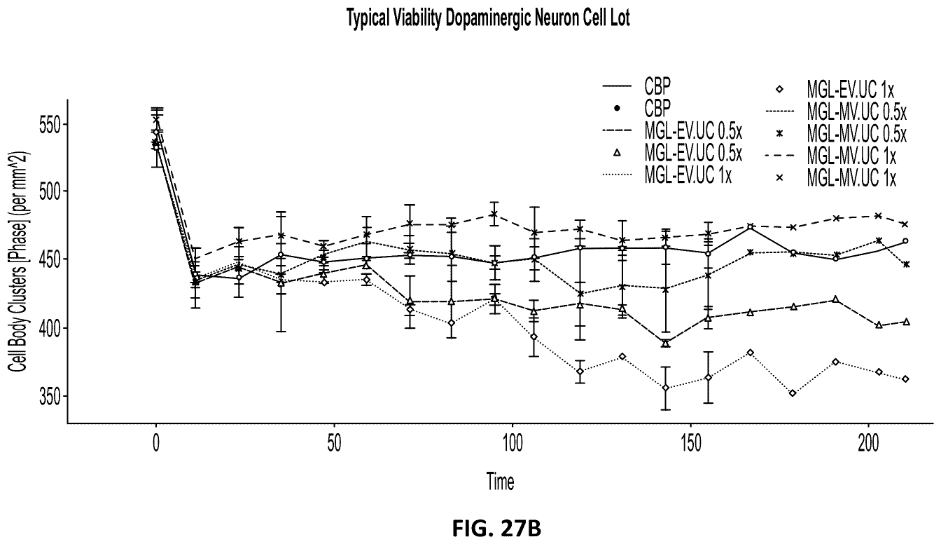

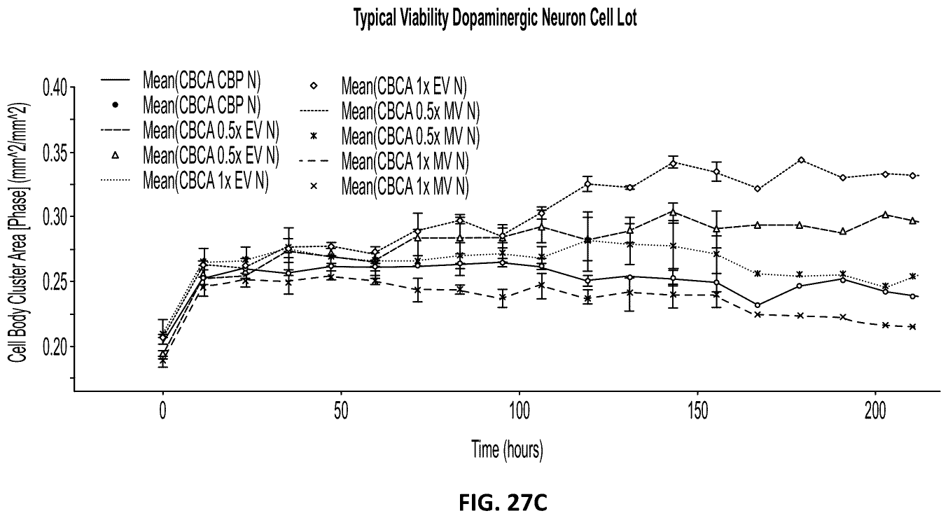

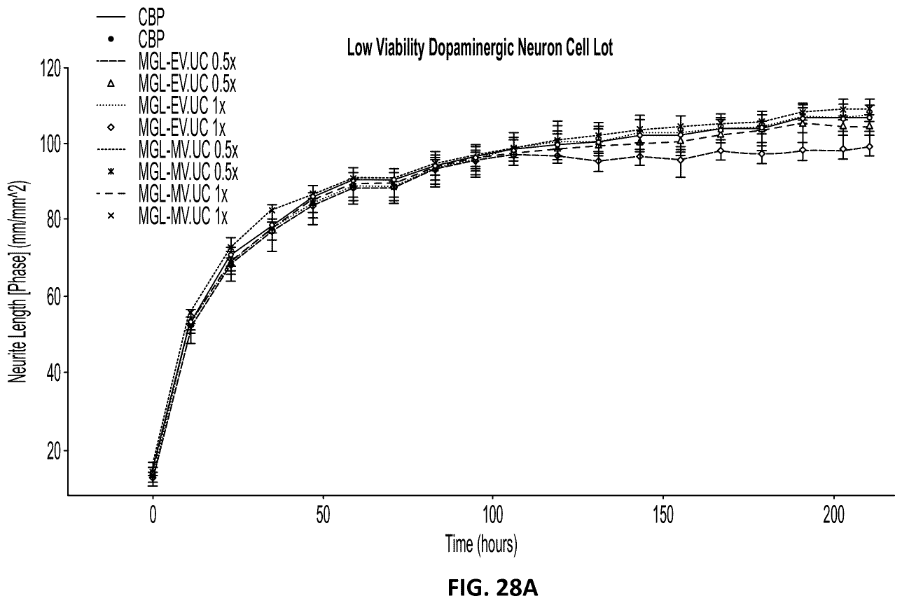

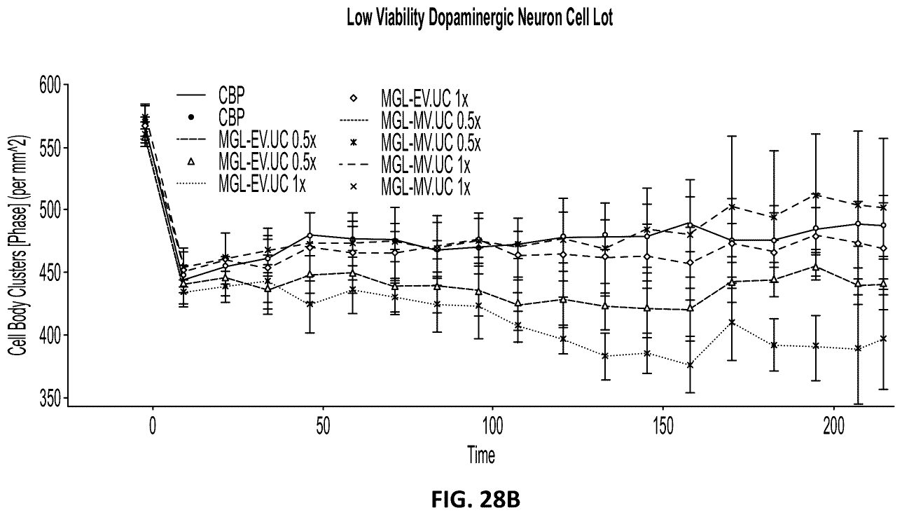

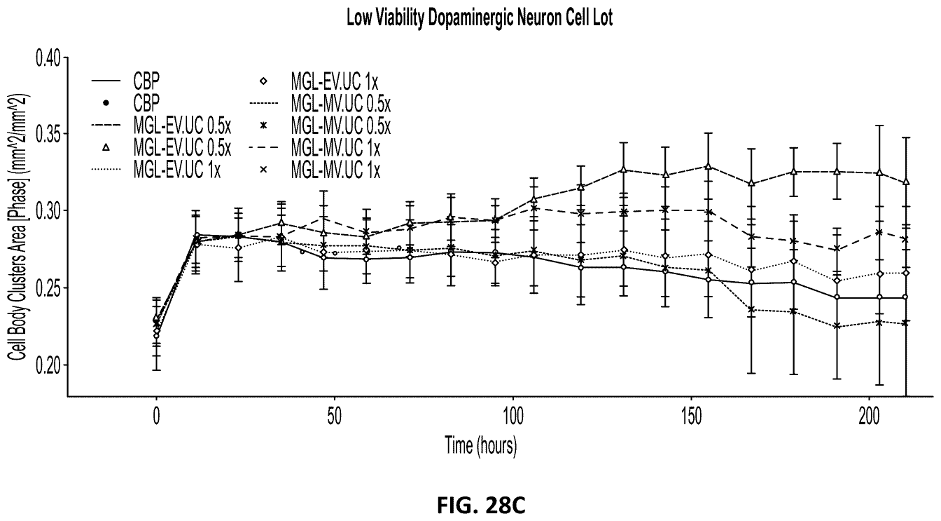

MGL-EV were tested on two different lots of iCell Dopaminergic Neuron cells for dopaminergic neuron viability and neurite outgrowth. One lot had a typical viability out of the thaw, and the other lot had a known low viability out of thaw. For both target cell lots, administration of MGL-EV resulted in an increase in dopaminergic neuron cell viability, shorter neurite length, decreased the number of cell body clusters, and increased cell body cluster area (FIGS. 27A, 27B, 27C, 28A, 28B, 28C).

TABLE 7
Effect of MGL-EVs on Dopaminergic neuron cell viability. iPSC-derived dopaminergic
neuron cells were plated in the presence or absence of MGL-EV.UC at two EV concentrations
(0.5 × MGL-EV.UC dose = the EV released from 32,000 microglia mother cells;
1 × MGL-EV.UC dose = the EV released from 64,000 microglia mother cells).
Additional MGL-EV.UC treatments were given on days 1, 3, and 5 post-plating. Viability
was accessed on day 7 when the cells were stained with YOYO-Iodide and Calcein AM
and evaluated on an Incucyte. Viable cell numbers and % viability was then calculated.

| | Day 7 Post-EV Treatment | | | Day 7 Post-EV Treatment | | |
|---|---|---|---|---|---|---|
| | Viable Cell number (% | | | % Viability (absolute | | |
| | increase over control) | | | increase over control) | | |
| | Untreated | 0.5 × MGL- | 1 × MGL- | Untreated | 0.5 × MGL- | 1 × MGL- |
| Target Cell Lot | Control | EV.UC | EV.UC | Control | EV.UC | EV.UC |
| Typical Viability Lot | 29118 — | 30772 (6%) | 32159 (10%) | 44% — | 48% (4%) | 46% (2%) |
| Low Viability Lot | 24163 — | 29215 (21%) | 28652 (19%) | 32% — | 37% (5%) | 36% (4%) |

TABLE 8
Effect of MGL-EV on neurite length in a typical viability dopaminergic neuron cell lot. Data used to generate the graph depicted in FIG. 27A.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 9.9 | 59.3 | 76.3 | 86.1 | 91.8 | 95.8 | 95.8 | 100.0 | 101.3 | 103.3 | 105.5 | 106.0 | 107.4 | 108.2 | 109.8 | 108.9 | 110.0 | 111.3 | 112.9 |
| MGL- | 9.9 | 61.9 | 79.4 | 86.8 | 93.4 | 97.6 | 97.6 | 99.7 | 101.3 | 101.9 | 103.3 | 103.5 | 102.3 | 104.5 | 104.1 | 104.7 | 105.3 | 104.5 | 105.2 |
| EV.UC 0.5× | | | | | | | | | | | | | | | | | | | |
| MGL- | 13.9 | 66.8 | 83.9 | 90.8 | 95.8 | 100.3 | 100.3 | 99.7 | 103.3 | 102.1 | 98.7 | 99.9 | 97.4 | 99.3 | 100.9 | 97.7 | 100.5 | 100.2 | 101.0 |
| EV.UC 1× | | | | | | | | | | | | | | | | | | | |
| MGL- | 10.8 | 61.8 | 78.7 | 85.6 | 92.4 | 96.6 | 96.6 | 100.5 | 101.8 | 103.8 | 103.3 | 103.9 | 105.0 | 106.4 | 107.8 | 108.9 | 109.1 | 110.9 | 109.6 |
| MV.UC 0.5× | | | | | | | | | | | | | | | | | | | |
| MGL- | 11.8 | 61.4 | 77.6 | 86.2 | 91.7 | 96.1 | 96.1 | 101.7 | 104.3 | 105.6 | 107.5 | 107.6 | 109.3 | 109.8 | 110.6 | 111.4 | 112.7 | 114.5 | 114.2 |
| MV.UC 1× | | | | | | | | | | | | | | | | | | | |

TABLE 9
Effect of MGL-EV on cell body clusters in a typical viability Dopa cell lot. Data used to generate graph depicted in FIG. 27B.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 533.1 | 439.5 | 436.4 | 454.0 | 449.0 | 451.4 | 453.9 | 452.8 | 448.2 | 451.7 | 458.9 | 458.4 | 459.2 | 454.9 | 473.6 | 454.9 | 450.7 | 457.1 | 463.5 |
| MGL- | 538.5 | 433.3 | 445.9 | 432.6 | 440.0 | 446.5 | 419.4 | 419.2 | 421.2 | 412.5 | 417.6 | 413.6 | 388.6 | 407.6 | 411.6 | 415.1 | 420.9 | 401.4 | 404.9 |
| EV.UC 0.5× | | | | | | | | | | | | | | | | | | | |
| MGL- | 552.9 | 436.7 | 448.4 | 435.3 | 432.9 | 435.7 | 413.5 | 403.0 | 421.1 | 393.1 | 367.5 | 378.3 | 355.1 | 363.0 | 381.4 | 351.5 | 374.7 | 366.7 | 361.7 |
| EV.UC 1× | | | | | | | | | | | | | | | | | | | |
| MGL- | 549.5 | 432.3 | 443.7 | 439.6 | 453.5 | 463.2 | 457.8 | 454.7 | 448.0 | 450.6 | 425.9 | 430.8 | 428.6 | 438.7 | 455.0 | 455.6 | 453.6 | 463.8 | 446.5 |
| MV.UC 0.5× | | | | | | | | | | | | | | | | | | | |
| MGL- | 541.6 | 450.4 | 463.5 | 468.3 | 460.1 | 469.0 | 476.8 | 475.9 | 483.9 | 470.1 | 472.8 | 464.9 | 466.6 | 469.5 | 475.4 | 473.9 | 481.7 | 482.7 | 476.4 |
| MV.UC 1× | | | | | | | | | | | | | | | | | | | |

TABLE 10
Effect of MGL-EV on cell body cluster area in a typical viability Dopa cell lot. Data used to generate graph depicted in FIG. 27C.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 0.204 | 0.253 | 0.260 | 0.256 | 0.261 | 0.261 | 0.262 | 0.263 | 0.265 | 0.262 | 0.251 | 0.254 | 0.253 | 0.249 | 0.232 | 0.246 | 0.252 | 0.242 | 0.239 |
| MGL- | 0.193 | 0.253 | 0.256 | 0.274 | 0.269 | 0.265 | 0.285 | 0.283 | 0.284 | 0.293 | 0.283 | 0.290 | 0.304 | 0.290 | 0.293 | 0.293 | 0.288 | 0.302 | 0.297 |
| EV.UC 0.5× | | | | | | | | | | | | | | | | | | | |
| MGL- | 0.205 | 0.263 | 0.262 | 0.276 | 0.278 | 0.272 | 0.290 | 0.297 | 0.285 | 0.303 | 0.326 | 0.323 | 0.342 | 0.335 | 0.322 | 0.344 | 0.329 | 0.334 | 0.331 |
| EV.UC 1× | | | | | | | | | | | | | | | | | | | |
| MGL- | 0.211 | 0.265 | 0.267 | 0.276 | 0.269 | 0.266 | 0.266 | 0.270 | 0.271 | 0.268 | 0.281 | 0.279 | 0.278 | 0.271 | 0.256 | 0.254 | 0.256 | 0.245 | 0.255 |
| MV.UC 0.5× | | | | | | | | | | | | | | | | | | | |
| MGL- | 0.188 | 0.246 | 0.250 | 0.250 | 0.255 | 0.250 | 0.243 | 0.244 | 0.237 | 0.247 | 0.238 | 0.242 | 0.239 | 0.239 | 0.223 | 0.224 | 0.221 | 0.216 | 0.215 |
| MV.UC 1× | | | | | | | | | | | | | | | | | | | |

TABLE 11
Effect of MGL-EVs on neurite length in a low viability Dopa cell lot. Data used to generate graph depicted in FIG. 28A.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 12.4 | 51.9 | 69.2 | 77.8 | 85.4 | 89.7 | 89.7 | 94.0 | 95.9 | 98.9 | 101.0 | 102.0 | 103.5 | 104.6 | 104.9 | 105.5 | 108.2 | 108.8 | 109.1 |
| MGL- | 13.5 | 52.5 | 70.5 | 78.7 | 86.0 | 90.7 | 90.7 | 94.3 | 96.1 | 97.2 | 98.9 | 99.4 | 99.7 | 100.7 | 102.2 | 103.5 | 105.5 | 104.3 | 104.6 |
| EV.UC 0.5× | | | | | | | | | | | | | | | | | | | |
| MGL- | 15.4 | 54.9 | 72.8 | 82.2 | 86.8 | 91.3 | 91.3 | 94.7 | 96.8 | 97.3 | 96.5 | 95.4 | 96.6 | 95.7 | 98.1 | 97.2 | 98.1 | 98.2 | 99.3 |
| EV.UC 1× | | | | | | | | | | | | | | | | | | | |
| MGL- | 13.7 | 51.7 | 69.0 | 76.6 | 84.5 | 88.4 | 88.4 | 93.5 | 96.0 | 98.5 | 99.4 | 100.5 | 102.3 | 102.1 | 104.0 | 104.3 | 106.6 | 106.9 | 107.2 |
| MV.UC 0.5× | | | | | | | | | | | | | | | | | | | |
| MGL- | 12.8 | 51.7 | 68.2 | 77.1 | 83.9 | 88.4 | 88.4 | 93.1 | 95.9 | 98.5 | 100.5 | 100.6 | 102.9 | 102.8 | 103.6 | 104.0 | 106.5 | 106.4 | 106.8 |
| MV.UC 1× | | | | | | | | | | | | | | | | | | | |

TABLE 12
Effect of MGL-EV on cell body clusters in a low viability Dopa cell lot. Data used to generate graph depicted in FIG. 28B.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 570.2 | 448.1 | 454.6 | 462.3 | 480.3 | 477.1 | 476.0 | 468.5 | 469.8 | 472.4 | 478.1 | 480.4 | 479.8 | 488.8 | 476.2 | 476.0 | 485.0 | 489.1 | 487.7 |
| MGL- | 563.9 | 440.9 | 445.7 | 436.9 | 448.8 | 450.9 | 438.8 | 440.1 | 436.0 | 424.5 | 429.1 | 423.4 | 421.4 | 421.0 | 442.6 | 444.5 | 455.4 | 439.6 | 441.2 |
| EV.UC 0.5× | | | | | | | | | | | | | | | | | | | |
| MGL- | 559.7 | 434.0 | 438.9 | 443.1 | 424.7 | 437.0 | 430.6 | 425.2 | 423.5 | 407.3 | 397.2 | 383.6 | 386.0 | 376.7 | 410.5 | 392.6 | 390.4 | 389.2 | 397.2 |
| EV.UC 1× | | | | | | | | | | | | | | | | | | | |
| MGL- | 564.7 | 450.6 | 461.3 | 453.1 | 470.7 | 465.7 | 466.0 | 470.3 | 475.8 | 463.5 | 465.2 | 462.3 | 463.2 | 457.4 | 473.9 | 467.1 | 480.0 | 474.1 | 469.0 |
| MV.UC 0.5× | | | | | | | | | | | | | | | | | | | |
| MGL- | 574.8 | 454.4 | 461.1 | 468.4 | 473.5 | 473.5 | 474.2 | 470.7 | 476.6 | 470.7 | 476.8 | 469.9 | 484.2 | 481.0 | 502.9 | 495.0 | 512.3 | 504.4 | 500.9 |
| MV.UC 1× | | | | | | | | | | | | | | | | | | | |

TABLE 13
Effect of MGL-EV on cell body cluster area in a low viability Dopa cell lot. Data used to generate graph depicted in FIG. 28C.

| | Time | | | | | | | | | | | | | | | | | | |
|---|---|---|---|---|---|---|---|---|---|---|---|---|---|---|---|---|---|---|---|
| | 0 | 11 | 23 | 35 | 47 | 59 | 71 | 83 | 95 | 106 | 119 | 131 | 143 | 155 | 167 | 179 | 191 | 203 | 210.5 |
| CBP | 0.217 | 0.284 | 0.283 | 0.279 | 0.269 | 0.268 | 0.269 | 0.273 | 0.272 | 0.269 | 0.263 | 0.263 | 0.260 | 0.255 | 0.252 | 0.253 | 0.243 | 0.243 | 0.243 |
| MGL- | 0.228 | 0.282 | 0.284 | 0.292 | 0.286 | 0.282 | 0.291 | 0.292 | 0.293 | 0.301 | 0.298 | 0.299 | 0.301 | 0.300 | 0.284 | 0.280 | 0.274 | 0.286 | 0.282 |
| EV.UC 0.5× | | | | | | | | | | | | | | | | | | | |
| MGL- | 0.226 | 0.280 | 0.283 | 0.282 | 0.295 | 0.285 | 0.289 | 0.295 | 0.293 | 0.307 | 0.314 | 0.326 | 0.322 | 0.329 | 0.317 | 0.325 | 0.325 | 0.324 | 0.318 |
| EV.UC 1× | | | | | | | | | | | | | | | | | | | |
| MGL- | 0.219 | 0.278 | 0.274 | 0.282 | 0.272 | 0.273 | 0.274 | 0.271 | 0.267 | 0.271 | 0.271 | 0.274 | 0.269 | 0.271 | 0.261 | 0.267 | 0.254 | 0.259 | 0.260 |
| MV.UC 0.5× | | | | | | | | | | | | | | | | | | | |
| MGL- | 0.224 | 0.281 | 0.282 | 0.279 | 0.277 | 0.277 | 0.275 | 0.276 | 0.270 | 0.273 | 0.267 | 0.271 | 0.262 | 0.261 | 0.235 | 0.234 | 0.224 | 0.227 | 0.227 |
| MV.UC 1× | | | | | | | | | | | | | | | | | | | |

NeuroSphere electrical activity was evaluated. FIG. 29 shows that when incubated with MGL-EV, the electrical traces were altered in several ways, notably, resulting in decreased amplitude.

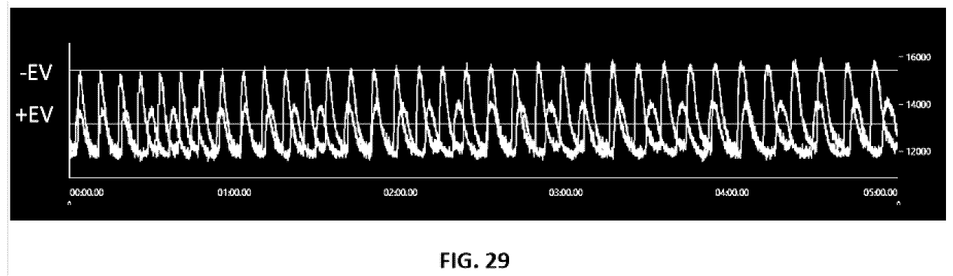

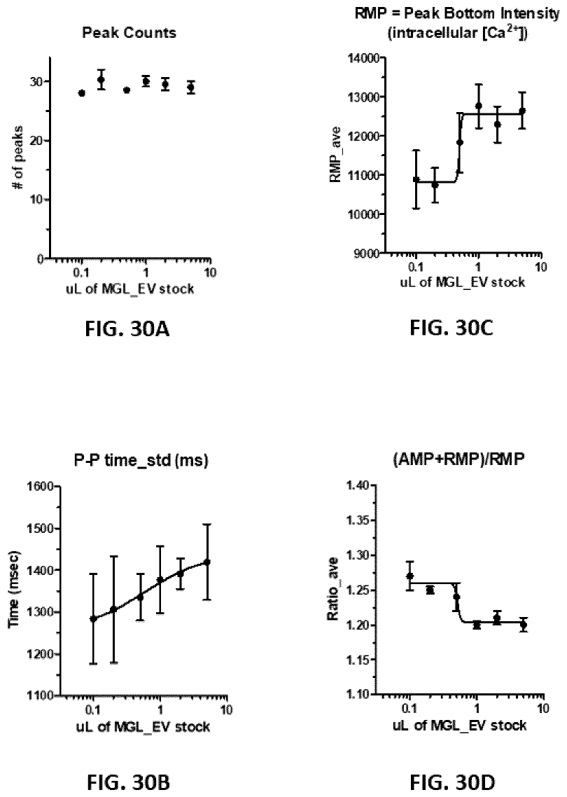

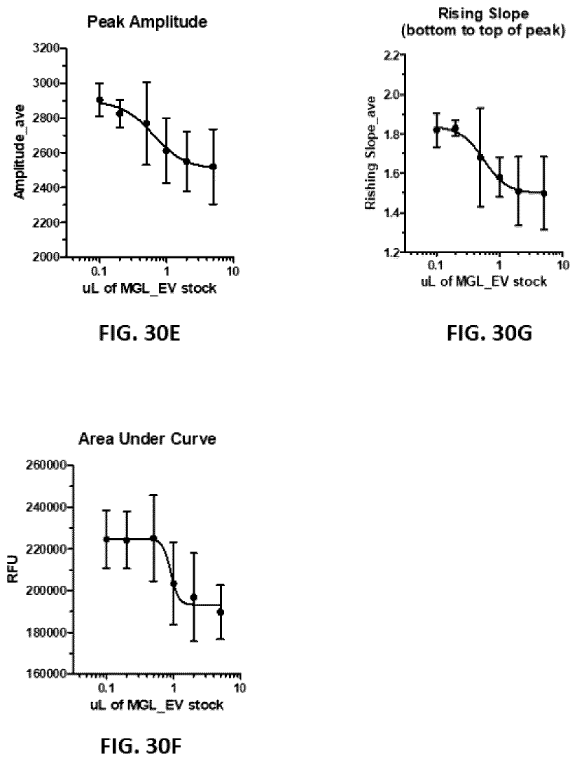

Numerous parameters were analyzed and shown to be affected by MGL-EV treatment, in a dose-dependent manner (FIG. 30A-30G).

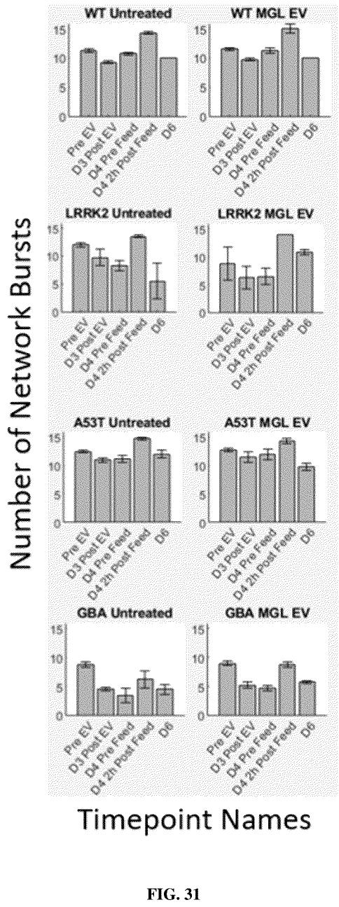

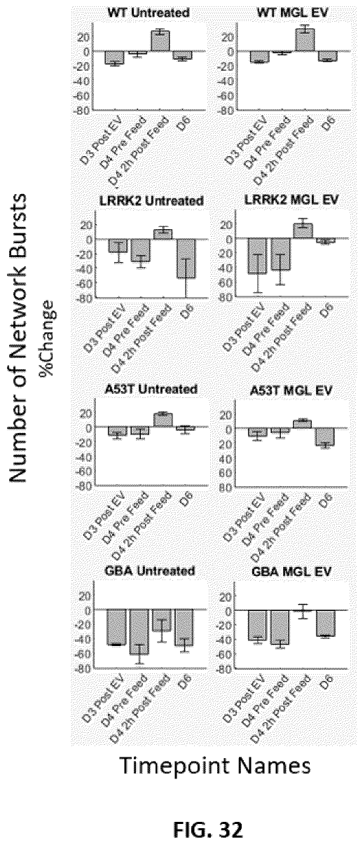

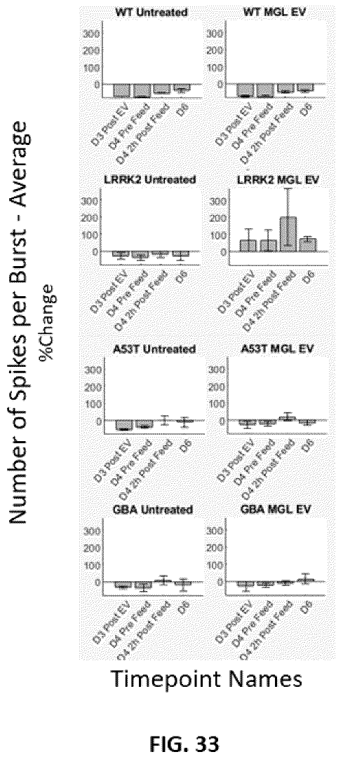

In addition, assessment of the impact of MGL-EVs on the electrophysiology of monocultures of AHN and iPSC-dopaminergic neurons with Parkinson's disease mutations was performed. MGL-EV increased the number of network bursts in LRRK2 mutant dopaminergic neurons especially 6 days after EV treatment (FIG. 31). An effect was also seen in this metric when normalized to baseline (“Pre EV” timepoint) values for each data set. (FIG. 32). Additionally, MGL-EV increased the number of spikes per burst relative to baseline, in LRRK2 mutants, compared to untreated LRKK2 controls when the data were normalized to baseline values (FIG. 33).

From the present studies, it was shown that MGL-EV are larger (median=112 nm) than expected compared to a diverse set of EV secreted by iPSCs and iPSC-derived cell types (median=104.05 nm) (FIG. 4C and FIG. 4D). In addition, MGL-EV have a unique tetraspanin signature that distinguished them from other iPSC-derived differentiated cell type-EV. When using the ELISA method 2 and the tetraspanin signature calculation method, iPSC-derived microglia EV occupy their own space on the plot (FIG. 9), and therefore have a unique signature, when compared to EVs from other iPSC-derived differentiated cells. The unique signature of iPSC-derived Microglia EV derived from this method is between 0.5 and 8% CD63, between 36 and 62% CD9, and between 34 and 60% CD81, wherein each individual sample's three values add up to 100%.

When using this ELISA method 2 and signature calculation, to be considered an iPSC-derived microglia EV the sample should have a signature that falls within this signature range (FIG. 9). When using this method to characterize EV from differentiated iPSC-derived cells, a CD63 value of 6% or below can be convincingly classified as an iPSC-derived microglia EV (FIG. 9). This ELISA method and signature calculation method shows that there is a signature specific to iPSC-derived microglia EV that is not dependent on specific iPSC line, phenotype, or genotype (FIG. 8A, FIG. 8B, FIG. 9). This ELISA method and signature calculation method shows that there is a signature specific to iPSC-derived microglia EV that is not dependent on the specific isolation method (FIG. 7A, FIG. 7B).

It was further shown that the proportion of cluster sub-types in MGL-EV was distinct from other iPSC-derived cell-EV (Table 3). The overall miR content of MGL-EV was distinct from other iPSC-EV and iPSC-derived cell-EV (FIG. 11). The top 25 most abundant miR identified in MGL-EV by small RNA sequencing were hsa-miR-16-5p, hsa-miR-223-3p, hsa-miR-93-5p, hsa-miR-146a-5p, hsa-miR-142-3p, hsa-miR-191-5p, hsa-miR-142-5p, hsa-miR-21-5p, hsa-miR-103a-3p/107, hsa-miR-26b-5p, hsa-miR-122-5p, hsa-miR-125b-5p, hsa-miR-25-3p, hsa-miR-146b-5p, hsa-miR-101-3p, hsa-miR-29a-3p, hsa-miR-30e-5p, hsa-let-7a-5p/7c-5p, hsa-miR-342-3p, hsa-miR-148b-3p, hsa-miR-27a-3p/27b-3p, hsa-miR-224-5p, hsa-let-7f-5p, hsa-miR-125a-5p, and/or hsa-miR-26a-5p (Table 4, Table 8).

The top 100 miR in MGL-EV were predicted to be localized to extracellular vesicles (FIG. 12A). The miR content of the MGL-EV was associated with extracellular vesicles; where 6 of the 10 terms are synonyms or subsets of extracellular vesicles (FIG. 12A). The predicted mRNA targets of the miR identified in MGL-EV were predicted to impact fundamental biological pathways, including “response to oxygen levels”, “intrinsic apoptotic signaling pathway” and “cell cycle arrest” (FIG. 12B).

MGL-EV comprise hsa-miR-4669 and hsa-miR-4777-3p, which is unique compared to a set of iPSC-EV and iPSC-derived cell-type-EV (FIG. 13).

MGL-EV comprise extracellular vesicles discernable by cryo-electron microscopy that contain a single lipid-bilayer (FIG. 14D), and extracellular vesicles discernable by cryo-electron microscopy that contain two (FIG. 14A) or more (FIG. 14G) nested lipid bilayer structures, including four nested and discernable lipid-bilayers (FIG. 14G) that may be approximately concentric, approximately spherical or clearly not spherical (FIG. 14C, FIG. 14E) (FIG. 14A-G). MGL-EV contain EV proteins, including CD9, CD63, CD81, beta-actin, Flottilin-1 (FIGS. 5A, 6, 7A, 7B, 8A, 8B, 9, 10, 15, 16. 17, and 18).

MGL-EV contain proteins, that when mutated, are associated with Parkinson's disease, including GBA (FIG. 21A and FIG. 21B), LRRK2 (FIG. 23), and phosphor-LRRK2 (FIG. 24). The GBA detected in MGL-cells is larger than the GBA detected in cells that are a non-MGL, iPSC-derived neuron cell-type, the latter having the expected GBA protein size (FIGS. 21A and B, and FIGS. 22A and B).

MGL-EV and MGL-cells contain both the full length TREM2 protein and a TREM2 C-terminal cleavage product (FIG. 19, FIG. 20). The intracellular domain cleavage product (C-terminal fragment) has been described in microglia, but has no reported function. Cleavage by the γ-secretase to release the intracellular domain occurs downstream of N-terminus shedding which releases the TREM2 signal peptide (soluble TREM2, sTREM2). Next further processing is needed to release binding partners (e.g., DAP12) so they can be available for binding to full length TREM2 (Wunderlich et al., 2013). After release of the sTREM2, the remaining cleavage products would need to be processed. The data show that they are enriched in EV. The presence of the TREM2 C-terminal fragment in EV could be a marker of functional TREM2 processing in microglia. Indeed, reduced signal peptide release has been shown to cause an increase in neuroinflammation in mouse models (Dhandapani et al., 2022). It is also possible that the C-terminal fragment has its own function. For example, the intracellular domain cleavage product of Notch (also cleaved by gamma secretase) acts as a transcription factor (Hur, 2022). EV deliver their content to target cells. The C-terminal fragment would also be delivered to MGL-EV target cells and could play an important paracrine signaling role. There may also be a connection between the sTREM2 peptide and EV since sTREM2 bind phosphatidyl serine (Sudom et al., 2022), which is on the surface of many extracellular vesicles.

MGL-EV contain 16 out of 37 surface markers interrogated by the MACSplex exosome kit (FIG. 25A). For all of the highly detected markers in MGL-EV the microglia cells were found to have high median RNA expression of the corresponding genes (FIG. 25B), further corroborating the presence of the protein on the EV. The inverse was also true, where for low/not detected markers in MGL-EV, the microglia cells were found to have low median RNA expression of the corresponding genes with three notable exceptions. Despite showing high RNA expression levels in the microglia cells, little to no HLA-ABC, CD86, or CD142 was detected on MGL-EV, suggesting that these proteins are actively excluded from the MGL-EV.

Further, the present studies showed that MGL-EV improve or restore endothelial cell health and biology. MGL-EV restored endothelial cell activation, proliferation and/or migration, resulting in restoration of up to 21.9% of scratch wound healing capabilities in stressed endothelial cells (FIG. 26A). MGL-EV also restored plating capability, viability and/or proliferation of stressed endothelial cells, restoring 34% of cell confluence at 24-hours post-plating (FIG. 26B) and up to 11% of cell number as measured by intracellular ATP (FIG. 26C.

MGL-EV also improved dopaminergic neuron cell health. MGL-EV increased dopaminergic neuron cell viability by up to 21% (Table 7). In addition, surprisingly and unexpectedly, MGL-EV promoted dopaminergic neuron clustering. Specifically, MGL-EV decreased the number of iPSC-derived dopaminergic neuron cell body clusters (FIG. 27B and FIG. 28B) and increased iPSC-derived dopaminergic neuron cell body cluster area (FIG. 27C and FIG. 28C). Further, MGL-EV affected neurite outgrowth of dopaminergic neurons. MGL-EV.UC decreased iPSC-derived dopaminergic neuron neurite length (FIG. 27A and FIG. 28A).

Further, MGL-EV affect the Ca²⁺ signaling and/or electrical signaling of neurons. Treatment of neurospheres with MGL-EV resulted in decreased peak amplitude and altered the rising slope (from the bottom to the top of the peak), indicating an effect on the calcium handling activity (FIG. 29), in a dose-dependent manner. Specifically, MGL-EV-treated neurospheres had increased “P-P time_std” (i.e., the standard deviation for peak-to-peak timing) which increased, meaning the irregularity of the oscillations increased (FIG. 30B). In addition, the MGL-EV-treated neurospheres had increased “RMP” (i.e., the peak bottom intensity, which is a measure of intracellular [Ca²⁺] concentration) (FIG. 30C), and decreased “(AMP+RMP)/RMP” (i.e., the ratio of peak amplitude to peak bottom) (FIG. 30D). “Peak Amplitude” (i.e., the difference in signal from top of the peak to the bottom) was found to decrease with an increased number of EV used (FIG. 30E) and “Area Under Curve” (i.e., the total peak area) also decreased with increased number of EV used (FIG. 30F). In addition, the “Rising Slope” (also known as the upstroke slope with respect to depolarization) was decreased/less sharp with increased number of EV used (FIG. 30G). It was found that MGL-EV impact the electrophysiology of AHN and iPSC-dopaminergic neurons with Parkinson's Disease mutations (FIG. 31-FIG. 32 and FIG. 33) and MGL-EV increase the number of spikes per burst in LRRK2 mutants, relative to pre-treatment values (FIG. 33).

TABLE 14
miRNA content of AHN MGL-EV. The table shows the log2 RPM (Reads Per Million mapped reads)
values for all known miRNA in the human genome from 4 MGL-EV.UC samples and 1 MGL-EV.TFF.SEC.LY
sample. If no reads were mapped to a miR, a value of 10e−6 was assigned to allow the
log2 to be taken. Samples with no reads detected have a value of −16.61 in the table.

| | RNA Sample Name (Preparation Method) | | | | |
|---|---|---|---|---|---|
| miRNA | 50evrna (TFF.SEC.LY) | 58evrna (UC) | 59evrna (UC) | 60evrna (UC) | 95evrna (UC) |
| hsa-let-7a-2-3p | −16.61 | −16.61 | 1.187 | −16.61 | −16.61 |
| hsa-let-7a-3p | 6.183 | 2.442 | 4.235 | 5.297 | −16.61 |
| hsa-let-7a-5p/7c-5p | 13.882 | 13.318 | 12.822 | 13.373 | 15.427 |
| hsa-let-7b-3p | −3.156 | −16.61 | −16.61 | −16.61 | −16.61 |
| hsa-let-7b-5p | 8.296 | 10.055 | 6.071 | 8.384 | 9.298 |
| hsa-let-7c-3p | 4.414 | 1.705 | 1.534 | 2.941 | −16.61 |
| hsa-let-7d-3p | 3.318 | 2.622 | 3.634 | 4.761 | 4.173 |
| hsa-let-7d-5p | 4.745 | 6.684 | 5.704 | 5.931 | 10.335 |
| hsa-let-7e-3p | 4.124 | 1.12 | 0.897 | −1.146 | 2.588 |
| hsa-let-7e-5p | 13.168 | 10.959 | 10.376 | 10.557 | 12.41 |
| hsa-let-7f-1-3p | −16.61 | −16.61 | 0.534 | −16.61 | −16.61 |
| hsa-let-7f-2-3p | −16.61 | −16.61 | −16.61 | 3.635 | −16.61 |
| hsa-let-7f-5p | 12.416 | 12.922 | 12.593 | 13.226 | 14.131 |
| hsa-let-7g-3p | 3.391 | 3.29 | 2.049 | 3.246 | 3.687 |
| hsa-let-7g-5p | 9.84 | 9.939 | 10.12 | 10.693 | 15.029 |
| hsa-let-7i-3p | −16.61 | 0.12 | 3.153 | 3.211 | −16.61 |
| hsa-let-7i-5p | 10.394 | 11.355 | 11.582 | 12.998 | 13.615 |
| hsa-miR-1-3p | 6.756 | 13.528 | 9.654 | 9.472 | 6.847 |
| hsa-miR-1-5p | −16.61 | 3.664 | −16.61 | −16.61 | −16.61 |
| hsa-miR-100-3p | 4.649 | 1.705 | 0.534 | −1.146 | −16.61 |
| hsa-miR-100-5p | 8.781 | 7.71 | 5.441 | 5.102 | 5.91 |
| hsa-miR-101-2-5p | −16.61 | −16.61 | 0.727 | 1.176 | −16.61 |
| hsa-miR-101-3p | 13.788 | 13.307 | 13.606 | 13.511 | 7.638 |
| hsa-miR-101-5p | 3.422 | 3.961 | 2.187 | 2.898 | −16.61 |
| hsa-miR-10226 | −16.61 | −16.61 | −16.61 | −16.61 | −16.61 |
| hsa-miR-10392-3p | −16.61 | −16.61 | −16.61 | −16.61 | −16.61 |
| hsa-miR-10392-5p | −16.61 | −16.61 | −16.61 | −16.61 | −16.61 |
| hsa-miR-10393-3p | 2.766 | −16.61 | 1.187 | 1.313 | −16.61 |
| hsa-miR-10393-5p | −16.61 | −16.61 | −16.61 | −16.61 | −16.61 |
| hsa-miR-10394-3p | −16.61 | −16.61 | −16.61 | −16.61 | −16.61 |
| hsa-miR-10394-5p | −16.61 | −16.61 | −16.61 | −16.61 | −16.61 |
| hsa-miR-10395-3p | 0.943 | −16.61 | −16.61 | 2.246 | −16.61 |
| hsa-miR-10395-5p | −16.61 | −16.61 | −16.61 | −16.61 | −16.61 |
| hsa-miR-10396a-3p/ | −16.61 | 0.857 | 0.049 | −16.61 | −16.61 |
| 10396b-3p | | | | | |
| hsa-miR-10396a-5p/ | −16.61 | −16.61 | 2.049 | −16.61 | −16.61 |
| 10396b-5p | | | | | |
| hsa-miR-10397-3p | −16.61 | −16.61 | −16.61 | −16.61 | −16.61 |
| hsa-miR-10397-5p | −16.61 | −16.61 | −16.61 | −16.61 | −16.61 |
| hsa-miR-10398-3p | −16.61 | −16.61 | 3.085 | 2.246 | 3.481 |
| hsa-miR-10398-5p | −16.61 | −16.61 | −16.61 | −16.61 | −16.61 |
| hsa-miR-10399-3p | 3.237 | −16.61 | 2.727 | 2.898 | 4.102 |
| hsa-miR-10399-5p | −16.61 | −16.61 | 3.012 | 3.941 | 5.78 |
| hsa-miR-103a-1-5p/ | 2.107 | 0.535 | 0.312 | 0.661 | −16.61 |
| 103a-2-5p | | | | | |
| hsa-miR-103a-3p/107 | 13.979 | 13.754 | 14.612 | 14.339 | 14.673 |
| hsa-miR-103b | −16.61 | −16.61 | −16.61 | −16.61 | −16.61 |
| hsa-miR-10400-3p | −16.61 | −16.61 | −16.61 | −16.61 | −16.61 |
| hsa-miR-10400-5p | −16.61 | −16.61 | −16.61 | −16.61 | −16.61 |
| hsa-miR-10401-3p | 2.452 | 4.535 | 1.897 | 1.761 | 3.481 |
| hsa-miR-10401-5p | −16.61 | −16.61 | −16.61 | −16.61 | −16.61 |
| hsa-miR-105-3p | 4.924 | 1.994 | −16.61 | −16.61 | −16.61 |
| hsa-miR-105-5p | 6.443 | 5.164 | 3.049 | 3.313 | −16.61 |
| hsa-miR-10522-5p | −16.61 | −16.61 | −16.61 | −16.61 | −16.61 |
| hsa-miR-10523-5p | −16.61 | −16.61 | −16.61 | −16.61 | −16.61 |
| hsa-miR-10524-5p | −16.61 | −16.61 | −16.61 | −16.61 | −16.61 |
| hsa-miR-10525-3p | −16.61 | −16.61 | 16.61 | −16.61 | −16.61 |
| hsa-miR-10526-3p | −16.61 | −16.61 | −16.61 | −16.61 | −16.61 |
| hsa-miR-10527-5p | 4.52 | 3.442 | 2.771 | 3.808 | 5.102 |
| hsa-miR-106a-3p | 3.996 | 4.465 | 4.049 | 4.003 | −16.61 |
| hsa-miR-106a-5p/17-5p | 8.456 | 8.889 | 8.068 | 8.034 | 8.973 |
| hsa-miR-106b-3p | 7.518 | 7.734 | 8.636 | 8.84 | 6.102 |
| hsa-miR-106b-5p | 6.182 | 5.235 | 5.622 | 4.962 | 9.07 |
| hsa-miR-10a-3p | 3.854 | 7.617 | 6.655 | 7.185 | 5.028 |
| hsa-miR-10a-5p | 7.037 | 11.275 | 10.024 | 9.796 | 9.657 |
| hsa-miR-10b-3p | 16.61 | 6.633 | 2.371 | 2.102 | −16.61 |
| hsa-miR-10b-5p | 2.503 | 9.93 | 5.83 | 5.476 | −16.61 |
| hsa-miR-11181-3p | −16.61 | −16.61 | −16.61 | −16.61 | −16.61 |
| hsa-miR-11181-5p | −16.61 | −16.61 | −16.61 | −16.61 | −16.61 |
| hsa-miR-11399 | −16.61 | −16.61 | −16.61 | 2.854 | −16.61 |
| hsa-miR-11400 | −16.61 | −16.61 | −16.61 | −16.61 | −16.61 |
| hsa-miR-11401 | −16.61 | −16.61 | −16.61 | −16.61 | −16.61 |
| hsa-miR-1178-3p | −16.61 | −16.61 | −16.61 | −16.61 | −16.61 |
| hsa-miR-1178-5p | −16.61 | −16.61 | −16.61 | −16.61 | −16.61 |
| hsa-miR-1179-3p | −16.61 | −16.61 | −16.61 | −16.61 | −16.61 |
| hsa-miR-1179-5p | 6.549 | 5.811 | 2.049 | 2.712 | 3.78 |
| hsa-miR-1180-3p | 6.342 | 4.978 | 2.187 | 2.554 | 5.453 |
| hsa-miR-1180-5p | −16.61 | −16.61 | −16.61 | −16.61 | −16.61 |
| hsa-miR-1181-3p | −16.61 | −16.61 | −16.61 | −16.61 | −16.61 |
| hsa-miR-1181-5p | −16.61 | −16.61 | −16.61 | −16.61 | −16.61 |
| hsa-miR-1182 | −16.61 | −16.61 | −16.61 | −16.61 | −16.61 |
| hsa-miR-1183 | −16.61 | −16.61 | −16.61 | −16.61 | −16.61 |
| hsa-miR-1184 | −16.61 | −16.61 | −16.61 | −16.61 | −16.61 |
| hsa-miR-1185-1-3p/ | 2.288 | 3.622 | −16.61 | −16.61 | −16.61 |
| 1185-2-3p | | | | | |
| hsa-miR-1185-5p | −16.61 | 0.857 | −16.61 | −16.61 | −16.61 |
| hsa-miR-1193-3p | −16.61 | −16.61 | −16.61 | −16.61 | −16.61 |
| hsa-miR-1193-5p | −16.61 | −16.61 | −16.61 | −16.61 | −16.61 |
| hsa-miR-1197 | 2.211 | 1.705 | −16.61 | −16.61 | −16.61 |
| hsa-miR-1199-3p | −16.61 | −16.61 | −16.61 | −16.61 | −16.61 |
| hsa-miR-1199-5p | −16.61 | −16.61 | −16.61 | −16.61 | −16.61 |
| hsa-miR-1200 | −16.61 | −16.61 | −16.61 | −16.61 | −16.61 |
| hsa-miR-1202 | −16.61 | −16.61 | −16.61 | −16.61 | −16.61 |
| hsa-miR-1203 | −16.61 | −16.61 | −16.61 | −16.61 | −16.61 |
| hsa-miR-1204 | −16.61 | −16.61 | −16.61 | −16.61 | −16.61 |
| hsa-miR-1205 | −16.61 | −16.61 | −16.61 | −16.61 | −16.61 |
| hsa-miR-1206 | −16.61 | −16.61 | −16.61 | −16.61 | −16.61 |
| hsa-miR-1207-3p | −16.61 | −16.61 | −16.61 | −16.61 | −16.61 |
| hsa-miR-1207-5p | −16.61 | −16.61 | −16.61 | −16.61 | −16.61 |
| hsa-miR-1208 | −16.61 | −16.61 | −16.61 | −16.61 | −16.61 |
| hsa-miR-12113 | −16.61 | −16.61 | −16.61 | −16.61 | −16.61 |
| hsa-miR-12114 | −16.61 | −16.61 | 0.897 | −16.61 | −16.61 |
| hsa-miR-12115 | −16.61 | −16.61 | −16.61 | −16.61 | −16.61 |
| hsa-miR-12116 | −16.61 | −16.61 | −16.61 | −16.61 | −16.61 |
| hsa-miR-12117 | −16.61 | −16.61 | −16.61 | −16.61 | −16.61 |
| hsa-miR-12118 | −16.61 | −16.61 | −16.61 | −16.61 | −16.61 |
| hsa-miR-12119 | −16.61 | −16.61 | −16.61 | −16.61 | −16.61 |
| hsa-miR-12120 | −16.61 | −16.61 | −16.61 | −16.61 | −16.61 |
| hsa-miR-12121 | −16.61 | −16.61 | −16.61 | −16.61 | −16.61 |
| hsa-miR-12122 | −16.61 | −16.61 | −16.61 | −16.61 | −16.61 |
| hsa-miR-12123 | −16.61 | −16.61 | −16.61 | −16.61 | −16.61 |
| hsa-miR-12124 | −16.61 | −16.61 | −16.61 | −16.61 | −16.61 |
| hsa-miR-12125 | −16.61 | −16.61 | −16.61 | −16.61 | −16.61 |
| hsa-miR-12126 | −16.61 | −16.61 | −16.61 | −16.61 | −16.61 |
| hsa-miR-12127 | −16.61 | −16.61 | −16.61 | −16.61 | −16.61 |
| hsa-miR-12128 | −16.61 | −16.61 | −16.61 | −16.61 | −16.61 |
| hsa-miR-12129 | 3.545 | −16.61 | −16.61 | −16.61 | −16.61 |
| hsa-miR-12130 | −16.61 | −16.61 | −16.61 | −16.61 | −16.61 |
| hsa-miR-12131 | −16.61 | −16.61 | −16.61 | −16.61 | −16.61 |
| hsa-miR-12132 | −16.61 | −16.61 | −16.61 | −16.61 | −16.61 |
| hsa-miR-12133 | −16.61 | −16.61 | −16.61 | −16.61 | −16.61 |
| hsa-miR-12135 | −16.61 | −16.61 | −16.61 | −16.61 | −16.61 |
| hsa-miR-12136 | 4.022 | 8.976 | 4.946 | 7.496 | 7.735 |
| hsa-miR-122-3p | −16.61 | 0.12 | 1.187 | −16.61 | −16.61 |
| hsa-miR-122-5p | 13.305 | 16.38 | 11.726 | 13.833 | 12.412 |
| hsa-miR-1224-3p | −16.61 | −16.61 | −16.61 | −16.61 | −16.61 |
| hsa-miR-1224-5p | 5.815 | 4.235 | 1.634 | −16.61 | −16.61 |
| hsa-miR-1225-3p | −16.61 | −16.61 | −16.61 | −16.61 | −16.61 |
| hsa-miR-1225-5p | −16.61 | −16.61 | −16.61 | −16.61 | −16.61 |
| hsa-miR-1226-3p | 7.892 | 4.927 | 4.441 | 4.784 | 5.272 |
| hsa-miR-1226-5p | −16.61 | −16.61 | −16.61 | −16.61 | −16.61 |
| hsa-miR-1227-3p | −16.61 | −16.61 | −16.61 | −16.61 | −16.61 |
| hsa-miR-1227-5p | −16.61 | −16.61 | −16.61 | −16.61 | −16.61 |
| hsa-miR-1228-3p | 2.207 | 0.535 | −16.61 | 2.102 | −16.61 |
| hsa-miR-1228-5p | −16.61 | −16.61 | −16.61 | −16.61 | −16.61 |
| hsa-miR-1229-3p | 3.277 | −16.61 | 2.119 | −16.61 | −16.61 |
| hsa-miR-1229-5p | −16.61 | −16.61 | −16.61 | −16.61 | −16.61 |
| hsa-miR-122b-3p | −16.61 | −16.61 | −16.61 | −16.61 | −16.61 |
| hsa-miR-122b-5p | −16.61 | 3.892 | 0.897 | 0.854 | −16.61 |
| hsa-miR-1231-3p | −16.61 | −16.61 | −16.61 | −16.61 | −16.61 |
| hsa-miR-1231-5p | −16.61 | −16.61 | −16.61 | −16.61 | −16.61 |
| hsa-miR-1233-3p | −16.61 | −16.61 | −16.61 | −16.61 | −16.61 |
| hsa-miR-1233-5p | −16.61 | −16.61 | −16.61 | −16.61 | −16.61 |
| hsa-miR-1234-3p | 3.012 | −16.61 | −16.61 | −16.61 | −16.61 |
| hsa-miR-1236-3p | −16.61 | −16.61 | −16.61 | −16.61 | −16.61 |
| hsa-miR-1236-5p | 4.792 | 1.857 | −16.61 | −16.61 | −16.61 |
| hsa-miR-1237-3p | −16.61 | −16.61 | −16.61 | −16.61 | −16.61 |
| hsa-miR-1237-5p | −16.61 | −16.61 | −16.61 | −16.61 | −16.61 |
| hsa-miR-1238-3p | −16.61 | −16.61 | −16.61 | −16.61 | −16.61 |
| hsa-miR-1238-5p | −16.61 | −16.61 | −16.61 | −16.61 | −16.61 |
| hsa-miR-124-3p | 7.597 | 6.866 | 3.153 | 3.582 | 5.78 |
| hsa-miR-124-5p | 5.964 | 4.783 | 1.975 | 1.554 | −16.61 |
| hsa-miR-1243 | −16.61 | −16.61 | −16.61 | −16.61 | 2.95 |
| hsa-miR-1244 | −16.61 | −16.61 | −16.61 | 2.498 | −16.61 |
| hsa-miR-1245a | −16.61 | −16.61 | −16.61 | −16.61 | −16.61 |
| hsa-miR-1245b-3p | −16.61 | −16.61 | −16.61 | −16.61 | −16.61 |
| hsa-miR-1245b-5p | −16.61 | −16.61 | −16.61 | −16.61 | −16.61 |
| hsa-miR-1246 | 9.109 | 11.173 | 13.329 | 13.843 | 14.919 |
| hsa-miR-1247-3p | 3.248 | −16.61 | −16.61 | −16.61 | −16.61 |
| hsa-miR-1247-5p | 5.333 | 4.801 | −16.61 | −16.61 | −16.61 |
| hsa-miR-1248 | −16.61 | −16.61 | 0.897 | −16.61 | −16.61 |
| hsa-miR-1249-3p | 2.312 | 0.535 | −16.61 | 2.176 | −16.61 |
| hsa-miR-1249-5p | −16.61 | −16.61 | −16.61 | −16.61 | 4.481 |
| hsa-miR-1250-3p | −16.61 | −16.61 | −16.61 | −16.61 | −16.61 |
| hsa-miR-1250-5p | 4.312 | 2.994 | 1.634 | 1.761 | −16.61 |
| hsa-miR-1251-3p | 6.434 | 4.12 | 1.049 | 2.712 | −16.61 |
| hsa-miR-1251-5p | 8.495 | 5.773 | 1.428 | 1.661 | −16.61 |
| hsa-miR-1252-3p | 2.579 | −16.61 | −16.61 | −16.61 | −16.61 |
| hsa-miR-1252-5p | 4.116 | 2.857 | −16.61 | −16.61 | −16.61 |
| hsa-miR-1253 | −16.61 | −16.61 | −16.61 | −16.61 | −16.61 |
| hsa-miR-1255a | −16.61 | 0.535 | 1.897 | 2.609 | 4.24 |
| hsa-miR-1255b-2-3p | 1.9 | −16.61 | −16.61 | −16.61 | −16.61 |
| hsa-miR-1255b-5p | −16.61 | 2.342 | 2.428 | 2.661 | 2.95 |
| hsa-miR-1256-3p | 4.064 | 1.12 | 2.585 | 3.246 | 4.825 |
| hsa-miR-1256-5p | −16.61 | −16.61 | 16.61 | −16.61 | −16.61 |
| hsa-miR-1257 | −16.61 | −16.61 | −16.61 | −16.61 | −16.61 |
| hsa-miR-1258 | −16.61 | −16.61 | −16.61 | −16.61 | −16.61 |
| hsa-miR-125a-3p | 5.636 | 5.207 | 3.917 | 4.512 | 7.155 |
| hsa-miR-125a-5p | 15.458 | 13.153 | 12.713 | 12.582 | 9.897 |
| hsa-miR-125b-1-3p | 6.309 | 4.465 | −16.61 | −16.61 | −16.61 |
| hsa-miR-125b-2-3p | 5.604 | 4.944 | 3.836 | 4.736 | 4.102 |
| hsa-miR-125b-5p | 17.541 | 14.418 | 13.335 | 13.405 | 9.066 |
| hsa-miR-126-3p | 11.529 | 12.669 | 11.879 | 12.041 | 11.24 |
| hsa-miR-126-5p | 9.051 | 9.556 | 9.409 | 9.302 | 8.93 |
| hsa-miR-1260a/1260b | 4.69 | 2.535 | −16.61 | 2.661 | 4.028 |
| hsa-miR-1261 | −16.61 | −16.61 | −16.61 | −16.61 | −16.61 |
| hsa-miR-1262 | 5.124 | 5.316 | 2.371 | 2.983 | 3.365 |
| hsa-miR-1263-3p | −16.61 | −16.61 | −16.61 | −16.61 | −16.61 |
| hsa-miR-1263-5p | −16.61 | −16.61 | −16.61 | −16.61 | −16.61 |
| hsa-miR-1264 | 7.403 | 5.633 | 5.266 | 3.139 | 4.424 |
| hsa-miR-1265-3p | −16.61 | −16.61 | −16.61 | −16.61 | −16.61 |
| hsa-miR-1265-5p | −16.61 | −16.61 | −16.61 | −16.61 | −16.61 |
| hsa-miR-1266-3p | −16.61 | −16.61 | −16.61 | −16.61 | −16.61 |
| hsa-miR-1266-5p | −16.61 | 1.705 | −16.61 | −16.61 | −16.61 |
| hsa-miR-1267 | 1.709 | −16.61 | −16.61 | −16.61 | −16.61 |
| hsa-miR-1268a/1268b | 3.431 | 4.91 | −16.61 | 3.211 | 8.3 |
| hsa-miR-1269a-3p | 10.47 | 7.417 | 3.771 | 3.102 | −16.61 |
| hsa-miR-1269a-5p | −16.61 | −16.61 | −16.61 | −16.61 | −16.61 |
| hsa-miR-1269b | −1.57 | 2.535 | −16.61 | −16.61 | −16.61 |
| hsa-miR-127-3p | 4.469 | 7.761 | 4.441 | 4.635 | 2.95 |
| hsa-miR-127-5p | 16.61 | 3.783 | −16.61 | 1.854 | −16.61 |
| hsa-miR-1270-3p | −16.61 | 0.12 | 2.049 | −16.61 | −16.61 |
| hsa-miR-1270-5p | 6.812 | 6.948 | 5.61 | 6.267 | 6.803 |
| hsa-miR-1271-3p | −16.61 | −16.61 | −16.61 | −16.61 | −16.61 |
| hsa-miR-1271-5p | 5.49 | 5.027 | 2.975 | 2.898 | 2.78 |
| hsa-miR-1272 | −16.61 | −16.61 | −16.61 | −16.61 | −16.61 |
| hsa-miR-1273c | 4.738 | 3.744 | 2.312 | 2.554 | −16.61 |
| hsa-miR-1273h-3p | 3.061 | −16.61 | −16.61 | 2.554 | −16.61 |
| hsa-miR-1273h-5p | 16.61 | 3.442 | 1.187 | 2.808 | −16.61 |
| hsa-miR-1275 | 7.863 | 5.249 | 2.681 | 4.246 | 4.481 |
| hsa-miR-1276 | −16.61 | 2.442 | −16.61 | 3.439 | 3.365 |
| hsa-miR-1277-3p | 5.502 | 3.535 | 4.877 | 4.993 | 3.588 |
| hsa-miR-1277-5p | 8.344 | 7.612 | 8.912 | 8.884 | 4.588 |
| hsa-miR-1278 | −2.833 | −16.61 | 1.634 | 1.313 | −16.61 |
| hsa-miR-1279 | −16.61 | −16.61 | −16.61 | −16.61 | −16.61 |
| hsa-miR-128-1-5p | 4.633 | 5.664 | 2.771 | 3.063 | −16.61 |
| hsa-miR-128-2-5p | −16.61 | −16.61 | −16.61 | −16.61 | −16.61 |
| hsa-miR-128-3p | 12.927 | 12.171 | 11.855 | 11.839 | 9.786 |
| hsa-miR-1281 | −16.61 | −16.61 | −16.61 | −16.61 | −16.61 |
| hsa-miR-1282 | −16.61 | −16.61 | −16.61 | −16.61 | −16.61 |
| hsa-miR-1283 | −16.61 | 1.535 | 0.727 | −16.61 | −16.61 |
| hsa-miR-1284-3p | 3.868 | 5.316 | 2.681 | 2.609 | −16.61 |
| hsa-miR-1284-5p | −16.61 | 3.393 | 2.634 | −16.61 | −16.61 |
| hsa-miR-1285-3p | −16.61 | −16.61 | −16.61 | −16.61 | 3.24 |
| hsa-miR-1285-5p | 3.3 | −16.61 | 0.049 | 1.176 | −16.61 |
| hsa-miR-1286-3p | 4.58 | 3.29 | 2.371 | 2.246 | −16.61 |
| hsa-miR-1286-5p | −16.61 | −16.61 | −16.61 | −16.61 | −16.61 |
| hsa-miR-1287-3p | −16.61 | −16.61 | −16.61 | −16.61 | −16.61 |
| hsa-miR-1287-5p | 6.836 | 6.638 | 3.428 | 3.761 | 3.588 |
| hsa-miR-1288-3p | −16.61 | −16.61 | −16.61 | 2.498 | 3.481 |
| hsa-miR-1288-5p | −16.61 | −16.61 | −16.61 | −16.61 | −16.61 |
| hsa-miR-1289-3p | −3.156 | −16.61 | −16.61 | −16.61 | −16.61 |
| hsa-miR-1289-5p | 2.02 | −16.61 | −16.61 | −16.61 | −16.61 |
| hsa-miR-129-1-3p/ | 5.721 | 3.705 | −16.61 | −16.61 | −16.61 |
| 129-2-3p | | | | | |
| hsa-miR-129-5p | 9.286 | 7.712 | 3.482 | 3.346 | 2.102 |
| hsa-miR-1290 | −16.61 | −16.61 | 4.031 | 4.377 | 4.735 |
| hsa-miR-1291 | 4.409 | 4.892 | −16.61 | 2.609 | −16.61 |
| hsa-miR-1292-3p | −16.61 | −16.61 | −16.61 | −16.61 | −16.61 |
| hsa-miR-1292-5p | 4.732 | 4.535 | 2.681 | 3.28 | 5.138 |
| hsa-miR-1293-3p | −16.61 | −16.61 | −16.61 | −16.61 | −16.61 |
| hsa-miR-1293-5p | −16.61 | −16.61 | −16.61 | −16.61 | −16.61 |
| hsa-miR-1294-3p | −16.61 | −16.61 | −16.61 | −16.61 | −16.61 |
| hsa-miR-1294-5p | 2.603 | −16.61 | 2.428 | −16.61 | 4.304 |
| hsa-miR-1295a | −16.61 | −16.61 | −16.61 | −16.61 | −16.61 |
| hsa-miR-1295b-3p | −16.61 | −16.61 | −16.61 | −16.61 | −16.61 |
| hsa-miR-1295b-5p | −16.61 | −16.61 | −16.61 | −16.61 | −16.61 |
| hsa-miR-1296-3p | 2.696 | −16.61 | −16.61 | −16.61 | −16.61 |
| hsa-miR-1296-5p | 6.364 | 5.38 | 3.085 | 3.408 | 2.588 |
| hsa-miR-1297 | −16.61 | −16.61 | −16.61 | −16.61 | −16.61 |
| hsa-miR-1298-3p | 5.05 | 2.235 | 2.428 | 2.176 | −16.61 |
| hsa-miR-1298-5p | 6.359 | 5.38 | 5.297 | 3.854 | 6.38 |
| hsa-miR-1299 | 3.604 | −16.61 | −16.61 | −16.61 | −16.61 |
| hsa-miR-1301-3p | 7.804 | 6.914 | 5.646 | 5.914 | 8.232 |
| hsa-miR-1301-5p | −16.61 | −16.61 | −16.61 | −16.61 | −16.61 |
| hsa-miR-1302-3p | 0.517 | 1.12 | 1.187 | −16.61 | −16.61 |
| hsa-miR-1302-5p | −16.61 | −16.61 | −16.61 | −16.61 | −16.61 |
| hsa-miR-1303-3p | 3.288 | 3.179 | 1.727 | 3.661 | 4.735 |
| hsa-miR-1303-5p | −16.61 | 1.535 | 0.897 | 1.439 | −16.61 |
| hsa-miR-1304-3p | −16.61 | −16.61 | −16.61 | −16.61 | −16.61 |
| hsa-miR-1304-5p | −16.61 | −16.61 | −16.61 | −16.61 | −16.61 |
| hsa-miR-1305-3p | −16.61 | 1.342 | −16.61 | −16.61 | −16.61 |
| hsa-miR-1305-5p | −16.61 | −16.61 | −16.61 | −16.61 | −16.61 |
| hsa-miR-1306-3p | −16.61 | 1.535 | −16.61 | −16.61 | −16.61 |
| hsa-miR-1306-5p | 5.958 | 6.923 | 3.975 | 2.377 | 3.481 |
| hsa-miR-1307-3p | 8.471 | 9.819 | 9.064 | 9.637 | 10.502 |
| hsa-miR-1307-5p | 7.838 | 8.06 | 7.876 | 7.705 | 6.522 |
| hsa-miR-130a-3p | 12.398 | 11.496 | 8.406 | 8.573 | 9.78 |
| hsa-miR-130a-5p | 3.314 | 2.622 | 1.975 | 1.439 | −16.61 |
| hsa-miR-130b-3p | 10.893 | 10.033 | 9.066 | 9.341 | 12.304 |
| hsa-miR-130b-5p | 5.916 | 3.393 | 2.897 | 2.554 | 5.424 |
| hsa-miR-132-3p | 6.668 | 5.29 | 6.987 | 6.699 | 7.343 |
| hsa-miR-132-5p | 3.481 | 1.994 | 3.917 | 3.408 | 4.687 |
| hsa-miR-1321 | −16.61 | −16.61 | −16.61 | −16.61 | −16.61 |
| hsa-miR-1322 | −16.61 | 1.342 | −16.61 | −16.61 | −16.61 |
| hsa-miR-1323 | −16.61 | 0.857 | −16.61 | −16.61 | −16.61 |
| hsa-miR-1324 | −16.61 | −16.61 | −16.61 | −16.61 | −16.61 |
| hsa-miR-133a-3p/133b | 4.154 | 10.465 | 6.485 | 6.148 | −16.61 |
| hsa-miR-133a-5p | −16.61 | 4.149 | 2.251 | 2.712 | −16.61 |
| hsa-miR-134-3p | −16.61 | −16.61 | −16.61 | −16.61 | −16.61 |
| hsa-miR-134-5p | 4.697 | 6.249 | 1.975 | 3.736 | −16.61 |
| hsa-miR-1343-3p | 4.205 | 0.857 | 0.897 | 0.439 | 1.365 |
| hsa-miR-1343-5p | −16.61 | −16.61 | −16.61 | −16.61 | −16.61 |
| hsa-miR-135a-2-3p | 7.246 | 4.417 | 1.312 | −16.61 | −16.61 |
| hsa-miR-135a-3p | 5.137 | 5.235 | 1.634 | 2.661 | 3.24 |
| hsa-miR-135a-5p | 11.193 | 8.761 | 4.727 | 4.424 | 2.365 |
| hsa-miR-135b-3p | −16.61 | 1.535 | −16.61 | −16.61 | −16.61 |
| hsa-miR-135b-5p | 6.928 | 6.066 | 2.534 | 1.761 | −16.61 |
| hsa-miR-136-3p | 3.786 | 5.263 | 2.727 | −16.61 | −16.61 |
| hsa-miR-136-5p | −16.61 | 2.12 | −16.61 | −16.61 | −16.61 |
| hsa-miR-137-3p | 10.508 | 9.15 | 4.716 | 5.263 | 6.663 |
| hsa-miR-137-5p | 3.75 | −16.61 | −16.61 | −16.61 | −16.61 |
| hsa-miR-138-1-3p | 3.202 | −16.61 | 0.534 | 2.609 | −16.61 |
| hsa-miR-138-2-3p | 4.43 | 2.535 | −16.61 | 1.024 | −16.61 |
| hsa-miR-138-5p | 6.697 | 5.221 | 4.049 | 5.802 | 9.16 |
| hsa-miR-139-3p | 3.501 | 4.512 | −16.61 | 1.176 | −16.61 |
| hsa-miR-139-5p | 2.414 | 2.342 | −16.61 | 1.024 | 4.535 |
| hsa-miR-140-3p | 11.969 | 11.956 | 12.859 | 12.815 | 9.413 |
| hsa-miR-140-5p | 8.189 | 8.462 | 8.504 | 8.67 | 8.308 |
| hsa-miR-141-3p | 5.633 | 6.638 | 6.247 | 6.373 | 5.138 |
| hsa-miR-141-5p | −16.61 | 2.235 | 2.049 | −16.61 | −16.61 |
| hsa-miR-142-3p | 13.001 | 14.335 | 15.753 | 15.445 | 14.22 |
| hsa-miR-142-5p | 13.688 | 14.09 | 15.45 | 14.936 | 13.888 |
| hsa-miR-143-3p | 10.234 | 13.226 | 12.141 | 11.311 | 12.297 |
| hsa-miR-143-5p | 4.555 | 7.054 | 6.247 | 5.102 | −16.61 |
| hsa-miR-144-3p | 2.135 | 7.546 | 4.219 | 4.526 | 5.138 |
| hsa-miR-144-5p | 3.078 | 6.523 | 3.312 | 4.297 | −16.61 |
| hsa-miR-145-3p | 4.264 | 5.579 | 4.495 | 4.024 | 2.95 |
| hsa-miR-145-5p | 6.33 | 8.18 | 7.085 | 6.333 | 5.24 |
| hsa-miR-1468-3p | −16.61 | −16.61 | −16.61 | −16.61 | −16.61 |
| hsa-miR-1468-5p | 4.891 | 5.654 | 3.877 | 3.346 | −16.61 |
| hsa-miR-1469 | −16.61 | −16.61 | −16.61 | −16.61 | −16.61 |
| hsa-miR-146a-3p | 4.177 | 3.622 | 2.428 | 4.044 | −16.61 |
| hsa-miR-146a-5p | 13.382 | 15.528 | 14.698 | 15.81 | 10.741 |
| hsa-miR-146b-3p | 7.838 | 7.872 | 8.543 | 8.791 | −16.61 |
| hsa-miR-146b-5p | 12.947 | 12.124 | 14.107 | 14.744 | 12.419 |
| hsa-miR-1470 | −16.61 | −16.61 | −16.61 | −16.61 | −16.61 |
| hsa-miR-1471 | −16.61 | −16.61 | −16.61 | −16.61 | −16.61 |
| hsa-miR-147a | −16.61 | −16.61 | −16.61 | −16.61 | −16.61 |
| hsa-miR-147b-3p | 3.237 | 1.535 | 3.119 | 5.063 | 7.028 |
| hsa-miR-147b-5p | 3.107 | 3.744 | 2.585 | 3.808 | −16.61 |
| hsa-miR-148a-3p | 11.642 | 12.992 | 11.324 | 12.02 | 4.735 |
| hsa-miR-148a-5p | 3.554 | 1.535 | 0.727 | 3.024 | −16.61 |
| hsa-miR-148b-3p | 12.861 | 13.121 | 12.973 | 13.114 | 7.12 |
| hsa-miR-148b-5p | 4.907 | 3.927 | 4.771 | 4.648 | 3.868 |
| hsa-miR-149-3p | 4.392 | −16.61 | −16.61 | −16.61 | −16.61 |
| hsa-miR-149-5p | 9.527 | 8.455 | 4.371 | 4.554 | 6.35 |
| hsa-miR-150-3p | −16.61 | −16.61 | −16.61 | −16.61 | −16.61 |
| hsa-miR-150-5p | 5.382 | 6.848 | 5.012 | 4.865 | 2.102 |
| hsa-miR-151a-3p | 12.378 | 11.969 | 10.275 | 10.363 | 8.72 |
| hsa-miR-151a-5p/151b | 8.256 | 7.614 | 5.877 | 5.554 | 11.913 |
| hsa-miR-152-3p | 10.958 | 10.324 | 8.202 | 8.672 | 3.24 |
| hsa-miR-152-5p | −3.156 | −16.61 | −16.61 | −16.61 | −16.61 |
| hsa-miR-153-3p | 10.336 | 7.329 | 3.012 | 3.139 | −16.61 |
| hsa-miR-153-5p | 6.498 | 3.994 | 0.312 | 1.176 | −16.61 |
| hsa-miR-1537-3p | −16.61 | −16.61 | 2.119 | 2.377 | 4.102 |
| hsa-miR-1537-5p | −16.61 | −16.61 | 2.815 | 2.313 | −16.61 |
| hsa-miR-1538 | 3.599 | −16.61 | −16.61 | 2.313 | 2.365 |
| hsa-miR-1539 | −16.61 | −16.61 | −16.61 | −16.61 | −16.61 |
| hsa-miR-154-3p | −16.61 | −16.61 | −16.61 | −16.61 | −16.61 |
| hsa-miR-154-5p | −16.61 | 2.12 | 0.534 | −16.61 | −16.61 |
| hsa-miR-155-3p | −2.249 | −16.61 | −16.61 | −16.61 | −16.61 |
| hsa-miR-155-5p | 9.394 | 10.831 | 10.856 | 12.027 | 14.054 |
| hsa-miR-1587 | −16.61 | −16.61 | −16.61 | −16.61 | −16.61 |
| hsa-miR-15a-3p | −16.61 | 0.12 | 2.585 | 1.176 | 3.588 |
| hsa-miR-15a-5p | 8.969 | 9.726 | 11.336 | 11.16 | 11.997 |
| hsa-miR-15b-3p | 7.509 | 7.801 | 8.056 | 8.086 | 6.699 |
| hsa-miR-15b-5p | 9.76 | 9.77 | 10.806 | 10.736 | 12.633 |
| hsa-miR-16-1-3p | 3.334 | 2.12 | 5.17 | 4.865 | −16.61 |
| hsa-miR-16-2-3p | 4.361 | 2.857 | 3.658 | 3.962 | −16.61 |
| hsa-miR-16-5p | 16.412 | 17.781 | 18.112 | 18.162 | 18.007 |
| hsa-miR-17-3p | 5.687 | 5.074 | 6.145 | 5.655 | 8.339 |
| hsa-miR-181a-2-3p | 9.341 | 7.568 | 7.024 | 7.061 | 7.515 |
| hsa-miR-181a-3p | 8.364 | 6.568 | 6.428 | 6.629 | 6.825 |
| hsa-miR-181a-5p | 11.723 | 9.915 | 9.345 | 9.147 | 10.392 |
| hsa-miR-181b-2-3p | 6.333 | 3.442 | 3.085 | 3.28 | 3.868 |
| hsa-miR-181b-3p | 6.978 | 3.927 | 3.994 | 4.083 | 2.102 |
| hsa-miR-181b-5p | 10.463 | 8.705 | 8.041 | 8.242 | 9.821 |
| hsa-miR-181c-3p | 8.195 | 6.773 | 4.522 | 3.28 | 5.868 |
| hsa-miR-181c-5p | 7.839 | 6.089 | 4.297 | 3.808 | 4.91 |
| hsa-miR-181d-3p | 4.294 | 1.535 | 0.312 | −16.61 | 2.78 |
| hsa-miR-181d-5p | 6.44 | 4.417 | 3.153 | 1.941 | 4.868 |
| hsa-miR-182-3p | −16.61 | −16.61 | −16.61 | 0.854 | −16.61 |
| hsa-miR-182-5p | 6.846 | 9.003 | 4.956 | 5.255 | −16.61 |
| hsa-miR-1825 | −16.61 | −16.61 | −16.61 | −16.61 | −16.61 |
| hsa-miR-1827 | 16.61 | −16.61 | −16.61 | −16.61 | −16.61 |
| hsa-miR-183-3p | −16.61 | −16.61 | −16.61 | −16.61 | −16.61 |
| hsa-miR-183-5p | 6.035 | 8.054 | 4.153 | 4.408 | −16.61 |
| hsa-miR-184 | 5.891 | 5.994 | 2.897 | 3.28 | 3.78 |
| hsa-miR-1843 | 3.332 | 4.892 | 4.547 | 5.053 | 6.155 |
| hsa-miR-185-3p | 3.956 | 2.705 | 3.994 | 5.044 | 4.028 |
| hsa-miR-185-5p | 9.639 | 9.914 | 10.403 | 10.728 | 13.69 |
| hsa-miR-186-3p | 3.969 | 2.235 | 4.342 | 4.622 | −16.61 |
| hsa-miR-186-5p | 11.528 | 11.037 | 11.394 | 11.469 | 10.364 |
| hsa-miR-187-3p | 5.488 | 6.914 | 5.441 | 6.668 | 7.508 |
| hsa-miR-187-5p | −16.61 | −16.61 | −16.61 | 0.439 | −16.61 |
| hsa-miR-188-3p | 4.307 | 2.342 | 2.856 | 2.024 | 3.102 |
| hsa-miR-188-5p | 4.171 | 5.546 | 5.334 | 5.431 | 6.19 |
| hsa-miR-18a-3p | 3.499 | 4.12 | 2.856 | 3.246 | −16.61 |
| hsa-miR-18a-5p | 6.29 | 8.014 | 6.828 | 6.749 | 9.021 |
| hsa-miR-18b-3p | −16.61 | 1.342 | 0.534 | −16.61 | −16.61 |
| hsa-miR-18b-5p | 3.487 | 4.535 | 3.508 | 3.377 | 4.102 |
| hsa-miR-1908-3p | −16.61 | −16.61 | 1.428 | −16.61 | −16.61 |
| hsa-miR-1908-5p | 3.849 | 5.477 | 3.56 | 1.439 | 5.173 |
| hsa-miR-1909-3p | −16.61 | −16.61 | −16.61 | −16.61 | −16.61 |
| hsa-miR-1909-5p | −16.61 | −16.61 | −16.61 | −16.61 | −16.61 |
| hsa-miR-190a-3p | 1.148 | 0.12 | −16.61 | −16.61 | −16.61 |
| hsa-miR-190a-5p | 7.159 | 8.193 | 5.675 | 5.561 | −16.61 |
| hsa-miR-190b-3p | −16.61 | −16.61 | −16.61 | −16.61 | −16.61 |
| hsa-miR-190b-5p | 7.326 | 7.157 | 6.628 | 6.884 | 4.735 |
| hsa-miR-191-3p | 5.803 | 6.442 | 6.573 | 6.944 | 6.98 |
| hsa-miR-191-5p | 15.665 | 14.557 | 14.993 | 15.011 | 15.541 |
| hsa-miR-1910-3p | −16.61 | −16.61 | −16.61 | −16.61 | −16.61 |
| hsa-miR-1910-5p | 2.727 | −16.61 | −16.61 | −16.61 | −16.61 |
| hsa-miR-1911-3p | −16.61 | −16.61 | −16.61 | −16.61 | −16.61 |
| hsa-miR-1911-5p | 5.495 | 3.664 | 5.877 | 4.582 | 5.588 |
| hsa-miR-1912-3p | 4.632 | 3.179 | 2.815 | 2.554 | 7.453 |
| hsa-miR-1912-5p | −16.61 | −16.61 | 1.534 | −16.61 | 3.481 |
| hsa-miR-1913-3p | −16.61 | −16.61 | −16.61 | −16.61 | −16.61 |
| hsa-miR-1913-5p | −16.61 | −16.61 | −16.61 | −16.61 | −16.61 |
| hsa-miR-1914-3p | 3.908 | −16.61 | −16.61 | −16.61 | −16.61 |
| hsa-miR-1914-5p | 2.662 | −16.61 | −16.61 | −16.61 | −16.61 |
| hsa-miR-1915-3p | 3.913 | 2.705 | 0.727 | −16.61 | −16.61 |
| hsa-miR-1915-5p | −16.61 | −16.61 | −16.61 | −16.61 | −16.61 |
| hsa-miR-192-3p | 3.296 | 2.927 | 1.815 | 2.712 | −16.61 |
| hsa-miR-192-5p/ | 11.075 | 12.36 | 8.782 | 9.605 | 5.173 |
| 215-5p | | | | | |
| hsa-miR-193a-3p | 3.064 | −16.61 | −16.61 | 2.246 | −16.61 |
| hsa-miR-193a-5p | 7.511 | 8.369 | 6.385 | 6.658 | 7.894 |
| hsa-miR-193b-3p | 3.745 | 2.622 | 2.119 | 0.176 | −16.61 |
| hsa-miR-193b-5p | 5.004 | 5.695 | 2.049 | 1.761 | 3.481 |
| hsa-miR-194-3p | −16.61 | −16.61 | −16.61 | 2.176 | −16.61 |
| hsa-miR-194-5p | 8.15 | 10.092 | 7.031 | 8.119 | 4.028 |
| hsa-miR-195-3p | −16.61 | −16.61 | −16.61 | 1.313 | −16.61 |
| hsa-miR-195-5p | 4.646 | 5.601 | 4.067 | 4.876 | −16.61 |
| hsa-miR-196a-1-3p | −16.61 | −16.61 | −16.61 | 0.439 | −16.61 |
| hsa-miR-196a-3p | −16.61 | −16.61 | −16.61 | −16.61 | −16.61 |
| hsa-miR-196a-5p | 2.485 | 1.12 | 3.4 | 4.024 | 4.868 |
| hsa-miR-196b-3p | −16.61 | −16.61 | −16.61 | −16.61 | −16.61 |
| hsa-miR-196b-5p | −16.61 | 4.12 | 3.877 | 4.346 | 2.95 |
| hsa-miR-197-3p | 7.839 | 7.175 | 7.139 | 6.42 | 7.232 |
| hsa-miR-197-5p | 4.081 | 2.857 | 3.153 | 3.102 | 4.304 |
| hsa-miR-1972 | −16.61 | −16.61 | −16.61 | −16.61 | 4.735 |
| hsa-miR-1973 | 16.61 | −16.61 | 0.049 | 4.582 | 6.711 |
| hsa-miR-1976 | −16.61 | 5.715 | 5.777 | 6.087 | 4.91 |
| hsa-miR-198 | −16.61 | −16.61 | −16.61 | −16.61 | −16.61 |
| hsa-miR-199a-3p/ | 10.493 | 10.693 | 9.903 | 9.994 | 7.752 |
| 199b-3p | | | | | |
| hsa-miR-199a-5p | 8.026 | 6.193 | 3.634 | 4.329 | 2.365 |
| hsa-miR-199b-5p | 7.912 | 8.885 | 9.239 | 8.019 | 5.535 |
| hsa-miR-19a-3p | 10.458 | 9.929 | 11.018 | 10.405 | 4.535 |
| hsa-miR-19a-5p | −16.61 | 3.342 | −16.61 | −16.61 | −16.61 |
| hsa-miR-19b-1-5p | −1.907 | −16.61 | 1.312 | −0.561 | 2.365 |
| hsa-miR-19b-2-5p | −16.61 | −16.61 | −16.61 | −16.61 | −16.61 |
| hsa-miR-19b-3p | 12.302 | 12.344 | 12.137 | 11.46 | 6.549 |
| hsa-miR-200a-3p | 3.596 | 7.313 | 6.017 | 5.661 | 3.588 |
| hsa-miR-200a-5p | −16.61 | 1.705 | −16.61 | 0.439 | −16.61 |
| hsa-miR-200b-3p | 4.145 | 6.2 | 5.258 | 5.393 | 4.173 |
| hsa-miR-200b-5p | −16.61 | 0.535 | −16.61 | −16.61 | −16.61 |
| hsa-miR-200c-3p | 6.077 | 6.465 | 5.777 | 5.962 | 7.111 |
| hsa-miR-200c-5p | −16.61 | 0.12 | 0.534 | −16.61 | −16.61 |
| hsa-miR-202-3p | −16.61 | −16.61 | −16.61 | −16.61 | −16.61 |
| hsa-miR-202-5p | −4.57 | −16.61 | 0.049 | −16.61 | −16.61 |
| hsa-miR-203a-3p | 6.432 | 10.405 | 6.889 | 6.854 | 5.588 |
| hsa-miR-203a-5p | −16.61 | −16.61 | −16.61 | −16.61 | −16.61 |
| hsa-miR-203b-3p | −16.61 | 2.994 | −16.61 | −16.61 | −16.61 |
| hsa-miR-203b-5p | −16.61 | −16.61 | −16.61 | −16.61 | −16.61 |
| hsa-miR-204-3p | −16.61 | 4.089 | −16.61 | −16.61 | −16.61 |
| hsa-miR-204-5p | 7.793 | 5.135 | 2.482 | 3.024 | −16.61 |
| hsa-miR-205-3p | −16.61 | −16.61 | −16.61 | −16.61 | −16.61 |
| hsa-miR-205-5p | 2.772 | 5.368 | 3.219 | 2.498 | 2.95 |
| hsa-miR-2052 | −16.61 | −16.61 | −16.61 | −16.61 | −16.61 |
| hsa-miR-2053 | −16.61 | −16.61 | −16.61 | −16.61 | −16.61 |
| hsa-miR-2054 | −16.61 | −16.61 | −16.61 | −16.61 | −16.61 |
| hsa-miR-206 | −16.61 | 4.29 | −16.61 | 2.439 | −16.61 |
| hsa-miR-208a-3p | −16.61 | 10.097 | 6.067 | 5.595 | −16.61 |
| hsa-miR-208a-5p | −16.61 | 7.707 | 3.585 | 3.024 | −16.61 |
| hsa-miR-208b-3p | 4.691 | 9.42 | 5.528 | 4.973 | −16.61 |
| hsa-miR-208b-5p | −16.61 | −16.61 | −16.61 | −16.61 | −16.61 |
| hsa-miR-20a-3p | 6.165 | 5.978 | 4.385 | 3.962 | 6.256 |
| hsa-miR-20a-5p | 11.299 | 10.823 | 11.036 | 10.98 | 12.555 |
| hsa-miR-20b-3p | −16.61 | −16.61 | −16.61 | −16.61 | −16.61 |
| hsa-miR-20b-5p | 9.594 | 11.286 | 9.691 | 9.207 | 10.085 |
| hsa-miR-21-3p | 9.045 | 7.931 | 7.062 | 8.041 | 7.857 |
| hsa-miR-21-5p | 15.045 | 14.956 | 14.377 | 15.045 | 13.677 |
| hsa-miR-210-3p | 8.568 | 8.762 | 8.484 | 8.222 | 9.25 |
| hsa-miR-210-5p | 2.808 | −16.61 | −16.61 | −16.61 | −16.61 |
| hsa-miR-211-3p | −16.61 | −16.61 | −16.61 | −16.61 | −16.61 |
| hsa-miR-211-5p | 1.271 | −16.61 | −16.61 | −16.61 | −16.61 |
| hsa-miR-2110 | 4.504 | 5.368 | 4.187 | 4.962 | 8.597 |
| hsa-miR-2113-3p | −16.61 | −16.61 | −16.61 | −16.61 | −16.61 |
| hsa-miR-2113-5p | −16.61 | −16.61 | −16.61 | −16.61 | −16.61 |
| hsa-miR-2114-3p | 2.945 | −16.61 | −16.61 | −16.61 | −16.61 |
| hsa-miR-2114-5p | −16.61 | −16.61 | −16.61 | −16.61 | −16.61 |
| hsa-miR-2115-3p | −16.61 | −16.61 | 1.897 | 1.554 | −16.61 |
| hsa-miR-2115-5p | −16.61 | −16.61 | −16.61 | −16.61 | −16.61 |
| hsa-miR-2116-3p | 3.18 | 4.12 | 0.897 | 2.498 | −16.61 |
| hsa-miR-2116-5p | 1.633 | −16.61 | 2.187 | 2.712 | −16.61 |
| hsa-miR-2117-3p | −16.61 | −16.61 | −16.61 | −16.61 | −16.61 |
| hsa-miR-2117-5p | −16.61 | −16.61 | −16.61 | −16.61 | −16.61 |
| hsa-miR-212-3p | 3.222 | 0.12 | 2.856 | 2.377 | 4.304 |
| hsa-miR-212-5p | −16.61 | 0.535 | 2.534 | 0.661 | −16.61 |
| hsa-miR-214-3p | 4.109 | 0.535 | 1.634 | −16.61 | −16.61 |
| hsa-miR-214-5p | 1.488 | 0.857 | −16.61 | −16.61 | −16.61 |
| hsa-miR-215-3p | −16.61 | −16.61 | −16.61 | −16.61 | −16.61 |
| hsa-miR-216a-3p | 4.781 | 7.396 | 3.956 | 3.92 | −16.61 |
| hsa-miR-216a-5p | 7.333 | 9.582 | 5.482 | 5.519 | 16.61 |
| hsa-miR-216b-3p | 3.624 | 6.269 | 2.856 | 1.439 | −16.61 |
| hsa-miR-216b-5p | −16.61 | 2.235 | −16.61 | −16.61 | −16.61 |
| hsa-miR-217-3p | −16.61 | 2.235 | −16.61 | 1.176 | −16.61 |
| hsa-miR-217-5p | 6.414 | 9.544 | 4.994 | 5.034 | −16.61 |
| hsa-miR-218-1-3p | 3.706 | 2.342 | −16.61 | −16.61 | −16.61 |
| hsa-miR-218-2-3p | 6.514 | 5.838 | 3.4 | 3.526 | −16.61 |
| hsa-miR-218-5p | 14.698 | 13.661 | 11.344 | 11.746 | 7.632 |
| hsa-miR-219a-1-3p | 6.746 | 5.316 | 2.681 | 1.176 | 2.78 |
| hsa-miR-219a-2-3p | 8.334 | 11.177 | 6.704 | 6.625 | −16.61 |
| hsa-miR-219a-5p | 9.709 | 10.492 | 6.421 | 6.13 | −16.61 |
| hsa-miR-219b-3p | −16.61 | −16.61 | −16.61 | −16.61 | −16.61 |
| hsa-miR-219b-5p | 1.816 | 0.857 | 0.897 | 1.554 | −16.61 |
| hsa-miR-22-3p | 9.598 | 9.864 | 10.337 | 10.823 | 11.107 |
| hsa-miR-22-5p | 4.73 | 3.892 | 4.049 | 4.687 | 6.495 |
| hsa-miR-221-3p | 9.491 | 10.982 | 11.941 | 12.161 | 11.563 |
| hsa-miR-221-5p | 3.771 | 5.042 | 4.771 | 5.053 | 5.453 |
| hsa-miR-222-3p | 6.428 | 6.734 | 7.927 | 8.177 | 7.084 |
| hsa-miR-222-5p | 0.822 | −16.61 | 1.187 | 3.377 | 5.95 |
| hsa-miR-223-3p | 14.182 | 16.261 | 16.799 | 15.603 | 13.819 |
| hsa-miR-223-5p | 7.632 | 10.002 | 10.58 | 10.177 | 11.071 |
| hsa-miR-224-3p | −16.61 | −16.61 | 0.049 | −16.61 | −16.61 |
| hsa-miR-224-5p | 11.109 | 12.429 | 13.498 | 12.881 | 13.261 |
| hsa-miR-2276-3p | 5.094 | 2.235 | −16.61 | 2.498 | 3.481 |
| hsa-miR-2276-5p | 2.784 | −16.61 | −16.61 | −16.61 | −16.61 |
| hsa-miR-2277-3p | 3.923 | 4.149 | 2.371 | 3.498 | 2.95 |
| hsa-miR-2277-5p | 3.454 | 1.857 | −16.61 | −16.61 | −16.61 |
| hsa-miR-2278-3p | 2.148 | −16.61 | −16.61 | −16.61 | −16.61 |
| hsa-miR-2278-5p | 1.422 | 1.705 | 0.534 | 2.439 | 5.481 |
| hsa-miR-2355-3p | 4.445 | 3.489 | 4.251 | 4.28 | 3.588 |
| hsa-miR-2355-5p | 2.755 | 1.12 | 2.312 | 2.377 | −16.61 |
| hsa-miR-2392 | −16.61 | −16.61 | −16.61 | −16.61 | −16.61 |
| hsa-miR-23a-3p/ | 12.967 | 12.105 | 12.391 | 12.384 | 11.877 |
| 23b-3p | | | | | |
| hsa-miR-23a-5p | 2.407 | 3.857 | 2.482 | 3.941 | −16.61 |
| hsa-miR-23b-5p | 3.388 | 3.579 | 1.312 | 2.176 | −16.61 |
| hsa-miR-23c | −16.61 | −16.61 | −16.61 | −16.61 | −16.61 |
| hsa-miR-24-1-5p | −16.61 | −16.61 | −16.61 | −16.61 | −16.61 |
| hsa-miR-24-2-5p | −16.61 | 4.838 | 1.634 | 2.102 | −16.61 |
| hsa-miR-24-3p | 13.019 | 12.391 | 12.082 | 12.397 | 13.781 |
| hsa-miR-2467-3p | −16.61 | −16.61 | −16.61 | −16.61 | −16.61 |
| hsa-miR-2467-5p | 1.336 | −16.61 | −16.61 | −16.61 | −16.61 |
| hsa-miR-25-3p | 13.299 | 13.45 | 13.918 | 13.75 | 11.446 |
| hsa-miR-25-5p | 4.277 | 5.149 | 3.428 | 3.526 | 5.066 |
| hsa-miR-2681-3p | −16.61 | 0.857 | −16.61 | −16.61 | −16.61 |
| hsa-miR-2681-5p | −16.61 | −16.61 | −16.61 | −16.61 | −16.61 |
| hsa-miR-2682-3p | 0.974 | 2.342 | −16.61 | −16.61 | −16.61 |
| hsa-miR-2682-5p | 2.224 | −0.465 | 1.634 | −16.61 | −16.61 |
| hsa-miR-26a-1-3p | 3.37 | 2.12 | 1.534 | 1.661 | −16.61 |
| hsa-miR-26a-2-3p | 4.994 | 1.535 | 3.56 | 2.498 | −16.61 |
| hsa-miR-26a-5p | 13.357 | 12.579 | 12.932 | 12.853 | 14.583 |
| hsa-miR-26b-3p | 4.544 | −16.61 | 4.984 | 4.993 | 4.687 |
| hsa-miR-26b-5p | 14.036 | 14.031 | 14.161 | 14.171 | 14.682 |
| hsa-miR-27a-3p/ | 13.988 | 13.401 | 12.928 | 12.822 | 11.862 |
| 27b-3p | | | | | |
| hsa-miR-27a-5p | 3.975 | 3.489 | 1.815 | 3.408 | 4.687 |
| hsa-miR-27b-5p | −16.61 | 3.961 | −16.61 | −16.61 | 3.95 |
| hsa-miR-28-3p | 11.723 | 11.785 | 11.309 | 11.619 | 11.397 |
| hsa-miR-28-5p | 4.651 | 3.783 | 3.856 | 3.408 | 8.41 |
| hsa-miR-2861-3p | −16.61 | −16.61 | −16.61 | −16.61 | −16.61 |
| hsa-miR-2861-5p | −16.61 | −16.61 | −16.61 | −16.61 | −16.61 |
| hsa-miR-2909 | −16.61 | −16.61 | −16.61 | −16.61 | −16.61 |
| hsa-miR-296-3p | 5.765 | 7.01 | 3.771 | 3.635 | 3.102 |
| hsa-miR-296-5p | 5.025 | 3.579 | 2.187 | 2.712 | −16.61 |
| hsa-miR-297 | −16.61 | −16.61 | −16.61 | −16.61 | −16.61 |
| hsa-miR-298-3p | −16.61 | −16.61 | −16.61 | −16.61 | −16.61 |
| hsa-miR-298-5p | −16.61 | −16.61 | −16.61 | −16.61 | −16.61 |
| hsa-miR-299-3p | 2.724 | 4.179 | −16.61 | 0.854 | −16.61 |
| hsa-miR-299-5p | 1.344 | 2.235 | −16.61 | 0.661 | −16.61 |
| hsa-miR-29a-3p | 12.535 | 12.635 | 13.705 | 13.698 | 13.42 |
| hsa-miR-29a-5p | 2.715 | 0.857 | 2.937 | 3.408 | 3.24 |
| hsa-miR-29b-1-5p | 2.157 | −16.61 | 1.897 | 1.439 | 3.102 |
| hsa-miR-29b-2-5p | 4.943 | 1.342 | 3.049 | 4.024 | 4.535 |
| hsa-miR-29b-3p | 10.324 | 11.098 | 11.103 | 10.811 | 10.901 |
| hsa-miR-29c-3p | 12.056 | 11.68 | 12.444 | 12.183 | 10.323 |
| hsa-miR-29c-5p | 6.261 | 5.179 | 5.085 | 4.876 | 5.24 |
| hsa-miR-300 | −16.61 | −16.61 | −16.61 | −16.61 | −16.61 |
| hsa-miR-301a-3p | 12.616 | 11.198 | 10.057 | 9.518 | 6.792 |
| hsa-miR-301a-5p | 6.897 | 6.082 | 5.61 | 5.305 | 0.78 |
| hsa-miR-301b-3p | 10.646 | 8.85 | 7.006 | 5.952 | 1.365 |
| hsa-miR-301b-5p | 4.533 | 3.535 | 1.312 | 2.246 | −16.61 |
| hsa-miR-302a-3p | 2.715 | 8.095 | 4.085 | 4.439 | −16.61 |
| hsa-miR-302a-5p | 5.492 | 13.147 | 9.203 | 8.973 | 3.24 |
| hsa-miR-302b-3p | 0.764 | 7.936 | 3.956 | 4.121 | 2.78 |
| hsa-miR-302b-5p | −16.61 | −16.61 | −16.61 | −16.61 | −16.61 |
| hsa-miR-302c-3p | −16.61 | 5.235 | 2.119 | 2.176 | −16.61 |
| hsa-miR-302c-5p | −16.61 | 3.235 | 0.312 | 1.176 | −16.61 |
| hsa-miR-302d-3p | −16.61 | 8.138 | 3.994 | 3.983 | −16.61 |
| hsa-miR-302d-5p | −16.61 | −16.61 | −16.61 | −16.61 | −16.61 |
| hsa-miR-302e | −16.61 | −16.61 | −16.61 | −16.61 | −16.61 |
| hsa-miR-302f | −16.61 | −16.61 | −16.61 | −16.61 | −16.61 |
| hsa-miR-3059-3p | −16.61 | −16.61 | −16.61 | −16.61 | −16.61 |
| hsa-miR-3059-5p | −16.61 | −16.61 | −16.61 | −16.61 | −16.61 |
| hsa-miR-3064-3p | −16.61 | −16.61 | −0.273 | 1.854 | −16.61 |
| hsa-miR-3064-5p | 3.594 | 1.342 | 2.585 | 2.808 | −16.61 |
| hsa-miR-3065-3p | 7.886 | 6.54 | 5.999 | 7.205 | −16.61 |
| hsa-miR-3065-5p | 9.328 | 8.195 | 7.479 | 9.206 | 6.24 |
| hsa-miR-3074-3p | −16.61 | −16.61 | −16.61 | −16.61 | −16.61 |
| hsa-miR-3074-5p | 3.925 | 4.783 | 2.856 | 2.024 | 2.78 |
| hsa-miR-3085-3p | −16.61 | −16.61 | −16.61 | −16.61 | −16.61 |
| hsa-miR-3085-5p | −16.61 | −16.61 | −16.61 | −16.61 | −16.61 |
| hsa-miR-30a-3p | 10.595 | 8.673 | 6.353 | 6.705 | 8.755 |
| hsa-miR-30a-5p | 12.878 | 11.022 | 9.841 | 10.391 | 11.307 |
| hsa-miR-30b-3p | 3.586 | −16.61 | 0.727 | −16.61 | 5.481 |
| hsa-miR-30b-5p/ | 12.365 | 10.992 | 11.376 | 10.819 | 10.054 |
| 30c-5p | | | | | |
| hsa-miR-30c-1-3p | 5.248 | 4.579 | 3.975 | 4.024 | 6.699 |
| hsa-miR-30c-2-3p | 7.136 | 5.734 | 2.771 | 4.003 | 4.91 |
| hsa-miR-30d-3p | 4.533 | 4.512 | 4.119 | 3.736 | 3.95 |
| hsa-miR-30d-5p | 11.972 | 11.993 | 11.696 | 11.914 | 12.462 |
| hsa-miR-30e-3p | 10.399 | 10.046 | 10.103 | 10.006 | 11.799 |
| hsa-miR-30e-5p | 12.699 | 12.634 | 13.613 | 13.576 | 14.484 |
| hsa-miR-31-3p | 4.766 | 2.342 | −16.61 | −16.61 | −16.61 |
| hsa-miR-31-5p | 9.061 | 7.402 | 4.704 | 3.761 | 4.868 |
| hsa-miR-3115 | 3.128 | 1.857 | 1.727 | 0.176 | −16.61 |
| hsa-miR-3116 | −16.61 | 0.535 | −16.61 | −16.61 | −16.61 |
| hsa-miR-3117-3p | 4.066 | 4.316 | 1.049 | −16.61 | −16.61 |
| hsa-miR-3117-5p | −16.61 | −16.61 | −16.61 | −16.61 | −16.61 |
| hsa-miR-3118 | −16.61 | −16.61 | −16.61 | −16.61 | −16.61 |
| hsa-miR-3119 | −16.61 | −16.61 | −16.61 | −16.61 | −16.61 |
| hsa-miR-3120-3p | −16.61 | −16.61 | −16.61 | −16.61 | −16.61 |
| hsa-miR-3120-5p | −16.61 | −16.61 | −16.61 | −16.61 | −16.61 |
| hsa-miR-3121-3p | −16.61 | −16.61 | −16.61 | −16.61 | −16.61 |
| hsa-miR-3121-5p | −16.61 | −16.61 | −16.61 | −16.61 | −16.61 |
| hsa-miR-3122 | −16.61 | −16.61 | −16.61 | −16.61 | −16.61 |
| hsa-miR-3123 | −16.61 | −16.61 | −16.61 | −16.61 | −16.61 |
| hsa-miR-3124-3p | −16.61 | −16.61 | −16.61 | −16.61 | −16.61 |
| hsa-miR-3124-5p | 4.177 | 2.442 | 2.634 | 1.761 | 1.78 |
| hsa-miR-3125-3p | −16.61 | −16.61 | −16.61 | 16.61 | −16.61 |
| hsa-miR-3125-5p | 5.436 | −16.61 | −16.61 | −16.61 | −16.61 |
| hsa-miR-3126-3p | 3.674 | 0.535 | 2.251 | −16.61 | −16.61 |
| hsa-miR-3126-5p | −16.61 | −16.61 | −16.61 | −16.61 | −16.61 |
| hsa-miR-3127-3p | −16.61 | −16.61 | −16.61 | −16.61 | −16.61 |
| hsa-miR-3127-5p | 4.721 | 4.342 | 3.187 | 4.176 | 4.102 |
| hsa-miR-3128 | −16.61 | −16.61 | 2.251 | 1.176 | −16.61 |
| hsa-miR-3129-3p | −16.61 | −16.61 | −16.61 | −16.61 | −16.61 |
| hsa-miR-3129-5p | 2.418 | 1.705 | −16.61 | −16.61 | −16.61 |
| hsa-miR-3130-3p | 1.524 | 4.027 | −16.61 | −16.61 | −16.61 |
| hsa-miR-3130-5p | −16.61 | −16.61 | −16.61 | −16.61 | −16.61 |
| hsa-miR-3131 | −16.61 | 3.12 | 1.312 | −16.61 | −16.61 |
| hsa-miR-3132 | −16.61 | −16.61 | −16.61 | −16.61 | −16.61 |
| hsa-miR-3133 | −16.61 | −16.61 | −16.61 | 2.854 | −16.61 |
| hsa-miR-3134 | −16.61 | −16.61 | −16.61 | −16.61 | −16.61 |
| hsa-miR-3135a | −16.61 | −16.61 | −16.61 | 2.246 | −16.61 |
| hsa-miR-3135b | −16.61 | −16.61 | −16.61 | −16.61 | −16.61 |
| hsa-miR-3136-3p | −16.61 | −16.61 | −16.61 | −16.61 | −16.61 |
| hsa-miR-3136-5p | −16.61 | −16.61 | 2.534 | 2.712 | 3.365 |
| hsa-miR-3137 | −16.61 | −16.61 | −16.61 | −16.61 | −16.61 |
| hsa-miR-3138-3p | 3.332 | 3.994 | −16.61 | 3.784 | 5.102 |
| hsa-miR-3138-5p | −16.61 | −16.61 | −16.61 | −16.61 | −16.61 |
| hsa-miR-3139-3p | −16.61 | −16.61 | −16.61 | −16.61 | −16.61 |
| hsa-miR-3139-5p | 4.265 | 1.857 | 0.897 | 1.661 | 2.365 |
| hsa-miR-3140-3p | −16.61 | 0.857 | 1.727 | 3.176 | −16.61 |
| hsa-miR-3140-5p | −16.61 | −16.61 | −16.61 | 2.808 | −16.61 |
| hsa-miR-3141 | −16.61 | −16.61 | −16.61 | −16.61 | −16.61 |
| hsa-miR-3142 | −16.61 | −16.61 | −16.61 | −16.61 | −16.61 |
| hsa-miR-3143 | −16.61 | 0.535 | 2.371 | 0.439 | 4.99 |
| hsa-miR-3144-3p | −16.61 | −16.61 | −16.61 | −16.61 | −16.61 |
| hsa-miR-3144-5p | −16.61 | −16.61 | −16.61 | −16.61 | −16.61 |
| hsa-miR-3145-3p | 3.292 | 3.489 | 2.119 | 3.346 | 4.102 |
| hsa-miR-3145-5p | 2.847 | −16.61 | −16.61 | −16.61 | −16.61 |
| hsa-miR-3146 | −16.61 | 5.135 | 2.897 | 3.554 | 3.868 |
| hsa-miR-3147 | −16.61 | −16.61 | −16.61 | −16.61 | −16.61 |
| hsa-miR-3148 | −16.61 | −16.61 | −16.61 | −16.61 | −16.61 |
| hsa-miR-3149 | 2.589 | −16.61 | −0.273 | 1.313 | 4.588 |
| hsa-miR-3150a-3p | −16.61 | 0.857 | 1.727 | 3.468 | −16.61 |
| hsa-miR-3150a-5p | −16.61 | −16.61 | −16.61 | −16.61 | −16.61 |
| hsa-miR-3150b-3p | −16.61 | −16.61 | 2.428 | 3.024 | −16.61 |
| hsa-miR-3150b-5p | −16.61 | −16.61 | −16.61 | −16.61 | −16.61 |
| hsa-miR-3151-3p | −16.61 | −16.61 | −16.61 | −16.61 | −16.61 |
| hsa-miR-3151-5p | −16.61 | 1.12 | −16.61 | −16.61 | −16.61 |
| hsa-miR-3152-3p | −16.61 | −16.61 | −16.61 | −16.61 | −16.61 |
| hsa-miR-3152-5p | −16.61 | −16.61 | −16.61 | −16.61 | −16.61 |
| hsa-miR-3153-3p | −16.61 | −16.61 | −16.61 | −16.61 | −16.61 |
| hsa-miR-3153-5p | −16.61 | −16.61 | −16.61 | −16.61 | −16.61 |
| hsa-miR-3154 | −16.61 | −16.61 | −16.61 | −16.61 | 4.868 |
| hsa-miR-3155a-3p/ | −16.61 | −16.61 | −16.61 | 2.609 | −16.61 |
| 3155b | | | | | |
| hsa-miR-3155a-5p | −16.61 | −16.61 | −16.61 | 2.176 | −16.61 |
| hsa-miR-3156-3p | −16.61 | −16.61 | −16.61 | −16.61 | −16.61 |
| hsa-miR-3156-5p | −16.61 | −16.61 | −16.61 | −16.61 | −16.61 |
| hsa-miR-3157-3p | −16.61 | −16.61 | −16.61 | 1.761 | −16.61 |
| hsa-miR-3157-5p | −16.61 | −0.465 | −16.61 | 2.854 | 3.102 |
| hsa-miR-3158-3p | −16.61 | −16.61 | −16.61 | −16.61 | 3.365 |
| hsa-miR-3158-5p | −16.61 | −16.61 | −16.61 | −16.61 | −16.61 |
| hsa-miR-3159 | −16.61 | −0.465 | −16.61 | −16.61 | 2.365 |
| hsa-miR-3160-3p | −16.61 | −16.61 | −16.61 | −16.61 | −16.61 |
| hsa-miR-3160-5p | −16.61 | −16.61 | −16.61 | −16.61 | −16.61 |
| hsa-miR-3161-3p | −16.61 | −16.61 | −16.61 | −16.61 | −16.61 |
| hsa-miR-3161-5p | −16.61 | −16.61 | −16.61 | −16.61 | −16.61 |
| hsa-miR-3162-3p | −16.61 | −16.61 | −16.61 | −16.61 | −16.61 |
| hsa-miR-3162-5p | −16.61 | −16.61 | −16.61 | −16.61 | −16.61 |
| hsa-miR-3163 | −16.61 | −16.61 | −16.61 | −16.61 | −16.61 |
| hsa-miR-3164-3p | −16.61 | −16.61 | 0.897 | −16.61 | −16.61 |
| hsa-miR-3164-5p | 2.215 | 0.857 | −16.61 | 2.609 | −16.61 |
| hsa-miR-3165-3p | −16.61 | −16.61 | −16.61 | −16.61 | −16.61 |
| hsa-miR-3165-5p | −16.61 | −16.61 | −16.61 | −16.61 | 2.95 |
| hsa-miR-3166 | −16.61 | −16.61 | −16.61 | −16.61 | −16.61 |
| hsa-miR-3167 | 2.258 | −16.61 | −16.61 | −16.61 | −16.61 |
| hsa-miR-3168 | −16.61 | −16.61 | −16.61 | −16.61 | −16.61 |
| hsa-miR-3169-3p | −16.61 | −16.61 | −16.61 | −16.61 | −16.61 |
| hsa-miR-3169-5p | −16.61 | −16.61 | −16.61 | −16.61 | −16.61 |
| hsa-miR-3170 | −16.61 | 0.535 | −16.61 | −16.61 | −16.61 |
| hsa-miR-3171 | −16.61 | −16.61 | −16.61 | −16.61 | −16.61 |
| hsa-miR-3173-3p | −16.61 | −16.61 | −16.61 | −16.61 | −16.61 |
| hsa-miR-3173-5p | 2.589 | −16.61 | 2.251 | 1.854 | 3.24 |
| hsa-miR-3174 | −16.61 | 0.857 | 3.634 | 3.661 | 5.758 |
| hsa-miR-3175-3p | −16.61 | −16.61 | −16.61 | −16.61 | −16.61 |
| hsa-miR-3175-5p | 5.582 | −16.61 | −16.61 | −16.61 | −16.61 |
| hsa-miR-3176-3p | −16.61 | 0.12 | −16.61 | 1.554 | −16.61 |
| hsa-miR-3176-5p | −16.61 | −16.61 | −16.61 | −16.61 | −16.61 |
| hsa-miR-3177-3p | 1.368 | −16.61 | −16.61 | −16.61 | −16.61 |
| hsa-miR-3177-5p | −16.61 | −16.61 | −16.61 | −16.61 | −16.61 |
| hsa-miR-3178 | −16.61 | −16.61 | −16.61 | −16.61 | −16.61 |
| hsa-miR-3179-3p | −16.61 | −16.61 | −16.61 | −16.61 | 3.588 |
| hsa-miR-3179-5p | −16.61 | −16.61 | −16.61 | −16.61 | −16.61 |
| hsa-miR-3180-5p | −16.61 | 1.12 | −16.61 | −16.61 | −16.61 |
| hsa-miR-3180/3180-3p | −16.61 | 2.235 | −16.61 | −16.61 | 2.102 |
| hsa-miR-3181 | −16.61 | −16.61 | −16.61 | −16.61 | −16.61 |
| hsa-miR-3182 | 3.069 | −16.61 | 4.522 | 4.635 | 4.735 |
| hsa-miR-3183-3p | 3.679 | −16.61 | 1.815 | −16.61 | −16.61 |
| hsa-miR-3183-5p | −16.61 | −16.61 | 1.727 | 2.024 | 2.78 |
| hsa-miR-3184-3p | −16.61 | −16.61 | −16.61 | −16.61 | −16.61 |
| hsa-miR-3184-5p | −16.61 | −16.61 | −16.61 | −16.61 | −16.61 |
| hsa-miR-3185 | −16.61 | −16.61 | −16.61 | −16.61 | −16.61 |
| hsa-miR-3186-3p | −16.61 | −16.61 | −16.61 | −16.61 | −16.61 |
| hsa-miR-3186-5p | −16.61 | −16.61 | −16.61 | −16.61 | −16.61 |
| hsa-miR-3187-3p | 3.382 | 4.027 | 3.119 | 2.808 | 2.95 |
| hsa-miR-3187-5p | 3.487 | −16.61 | −16.61 | −16.61 | −16.61 |
| hsa-miR-3188-3p | −16.61 | 1.535 | 0.727 | −0.146 | 4.687 |
| hsa-miR-3188-5p | −16.61 | −16.61 | −16.61 | −16.61 | −16.61 |
| hsa-miR-3189-3p | −16.61 | −16.61 | −16.61 | −16.61 | −16.61 |
| hsa-miR-3189-5p | −16.61 | −16.61 | −16.61 | −16.61 | −16.61 |
| hsa-miR-3190-3p | −16.61 | 1.994 | 1.815 | 3.102 | 4.535 |
| hsa-miR-3190-5p | 16.61 | −16.61 | −16.61 | −16.61 | −16.61 |
| hsa-miR-3191-3p | −16.61 | −16.61 | 1.975 | −16.61 | 2.588 |
| hsa-miR-3191-5p | −16.61 | −16.61 | 0.049 | −16.61 | −16.61 |
| hsa-miR-3192-3p | −16.61 | −16.61 | −16.61 | −16.61 | −16.61 |
| hsa-miR-3192-5p | −16.61 | 3.994 | 1.815 | −16.61 | 5.102 |
| hsa-miR-3193 | −16.61 | 0.535 | 1.727 | −16.61 | −16.61 |
| hsa-miR-3194-3p | −16.61 | −16.61 | −16.61 | −16.61 | −16.61 |
| hsa-miR-3194-5p | −16.61 | 0.12 | 2.049 | 0.661 | 4.173 |
| hsa-miR-3195 | 4.238 | 6.01 | 6.166 | 6.393 | 6.857 |
| hsa-miR-3196 | −16.61 | −16.61 | −16.61 | −16.61 | −16.61 |
| hsa-miR-3197-3p | −16.61 | −16.61 | −16.61 | −16.61 | −16.61 |
| hsa-miR-3197-5p | −16.61 | −16.61 | −16.61 | −16.61 | −16.61 |
| hsa-miR-3198-3p | −16.61 | 4.442 | 2.727 | 1.854 | 3.687 |
| hsa-miR-3198-5p | −16.61 | −16.61 | −16.61 | −16.61 | −16.61 |
| hsa-miR-3199-3p | −16.61 | −16.61 | −16.61 | −16.61 | 3.588 |
| hsa-miR-3199-5p | −16.61 | −16.61 | 2.119 | −16.61 | −16.61 |
| hsa-miR-32-3p | 4.949 | 6.12 | 4.455 | 4.468 | 9.272 |
| hsa-miR-32-5p | 7.539 | 7.093 | 7.786 | 7.876 | 5.173 |
| hsa-miR-3200-3p | 7.658 | 5.744 | 1.727 | 2.313 | 2.588 |
| hsa-miR-3200-5p | 2.681 | −16.61 | −16.61 | −16.61 | −16.61 |
| hsa-miR-3201 | −16.61 | −16.61 | −16.61 | −16.61 | −16.61 |
| hsa-miR-3202 | −16.61 | −16.61 | −16.61 | −16.61 | 3.24 |
| hsa-miR-320a-3p/ | 10.731 | 11.294 | 10.122 | 10.76 | 14.778 |
| 320b/320c/320d/320e | | | | | |
| hsa-miR-320a-5p | −16.61 | −16.61 | −16.61 | −16.61 | −16.61 |
| hsa-miR-323a-3p | 4.679 | 4.725 | 0.534 | 0.439 | −16.61 |
| hsa-miR-323a-5p | −16.61 | −16.61 | −16.61 | −16.61 | −16.61 |
| hsa-miR-323b-3p | 4.264 | 4.512 | 0.534 | −0.146 | −16.61 |
| hsa-miR-323b-5p | −16.61 | −16.61 | −16.61 | −16.61 | −16.61 |
| hsa-miR-324-3p | 8.422 | 7.908 | 7.646 | 7.612 | 6.803 |
| hsa-miR-324-5p | 9.395 | 8.445 | 8.818 | 8.131 | 11.234 |
| hsa-miR-325-3p | −16.61 | −16.61 | −16.61 | −16.61 | −16.61 |
| hsa-miR-325-5p | −16.61 | −16.61 | −16.61 | −16.61 | −16.61 |
| hsa-miR-326-3p | −16.61 | −16.61 | −16.61 | −16.61 | −16.61 |
| hsa-miR-326-5p | −16.61 | −16.61 | −16.61 | −16.61 | −16.61 |
| hsa-miR-328-3p | 8.195 | 7.702 | 6.389 | 5.967 | 4.687 |
| hsa-miR-328-5p | −16.61 | −16.61 | −16.61 | −16.61 | −16.61 |
| hsa-miR-329-3p | 2.781 | 2.342 | 1.727 | 0.661 | −16.61 |
| hsa-miR-329-5p | 3.226 | 1.12 | −16.61 | −16.61 | −16.61 |
| hsa-miR-330-3p | 5.88 | 5.801 | 3.585 | 4.211 | 4.687 |
| hsa-miR-330-5p | 5.118 | 4.207 | 4.681 | 4.983 | 4.588 |
| hsa-miR-331-3p | 8.804 | 6.512 | 6.32 | 5.773 | 5.535 |
| hsa-miR-331-5p | 2.407 | 1.342 | 1.049 | 1.661 | −16.61 |
| hsa-miR-335-3p | 5.432 | 4.705 | 1.897 | 2.102 | −16.61 |
| hsa-miR-335-5p | 15.424 | 12.651 | 9.315 | 9.186 | 10.815 |
| hsa-miR-337-3p | −16.61 | 0.535 | −0.688 | −16.61 | −16.61 |
| hsa-miR-337-5p | −16.61 | 4.12 | 1.428 | 2.176 | −16.61 |
| hsa-miR-338-3p | 8.856 | 8.485 | 7.903 | 7.905 | 5.481 |
| hsa-miR-338-5p | 3.579 | −16.61 | −16.61 | −16.61 | −16.61 |
| hsa-miR-339-3p | 8.089 | 6.815 | 5.721 | 6.263 | 9.248 |
| hsa-miR-339-5p | 9.111 | 8.413 | 8.986 | 9.52 | 9.472 |
| hsa-miR-33a-3p | 3.699 | 1.342 | 3.455 | 2.983 | 4.304 |
| hsa-miR-33a-5p | 7.475 | 7.593 | 8.604 | 8.098 | 5.102 |
| hsa-miR-33b-3p | −16.61 | −16.61 | −16.61 | −16.61 | 2.365 |
| hsa-miR-33b-5p | 3.666 | 3.857 | 3.975 | 2.854 | 2.365 |
| hsa-miR-340-3p | 4.676 | 4.622 | 4.385 | 4.761 | 3.481 |
| hsa-miR-340-5p | 11.198 | 11.153 | 11.18 | 11.82 | 11.691 |
| hsa-miR-342-3p | 13.609 | 13.362 | 12.832 | 13.13 | 12.799 |
| hsa-miR-342-5p | 6.159 | 5.454 | 5.793 | 6.259 | 9.496 |
| hsa-miR-345-3p | 4.183 | −16.61 | 2.049 | 3.854 | −16.61 |
| hsa-miR-345-5p | 8.292 | 8.291 | 8.223 | 8.416 | 8.343 |
| hsa-miR-346 | 3.204 | 1.857 | −16.61 | −16.61 | −16.61 |
| hsa-miR-34a-3p | 5.281 | 2.857 | 1.975 | 4.54 | 3.687 |
| hsa-miR-34a-5p | 12.596 | 12.7 | 10.776 | 12.389 | 12.806 |
| hsa-miR-34b-3p | 3.596 | 3.961 | 1.187 | 2.246 | −16.61 |
| hsa-miR-34b-5p | 1.709 | 1.535 | 0.727 | −0.146 | 2.78 |
| hsa-miR-34c-3p | 4.031 | −16.61 | −16.61 | −16.61 | −16.61 |
| hsa-miR-34c-5p | 3.393 | 3.489 | 2.312 | 2.854 | 3.365 |
| hsa-miR-3529-3p | −16.61 | −16.61 | −16.61 | −16.61 | −16.61 |
| hsa-miR-3529-5p | 3.655 | −16.61 | −16.61 | −16.61 | −16.61 |
| hsa-miR-3605-3p | 4.531 | 4.783 | 5.04 | 4.687 | 5.173 |
| hsa-miR-3605-5p | 4.375 | 4.29 | 3.049 | 4.024 | 5.95 |
| hsa-miR-3606-3p | −16.61 | −16.61 | −16.61 | −16.61 | −16.61 |
| hsa-miR-3606-5p | −16.61 | −16.61 | −16.61 | −16.61 | −16.61 |
| hsa-miR-3609 | 3.964 | −16.61 | 2.534 | 0.854 | 3.95 |
| hsa-miR-361-3p | 9.751 | 9.559 | 9.7 | 9.796 | 5.272 |
| hsa-miR-361-5p | 9.636 | 9.279 | 9.374 | 9.824 | 10.558 |
| hsa-miR-3610 | −16.61 | −16.61 | −16.61 | −16.61 | −16.61 |
| hsa-miR-3611-3p | 4.303 | 3.664 | 4.153 | 4.568 | 7.028 |
| hsa-miR-3611-5p | −16.61 | −16.61 | 16.61 | −16.61 | −16.61 |
| hsa-miR-3612 | 4.156 | −16.61 | 16.61 | −16.61 | −16.61 |
| hsa-miR-3613-3p | −16.61 | 2.342 | 1.312 | 2.176 | −16.61 |
| hsa-miR-3613-5p | 6.858 | 7.761 | 7.758 | 7.582 | 4.868 |
| hsa-miR-3614-3p | −16.61 | −16.61 | −16.61 | −16.61 | −16.61 |
| hsa-miR-3614-5p | −16.61 | −16.61 | −16.61 | 2.176 | 3.687 |
| hsa-miR-3615-3p | 3.871 | 4.978 | 4.877 | 4.121 | 5.424 |
| hsa-miR-3615-5p | −16.61 | −16.61 | −16.61 | −16.61 | −16.61 |
| hsa-miR-3616-3p | 2.924 | −16.61 | −16.61 | −16.61 | −16.61 |
| hsa-miR-3616-5p | −16.61 | −16.61 | −16.61 | 2.176 | −16.61 |
| hsa-miR-3617-3p | −16.61 | −16.61 | −16.61 | −16.61 | −16.61 |
| hsa-miR-3617-5p | −16.61 | −16.61 | 1.428 | −16.61 | −16.61 |
| hsa-miR-3618 | −16.61 | −16.61 | −16.61 | −16.61 | −16.61 |
| hsa-miR-3619-3p | −16.61 | −16.61 | −16.61 | −16.61 | −16.61 |
| hsa-miR-3619-5p | −16.61 | −16.61 | −16.61 | −16.61 | −16.61 |
| hsa-miR-362-3p | 4.441 | 2.235 | 3.836 | 2.313 | −16.61 |
| hsa-miR-362-5p | 5.176 | 4.149 | 6.049 | 6.078 | 8.391 |
| hsa-miR-3620-3p | −16.61 | −16.61 | −16.61 | −16.61 | −16.61 |
| hsa-miR-3620-5p | −16.61 | −16.61 | −16.61 | −16.61 | −16.61 |
| hsa-miR-3621 | −16.61 | −16.61 | −16.61 | −16.61 | −16.61 |
| hsa-miR-3622a-3p | −16.61 | −16.61 | −16.61 | −16.61 | −16.61 |
| hsa-miR-3622a-5p | −16.61 | −16.61 | −16.61 | −16.61 | −16.61 |
| hsa-miR-3622b-3p | −16.61 | −16.61 | −16.61 | −16.61 | −16.61 |
| hsa-miR-3622b-5p | −16.61 | −16.61 | −16.61 | −16.61 | −16.61 |
| hsa-miR-363-3p | 7.71 | 8.295 | 7.738 | 7.352 | 5.735 |
| hsa-miR-363-5p | 4.654 | 2.12 | −16.61 | 2.176 | −16.61 |
| hsa-miR-3646 | 16.61 | −16.61 | −16.61 | −16.61 | −16.61 |
| hsa-miR-3648-3p | −16.61 | −16.61 | −16.61 | −16.61 | −16.61 |
| hsa-miR-3648-5p | −16.61 | −16.61 | −16.61 | −16.61 | −16.61 |
| hsa-miR-3649 | −16.61 | −16.61 | −16.61 | −16.61 | −16.61 |
| hsa-miR-3650 | −16.61 | −16.61 | −16.61 | −16.61 | −16.61 |
| hsa-miR-3651 | −16.61 | −16.61 | −16.61 | 2.246 | −16.61 |
| hsa-miR-3652-3p | −16.61 | −16.61 | −16.61 | −16.61 | −16.61 |
| hsa-miR-3652-5p | −16.61 | −16.61 | −16.61 | −16.61 | −16.61 |
| hsa-miR-3654 | −16.61 | −16.61 | −16.61 | −16.61 | −16.61 |
| hsa-miR-3655 | −16.61 | −16.61 | −16.61 | −16.61 | −16.61 |
| hsa-miR-3657 | −16.61 | −16.61 | 0.534 | 0.854 | −16.61 |
| hsa-miR-3658 | −16.61 | −16.61 | −16.61 | −16.61 | −16.61 |
| hsa-miR-3659-3p | −2.696 | −16.61 | −16.61 | −16.61 | −16.61 |
| hsa-miR-3659-5p | −16.61 | −16.61 | −16.61 | −16.61 | −16.61 |
| hsa-miR-365a-3p/ | 7.621 | 7.348 | 5.364 | 6.325 | 7.687 |
| 365b-3p | | | | | |
| hsa-miR-365a-5p | −16.61 | −16.61 | −1.273 | −16.61 | 2.365 |
| hsa-miR-365b-5p | 4.29 | 4.442 | 0.727 | 2.246 | 3.102 |
| hsa-miR-3660 | 4.291 | −16.61 | 1.897 | 1.854 | 3.24 |
| hsa-miR-3661 | 16.61 | 1.342 | −16.61 | −16.61 | −16.61 |
| hsa-miR-3662 | −16.61 | −16.61 | −16.61 | −16.61 | −16.61 |
| hsa-miR-3663-3p | −16.61 | −16.61 | −16.61 | −16.61 | −16.61 |
| hsa-miR-3663-5p | −16.61 | −16.61 | −16.61 | −16.61 | −16.61 |
| hsa-miR-3664-3p | 4.059 | −16.61 | −16.61 | −16.61 | −16.61 |
| hsa-miR-3664-5p | −16.61 | −16.61 | −16.61 | −16.61 | −16.61 |
| hsa-miR-3665 | −16.61 | −16.61 | −16.61 | −16.61 | −16.61 |
| hsa-miR-3666 | −16.61 | −16.61 | −16.61 | −16.61 | −16.61 |
| hsa-miR-3667-3p | −16.61 | −16.61 | −16.61 | −16.61 | −16.61 |
| hsa-miR-3667-5p | −16.61 | −16.61 | −16.61 | −16.61 | −16.61 |
| hsa-miR-3668-3p | −16.61 | −16.61 | −16.61 | −16.61 | −16.61 |
| hsa-miR-3668-5p | −16.61 | −16.61 | −16.61 | −16.61 | −16.61 |
| hsa-miR-367-3p | −16.61 | 3.579 | −0.273 | −0.561 | −16.61 |
| hsa-miR-367-5p | −16.61 | −16.61 | −16.61 | −16.61 | −16.61 |
| hsa-miR-3670 | −16.61 | −16.61 | −16.61 | −16.61 | −16.61 |
| hsa-miR-3671 | −16.61 | −16.61 | −16.61 | −16.61 | −16.61 |
| hsa-miR-3672-3p | −16.61 | −16.61 | −16.61 | −16.61 | −16.61 |
| hsa-miR-3672-5p | −16.61 | −16.61 | −16.61 | −16.61 | −16.61 |
| hsa-miR-3674 | −16.61 | −16.61 | −16.61 | −16.61 | −16.61 |
| hsa-miR-3675-3p | −16.61 | −16.61 | −16.61 | −16.61 | −16.61 |
| hsa-miR-3675-5p | −16.61 | −16.61 | −16.61 | −16.61 | 3.24 |
| hsa-miR-3677-3p | −16.61 | −16.61 | −16.61 | −16.61 | −16.61 |
| hsa-miR-3677-5p | −16.61 | −16.61 | −16.61 | −16.61 | −16.61 |
| hsa-miR-3678-3p | −16.61 | −16.61 | −16.61 | 1.439 | −16.61 |
| hsa-miR-3678-5p | −16.61 | −16.61 | −16.61 | −16.61 | −16.61 |
| hsa-miR-3679-3p | 2.368 | −0.465 | −16.61 | −16.61 | −16.61 |
| hsa-miR-3679-5p | 5.021 | 6.568 | 3.012 | 5.003 | 5.99 |
| hsa-miR-3680-3p | −16.61 | −16.61 | −16.61 | −16.61 | −16.61 |
| hsa-miR-3680-5p | −16.61 | −16.61 | −16.61 | 2.439 | −16.61 |
| hsa-miR-3681-3p | −16.61 | −16.61 | −16.61 | −16.61 | −16.61 |
| hsa-miR-3681-5p | 0.626 | −16.61 | 1.727 | −16.61 | −16.61 |
| hsa-miR-3682-3p | −16.61 | −16.61 | −16.61 | −16.61 | −16.61 |
| hsa-miR-3682-5p | −16.61 | −16.61 | −16.61 | −16.61 | −16.61 |
| hsa-miR-3683-3p | −16.61 | −16.61 | −16.61 | −16.61 | −16.61 |
| hsa-miR-3683-5p | −16.61 | −16.61 | −16.61 | −16.61 | −16.61 |
| hsa-miR-3684 | −16.61 | −16.61 | 1.312 | 0.439 | −16.61 |
| hsa-miR-3685 | −16.61 | −16.61 | −16.61 | −16.61 | −16.61 |
| hsa-miR-3686 | −16.61 | −16.61 | −16.61 | −16.61 | −16.61 |
| hsa-miR-3688-3p | 3.171 | 3.058 | 1.815 | 2.024 | 3.24 |
| hsa-miR-3688-5p | −16.61 | −16.61 | −16.61 | −16.61 | −16.61 |
| hsa-miR-3689a-3p | −16.61 | −16.61 | −16.61 | −16.61 | −16.61 |
| hsa-miR-3689a-5p/ | −16.61 | −16.61 | −16.61 | −16.61 | −16.61 |
| 3689b-5p/3689e | | | | | |
| hsa-miR-3689b-3p/ | −16.61 | −16.61 | −16.61 | −16.61 | −16.61 |
| 3689c | | | | | |
| hsa-miR-3689d | −16.61 | −16.61 | −16.61 | −16.61 | −16.61 |
| hsa-miR-3689f | −16.61 | −16.61 | −16.61 | −16.61 | −16.61 |
| hsa-miR-369-3p | 4.144 | 4.801 | 2.428 | 2.808 | −16.61 |
| hsa-miR-369-5p | 2.847 | 3.705 | 0.049 | 1.439 | 1.365 |
| hsa-miR-3690 | −16.61 | −16.61 | 0.897 | 3.063 | 0.78 |
| hsa-miR-3691-3p | −16.61 | −16.61 | −16.61 | −16.61 | −16.61 |
| hsa-miR-3691-5p | 3.896 | 1.535 | −16.61 | 1.176 | 3.588 |
| hsa-miR-3692-3p | −16.61 | −16.61 | −16.61 | −16.61 | −16.61 |
| hsa-miR-3692-5p | −16.61 | −16.61 | −16.61 | 2.983 | −16.61 |
| hsa-miR-370-3p | 2.935 | 5.978 | 1.727 | 2.102 | −16.61 |
| hsa-miR-370-5p | −16.61 | 0.857 | −16.61 | −16.61 | −16.61 |
| hsa-miR-3713 | −16.61 | −16.61 | −16.61 | −16.61 | −16.61 |
| hsa-miR-3714-3p | −16.61 | −16.61 | −16.61 | −16.61 | −16.61 |
| hsa-miR-3714-5p | −16.61 | −16.61 | −16.61 | −16.61 | −16.61 |
| hsa-miR-371a-3p | −16.61 | 2.442 | −16.61 | −16.61 | −16.61 |
| hsa-miR-371a-5p | −16.61 | 3.342 | 1.049 | −16.61 | −16.61 |
| hsa-miR-371b-3p | −16.61 | −16.61 | −16.61 | −16.61 | −16.61 |
| hsa-miR-371b-5p | −16.61 | −16.61 | −16.61 | −16.61 | −16.61 |
| hsa-miR-372-3p | −16.61 | 3.857 | 1.534 | 0.854 | −16.61 |
| hsa-miR-372-5p | −16.61 | 2.535 | −16.61 | −16.61 | −16.61 |
| hsa-miR-373-3p | −16.61 | 6.477 | 2.727 | 2.941 | −16.61 |
| hsa-miR-373-5p | −16.61 | −16.61 | −16.61 | −16.61 | −16.61 |
| hsa-miR-374a-3p | 9.588 | 8.283 | 8.062 | 7.973 | 6.78 |
| hsa-miR-374a-5p | 9.826 | 8.623 | 9.02 | 9.092 | 7.723 |
| hsa-miR-374b-3p | 6.636 | 5.368 | 4.495 | 4.194 | 5.663 |
| hsa-miR-374b-5p | 7.834 | 6.552 | 6.752 | 6.599 | 7.129 |
| hsa-miR-374c-3p | −16.61 | −16.61 | −16.61 | −16.61 | −16.61 |
| hsa-miR-374c-5p | −16.61 | −16.61 | −16.61 | −16.61 | −16.61 |
| hsa-miR-375-3p | 5.686 | 6.05 | 1.634 | 2.898 | −16.61 |
| hsa-miR-375-5p | −16.61 | −16.61 | −16.61 | −16.61 | −16.61 |
| hsa-miR-376a-2-5p | −16.61 | 1.12 | −16.61 | −16.61 | −16.61 |
| hsa-miR-376a-3p/ | 6.007 | 6.411 | 3.187 | 2.246 | 2.588 |
| 376b-3p | | | | | |
| hsa-miR-376a-5p | −16.61 | −16.61 | −16.61 | −16.61 | −16.61 |
| hsa-miR-376b-5p/ | 2.674 | 1.342 | −16.61 | −16.61 | −16.61 |
| 376c-5p | | | | | |
| hsa-miR-376c-3p | 5.069 | 4.089 | 1.312 | 1.761 | 3.24 |
| hsa-miR-377-3p | −16.61 | 1.857 | −16.61 | −16.61 | −16.61 |
| hsa-miR-377-5p | −16.61 | −16.61 | −16.61 | −16.61 | −16.61 |
| hsa-miR-378a-3p/ | 9.194 | 11.9 | 12.154 | 13.031 | 13.907 |
| 378c/378d/378e | | | | | |
| hsa-miR-378a-5p | 6.867 | 7.399 | 7.884 | 8.733 | 7.995 |
| hsa-miR-378b | −16.61 | −16.61 | −16.61 | −16.61 | −16.61 |
| hsa-miR-378f | −16.61 | −16.61 | −16.61 | −16.61 | −16.61 |
| hsa-miR-378g | −16.61 | 2.705 | 3.56 | −16.61 | −16.61 |
| hsa-miR-378h | −16.61 | −16.61 | −16.61 | −16.61 | −16.61 |
| hsa-miR-378i | −16.61 | 2.235 | 2.634 | 4.024 | 3.95 |
| hsa-miR-378j | −16.61 | −16.61 | −16.61 | −16.61 | −16.61 |
| hsa-miR-379-3p | −16.61 | −16.61 | −16.61 | −16.61 | −16.61 |
| hsa-miR-379-5p | 4.721 | 7.471 | 3.634 | 3.408 | −16.61 |
| hsa-miR-380-3p | −16.61 | −16.61 | −16.61 | −16.61 | −16.61 |
| hsa-miR-380-5p | −16.61 | 3.393 | −16.61 | −16.61 | −16.61 |
| hsa-miR-381-3p | 4.954 | 6.303 | 2.482 | 2.941 | −16.61 |
| hsa-miR-381-5p | −16.61 | −16.61 | −16.61 | −16.61 | −16.61 |
| hsa-miR-382-3p | 4.343 | 3.994 | −16.61 | −16.61 | −16.61 |
| hsa-miR-382-5p | 6.363 | 7.617 | 3.771 | 3.898 | 4.481 |
| hsa-miR-383-3p | −16.61 | −16.61 | −16.61 | −16.61 | −16.61 |
| hsa-miR-383-5p | 4.224 | 3.393 | 0.534 | −16.61 | −16.61 |
| hsa-miR-384 | −16.61 | −16.61 | −16.61 | −16.61 | −16.61 |
| hsa-miR-3907 | −16.61 | −16.61 | −16.61 | −16.61 | −16.61 |
| hsa-miR-3908 | −16.61 | −16.61 | −16.61 | −16.61 | −16.61 |
| hsa-miR-3909-3p | −16.61 | −16.61 | −16.61 | −16.61 | −16.61 |
| hsa-miR-3909-5p | −16.61 | −16.61 | −16.61 | −16.61 | −16.61 |
| hsa-miR-3910 | −16.61 | −16.61 | −16.61 | −16.61 | −16.61 |
| hsa-miR-3911 | −16.61 | −16.61 | −16.61 | −16.61 | −16.61 |
| hsa-miR-3912-3p | 2.684 | 0.857 | 2.482 | 2.439 | −16.61 |
| hsa-miR-3912-5p | −16.61 | −16.61 | −16.61 | −16.61 | −16.61 |
| hsa-miR-3913-3p | −16.61 | 0.535 | −16.61 | −16.61 | −16.61 |
| hsa-miR-3913-5p | 3.875 | 2.235 | 2.975 | 2.498 | 3.78 |
| hsa-miR-3914 | −16.61 | −16.61 | −16.61 | −16.61 | −16.61 |
| hsa-miR-3915 | −16.61 | −16.61 | −16.61 | −16.61 | −16.61 |
| hsa-miR-3916 | −16.61 | −16.61 | 2.251 | −16.61 | −16.61 |
| hsa-miR-3917 | −16.61 | −16.61 | −16.61 | −16.61 | −16.61 |
| hsa-miR-3918-3p | −16.61 | −16.61 | −16.61 | −16.61 | −16.61 |
| hsa-miR-3918-5p | −16.61 | −16.61 | −16.61 | −16.61 | −16.61 |
| hsa-miR-3919 | 4.49 | 0.12 | −16.61 | −16.61 | −16.61 |
| hsa-miR-3920 | −16.61 | −16.61 | −16.61 | −16.61 | −16.61 |
| hsa-miR-3921-3p | −16.61 | −16.61 | −16.61 | −16.61 | −16.61 |
| hsa-miR-3921-5p | −16.61 | −16.61 | −16.61 | −16.61 | −16.61 |
| hsa-miR-3922-3p | −16.61 | −16.61 | −16.61 | −16.61 | −16.61 |
| hsa-miR-3922-5p | −16.61 | −16.61 | −16.61 | −16.61 | −16.61 |
| hsa-miR-3923 | −16.61 | −16.61 | −16.61 | −16.61 | −16.61 |
| hsa-miR-3924-3p | −16.61 | −16.61 | −16.61 | −16.61 | −16.61 |
| hsa-miR-3924-5p | −16.61 | −16.61 | −16.61 | −16.61 | −16.61 |
| hsa-miR-3925-3p | −16.61 | −16.61 | −16.61 | −16.61 | −16.61 |
| hsa-miR-3925-5p | −16.61 | −16.61 | −16.61 | 1.313 | −16.61 |
| hsa-miR-3926 | −16.61 | −16.61 | −16.61 | −16.61 | −16.61 |
| hsa-miR-3927-3p | −16.61 | −16.61 | −16.61 | −16.61 | −16.61 |
| hsa-miR-3927-5p | −16.61 | −16.61 | −16.61 | −16.61 | −16.61 |
| hsa-miR-3928-3p | 3.42 | 4.316 | 3.793 | 4.736 | 5.102 |
| hsa-miR-3928-5p | −16.61 | −16.61 | −16.61 | −16.61 | −16.61 |
| hsa-miR-3929 | 3.965 | 3.12 | −16.61 | −16.61 | −16.61 |
| hsa-miR-3934-3p | −16.61 | −16.61 | −16.61 | −16.61 | −16.61 |
| hsa-miR-3934-5p | −16.61 | −16.61 | −16.61 | −16.61 | −16.61 |
| hsa-miR-3935 | −16.61 | −16.61 | −16.61 | −16.61 | −16.61 |
| hsa-miR-3936-3p | −16.61 | −16.61 | −16.61 | −16.61 | −16.61 |
| hsa-miR-3936-5p | −16.61 | −16.61 | −16.61 | −16.61 | −16.61 |
| hsa-miR-3937-3p | −16.61 | 0.12 | −16.61 | −16.61 | −16.61 |
| hsa-miR-3937-5p | −16.61 | −16.61 | −16.61 | −16.61 | −16.61 |
| hsa-miR-3938 | −16.61 | −16.61 | −16.61 | −16.61 | −16.61 |
| hsa-miR-3939-3p | −16.61 | −16.61 | −16.61 | −16.61 | −16.61 |
| hsa-miR-3939-5p | −16.61 | −16.61 | −16.61 | −16.61 | −16.61 |
| hsa-miR-3940-3p | −16.61 | −16.61 | 1.428 | −16.61 | −16.61 |
| hsa-miR-3940-5p | −16.61 | −16.61 | −16.61 | −16.61 | −16.61 |
| hsa-miR-3941-3p | −16.61 | −16.61 | −16.61 | −16.61 | −16.61 |
| hsa-miR-3941-5p | −16.61 | −16.61 | −16.61 | −16.61 | −16.61 |
| hsa-miR-3942-3p | −16.61 | −16.61 | −16.61 | −16.61 | −16.61 |
| hsa-miR-3942-5p | −16.61 | −16.61 | −16.61 | −16.61 | −16.61 |
| hsa-miR-3943-3p | −16.61 | −16.61 | −16.61 | −16.61 | −16.61 |
| hsa-miR-3943-5p | 5.042 | 3.442 | −16.61 | 1.554 | −16.61 |
| hsa-miR-3944-3p | 16.61 | −16.61 | −0.273 | −16.61 | −16.61 |
| hsa-miR-3944-5p | −16.61 | −16.61 | −16.61 | −16.61 | 2.588 |
| hsa-miR-3945-3p | −16.61 | −16.61 | −16.61 | 2.554 | −16.61 |
| hsa-miR-3945-5p | −16.61 | −16.61 | −16.61 | −16.61 | −16.61 |
| hsa-miR-3960 | −16.61 | −16.61 | −16.61 | −16.61 | −16.61 |
| hsa-miR-3972 | −16.61 | −16.61 | −16.61 | −16.61 | −16.61 |
| hsa-miR-3973 | −16.61 | −16.61 | −16.61 | −16.61 | −16.61 |
| hsa-miR-3974 | −16.61 | −16.61 | −16.61 | −16.61 | −16.61 |
| hsa-miR-3975 | −16.61 | −16.61 | −16.61 | −16.61 | −16.61 |
| hsa-miR-3976 | −16.61 | −16.61 | −16.61 | −16.61 | −16.61 |
| hsa-miR-3977 | −16.61 | −16.61 | −16.61 | −16.61 | −16.61 |
| hsa-miR-3978 | −16.61 | −16.61 | −16.61 | −16.61 | −16.61 |
| hsa-miR-409-3p | 5.586 | 7.361 | 3.836 | 4.263 | 2.78 |
| hsa-miR-409-5p | −16.61 | 1.342 | −16.61 | 1.439 | −16.61 |
| hsa-miR-410-3p | −16.61 | −16.61 | −16.61 | −16.61 | −16.61 |
| hsa-miR-410-5p | −16.61 | −16.61 | −16.61 | −16.61 | −16.61 |
| hsa-miR-411-3p | −16.61 | −16.61 | −16.61 | −16.61 | −16.61 |
| hsa-miR-411-5p | 4.679 | 6.585 | 3.153 | 3.526 | 3.481 |
| hsa-miR-412-3p | −16.61 | −16.61 | −16.61 | −16.61 | −16.61 |
| hsa-miR-412-5p | −16.61 | 2.442 | −16.61 | −16.61 | −16.61 |
| hsa-miR-421 | 10.036 | 9.375 | 9.459 | 9.982 | 12.3 |
| hsa-miR-422a | −16.61 | −16.61 | −16.61 | −16.61 | −16.61 |
| hsa-miR-423-3p | 8.089 | 8.254 | 8.47 | 8.559 | 7.424 |
| hsa-miR-423-5p | 9.785 | 11.893 | 10.451 | 10.784 | 14.128 |
| hsa-miR-424-3p | 4.448 | 3.179 | 5.716 | 5.568 | 9.597 |
| hsa-miR-424-5p | 10.711 | 12.398 | 12.809 | 13.048 | 13.203 |
| hsa-miR-425-3p | 8.444 | 7.827 | 8.899 | 8.724 | 10.32 |
| hsa-miR-425-5p | 9.216 | 9.503 | 10.528 | 10.387 | 11.271 |
| hsa-miR-4251 | −16.61 | −16.61 | −16.61 | −16.61 | −16.61 |
| hsa-miR-4252 | −16.61 | −16.61 | −16.61 | −16.61 | −16.61 |
| hsa-miR-4253 | −16.61 | −16.61 | −16.61 | −16.61 | −16.61 |
| hsa-miR-4254-3p | −16.61 | −16.61 | −16.61 | −16.61 | −16.61 |
| hsa-miR-4254-5p | −16.61 | −16.61 | −16.61 | −16.61 | −16.61 |
| hsa-miR-4255 | −16.61 | −16.61 | −16.61 | −16.61 | −16.61 |
| hsa-miR-4256 | −16.61 | −16.61 | −16.61 | −16.61 | −16.61 |
| hsa-miR-4257 | −16.61 | −16.61 | −16.61 | −16.61 | −16.61 |
| hsa-miR-4258-3p | −16.61 | −16.61 | −16.61 | −16.61 | −16.61 |
| hsa-miR-4258-5p | −16.61 | −16.61 | −16.61 | −16.61 | −16.61 |
| hsa-miR-4259 | −16.61 | −16.61 | −16.61 | −16.61 | −16.61 |
| hsa-miR-4260 | −16.61 | −16.61 | −16.61 | −16.61 | −16.61 |
| hsa-miR-4261 | −16.61 | −16.61 | −16.61 | −16.61 | −16.61 |
| hsa-miR-4262 | −16.61 | −16.61 | −16.61 | −16.61 | −16.61 |
| hsa-miR-4263 | −16.61 | −16.61 | −16.61 | −16.61 | −16.61 |
| hsa-miR-4264 | −16.61 | −16.61 | −16.61 | −16.61 | −16.61 |
| hsa-miR-4265 | −16.61 | −16.61 | −16.61 | −16.61 | −16.61 |
| hsa-miR-4266 | −16.61 | −16.61 | −16.61 | −16.61 | −16.61 |
| hsa-miR-4267 | −16.61 | −16.61 | −16.61 | −16.61 | −16.61 |
| hsa-miR-4268 | −16.61 | −16.61 | −16.61 | −16.61 | −16.61 |
| hsa-miR-4269 | −16.61 | −16.61 | −16.61 | −16.61 | −16.61 |
| hsa-miR-4270 | −16.61 | 16.61 | −16.61 | −16.61 | −16.61 |
| hsa-miR-4271 | −16.61 | −16.61 | −16.61 | −16.61 | −16.61 |
| hsa-miR-4272 | −16.61 | −16.61 | −16.61 | −16.61 | −16.61 |
| hsa-miR-4273 | −16.61 | −16.61 | −16.61 | −16.61 | −16.61 |
| hsa-miR-4274 | −16.61 | −16.61 | −16.61 | −16.61 | −16.61 |
| hsa-miR-4275 | −16.61 | −16.61 | −16.61 | −16.61 | −16.61 |
| hsa-miR-4276 | −16.61 | −16.61 | −16.61 | −16.61 | −16.61 |
| hsa-miR-4277 | −16.61 | −16.61 | −16.61 | −16.61 | −16.61 |
| hsa-miR-4278 | −16.61 | −16.61 | −16.61 | −16.61 | −16.61 |
| hsa-miR-4279 | −16.61 | −16.61 | −16.61 | −16.61 | −16.61 |
| hsa-miR-4280 | −16.61 | −16.61 | −16.61 | −16.61 | −16.61 |
| hsa-miR-4281 | −16.61 | −16.61 | −16.61 | −16.61 | −16.61 |
| hsa-miR-4282 | −16.61 | −16.61 | −16.61 | −16.61 | −16.61 |
| hsa-miR-4283 | −16.61 | −16.61 | −16.61 | −16.61 | −16.61 |
| hsa-miR-4284 | −16.61 | −16.61 | 0.534 | −16.61 | −16.61 |
| hsa-miR-4285 | −16.61 | −16.61 | −16.61 | −16.61 | −16.61 |
| hsa-miR-4286 | 6.111 | 4.579 | 3.219 | 3.736 | 5.638 |
| hsa-miR-4287 | −16.61 | −16.61 | −16.61 | −16.61 | −16.61 |
| hsa-miR-4288 | −16.61 | −16.61 | −16.61 | −16.61 | −16.61 |
| hsa-miR-4289 | −16.61 | −16.61 | −16.61 | −16.61 | −16.61 |
| hsa-miR-429 | 3.792 | 6.142 | 4.727 | 3.831 | 3.588 |
| hsa-miR-4290 | −16.61 | −16.61 | −16.61 | −16.61 | −16.61 |
| hsa-miR-4291 | −16.61 | −16.61 | −16.61 | −16.61 | −16.61 |
| hsa-miR-4292 | −16.61 | −16.61 | −16.61 | −16.61 | −16.61 |
| hsa-miR-4293 | −16.61 | −16.61 | −16.61 | −16.61 | −16.61 |
| hsa-miR-4294 | −16.61 | −16.61 | −16.61 | −16.61 | −16.61 |
| hsa-miR-4295 | −16.61 | −16.61 | −16.61 | −16.61 | −16.61 |
| hsa-miR-4296 | −16.61 | −16.61 | −16.61 | −16.61 | −16.61 |
| hsa-miR-4297 | −16.61 | −16.61 | −16.61 | −16.61 | −16.61 |
| hsa-miR-4298 | −16.61 | −16.61 | −16.61 | −16.61 | −16.61 |
| hsa-miR-4299 | −16.61 | −16.61 | −16.61 | −16.61 | −16.61 |
| hsa-miR-4300 | −16.61 | −16.61 | −16.61 | −16.61 | −16.61 |
| hsa-miR-4301 | −16.61 | −16.61 | −16.61 | −16.61 | −16.61 |
| hsa-miR-4302 | −16.61 | −16.61 | −16.61 | −16.61 | −16.61 |
| hsa-miR-4303 | −16.61 | −16.61 | −16.61 | −16.61 | −16.61 |
| hsa-miR-4304 | −16.61 | −16.61 | −16.61 | −16.61 | −16.61 |
| hsa-miR-4305 | −16.61 | −16.61 | −16.61 | −16.61 | −16.61 |
| hsa-miR-4306 | −16.61 | −16.61 | −16.61 | −16.61 | −16.61 |
| hsa-miR-4307 | −16.61 | −16.61 | −16.61 | −16.61 | −16.61 |
| hsa-miR-4308 | −16.61 | −16.61 | −16.61 | −16.61 | −16.61 |
| hsa-miR-4309 | −16.61 | −16.61 | −16.61 | −16.61 | −16.61 |
| hsa-miR-431-3p | −16.61 | 3.29 | −16.61 | −16.61 | −16.61 |
| hsa-miR-431-5p | −16.61 | 2.783 | −0.688 | −16.61 | −16.61 |
| hsa-miR-4310-3p | −16.61 | −16.61 | −16.61 | −16.61 | −16.61 |
| hsa-miR-4310-5p | −16.61 | −16.61 | −16.61 | −16.61 | −16.61 |
| hsa-miR-4311 | −16.61 | −16.61 | −16.61 | −16.61 | −16.61 |
| hsa-miR-4312 | −16.61 | −16.61 | −16.61 | −16.61 | −16.61 |
| hsa-miR-4313 | −16.61 | −16.61 | −16.61 | −16.61 | −16.61 |
| hsa-miR-4314 | −16.61 | −16.61 | −16.61 | −16.61 | −16.61 |
| hsa-miR-4315-3p | −16.61 | −16.61 | −16.61 | −16.61 | −16.61 |
| hsa-miR-4315-5p | −16.61 | −16.61 | −16.61 | −16.61 | −16.61 |
| hsa-miR-4316 | −16.61 | −16.61 | −16.61 | −16.61 | −16.61 |
| hsa-miR-4317 | −16.61 | −16.61 | −16.61 | −16.61 | −16.61 |
| hsa-miR-4318 | −16.61 | −16.61 | −16.61 | −16.61 | −16.61 |
| hsa-miR-4319 | −16.61 | −16.61 | −16.61 | −16.61 | −16.61 |
| hsa-miR-432-3p | −16.61 | −16.61 | −16.61 | −16.61 | −16.61 |
| hsa-miR-432-5p | −16.61 | 8.613 | 4.704 | 4.761 | −16.61 |
| hsa-miR-4320 | −16.61 | −16.61 | −16.61 | −16.61 | −16.61 |
| hsa-miR-4321 | −16.61 | −16.61 | −16.61 | −16.61 | −16.61 |
| hsa-miR-4322 | −16.61 | −16.61 | −16.61 | −16.61 | −16.61 |
| hsa-miR-4323 | −16.61 | −16.61 | −16.61 | −16.61 | −16.61 |
| hsa-miR-4324 | −16.61 | −16.61 | −16.61 | −16.61 | −16.61 |
| hsa-miR-4325 | −16.61 | −16.61 | −16.61 | −16.61 | −16.61 |
| hsa-miR-4326 | 2.79 | −16.61 | 2.897 | 2.498 | −16.61 |
| hsa-miR-4327 | −16.61 | −16.61 | −16.61 | −16.61 | −16.61 |
| hsa-miR-4328 | −16.61 | −16.61 | −16.61 | −16.61 | −16.61 |
| hsa-miR-4329 | −16.61 | −16.61 | −16.61 | −16.61 | −16.61 |
| hsa-miR-433-3p | −16.61 | −16.61 | −16.61 | −16.61 | −16.61 |
| hsa-miR-433-5p | −16.61 | −16.61 | −16.61 | −16.61 | −16.61 |
| hsa-miR-4330 | −16.61 | −16.61 | −16.61 | −16.61 | −16.61 |
| hsa-miR-4418 | −16.61 | −16.61 | −16.61 | −16.61 | −16.61 |
| hsa-miR-4420-3p | −16.61 | −16.61 | −16.61 | −16.61 | −16.61 |
| hsa-miR-4420-5p | −16.61 | −16.61 | −16.61 | −16.61 | −16.61 |
| hsa-miR-4421 | −16.61 | −16.61 | −16.61 | −16.61 | −16.61 |
| hsa-miR-4422-3p | −16.61 | −16.61 | −16.61 | −16.61 | −16.61 |
| hsa-miR-4422-5p | −16.61 | −16.61 | −16.61 | −16.61 | −16.61 |
| hsa-miR-4423-3p | −16.61 | −16.61 | −16.61 | −16.61 | −16.61 |
| hsa-miR-4423-5p | −16.61 | −16.61 | −16.61 | −16.61 | −16.61 |
| hsa-miR-4424 | −16.61 | −16.61 | −16.61 | −16.61 | −16.61 |
| hsa-miR-4425-3p | −16.61 | −16.61 | 3.153 | 2.898 | 2.102 |
| hsa-miR-4425-5p | −16.61 | −16.61 | −16.61 | −16.61 | −16.61 |
| hsa-miR-4426 | −16.61 | −16.61 | −16.61 | −16.61 | −16.61 |
| hsa-miR-4427 | −16.61 | −16.61 | −16.61 | −16.61 | −16.61 |
| hsa-miR-4428 | −16.61 | −16.61 | −16.61 | −16.61 | −16.61 |
| hsa-miR-4429 | −16.61 | 1.12 | −16.61 | −16.61 | 3.102 |
| hsa-miR-4430 | −16.61 | −16.61 | −16.61 | −16.61 | −16.61 |
| hsa-miR-4431-3p | 1.867 | −16.61 | −16.61 | −16.61 | −16.61 |
| hsa-miR-4431-5p | −16.61 | −16.61 | −16.61 | −16.61 | −16.61 |
| hsa-miR-4432 | −16.61 | −16.61 | −16.61 | −16.61 | −16.61 |
| hsa-miR-4433a-3p | −16.61 | −16.61 | −16.61 | −16.61 | −16.61 |
| hsa-miR-4433a-5p | −16.61 | −16.61 | −16.61 | −16.61 | −16.61 |
| hsa-miR-4433b-3p | −16.61 | −16.61 | −16.61 | −16.61 | −16.61 |
| hsa-miR-4433b-5p | −16.61 | −16.61 | −16.61 | −16.61 | −16.61 |
| hsa-miR-4434 | −16.61 | −16.61 | −16.61 | −16.61 | −16.61 |
| hsa-miR-4435-3p | −16.61 | −16.61 | −16.61 | −16.61 | −16.61 |
| hsa-miR-4435-5p | −16.61 | −16.61 | 16.61 | −16.61 | −16.61 |
| hsa-miR-4436a | −16.61 | −16.61 | −16.61 | −16.61 | −16.61 |
| hsa-miR-4436b-3p | −16.61 | −16.61 | −16.61 | −16.61 | −16.61 |
| hsa-miR-4436b-5p | −16.61 | −16.61 | −16.61 | −16.61 | −16.61 |
| hsa-miR-4437 | −16.61 | −16.61 | −16.61 | −16.61 | −16.61 |
| hsa-miR-4438 | −16.61 | −16.61 | −16.61 | −16.61 | −16.61 |
| hsa-miR-4439 | −16.61 | −16.61 | −16.61 | −16.61 | −16.61 |
| hsa-miR-4440 | −16.61 | −16.61 | −16.61 | 2.176 | −16.61 |
| hsa-miR-4441 | −16.61 | −16.61 | −16.61 | −16.61 | −16.61 |
| hsa-miR-4442 | −16.61 | −16.61 | −16.61 | −16.61 | −16.61 |
| hsa-miR-4443 | −16.61 | −16.61 | −16.61 | 3.024 | 5.481 |
| hsa-miR-4444 | −16.61 | −16.61 | −16.61 | −16.61 | 3.481 |
| hsa-miR-4445-3p | −16.61 | −16.61 | −16.61 | −16.61 | −16.61 |
| hsa-miR-4445-5p | −16.61 | −16.61 | −16.61 | −16.61 | −16.61 |
| hsa-miR-4446-3p | −16.61 | −16.61 | −16.61 | −16.61 | −16.61 |
| hsa-miR-4446-5p | −16.61 | −16.61 | −16.61 | −16.61 | −16.61 |
| hsa-miR-4447 | −16.61 | −16.61 | −16.61 | −16.61 | −16.61 |
| hsa-miR-4448 | −16.61 | −16.61 | −16.61 | −16.61 | −16.61 |
| hsa-miR-4449 | 1.139 | 4.316 | 1.897 | 0.439 | 3.24 |
| hsa-miR-4450 | 5.041 | 1.994 | −16.61 | −16.61 | −16.61 |
| hsa-miR-4451 | −16.61 | −16.61 | −16.61 | −16.61 | −16.61 |
| hsa-miR-4452-3p | −16.61 | −16.61 | −16.61 | −16.61 | −16.61 |
| hsa-miR-4452-5p | −16.61 | −16.61 | −16.61 | −16.61 | −16.61 |
| hsa-miR-4453 | −16.61 | −16.61 | −16.61 | −16.61 | −16.61 |
| hsa-miR-4454 | 6.352 | 6.027 | 5.634 | 5.176 | 3.588 |
| hsa-miR-4455 | −16.61 | −16.61 | −16.61 | −16.61 | −16.61 |
| hsa-miR-4456 | 16.61 | −16.61 | −16.61 | −16.61 | −16.61 |
| hsa-miR-4457 | −16.61 | −16.61 | −16.61 | −16.61 | −16.61 |
| hsa-miR-4458 | −16.61 | −16.61 | −16.61 | −16.61 | −16.61 |
| hsa-miR-4460 | −16.61 | −16.61 | −16.61 | −16.61 | −16.61 |
| hsa-miR-4462 | −16.61 | −16.61 | −16.61 | −16.61 | −16.61 |
| hsa-miR-4463 | −16.61 | −16.61 | −16.61 | −16.61 | −16.61 |
| hsa-miR-4464-3p | −16.61 | −16.61 | −16.61 | −16.61 | −16.61 |
| hsa-miR-4464-5p | −16.61 | −16.61 | −16.61 | −16.61 | −16.61 |
| hsa-miR-4465 | −16.61 | −16.61 | −16.61 | −16.61 | −16.61 |
| hsa-miR-4466 | −16.61 | −16.61 | −16.61 | −16.61 | −16.61 |
| hsa-miR-4467-3p | −16.61 | −16.61 | −16.61 | −16.61 | −16.61 |
| hsa-miR-4467-5p | −16.61 | −16.61 | −16.61 | −16.61 | −16.61 |
| hsa-miR-4468 | −16.61 | −16.61 | −16.61 | −16.61 | −16.61 |
| hsa-miR-4469-3p | −16.61 | −16.61 | −16.61 | −16.61 | −16.61 |
| hsa-miR-4469-5p | −16.61 | −16.61 | −16.61 | −16.61 | −16.61 |
| hsa-miR-4470-3p | −16.61 | 3.783 | 1.049 | 0.854 | −16.61 |
| hsa-miR-4470-5p | −16.61 | −16.61 | −16.61 | −16.61 | −16.61 |
| hsa-miR-4471 | −16.61 | −16.61 | −16.61 | −16.61 | −16.61 |
| hsa-miR-4472 | −16.61 | −16.61 | −16.61 | −16.61 | −16.61 |
| hsa-miR-4473-3p | 2.296 | −0.465 | −16.61 | −16.61 | −16.61 |
| hsa-miR-4473-5p | 4.337 | 1.12 | −16.61 | −16.61 | −16.61 |
| hsa-miR-4474-3p | 3.118 | 0.857 | −16.61 | −16.61 | 2.365 |
| hsa-miR-4474-5p | −16.61 | −16.61 | −16.61 | −16.61 | −16.61 |
| hsa-miR-4475 | −16.61 | −16.61 | −16.61 | −16.61 | −16.61 |
| hsa-miR-4476 | −16.61 | −16.61 | −16.61 | −16.61 | −16.61 |
| hsa-miR-4477a | −16.61 | −16.61 | −16.61 | −16.61 | −16.61 |
| hsa-miR-4477b-3p | 3.139 | 0.12 | 2.251 | 0.854 | 2.95 |
| hsa-miR-4477b-5p | −16.61 | −16.61 | −16.61 | −16.61 | −16.61 |
| hsa-miR-4478 | −16.61 | −16.61 | −16.61 | −16.61 | −16.61 |
| hsa-miR-4479-3p | −16.61 | −16.61 | 1.187 | −16.61 | −16.61 |
| hsa-miR-4479-5p | −16.61 | −16.61 | −16.61 | −16.61 | −16.61 |
| hsa-miR-448 | 9.114 | 6.825 | 3.312 | 2.712 | 3.95 |
| hsa-miR-4480 | −16.61 | −16.61 | −16.61 | −16.61 | −16.61 |
| hsa-miR-4481 | −16.61 | −16.61 | −16.61 | −16.61 | −16.61 |
| hsa-miR-4482-3p | −16.61 | −16.61 | −16.61 | 1.661 | −16.61 |
| hsa-miR-4482-5p | −16.61 | −16.61 | −16.61 | −16.61 | −16.61 |
| hsa-miR-4483 | −16.61 | −16.61 | −16.61 | −16.61 | −16.61 |
| hsa-miR-4484 | −16.61 | −16.61 | −16.61 | 3.784 | 7.24 |
| hsa-miR-4485-3p | 3.3 | 5.674 | 3.282 | 6.125 | 4.868 |
| hsa-miR-4485-5p | 3.171 | −16.61 | 1.534 | −16.61 | −16.61 |
| hsa-miR-4486 | −16.61 | −16.61 | −16.61 | −16.61 | −16.61 |
| hsa-miR-4487 | −16.61 | −16.61 | 2.428 | 2.176 | 3.588 |
| hsa-miR-4488 | 3.608 | 4.316 | 2.815 | 4.044 | 6.335 |
| hsa-miR-4489 | −16.61 | −16.61 | 0.897 | −16.61 | −16.61 |
| hsa-miR-4490 | −16.61 | −16.61 | −16.61 | −16.61 | −16.61 |
| hsa-miR-4491 | −16.61 | −16.61 | −16.61 | −16.61 | −16.61 |
| hsa-miR-4492 | 5.227 | −16.61 | −16.61 | −16.61 | 8.057 |
| hsa-miR-4493 | −16.61 | −16.61 | −16.61 | −16.61 | −16.61 |
| hsa-miR-4494 | −16.61 | −16.61 | −16.61 | −16.61 | −16.61 |
| hsa-miR-4495 | −16.61 | −16.61 | −16.61 | −16.61 | −16.61 |
| hsa-miR-4496 | −16.61 | −16.61 | −16.61 | −16.61 | −16.61 |
| hsa-miR-4497 | 3.403 | 4.179 | 3.428 | 4.102 | 4.99 |
| hsa-miR-4498 | −16.61 | −16.61 | −16.61 | −16.61 | −16.61 |
| hsa-miR-4499 | −16.61 | −16.61 | −16.61 | −16.61 | −16.61 |
| hsa-miR-449a/449b-5p | 3.626 | 4.368 | 3.371 | 4.512 | 5.91 |
| hsa-miR-449b-3p | −16.61 | −16.61 | −16.61 | −16.61 | −16.61 |
| hsa-miR-449c-3p | −16.61 | −16.61 | −16.61 | −16.61 | −16.61 |
| hsa-miR-449c-5p | 2.107 | −16.61 | 1.312 | −16.61 | −16.61 |
| hsa-miR-4500 | −16.61 | −16.61 | −16.61 | −16.61 | −16.61 |
| hsa-miR-4501 | −16.61 | −16.61 | −16.61 | −16.61 | −16.61 |
| hsa-miR-4502 | −16.61 | −16.61 | −16.61 | −16.61 | −16.61 |
| hsa-miR-4503 | −16.61 | −16.61 | −16.61 | −16.61 | −16.61 |
| hsa-miR-4504-3p | −16.61 | 0.535 | 1.534 | 2.554 | −16.61 |
| hsa-miR-4504-5p | −16.61 | −16.61 | −16.61 | −16.61 | −16.61 |
| hsa-miR-4505 | −16.61 | −16.61 | −16.61 | −16.61 | −16.61 |
| hsa-miR-4506 | −16.61 | −16.61 | −16.61 | −16.61 | −16.61 |
| hsa-miR-4507 | −16.61 | −16.61 | −16.61 | −16.61 | −16.61 |
| hsa-miR-4508 | 4.857 | 6.557 | 6.622 | 6.229 | 7.575 |
| hsa-miR-4509 | −16.61 | −16.61 | −16.61 | −16.61 | −16.61 |
| hsa-miR-450a-1-3p | 2.721 | 3.961 | 4.547 | 4.609 | 4.535 |
| hsa-miR-450a-2-3p | 2.521 | 4.91 | 3.836 | 4.102 | 3.868 |
| hsa-miR-450a-5p | 3.16 | 0.12 | 3.312 | 3.408 | 8.505 |
| hsa-miR-450b-3p | −16.61 | −16.61 | 0.049 | −16.61 | 2.102 |
| hsa-miR-450b-5p | 5.725 | 7.697 | 7.924 | 8.479 | 5.24 |
| hsa-miR-4510 | −16.61 | −16.61 | −16.61 | −16.61 | −16.61 |
| hsa-miR-4511-3p | −16.61 | −16.61 | −0.688 | −16.61 | −16.61 |
| hsa-miR-4511-5p | −16.61 | −16.61 | 1.187 | 1.761 | −16.61 |
| hsa-miR-4512-3p | −16.61 | −16.61 | −16.61 | −16.61 | −16.61 |
| hsa-miR-4512-5p | −16.61 | −16.61 | −16.61 | 1.761 | −16.61 |
| hsa-miR-4513 | −16.61 | −16.61 | −16.61 | −16.61 | −16.61 |
| hsa-miR-4514 | −16.61 | −16.61 | −16.61 | −16.61 | −16.61 |
| hsa-miR-4515 | −16.61 | −16.61 | −16.61 | −16.61 | −16.61 |
| hsa-miR-4516 | 8.917 | 10.056 | 8.691 | 9.067 | −16.61 |
| hsa-miR-4517 | −16.61 | −16.61 | −16.61 | −16.61 | −16.61 |
| hsa-miR-4518 | −16.61 | −16.61 | −16.61 | −16.61 | −16.61 |
| hsa-miR-4519 | −16.61 | −16.61 | −16.61 | −16.61 | −16.61 |
| hsa-miR-451a | 1.559 | 7.12 | 3.749 | 3.661 | 3.24 |
| hsa-miR-451b-3p | −16.61 | −16.61 | −16.61 | −16.61 | −16.61 |
| hsa-miR-451b-5p | −16.61 | −16.61 | −16.61 | −16.61 | −16.61 |
| hsa-miR-452-3p | −16.61 | −16.61 | −16.61 | −16.61 | −16.61 |
| hsa-miR-452-5p | 7.897 | 10.574 | 11.152 | 11.29 | 10.838 |
| hsa-miR-4520-2-3p/ | −16.61 | −16.61 | −16.61 | −16.61 | −16.61 |
| 4520-3p | | | | | |
| hsa-miR-4520-5p | −16.61 | −16.61 | −16.61 | −16.61 | −16.61 |
| hsa-miR-4521 | 3.944 | 4.783 | 1.634 | 1.313 | 2.78 |
| hsa-miR-4522 | −16.61 | −16.61 | −16.61 | −16.61 | −16.61 |
| hsa-miR-4523-3p | −16.61 | −16.61 | −16.61 | −16.61 | −16.61 |
| hsa-miR-4523-5p | −16.61 | 1.535 | 1.634 | 1.439 | −16.61 |
| hsa-miR-4524a-3p | −16.61 | −16.61 | −16.61 | −16.61 | −16.61 |
| hsa-miR-4524a-5p | −16.61 | −16.61 | −16.61 | −16.61 | −16.61 |
| hsa-miR-4524b-3p | −16.61 | −16.61 | −16.61 | −16.61 | −16.61 |
| hsa-miR-4524b-5p | −16.61 | −16.61 | −16.61 | −16.61 | −16.61 |
| hsa-miR-4525-3p | −16.61 | −16.61 | −16.61 | −16.61 | −16.61 |
| hsa-miR-4525-5p | 1.677 | −0.465 | 0.534 | −16.61 | −16.61 |
| hsa-miR-4526-3p | 3.334 | −16.61 | 2.975 | 3.313 | −16.61 |
| hsa-miR-4526-5p | −16.61 | −16.61 | −16.61 | −16.61 | −16.61 |
| hsa-miR-4527 | −16.61 | −16.61 | −16.61 | −16.61 | −16.61 |
| hsa-miR-4528-3p | −16.61 | −16.61 | −16.61 | −16.61 | −16.61 |
| hsa-miR-4528-5p | −16.61 | −16.61 | −16.61 | −16.61 | −16.61 |
| hsa-miR-4529-3p | −16.61 | −16.61 | −16.61 | −16.61 | −16.61 |
| hsa-miR-4529-5p | −16.61 | −16.61 | −16.61 | −16.61 | −16.61 |
| hsa-miR-4530 | −16.61 | −16.61 | −16.61 | −16.61 | −16.61 |
| hsa-miR-4531 | −16.61 | −16.61 | −16.61 | −16.61 | −16.61 |
| hsa-miR-4533-3p | −16.61 | −16.61 | −16.61 | −16.61 | −16.61 |
| hsa-miR-4533-5p | −16.61 | −16.61 | −16.61 | −16.61 | −16.61 |
| hsa-miR-4534 | −16.61 | −16.61 | −16.61 | −16.61 | −16.61 |
| hsa-miR-4535 | −16.61 | −16.61 | −16.61 | −16.61 | −16.61 |
| hsa-miR-4536-3p | −16.61 | −16.61 | −16.61 | −16.61 | −16.61 |
| hsa-miR-4536-5p | −16.61 | −16.61 | −16.61 | 2.377 | −16.61 |
| hsa-miR-4537 | −16.61 | −16.61 | −16.61 | −16.61 | −16.61 |
| hsa-miR-4538 | −16.61 | −16.61 | −16.61 | −16.61 | −16.61 |
| hsa-miR-4539 | −16.61 | −16.61 | −16.61 | −16.61 | −16.61 |
| hsa-miR-454-3p | 9.857 | 9.83 | 9.9 | 9.644 | 7.568 |
| hsa-miR-454-5p | 4.115 | 4.489 | 4.282 | 4.329 | −16.61 |
| hsa-miR-4540 | −16.61 | −16.61 | −16.61 | −16.61 | −16.61 |
| hsa-miR-455-3p | 6.961 | 4.801 | 2.049 | 1.761 | 0.78 |
| hsa-miR-455-5p | 9.555 | 7.684 | 3.897 | 4.044 | −16.61 |
| hsa-miR-4632-3p | −16.61 | −16.61 | −0.688 | −16.61 | −16.61 |
| hsa-miR-4632-5p | −16.61 | −16.61 | −16.61 | −16.61 | −16.61 |
| hsa-miR-4633-3p | −16.61 | −16.61 | −16.61 | −16.61 | −16.61 |
| hsa-miR-4633-5p | −16.61 | −16.61 | −16.61 | −16.61 | −16.61 |
| hsa-miR-4634 | −16.61 | −16.61 | −16.61 | −16.61 | −16.61 |
| hsa-miR-4635 | −16.61 | −16.61 | −16.61 | 3.024 | −16.61 |
| hsa-miR-4636-3p | −16.61 | −16.61 | −16.61 | −16.61 | −16.61 |
| hsa-miR-4636-5p | −16.61 | −0.465 | −16.61 | −16.61 | −16.61 |
| hsa-miR-4637 | −16.61 | −16.61 | −16.61 | −16.61 | −16.61 |
| hsa-miR-4638-3p | −16.61 | −16.61 | −16.61 | −16.61 | −16.61 |
| hsa-miR-4638-5p | −16.61 | −16.61 | −16.61 | −16.61 | −16.61 |
| hsa-miR-4639-3p | −16.61 | −16.61 | −16.61 | −16.61 | −16.61 |
| hsa-miR-4639-5p | −16.61 | −16.61 | −16.61 | −16.61 | −16.61 |
| hsa-miR-4640-3p | 2.549 | −16.61 | −16.61 | −16.61 | −16.61 |
| hsa-miR-4640-5p | −16.61 | −16.61 | −16.61 | −16.61 | −16.61 |
| hsa-miR-4641 | 1.677 | −16.61 | −16.61 | −16.61 | −16.61 |
| hsa-miR-4642 | −16.61 | −16.61 | −16.61 | −16.61 | −16.61 |
| hsa-miR-4643 | −16.61 | −16.61 | −16.61 | −16.61 | −16.61 |
| hsa-miR-4644-3p | −16.61 | −16.61 | −16.61 | −16.61 | −16.61 |
| hsa-miR-4644-5p | −16.61 | −16.61 | −16.61 | −16.61 | −16.61 |
| hsa-miR-4645-3p | 2.772 | 3.29 | 3.219 | 2.246 | 3.365 |
| hsa-miR-4645-5p | −16.61 | −16.61 | −16.61 | −16.61 | −16.61 |
| hsa-miR-4646-3p | −16.61 | −16.61 | −16.61 | −16.61 | −16.61 |
| hsa-miR-4646-5p | −16.61 | −16.61 | −16.61 | −16.61 | −16.61 |
| hsa-miR-4647-3p | −16.61 | −16.61 | −16.61 | −16.61 | −16.61 |
| hsa-miR-4647-5p | −16.61 | −16.61 | −16.61 | −16.61 | −16.61 |
| hsa-miR-4648 | −16.61 | −16.61 | 0.049 | −16.61 | 2.588 |
| hsa-miR-4649-3p | −16.61 | −16.61 | −16.61 | −16.61 | −16.61 |
| hsa-miR-4649-5p | −16.61 | −16.61 | −16.61 | −16.61 | −16.61 |
| hsa-miR-4650-3p | −16.61 | −16.61 | −16.61 | −16.61 | −16.61 |
| hsa-miR-4650-5p | −16.61 | −16.61 | −16.61 | −16.61 | −16.61 |
| hsa-miR-4651 | −16.61 | −16.61 | −16.61 | −16.61 | −16.61 |
| hsa-miR-4652-3p | −16.61 | −16.61 | −16.61 | −16.61 | −16.61 |
| hsa-miR-4652-5p | −16.61 | −16.61 | −16.61 | −16.61 | −16.61 |
| hsa-miR-4653-3p | −16.61 | 1.857 | −16.61 | 3.063 | −16.61 |
| hsa-miR-4653-5p | −16.61 | −16.61 | −16.61 | −16.61 | −16.61 |
| hsa-miR-4654 | −16.61 | −16.61 | −16.61 | −16.61 | −16.61 |
| hsa-miR-4655-3p | −16.61 | −16.61 | −16.61 | −16.61 | −16.61 |
| hsa-miR-4655-5p | −16.61 | −16.61 | −16.61 | −16.61 | −16.61 |
| hsa-miR-4656 | 2.391 | −16.61 | −16.61 | −16.61 | −16.61 |
| hsa-miR-4657 | −16.61 | −16.61 | −16.61 | −16.61 | −16.61 |
| hsa-miR-4658 | −16.61 | −16.61 | −16.61 | −16.61 | −16.61 |
| hsa-miR-4659a-3p/ | −16.61 | 0.535 | −16.61 | −16.61 | −16.61 |
| 4659b-3p | | | | | |
| hsa-miR-4659a-5p/ | −16.61 | −16.61 | −16.61 | −16.61 | −16.61 |
| 4659b-5p | | | | | |
| hsa-miR-466 | −16.61 | −16.61 | −16.61 | −16.61 | −16.61 |
| hsa-miR-4660 | −16.61 | 4.417 | −16.61 | 3.211 | 3.365 |
| hsa-miR-4661-3p | −16.61 | −16.61 | −16.61 | −16.61 | −16.61 |
| hsa-miR-4661-5p | −16.61 | −16.61 | −16.61 | −16.61 | −16.61 |
| hsa-miR-4662a-3p | −16.61 | −16.61 | −16.61 | −16.61 | −16.61 |
| hsa-miR-4662a-5p | 4.836 | 2.783 | 1.428 | 3.346 | −16.61 |
| hsa-miR-4662b | −16.61 | −16.61 | −16.61 | −16.61 | −16.61 |
| hsa-miR-4663 | −16.61 | −16.61 | −16.61 | −16.61 | −16.61 |
| hsa-miR-4664-3p | −16.61 | −16.61 | −16.61 | −16.61 | −16.61 |
| hsa-miR-4664-5p | −16.61 | −16.61 | −16.61 | −16.61 | −16.61 |
| hsa-miR-4665-3p | −16.61 | −16.61 | −16.61 | −16.61 | −16.61 |
| hsa-miR-4665-5p | −16.61 | 3.12 | −16.61 | 1.661 | 2.78 |
| hsa-miR-4666a-3p | −16.61 | −16.61 | −16.61 | −16.61 | −16.61 |
| hsa-miR-4666a-5p | −16.61 | −16.61 | −16.61 | −16.61 | −16.61 |
| hsa-miR-4666b | −16.61 | −16.61 | −16.61 | −16.61 | −16.61 |
| hsa-miR-4667-3p | −16.61 | −16.61 | −16.61 | −16.61 | −16.61 |
| hsa-miR-4667-5p | −16.61 | −16.61 | −16.61 | −16.61 | 4.24 |
| hsa-miR-4668-3p | −16.61 | −16.61 | −16.61 | −16.61 | −16.61 |
| hsa-miR-4668-5p | −16.61 | −16.61 | −16.61 | −16.61 | −16.61 |
| hsa-miR-4669 | −16.61 | 4.235 | 2.482 | 3.526 | 4.173 |
| hsa-miR-4670-3p | −16.61 | −16.61 | −16.61 | −16.61 | −16.61 |
| hsa-miR-4670-5p | −16.61 | −16.61 | −16.61 | −16.61 | −16.61 |
| hsa-miR-4671-3p | −16.61 | −16.61 | −16.61 | −16.61 | −16.61 |
| hsa-miR-4671-5p | −16.61 | −16.61 | −16.61 | −16.61 | −16.61 |
| hsa-miR-4672-3p | 4.2 | −16.61 | −16.61 | −16.61 | −16.61 |
| hsa-miR-4672-5p | −16.61 | −16.61 | −16.61 | −16.61 | −16.61 |
| hsa-miR-4673 | −16.61 | −16.61 | −16.61 | −16.61 | −16.61 |
| hsa-miR-4674 | −16.61 | −16.61 | −16.61 | −16.61 | −16.61 |
| hsa-miR-4675 | −16.61 | −16.61 | −16.61 | −16.61 | −16.61 |
| hsa-miR-4676-3p | −16.61 | −16.61 | −16.61 | −16.61 | 2.78 |
| hsa-miR-4676-5p | −16.61 | −16.61 | −16.61 | −16.61 | −16.61 |
| hsa-miR-4677-3p | 4.751 | 5.454 | 5.342 | 5.742 | 4.028 |
| hsa-miR-4677-5p | 2.228 | 0.12 | 2.049 | 1.941 | −16.61 |
| hsa-miR-4678-3p | −16.61 | −16.61 | −16.61 | −16.61 | −16.61 |
| hsa-miR-4678-5p | −16.61 | −16.61 | −16.61 | −16.61 | −16.61 |
| hsa-miR-4679 | −16.61 | −16.61 | −16.61 | −16.61 | −16.61 |
| hsa-miR-4680-3p | 3.501 | −16.61 | −16.61 | −16.61 | −16.61 |
| hsa-miR-4680-5p | −16.61 | −16.61 | −16.61 | −16.61 | −16.61 |
| hsa-miR-4681-3p | −16.61 | −16.61 | −16.61 | −16.61 | −16.61 |
| hsa-miR-4681-5p | −16.61 | −16.61 | −16.61 | −16.61 | −16.61 |
| hsa-miR-4682-3p | −16.61 | −16.61 | −16.61 | −16.61 | −16.61 |
| hsa-miR-4682-5p | −16.61 | −16.61 | −16.61 | −16.61 | −16.61 |
| hsa-miR-4683 | −16.61 | −16.61 | −16.61 | −16.61 | −16.61 |
| hsa-miR-4684-3p | −16.61 | −16.61 | −16.61 | −16.61 | −16.61 |
| hsa-miR-4684-5p | −16.61 | −16.61 | −16.61 | −16.61 | −16.61 |
| hsa-miR-4685-3p | −16.61 | −16.61 | −16.61 | 0.854 | 1.365 |
| hsa-miR-4685-5p | −16.61 | −16.61 | −16.61 | −16.61 | −16.61 |
| hsa-miR-4686 | −16.61 | −16.61 | −16.61 | −16.61 | −16.61 |
| hsa-miR-4687-3p | −16.61 | −16.61 | −16.61 | −16.61 | −16.61 |
| hsa-miR-4687-5p | 2.207 | −16.61 | 0.312 | −16.61 | −16.61 |
| hsa-miR-4688-3p/ | −16.61 | −16.61 | −16.61 | −16.61 | −16.61 |
| 4688-5p | | | | | |
| hsa-miR-4689-3p | −16.61 | −16.61 | −16.61 | −16.61 | −16.61 |
| hsa-miR-4689-5p | −16.61 | −16.61 | −16.61 | −16.61 | −16.61 |
| hsa-miR-4690-3p | 0.384 | 0.857 | 1.634 | 2.102 | −16.61 |
| hsa-miR-4690-5p | −16.61 | −16.61 | −16.61 | −16.61 | −16.61 |
| hsa-miR-4691-3p | −16.61 | −16.61 | −16.61 | −16.61 | −16.61 |
| hsa-miR-4691-5p | −16.61 | −16.61 | −16.61 | −16.61 | −16.61 |
| hsa-miR-4692 | −16.61 | −16.61 | −16.61 | −16.61 | −16.61 |
| hsa-miR-4693-3p | −16.61 | −16.61 | −16.61 | −16.61 | −16.61 |
| hsa-miR-4693-5p | −16.61 | −16.61 | −16.61 | −16.61 | −16.61 |
| hsa-miR-4694-3p | −16.61 | −16.61 | −16.61 | −16.61 | −16.61 |
| hsa-miR-4694-5p | −16.61 | −16.61 | −16.61 | −16.61 | −16.61 |
| hsa-miR-4695-3p | −16.61 | −16.61 | −16.61 | −16.61 | −16.61 |
| hsa-miR-4695-5p | −16.61 | −16.61 | −16.61 | −16.61 | −16.61 |
| hsa-miR-4696 | −16.61 | −16.61 | −16.61 | −16.61 | −16.61 |
| hsa-miR-4697-3p | −16.61 | −16.61 | −16.61 | −16.61 | −16.61 |
| hsa-miR-4697-5p | −16.61 | −16.61 | −16.61 | −16.61 | −16.61 |
| hsa-miR-4698 | −16.61 | −16.61 | −16.61 | −16.61 | −16.61 |
| hsa-miR-4699-3p | −16.61 | −16.61 | −16.61 | −16.61 | −16.61 |
| hsa-miR-4699-5p | −16.61 | 0.12 | −16.61 | −16.61 | −16.61 |
| hsa-miR-4700-3p | −16.61 | −16.61 | −16.61 | −16.61 | −16.61 |
| hsa-miR-4700-5p | −16.61 | −16.61 | −16.61 | −16.61 | −16.61 |
| hsa-miR-4701-3p | −16.61 | −16.61 | −16.61 | −16.61 | −16.61 |
| hsa-miR-4701-5p | −16.61 | −16.61 | −16.61 | −16.61 | −16.61 |
| hsa-miR-4703-3p | −16.61 | −16.61 | −16.61 | 2.102 | −16.61 |
| hsa-miR-4703-5p | −16.61 | −16.61 | −16.61 | −16.61 | −16.61 |
| hsa-miR-4704-3p | −16.61 | −16.61 | −16.61 | −16.61 | −16.61 |
| hsa-miR-4704-5p | −16.61 | −16.61 | −16.61 | −16.61 | −16.61 |
| hsa-miR-4705 | 4.256 | 4.263 | 1.975 | 2.439 | −16.61 |
| hsa-miR-4706-3p | −16.61 | −16.61 | −16.61 | −16.61 | −16.61 |
| hsa-miR-4706-5p | −16.61 | −16.61 | −16.61 | −16.61 | 2.588 |
| hsa-miR-4707-3p | −16.61 | −16.61 | −16.61 | −16.61 | −16.61 |
| hsa-miR-4707-5p | −16.61 | −16.61 | −16.61 | −16.61 | −16.61 |
| hsa-miR-4708-3p | −16.61 | −16.61 | −16.61 | −16.61 | −16.61 |
| hsa-miR-4708-5p | −16.61 | −16.61 | −16.61 | −16.61 | −16.61 |
| hsa-miR-4709-3p | 3.736 | 0.535 | 2.119 | 1.854 | 4.424 |
| hsa-miR-4709-5p | 2.953 | −16.61 | −0.688 | 2.498 | −16.61 |
| hsa-miR-4710 | 2.18 | −16.61 | 1.187 | 2.246 | 4.424 |
| hsa-miR-4711-3p | −16.61 | −16.61 | −16.61 | −16.61 | −16.61 |
| hsa-miR-4711-5p | −16.61 | −16.61 | −16.61 | −16.61 | −16.61 |
| hsa-miR-4712-3p | −16.61 | −16.61 | −16.61 | −16.61 | −16.61 |
| hsa-miR-4712-5p | −16.61 | −16.61 | −16.61 | −16.61 | −16.61 |
| hsa-miR-4713-3p | −16.61 | −16.61 | −16.61 | −16.61 | −16.61 |
| hsa-miR-4713-5p | −16.61 | −16.61 | −16.61 | −16.61 | −16.61 |
| hsa-miR-4714-3p | 2.878 | −16.61 | 2.371 | 1.941 | −16.61 |
| hsa-miR-4714-5p | −16.61 | −16.61 | −16.61 | −16.61 | −16.61 |
| hsa-miR-4715-3p | −16.61 | −16.61 | −16.61 | −16.61 | −16.61 |
| hsa-miR-4715-5p | −16.61 | −16.61 | 1.815 | −16.61 | −16.61 |
| hsa-miR-4716-3p | −16.61 | −16.61 | −16.61 | −16.61 | −16.61 |
| hsa-miR-4716-5p | −16.61 | −16.61 | −16.61 | −16.61 | −16.61 |
| hsa-miR-4717-3p | −16.61 | −16.61 | 1.049 | −16.61 | −16.61 |
| hsa-miR-4717-5p | −16.61 | −16.61 | −16.61 | −16.61 | −16.61 |
| hsa-miR-4718 | −16.61 | −16.61 | −16.61 | −16.61 | −16.61 |
| hsa-miR-4719-3p | 3.262 | −16.61 | −16.61 | −16.61 | −16.61 |
| hsa-miR-4719-5p | −16.61 | −16.61 | −16.61 | −16.61 | −16.61 |
| hsa-miR-4720-3p | −16.61 | −16.61 | −16.61 | −16.61 | −16.61 |
| hsa-miR-4720-5p | −16.61 | −16.61 | −16.61 | −16.61 | −16.61 |
| hsa-miR-4721-3p/ | −16.61 | −16.61 | −16.61 | −16.61 | −16.61 |
| 4721-5p | | | | | |
| hsa-miR-4722-3p | −16.61 | −16.61 | −16.61 | −16.61 | −16.61 |
| hsa-miR-4722-5p | −16.61 | −16.61 | −16.61 | −16.61 | −16.61 |
| hsa-miR-4723-3p | −16.61 | −16.61 | −16.61 | −16.61 | −16.61 |
| hsa-miR-4723-5p | −16.61 | −16.61 | −16.61 | −16.61 | 4.028 |
| hsa-miR-4724-3p | 1.328 | −16.61 | −16.61 | −16.61 | −16.61 |
| hsa-miR-4724-5p | 2.943 | −16.61 | 1.049 | 0.661 | −16.61 |
| hsa-miR-4725-3p | 4.137 | −16.61 | 0.534 | 3.408 | 5.272 |
| hsa-miR-4725-5p | −16.61 | −16.61 | −16.61 | −16.61 | −16.61 |
| hsa-miR-4726-3p | 2.639 | −16.61 | −16.61 | −16.61 | −16.61 |
| hsa-miR-4726-5p | −16.61 | −16.61 | 1.897 | 1.761 | −16.61 |
| hsa-miR-4727-3p | −16.61 | −16.61 | −16.61 | −16.61 | −16.61 |
| hsa-miR-4727-5p | −16.61 | −16.61 | −16.61 | −16.61 | −16.61 |
| hsa-miR-4728-3p | −16.61 | 0.857 | −16.61 | −16.61 | −16.61 |
| hsa-miR-4728-5p | −16.61 | −16.61 | −16.61 | −16.61 | −16.61 |
| hsa-miR-4729-3p | −16.61 | −16.61 | −16.61 | −16.61 | −16.61 |
| hsa-miR-4729-5p | −16.61 | −16.61 | −16.61 | −16.61 | −16.61 |
| hsa-miR-4730-3p | −16.61 | −16.61 | −16.61 | −16.61 | −16.61 |
| hsa-miR-4730-5p | 2.078 | −16.61 | −16.61 | −16.61 | −16.61 |
| hsa-miR-4731-3p | −16.61 | −16.61 | −16.61 | −16.61 | −16.61 |
| hsa-miR-4731-5p | −16.61 | −16.61 | −16.61 | −16.61 | 3.481 |
| hsa-miR-4732-3p | −16.61 | −16.61 | −16.61 | −16.61 | −16.61 |
| hsa-miR-4732-5p | −16.61 | −16.61 | −16.61 | −16.61 | −16.61 |
| hsa-miR-4733-3p | −16.61 | −16.61 | −16.61 | −16.61 | −16.61 |
| hsa-miR-4733-5p | 1.816 | −16.61 | −16.61 | −0.146 | −16.61 |
| hsa-miR-4734-3p | −16.61 | −16.61 | −16.61 | −16.61 | −16.61 |
| hsa-miR-4734-5p | −16.61 | −16.61 | −16.61 | −16.61 | −16.61 |
| hsa-miR-4735-3p | −16.61 | −16.61 | −16.61 | −16.61 | −16.61 |
| hsa-miR-4735-5p | 2.292 | 0.857 | −16.61 | −16.61 | −16.61 |
| hsa-miR-4736 | −16.61 | −16.61 | −16.61 | −16.61 | −16.61 |
| hsa-miR-4737-3p | −16.61 | −16.61 | −16.61 | −16.61 | −16.61 |
| hsa-miR-4737-5p | −16.61 | −16.61 | −16.61 | −16.61 | 1.365 |
| hsa-miR-4738-3p | 3.769 | −16.61 | −16.61 | −16.61 | −16.61 |
| hsa-miR-4738-5p | −16.61 | −16.61 | −16.61 | −16.61 | −16.61 |
| hsa-miR-4739 | −16.61 | −16.61 | −16.61 | −16.61 | 3.868 |
| hsa-miR-4740-3p | −16.61 | −16.61 | −16.61 | −16.61 | −16.61 |
| hsa-miR-4740-5p | −16.61 | −16.61 | −16.61 | −16.61 | −16.61 |
| hsa-miR-4741 | −16.61 | 0.857 | −16.61 | −16.61 | 3.24 |
| hsa-miR-4742-3p | 1.531 | −16.61 | −16.61 | −16.61 | −16.61 |
| hsa-miR-4742-5p | 2.422 | 3.961 | −16.61 | 2.439 | −16.61 |
| hsa-miR-4743-3p | −16.61 | −16.61 | −16.61 | −16.61 | −16.61 |
| hsa-miR-4743-5p | −16.61 | 0.857 | 1.187 | −16.61 | −16.61 |
| hsa-miR-4744 | −16.61 | −16.61 | −16.61 | 0.854 | −16.61 |
| hsa-miR-4745-3p | 3.431 | −16.61 | −16.61 | −16.61 | −16.61 |
| hsa-miR-4745-5p | −16.61 | −16.61 | −16.61 | −16.61 | −16.61 |
| hsa-miR-4746-3p | −16.61 | −16.61 | −16.61 | −16.61 | −16.61 |
| hsa-miR-4746-5p | −16.61 | 2.12 | 0.534 | 2.554 | −16.61 |
| hsa-miR-4747-3p | −16.61 | −16.61 | −16.61 | −16.61 | −16.61 |
| hsa-miR-4747-5p | 1.445 | −16.61 | −16.61 | −16.61 | −16.61 |
| hsa-miR-4748-3p | −16.61 | −16.61 | −16.61 | −16.61 | −16.61 |
| hsa-miR-4748-5p | 3.37 | −16.61 | 0.897 | −16.61 | −16.61 |
| hsa-miR-4749-3p | −16.61 | −16.61 | −16.61 | −1.146 | −16.61 |
| hsa-miR-4749-5p | −16.61 | −16.61 | −16.61 | −16.61 | −16.61 |
| hsa-miR-4750-3p | −16.61 | −16.61 | −16.61 | −16.61 | −16.61 |
| hsa-miR-4750-5p | −16.61 | −16.61 | −16.61 | 2.808 | −16.61 |
| hsa-miR-4751 | −16.61 | −16.61 | −16.61 | −16.61 | −16.61 |
| hsa-miR-4752 | −16.61 | −16.61 | −16.61 | −16.61 | −16.61 |
| hsa-miR-4753-3p | −16.61 | −16.61 | −16.61 | −16.61 | −16.61 |
| hsa-miR-4753-5p | −16.61 | 3.058 | −16.61 | −16.61 | −16.61 |
| hsa-miR-4754-3p | −16.61 | −16.61 | −16.61 | −16.61 | −16.61 |
| hsa-miR-4754-5p | −16.61 | −16.61 | −16.61 | −16.61 | −16.61 |
| hsa-miR-4755-3p | −16.61 | −16.61 | 2.534 | −16.61 | −16.61 |
| hsa-miR-4755-5p | −16.61 | −16.61 | −16.61 | −16.61 | −16.61 |
| hsa-miR-4756-3p | −16.61 | −16.61 | −16.61 | −16.61 | −16.61 |
| hsa-miR-4756-5p | −16.61 | −16.61 | −16.61 | −16.61 | −16.61 |
| hsa-miR-4757-3p | −16.61 | −16.61 | −16.61 | −16.61 | −16.61 |
| hsa-miR-4757-5p | −16.61 | −16.61 | −16.61 | −16.61 | −16.61 |
| hsa-miR-4758-3p | 2.513 | −16.61 | −16.61 | −16.61 | −16.61 |
| hsa-miR-4758-5p | −16.61 | −16.61 | −16.61 | −16.61 | −16.61 |
| hsa-miR-4759 | −16.61 | −16.61 | −16.61 | −16.61 | −16.61 |
| hsa-miR-4760-3p | −16.61 | −16.61 | −16.61 | −16.61 | −16.61 |
| hsa-miR-4760-5p | −16.61 | −16.61 | −16.61 | −16.61 | −16.61 |
| hsa-miR-4761-3p | −16.61 | −16.61 | −16.61 | −16.61 | −16.61 |
| hsa-miR-4761-5p | −16.61 | −16.61 | −16.61 | −16.61 | −16.61 |
| hsa-miR-4762-3p | 16.61 | −16.61 | −16.61 | −16.61 | −16.61 |
| hsa-miR-4762-5p | 3.116 | −16.61 | 0.312 | −16.61 | −16.61 |
| hsa-miR-4763-3p | −16.61 | −16.61 | −16.61 | −16.61 | −16.61 |
| hsa-miR-4763-5p | −16.61 | −16.61 | −16.61 | −16.61 | −16.61 |
| hsa-miR-4764-3p | −16.61 | −16.61 | −16.61 | −16.61 | −16.61 |
| hsa-miR-4764-5p | −16.61 | −16.61 | −16.61 | −16.61 | −16.61 |
| hsa-miR-4765-3p | −16.61 | −16.61 | −16.61 | −16.61 | −16.61 |
| hsa-miR-4765-5p | −16.61 | −16.61 | −16.61 | −16.61 | −16.61 |
| hsa-miR-4766-3p | −16.61 | −16.61 | 2.428 | 2.761 | 4.304 |
| hsa-miR-4766-5p | −16.61 | −16.61 | −16.61 | −16.61 | −16.61 |
| hsa-miR-4767 | 16.61 | −16.61 | −16.61 | −16.61 | −16.61 |
| hsa-miR-4768-3p | 16.61 | −16.61 | −16.61 | −16.61 | −16.61 |
| hsa-miR-4768-5p | −16.61 | −16.61 | −16.61 | −16.61 | −16.61 |
| hsa-miR-4769-3p | −16.61 | −16.61 | −16.61 | −16.61 | −16.61 |
| hsa-miR-4769-5p | −16.61 | −16.61 | −16.61 | −16.61 | −16.61 |
| hsa-miR-4770 | −16.61 | −16.61 | −16.61 | −16.61 | −16.61 |
| hsa-miR-4771-3p | −16.61 | −16.61 | −16.61 | −16.61 | −16.61 |
| hsa-miR-4771-5p | −16.61 | −16.61 | −16.61 | −16.61 | −16.61 |
| hsa-miR-4772-3p | 2.668 | 0.535 | 1.534 | 1.554 | −16.61 |
| hsa-miR-4772-5p | −16.61 | −16.61 | −16.61 | −16.61 | −16.61 |
| hsa-miR-4773 | −16.61 | −16.61 | −16.61 | −16.61 | −16.61 |
| hsa-miR-4774-3p | 16.61 | −16.61 | −16.61 | −16.61 | −16.61 |
| hsa-miR-4774-5p | −16.61 | −16.61 | 2.585 | −16.61 | −16.61 |
| hsa-miR-4775 | 2.157 | −16.61 | −16.61 | 0.854 | −16.61 |
| hsa-miR-4776-3p | −16.61 | −16.61 | −16.61 | −16.61 | −16.61 |
| hsa-miR-4776-5p | −16.61 | −16.61 | −16.61 | −16.61 | −16.61 |
| hsa-miR-4777-3p | 1.496 | 3.489 | 3.049 | 2.854 | −16.61 |
| hsa-miR-4777-5p | −16.61 | −16.61 | −16.61 | −16.61 | −16.61 |
| hsa-miR-4778-3p | −16.61 | −16.61 | −16.61 | −16.61 | −16.61 |
| hsa-miR-4778-5p | −16.61 | −16.61 | −16.61 | −16.61 | −16.61 |
| hsa-miR-4779-3p | −16.61 | −16.61 | −16.61 | −16.61 | −16.61 |
| hsa-miR-4779-5p | −16.61 | −16.61 | −16.61 | −16.61 | −16.61 |
| hsa-miR-4780 | −16.61 | −16.61 | −16.61 | −16.61 | −16.61 |
| hsa-miR-4781-3p | −16.61 | −16.61 | −16.61 | 2.854 | 2.588 |
| hsa-miR-4781-5p | −16.61 | −16.61 | −16.61 | −16.61 | −16.61 |
| hsa-miR-4782-3p | −16.61 | −16.61 | −16.61 | −16.61 | −16.61 |
| hsa-miR-4782-5p | −16.61 | −16.61 | −16.61 | −16.61 | −16.61 |
| hsa-miR-4783-3p | 4.129 | 2.927 | −16.61 | −16.61 | 3.687 |
| hsa-miR-4783-5p | −16.61 | −16.61 | −16.61 | −16.61 | −16.61 |
| hsa-miR-4784 | −16.61 | −16.61 | −16.61 | −16.61 | −16.61 |
| hsa-miR-4785 | −16.61 | −16.61 | 1.312 | −16.61 | 2.365 |
| hsa-miR-4786-3p | −16.61 | −16.61 | −16.61 | −16.61 | −16.61 |
| hsa-miR-4786-5p | −16.61 | −0.465 | −16.61 | −16.61 | −16.61 |
| hsa-miR-4787-3p | 3.386 | 2.235 | 1.312 | 2.439 | 3.95 |
| hsa-miR-4787-5p | −16.61 | −16.61 | −16.61 | −16.61 | −16.61 |
| hsa-miR-4788 | −16.61 | −16.61 | −16.61 | −16.61 | −16.61 |
| hsa-miR-4789-3p | 3.176 | −16.61 | −16.61 | −16.61 | −16.61 |
| hsa-miR-4789-5p | −16.61 | −16.61 | −16.61 | −16.61 | −16.61 |
| hsa-miR-4790-3p | 16.61 | −16.61 | −16.61 | −16.61 | −16.61 |
| hsa-miR-4790-5p | −16.61 | −16.61 | −16.61 | −16.61 | −16.61 |
| hsa-miR-4791 | 4.381 | 0.857 | 2.634 | 2.246 | −16.61 |
| hsa-miR-4793-3p | −16.61 | −16.61 | −16.61 | −16.61 | −16.61 |
| hsa-miR-4793-5p | −16.61 | −16.61 | −16.61 | −16.61 | −16.61 |
| hsa-miR-4794 | −16.61 | −16.61 | −16.61 | −16.61 | −16.61 |
| hsa-miR-4795-3p | −16.61 | −16.61 | 1.534 | −16.61 | −16.61 |
| hsa-miR-4795-5p | −16.61 | −16.61 | −16.61 | −16.61 | −16.61 |
| hsa-miR-4796-3p | −16.61 | −16.61 | −16.61 | −16.61 | −16.61 |
| hsa-miR-4796-5p | −16.61 | −16.61 | −16.61 | −16.61 | −16.61 |
| hsa-miR-4797-3p | −16.61 | −16.61 | 1.897 | −16.61 | −16.61 |
| hsa-miR-4797-5p | −16.61 | −16.61 | −16.61 | −16.61 | −16.61 |
| hsa-miR-4798-3p | −16.61 | −16.61 | −16.61 | −16.61 | −16.61 |
| hsa-miR-4798-5p | −16.61 | −16.61 | −16.61 | −16.61 | −16.61 |
| hsa-miR-4799-3p | −16.61 | −16.61 | −16.61 | −16.61 | −16.61 |
| hsa-miR-4799-5p | 0.459 | −16.61 | 0.897 | −16.61 | −16.61 |
| hsa-miR-4800-3p | −16.61 | −16.61 | −16.61 | −16.61 | −16.61 |
| hsa-miR-4800-5p | −16.61 | −16.61 | 1.727 | −16.61 | −16.61 |
| hsa-miR-4801-3p | −16.61 | −16.61 | −16.61 | −16.61 | −16.61 |
| hsa-miR-4801-5p | −16.61 | −16.61 | −16.61 | −16.61 | −16.61 |
| hsa-miR-4802-3p | −16.61 | −16.61 | 1.187 | −16.61 | −16.61 |
| hsa-miR-4802-5p | −1.348 | −16.61 | 2.585 | 1.176 | 2.78 |
| hsa-miR-4803 | −16.61 | −0.465 | −16.61 | −16.61 | −16.61 |
| hsa-miR-4804-3p | −16.61 | −16.61 | −16.61 | −16.61 | −16.61 |
| hsa-miR-4804-5p | 3.792 | 2.12 | 1.428 | 2.024 | 3.687 |
| hsa-miR-483-3p | 5.838 | 3.12 | 1.312 | 2.661 | −16.61 |
| hsa-miR-483-5p | −16.61 | 10.059 | 5.119 | 5.393 | 5.91 |
| hsa-miR-484 | 3.226 | 5.249 | 4.495 | 3.808 | 8.714 |
| hsa-miR-485-3p | 3.762 | 2.622 | 1.727 | −16.61 | −16.61 |
| hsa-miR-485-5p | −16.61 | 1.342 | −16.61 | −16.61 | 2.95 |
| hsa-miR-486-3p | −16.61 | −16.61 | −16.61 | −16.61 | 2.588 |
| hsa-miR-486-5p | 5.557 | 9.216 | 5.897 | 6.588 | 6.495 |
| hsa-miR-487a-3p | −16.61 | −16.61 | −16.61 | −16.61 | −16.61 |
| hsa-miR-487a-5p | −3.348 | −16.61 | 1.187 | −16.61 | −16.61 |
| hsa-miR-487b-3p | 2.813 | 3.664 | 0.049 | −16.61 | −16.61 |
| hsa-miR-487b-5p | −16.61 | −16.61 | −16.61 | −16.61 | −16.61 |
| hsa-miR-488-3p | 5.527 | 4.684 | 1.428 | 2.313 | −16.61 |
| hsa-miR-488-5p | 4.081 | 3.058 | −16.61 | −16.61 | −16.61 |
| hsa-miR-489-3p | 5.006 | 4.29 | 2.371 | 1.439 | 3.365 |
| hsa-miR-489-5p | −16.61 | −16.61 | −16.61 | −16.61 | −16.61 |
| hsa-miR-490-3p | 8.801 | 8.108 | 4.153 | 4.044 | −16.61 |
| hsa-miR-490-5p | 7.007 | 6.72 | 2.681 | 2.609 | −16.61 |
| hsa-miR-491-3p | 3.547 | −16.61 | 1.727 | 1.313 | −16.61 |
| hsa-miR-491-5p | 6.738 | 6.563 | 4.917 | 4.973 | 4.868 |
| hsa-miR-492 | −16.61 | −16.61 | −16.61 | −16.61 | −16.61 |
| hsa-miR-493-3p | −16.61 | 5.38 | 1.187 | 1.661 | −16.61 |
| hsa-miR-493-5p | −16.61 | 3.393 | −0.273 | 1.313 | −16.61 |
| hsa-miR-494-3p | 4.371 | 5.12 | 2.119 | 2.102 | −16.61 |
| hsa-miR-494-5p | −16.61 | −16.61 | −16.61 | −16.61 | −16.61 |
| hsa-miR-495-3p | −16.61 | 0.535 | −16.61 | −16.61 | −16.61 |
| hsa-miR-495-5p | −16.61 | −16.61 | −16.61 | −16.61 | −16.61 |
| hsa-miR-496 | −16.61 | 0.535 | −16.61 | −16.61 | −16.61 |
| hsa-miR-497-3p | −16.61 | −16.61 | −16.61 | −16.61 | −16.61 |
| hsa-miR-497-5p | 5.055 | 5.089 | 3.187 | 3.582 | 3.95 |
| hsa-miR-498-3p | −16.61 | −16.61 | −16.61 | −16.61 | −16.61 |
| hsa-miR-498-5p | −16.61 | −16.61 | −16.61 | −16.61 | −16.61 |
| hsa-miR-4999-3p | −16.61 | −16.61 | −16.61 | −0.146 | −16.61 |
| hsa-miR-4999-5p | −16.61 | −16.61 | 3.727 | 1.313 | 3.95 |
| hsa-miR-499a-3p | −16.61 | 1.857 | −16.61 | −16.61 | −16.61 |
| hsa-miR-499a-5p | 3.663 | 8.352 | 5.136 | 4.796 | 6.173 |
| hsa-miR-499b-3p | −16.61 | −16.61 | −16.61 | −16.61 | −16.61 |
| hsa-miR-499b-5p | −16.61 | −16.61 | −16.61 | −16.61 | −16.61 |
| hsa-miR-5000-3p | −16.61 | −16.61 | 1.897 | −16.61 | −16.61 |
| hsa-miR-5000-5p | −16.61 | −16.61 | −16.61 | −16.61 | −16.61 |
| hsa-miR-5001-3p | −16.61 | −16.61 | 2.727 | −16.61 | −16.61 |
| hsa-miR-5001-5p | −16.61 | −16.61 | −16.61 | −16.61 | 3.102 |
| hsa-miR-5002-3p | −16.61 | −16.61 | −16.61 | −16.61 | −16.61 |
| hsa-miR-5002-5p | −16.61 | −16.61 | −16.61 | −16.61 | −16.61 |
| hsa-miR-5003-3p | −16.61 | −16.61 | −16.61 | −16.61 | −16.61 |
| hsa-miR-5003-5p | −16.61 | −16.61 | −16.61 | 2.808 | −16.61 |
| hsa-miR-5004-3p | −16.61 | −16.61 | −16.61 | −16.61 | −16.61 |
| hsa-miR-5004-5p | −16.61 | 0.12 | −16.61 | −16.61 | −16.61 |
| hsa-miR-5006-3p | −16.61 | −16.61 | −16.61 | −16.61 | −16.61 |
| hsa-miR-5006-5p | −16.61 | −16.61 | −16.61 | −16.61 | −16.61 |
| hsa-miR-5007-3p | −16.61 | −16.61 | −16.61 | −16.61 | −16.61 |
| hsa-miR-5007-5p | 16.61 | −16.61 | −16.61 | −16.61 | −16.61 |
| hsa-miR-5008-3p | −16.61 | −16.61 | −16.61 | −16.61 | −16.61 |
| hsa-miR-5008-5p | −16.61 | −16.61 | −16.61 | −16.61 | −16.61 |
| hsa-miR-5009-3p | −16.61 | −16.61 | −16.61 | −16.61 | −16.61 |
| hsa-miR-5009-5p | −16.61 | −16.61 | 0.534 | 2.246 | 3.687 |
| hsa-miR-500a-3p/ | 8.141 | 9.109 | 9.634 | 9.881 | 7.207 |
| 502-3p | | | | | |
| hsa-miR-500a-5p/ | 3.772 | 2.994 | 4.693 | 4.194 | 6.803 |
| 500b-5p | | | | | |
| hsa-miR-500b-3p | 2.712 | 1.535 | 2.119 | 2.102 | −16.61 |
| hsa-miR-501-3p | 6.307 | 7.042 | 6.681 | 7.198 | 5.207 |
| hsa-miR-501-5p | 3.756 | 1.857 | 3.4 | 3.526 | 6.102 |
| hsa-miR-5010-3p | 2.399 | −16.61 | 1.049 | −0.146 | −16.61 |
| hsa-miR-5010-5p | 3.745 | 3.393 | 0.897 | 1.313 | 4.78 |
| hsa-miR-5011-3p | −16.61 | −16.61 | −16.61 | −16.61 | −16.61 |
| hsa-miR-5011-5p | −16.61 | −16.61 | −16.61 | −16.61 | −16.61 |
| hsa-miR-502-5p | −16.61 | −16.61 | −16.61 | 0.854 | 4.102 |
| hsa-miR-503-3p | −16.61 | 3.664 | 2.634 | 3.346 | 1.78 |
| hsa-miR-503-5p | 8.453 | 10.878 | 10.307 | 10.588 | 10.597 |
| hsa-miR-504-3p | −16.61 | −16.61 | −16.61 | −16.61 | −16.61 |
| hsa-miR-504-5p | 8.505 | 5.866 | 3.371 | 2.661 | −16.61 |
| hsa-miR-5047 | −16.61 | −16.61 | −16.61 | −16.61 | −16.61 |
| hsa-miR-505-3p | 8.061 | 6.778 | 6.026 | 5.533 | 6.395 |
| hsa-miR-505-5p | 6.587 | 5.622 | 2.897 | 3.468 | 7.417 |
| hsa-miR-506-3p | −16.61 | −16.61 | 0.897 | 1.024 | −16.61 |
| hsa-miR-506-5p | −16.61 | −16.61 | −16.61 | −16.61 | −16.61 |
| hsa-miR-507 | −16.61 | −16.61 | −16.61 | −16.61 | −16.61 |
| hsa-miR-508-3p | 2.709 | 0.857 | 2.937 | 2.313 | 3.365 |
| hsa-miR-508-5p | −16.61 | −16.61 | −16.61 | −16.61 | −16.61 |
| hsa-miR-5087 | −16.61 | −16.61 | −16.61 | −16.61 | −16.61 |
| hsa-miR-5088-3p | −16.61 | −16.61 | −16.61 | −16.61 | −16.61 |
| hsa-miR-5088-5p | 2.999 | −16.61 | 1.727 | −16.61 | −16.61 |
| hsa-miR-5089-3p | −16.61 | −16.61 | −16.61 | −16.61 | −16.61 |
| hsa-miR-5089-5p | −16.61 | −16.61 | −16.61 | −16.61 | −16.61 |
| hsa-miR-509-3-5p | −16.61 | −16.61 | −16.61 | −16.61 | −16.61 |
| hsa-miR-509-3p | 4.122 | 5.058 | 2.771 | 1.941 | −16.61 |
| hsa-miR-509-5p | −16.61 | −16.61 | 2.371 | −16.61 | −16.61 |
| hsa-miR-5090-3p | −16.61 | −16.61 | −16.61 | −16.61 | −16.61 |
| hsa-miR-5090-5p | −16.61 | −16.61 | −16.61 | −16.61 | −16.61 |
| hsa-miR-5091 | −16.61 | −16.61 | −16.61 | −16.61 | −16.61 |
| hsa-miR-5092-3p | −16.61 | −16.61 | −16.61 | −16.61 | −16.61 |
| hsa-miR-5092-5p | −16.61 | −16.61 | −16.61 | −16.61 | −16.61 |
| hsa-miR-5093 | −16.61 | −16.61 | −16.61 | −16.61 | −16.61 |
| hsa-miR-5094-3p | −16.61 | −16.61 | −16.61 | −16.61 | 16.61 |
| hsa-miR-5094-5p | −16.61 | −16.61 | 1.534 | 2.554 | −16.61 |
| hsa-miR-510-3p | −16.61 | −16.61 | −16.61 | −16.61 | −16.61 |
| hsa-miR-510-5p | −16.61 | −16.61 | −16.61 | −16.61 | −16.61 |
| hsa-miR-5100 | −16.61 | −16.61 | 0.049 | −16.61 | −16.61 |
| hsa-miR-511-3p | 4.501 | −16.61 | 3.312 | 4.82 | 8.675 |
| hsa-miR-511-5p | 4.215 | 5.901 | 4.266 | 5.814 | 5.663 |
| hsa-miR-512-3p | −16.61 | 0.12 | −16.61 | −16.61 | −16.61 |
| hsa-miR-512-5p | −16.61 | −16.61 | −16.61 | −16.61 | −16.61 |
| hsa-miR-513a-3p/ | −16.61 | −16.61 | −16.61 | −16.61 | −16.61 |
| 513c-3p | | | | | |
| hsa-miR-513a-5p | 3.292 | −16.61 | −16.61 | −16.61 | 3.588 |
| hsa-miR-513b-3p | 16.61 | −16.61 | 1.634 | −16.61 | −16.61 |
| hsa-miR-513b-5p | −16.61 | −16.61 | 0.727 | −16.61 | −16.61 |
| hsa-miR-513c-5p | 3.588 | −16.61 | 1.534 | −16.61 | −16.61 |
| hsa-miR-514a-3p | 4.364 | 4.058 | 3.428 | 3.526 | 2.102 |
| hsa-miR-514a-5p | −16.61 | 2.705 | −16.61 | −16.61 | −16.61 |
| hsa-miR-514b-3p | −16.61 | −16.61 | −16.61 | −16.61 | −16.61 |
| hsa-miR-514b-5p | −16.61 | −16.61 | −16.61 | −16.61 | −16.61 |
| hsa-miR-515-3p | −16.61 | −16.61 | −16.61 | −16.61 | −16.61 |
| hsa-miR-515-5p | −16.61 | −16.61 | −16.61 | −16.61 | −16.61 |
| hsa-miR-516a-3p/ | −16.61 | −16.61 | −16.61 | −16.61 | −16.61 |
| 516b-3p | | | | | |
| hsa-miR-516a-5p | 4.917 | 4.179 | 0.049 | 3.211 | −16.61 |
| hsa-miR-516b-5p | −16.61 | 1.342 | −16.61 | −16.61 | −16.61 |
| hsa-miR-517-5p | −16.61 | −16.61 | −16.61 | −16.61 | −16.61 |
| hsa-miR-517a-3p/ | −16.61 | −16.61 | −16.61 | −1.146 | −16.61 |
| 517b-3p | | | | | |
| hsa-miR-517c-3p | −16.61 | −16.61 | −16.61 | −16.61 | −16.61 |
| hsa-miR-5186 | −16.61 | −16.61 | −16.61 | −16.61 | −16.61 |
| hsa-miR-5187-3p | −16.61 | −0.465 | −16.61 | 1.554 | −16.61 |
| hsa-miR-5187-5p | 4.361 | 5.207 | 4.102 | 4.044 | 5.562 |
| hsa-miR-5188-3p | −16.61 | −16.61 | 0.049 | −16.61 | −16.61 |
| hsa-miR-5188-5p | −16.61 | −16.61 | −16.61 | −16.61 | −16.61 |
| hsa-miR-5189-3p | −16.61 | −16.61 | −16.61 | −16.61 | −16.61 |
| hsa-miR-5189-5p | −16.61 | −16.61 | −16.61 | −16.61 | −16.61 |
| hsa-miR-518a-3p | −16.61 | −16.61 | −16.61 | −16.61 | −16.61 |
| hsa-miR-518a-5p/ | −16.61 | −16.61 | −16.61 | −16.61 | −16.61 |
| 527-5p | | | | | |
| hsa-miR-518b | −16.61 | 0.857 | −0.688 | −16.61 | −16.61 |
| hsa-miR-518c-3p/ | −16.61 | 1.857 | −16.61 | −16.61 | −16.61 |
| 518f-3p | | | | | |
| hsa-miR-518c-5p | −16.61 | −16.61 | −16.61 | −16.61 | −16.61 |
| hsa-miR-518d-3p | −16.61 | −16.61 | −16.61 | −16.61 | −16.61 |
| hsa-miR-518d-5p/ | −16.61 | −0.465 | −16.61 | −1.146 | −16.61 |
| 518f-5p/520c-5p/ | | | | | |
| 526a-5p | | | | | |
| hsa-miR-518e-3p | −16.61 | −16.61 | −16.61 | −16.61 | −16.61 |
| hsa-miR-518e-5p/ | −16.61 | −16.61 | −16.61 | −16.61 | −16.61 |
| 519a-5p/519b-5p/ | | | | | |
| 519c-5p/522-5p/ | | | | | |
| 523-5p | | | | | |
| hsa-miR-5190-3p | −16.61 | −16.61 | −16.61 | −16.61 | −16.61 |
| hsa-miR-5190-5p | −2.833 | −16.61 | −16.61 | −16.61 | −16.61 |
| hsa-miR-5191-3p | −16.61 | −16.61 | −16.61 | 1.554 | −16.61 |
| hsa-miR-5191-5p | −16.61 | −16.61 | −16.61 | −16.61 | −16.61 |
| hsa-miR-5192-3p | −16.61 | −16.61 | −16.61 | −16.61 | −16.61 |
| hsa-miR-5192-5p | −16.61 | −16.61 | −16.61 | −16.61 | −16.61 |
| hsa-miR-5193-3p | −16.61 | −16.61 | −16.61 | −16.61 | −16.61 |
| hsa-miR-5193-5p | −16.61 | −16.61 | −16.61 | −16.61 | −16.61 |
| hsa-miR-5194-3p | −16.61 | −16.61 | −16.61 | −16.61 | 3.868 |
| hsa-miR-5194-5p | −16.61 | −16.61 | −16.61 | −16.61 | −16.61 |
| hsa-miR-5195-3p | −16.61 | −16.61 | −16.61 | −16.61 | −16.61 |
| hsa-miR-5195-5p | −16.61 | −16.61 | −16.61 | −16.61 | −16.61 |
| hsa-miR-5196-3p | −16.61 | −16.61 | −16.61 | −16.61 | −16.61 |
| hsa-miR-5196-5p | −16.61 | −16.61 | −16.61 | −16.61 | −16.61 |
| hsa-miR-5197-3p | −16.61 | −16.61 | −16.61 | −16.61 | −16.61 |
| hsa-miR-5197-5p | −16.61 | −16.61 | −16.61 | −16.61 | −16.61 |
| hsa-miR-519a-2-5p/ | −16.61 | −16.61 | −16.61 | −16.61 | −16.61 |
| 520b-5p | | | | | |
| hsa-miR-519a-3p/ | −16.61 | −16.61 | −16.61 | −16.61 | −16.61 |
| 519b-3p | | | | | |
| hsa-miR-519c-3p | −16.61 | −16.61 | −16.61 | −16.61 | −16.61 |
| hsa-miR-519d-3p | 0.559 | −0.465 | −16.61 | −16.61 | −16.61 |
| hsa-miR-519d-5p | −16.61 | −16.61 | −16.61 | −16.61 | −16.61 |
| hsa-miR-519e-3p | −16.61 | −16.61 | −16.61 | −16.61 | −16.61 |
| hsa-miR-519e-5p | −16.61 | −16.61 | −16.61 | −16.61 | −16.61 |
| hsa-miR-520a-3p | −16.61 | −16.61 | −16.61 | −16.61 | −16.61 |
| hsa-miR-520a-5p | −16.61 | −16.61 | −16.61 | −16.61 | −16.61 |
| hsa-miR-520b-3p/ | −16.61 | −16.61 | −16.61 | −16.61 | −16.61 |
| 520c-3p/520f-3p/ | | | | | |
| 526b-3p | | | | | |
| hsa-miR-520d-3p | −16.61 | −16.61 | −16.61 | −16.61 | −16.61 |
| hsa-miR-520d-5p | −16.61 | −16.61 | −16.61 | −16.61 | −16.61 |
| hsa-miR-520e-3p | −16.61 | −16.61 | −16.61 | −16.61 | −16.61 |
| hsa-miR-520e-5p | −16.61 | −16.61 | −16.61 | −16.61 | −16.61 |
| hsa-miR-520f-5p | −16.61 | −16.61 | −16.61 | −16.61 | −16.61 |
| hsa-miR-520g-3p/ | −16.61 | −16.61 | −16.61 | −16.61 | −16.61 |
| 520h | | | | | |
| hsa-miR-520g-5p | −16.61 | −16.61 | −16.61 | −16.61 | −16.61 |
| hsa-miR-521-3p | −16.61 | −16.61 | −16.61 | −16.61 | −16.61 |
| hsa-miR-521-5p | −16.61 | −16.61 | −16.61 | −16.61 | −16.61 |
| hsa-miR-522-3p | −16.61 | −16.61 | −16.61 | −16.61 | −16.61 |
| hsa-miR-523-3p | −16.61 | −16.61 | −16.61 | −16.61 | −16.61 |
| hsa-miR-524-3p | −16.61 | −16.61 | −16.61 | −16.61 | −16.61 |
| hsa-miR-524-5p | −16.61 | −16.61 | −16.61 | −16.61 | −16.61 |
| hsa-miR-525-3p | −16.61 | −16.61 | −16.61 | −16.61 | −16.61 |
| hsa-miR-525-5p | −16.61 | −16.61 | −16.61 | −16.61 | −16.61 |
| hsa-miR-526a-3p | −16.61 | −16.61 | −16.61 | −16.61 | −16.61 |
| hsa-miR-526b-5p | −16.61 | 1.342 | −16.61 | −16.61 | −16.61 |
| hsa-miR-527-3p | −16.61 | −16.61 | −16.61 | −16.61 | −16.61 |
| hsa-miR-532-3p | 7.363 | 6.829 | 7.291 | 7.167 | 6.94 |
| hsa-miR-532-5p | 12.194 | 12.241 | 12.77 | 12.922 | 12.755 |
| hsa-miR-539-3p | −16.61 | 2.927 | −16.61 | −16.61 | −16.61 |
| hsa-miR-539-5p | −16.61 | −16.61 | −16.61 | −16.61 | −16.61 |
| hsa-miR-541-3p | −16.61 | −16.61 | −16.61 | −16.61 | −16.61 |
| hsa-miR-541-5p | −16.61 | 0.12 | −16.61 | −16.61 | −16.61 |
| hsa-miR-542-3p | 8.654 | 10.265 | 10.684 | 10.551 | 6.207 |
| hsa-miR-542-5p | 6.303 | 7.303 | 7.766 | 7.472 | 6.223 |
| hsa-miR-543 | 2.279 | −16.61 | −16.61 | −16.61 | −16.61 |
| hsa-miR-544a | −16.61 | −16.61 | −16.61 | −16.61 | −16.61 |
| hsa-miR-544b-3p | −16.61 | −16.61 | −16.61 | −16.61 | −16.61 |
| hsa-miR-544b-5p | −16.61 | −16.61 | −16.61 | −16.61 | −16.61 |
| hsa-miR-545-3p | 5.304 | 2.783 | 3.815 | 3.063 | −16.61 |
| hsa-miR-545-5p | 6.265 | 5.857 | 4.585 | 4.661 | 4.481 |
| hsa-miR-548a-3p | 6.515 | 5.829 | 6.752 | 7.083 | 2.78 |
| hsa-miR-548a-5p | 2.189 | −16.61 | 0.049 | 0.439 | 3.868 |
| hsa-miR-548aa/ | 1.566 | −16.61 | 1.049 | 1.176 | −16.61 |
| 548ap-3p/548t-3p | | | | | |
| hsa-miR-548ab/548n | 4.365 | 4.764 | 3.634 | 4.044 | 3.481 |
| hsa-miR-548ac | −16.61 | −16.61 | 1.312 | −16.61 | −16.61 |
| hsa-miR-548ad-3p | −16.61 | −16.61 | −16.61 | −16.61 | −16.61 |
| hsa-miR-548ad-5p/ | 5.597 | 6.529 | 5.421 | 5.802 | 4.173 |
| 548ae-5p/548ay-5p/ | | | | | |
| 548b-5p/548d-5p | | | | | |
| hsa-miR-548ae-3p/ | 4.589 | 5.01 | 4.312 | 4.648 | −16.61 |
| 548aj-3p/548aq-3p/ | | | | | |
| 548x-3p | | | | | |
| hsa-miR-548ag-3p | 3.085 | −16.61 | −16.61 | −16.61 | −16.61 |
| hsa-miR-548ag-5p | −16.61 | −16.61 | −16.61 | −16.61 | −16.61 |
| hsa-miR-548ah-3p/ | −16.61 | −16.61 | −16.61 | −16.61 | −16.61 |
| 548am-3p | | | | | |
| hsa-miR-548ah-5p | −16.61 | −16.61 | −16.61 | −16.61 | −16.61 |
| hsa-miR-548ai/570-5p | −16.61 | −16.61 | −16.61 | −16.61 | −16.61 |
| hsa-miR-548aj-5p/ | 0.054 | 1.535 | −0.273 | 0.439 | −16.61 |
| 548g-5p/548x-5p | | | | | |
| hsa-miR-548ak/548w | 2.81 | −0.465 | 0.312 | 1.024 | 1.365 |
| hsa-miR-548al | 3.324 | −16.61 | −16.61 | −16.61 | −16.61 |
| hsa-miR-548am-5p/ | 3.803 | 2.12 | 3.085 | 3.554 | 5.847 |
| 548au-5p/548c-5p/ | | | | | |
| 548o-5p | | | | | |
| hsa-miR-548an | 16.61 | −16.61 | −16.61 | −16.61 | −16.61 |
| hsa-miR-548ao-3p | −16.61 | −16.61 | −16.61 | −16.61 | −16.61 |
| hsa-miR-548ao-5p | −16.61 | −16.61 | −16.61 | −16.61 | −16.61 |
| hsa-miR-548ap-5p/ | 3.279 | 4.725 | 5.136 | 5.588 | 5.508 |
| 548j-5p | | | | | |
| hsa-miR-548aq-5p | 3.167 | −16.61 | 0.534 | 0.854 | 2.102 |
| hsa-miR-548ar-3p | −16.61 | −16.61 | 0.049 | −16.61 | −16.61 |
| hsa-miR-548ar-5p | −16.61 | −16.61 | −16.61 | −16.61 | −16.61 |
| hsa-miR-548as-3p | −16.61 | −16.61 | −16.61 | −16.61 | −16.61 |
| hsa-miR-548as-5p | −16.61 | −16.61 | −16.61 | −16.61 | −16.61 |
| hsa-miR-548at-3p | −16.61 | −16.61 | −16.61 | −16.61 | −16.61 |
| hsa-miR-548at-5p | −16.61 | −16.61 | −16.61 | 3.377 | 2.102 |
| hsa-miR-548au-3p | −16.61 | −16.61 | −16.61 | −16.61 | 1.365 |
| hsa-miR-548av-3p | −16.61 | −16.61 | −16.61 | −16.61 | −16.61 |
| hsa-miR-548av-5p/548k | −16.61 | 3.622 | 2.187 | 2.761 | 4.102 |
| hsa-miR-548aw | 3.611 | 0.857 | 1.897 | 2.439 | −16.61 |
| hsa-miR-548ax | −16.61 | 0.12 | −16.61 | −16.61 | 3.365 |
| hsa-miR-548ay-3p | 3.868 | 1.705 | 4.012 | 2.898 | −16.61 |
| hsa-miR-548az-3p | −16.61 | −16.61 | 0.688 | 2.024 | −16.61 |
| hsa-miR-548az-5p | −16.61 | −16.61 | −16.61 | 2.377 | −16.61 |
| hsa-miR-548b-3p | 4.121 | 3.579 | 1.428 | 1.176 | −16.61 |
| hsa-miR-548ba | 3.699 | 0.12 | −16.61 | −16.61 | −16.61 |
| hsa-miR-548bb-3p | −16.61 | −16.61 | −16.61 | −16.61 | −16.61 |
| hsa-miR-548bb-5p | −16.61 | −16.61 | −16.61 | −16.61 | −16.61 |
| hsa-miR-548bc | 4.81 | 3.927 | 4.342 | 4.229 | −16.61 |
| hsa-miR-548c-3p | −16.61 | −16.61 | 1.312 | 1.854 | −16.61 |
| hsa-miR-548d-3p | 1.524 | 0.12 | 0.897 | 2.439 | −16.61 |
| hsa-miR-548e-3p | 3.753 | 5.179 | 4.508 | 5.13 | −16.61 |
| hsa-miR-548e-5p | 4.842 | 4.235 | 5.56 | 5.661 | 3.24 |
| hsa-miR-548f-3p/ | −16.61 | −16.61 | 1.049 | 2.661 | −16.61 |
| 548g-3p | | | | | |
| hsa-miR-548f-5p | −16.61 | −16.61 | −16.61 | −16.61 | −16.61 |
| hsa-miR-548h-3p/548z | 4.168 | 4.579 | 4.136 | 3.808 | −16.61 |
| hsa-miR-548h-5p | 3.681 | 2.442 | −16.61 | −16.61 | −16.61 |
| hsa-miR-548i | −16.61 | 0.12 | −16.61 | −0.561 | −16.61 |
| hsa-miR-548j-3p | 0.503 | 1.342 | 2.727 | 3.28 | −16.61 |
| hsa-miR-548l | 3.283 | −16.61 | 1.312 | 1.176 | 3.78 |
| hsa-miR-548m | −16.61 | −16.61 | −16.61 | −16.61 | −16.61 |
| hsa-miR-548o-3p | 3.454 | 4.235 | 1.428 | −16.61 | −16.61 |
| hsa-miR-548p | 1.845 | −16.61 | −16.61 | −16.61 | −16.61 |
| hsa-miR-548q | 3.45 | 3.744 | 3.282 | 3.102 | 4.102 |
| hsa-miR-548s | −16.61 | −16.61 | −16.61 | −16.61 | −16.61 |
| hsa-miR-548t-5p | −16.61 | 4.764 | 3.049 | 3.313 | 4.535 |
| hsa-miR-548u-3p | 2.699 | 1.12 | 2.815 | 2.377 | −16.61 |
| hsa-miR-548u-5p | −16.61 | −16.61 | −16.61 | −16.61 | −16.61 |
| hsa-miR-548v | −16.61 | −16.61 | −16.61 | −16.61 | −16.61 |
| hsa-miR-548y | −16.61 | −16.61 | −16.61 | −16.61 | −16.61 |
| hsa-miR-549a-3p | −16.61 | −16.61 | −16.61 | −16.61 | −16.61 |
| hsa-miR-549a-5p | −16.61 | −16.61 | −16.61 | −16.61 | −16.61 |
| hsa-miR-550a-3-5p/ | −16.61 | 0.12 | 2.534 | 1.854 | 5.173 |
| 550a-5p/550b-2-5p | | | | | |
| hsa-miR-550a-3p | −16.61 | −16.61 | 1.312 | −16.61 | −16.61 |
| hsa-miR-550b-3p | −16.61 | −16.61 | −16.61 | −16.61 | −16.61 |
| hsa-miR-551a | 4.698 | 4.91 | 4.975 | 3.854 | 2.95 |
| hsa-miR-551b-3p | 9.492 | 7.309 | 3.012 | 2.898 | −16.61 |
| hsa-miR-551b-5p | 4.563 | 3.29 | −16.61 | 2.498 | 3.481 |
| hsa-miR-552-3p | −16.61 | −16.61 | −16.61 | −16.61 | −16.61 |
| hsa-miR-552-5p | 3.083 | −16.61 | −16.61 | −16.61 | −16.61 |
| hsa-miR-553 | −16.61 | −16.61 | −16.61 | −16.61 | −16.61 |
| hsa-miR-554 | −16.61 | −16.61 | −16.61 | −16.61 | −16.61 |
| hsa-miR-555 | −16.61 | −16.61 | −16.61 | −16.61 | −16.61 |
| hsa-miR-556-3p | 3.956 | 1.12 | −16.61 | −16.61 | −16.61 |
| hsa-miR-556-5p | 3.202 | 0.857 | −16.61 | −1.146 | 3.365 |
| hsa-miR-557 | −16.61 | −16.61 | −16.61 | −16.61 | −16.61 |
| hsa-miR-5571-3p | −16.61 | −16.61 | −16.61 | −16.61 | −16.61 |
| hsa-miR-5571-5p | −16.61 | −16.61 | −16.61 | −16.61 | −16.61 |
| hsa-miR-5572 | 16.61 | −16.61 | −16.61 | −16.61 | −16.61 |
| hsa-miR-5579-3p | 2.275 | −16.61 | 1.049 | −16.61 | 2.102 |
| hsa-miR-5579-5p | −16.61 | −16.61 | −16.61 | −16.61 | −16.61 |
| hsa-miR-558 | −16.61 | −16.61 | −16.61 | −16.61 | −16.61 |
| hsa-miR-5580-3p | −16.61 | −16.61 | −16.61 | −16.61 | −16.61 |
| hsa-miR-5580-5p | −16.61 | −16.61 | −16.61 | −16.61 | −16.61 |
| hsa-miR-5581-3p | −16.61 | −16.61 | −16.61 | −16.61 | −16.61 |
| hsa-miR-5581-5p | 3.332 | −16.61 | −16.61 | −16.61 | −16.61 |
| hsa-miR-5582-3p | −16.61 | −16.61 | −16.61 | −16.61 | −16.61 |
| hsa-miR-5582-5p | −16.61 | −16.61 | −16.61 | −16.61 | −16.61 |
| hsa-miR-5583-3p | −16.61 | 3.892 | 1.049 | 2.712 | −16.61 |
| hsa-miR-5583-5p | −16.61 | −16.61 | −16.61 | −16.61 | −16.61 |
| hsa-miR-5584-3p | −16.61 | −16.61 | −16.61 | −16.61 | −16.61 |
| hsa-miR-5584-5p | 5.196 | 1.994 | −16.61 | −16.61 | −16.61 |
| hsa-miR-5585-3p | −16.61 | 0.12 | 1.312 | −16.61 | 3.365 |
| hsa-miR-5585-5p | −16.61 | −16.61 | 2.482 | −16.61 | −16.61 |
| hsa-miR-5586-3p | −16.61 | −16.61 | 1.727 | 2.712 | 4.78 |
| hsa-miR-5586-5p | 3.235 | 4.235 | 4.761 | 4.609 | 4.24 |
| hsa-miR-5587-3p | −16.61 | −16.61 | −16.61 | −16.61 | −16.61 |
| hsa-miR-5587-5p | −16.61 | −16.61 | −16.61 | −16.61 | −16.61 |
| hsa-miR-5588-3p | −16.61 | −16.61 | −16.61 | −16.61 | −16.61 |
| hsa-miR-5588-5p | −16.61 | −16.61 | −16.61 | −16.61 | −16.61 |
| hsa-miR-5589-3p | −16.61 | −16.61 | −16.61 | −16.61 | −16.61 |
| hsa-miR-5589-5p | −16.61 | −16.61 | −16.61 | −16.61 | −16.61 |
| hsa-miR-559-3p | 16.61 | −16.61 | −16.61 | −16.61 | −16.61 |
| hsa-miR-559-5p | −16.61 | −16.61 | −16.61 | −16.61 | −16.61 |
| hsa-miR-5590-3p | −16.61 | −16.61 | −16.61 | −16.61 | −16.61 |
| hsa-miR-5590-5p | −16.61 | −16.61 | −16.61 | −16.61 | −16.61 |
| hsa-miR-5591-3p | −16.61 | −16.61 | −16.61 | −16.61 | −16.61 |
| hsa-miR-5591-5p | −16.61 | −16.61 | −16.61 | −16.61 | −16.61 |
| hsa-miR-561-3p | 2.332 | 1.12 | −16.61 | −16.61 | −16.61 |
| hsa-miR-561-5p | 6.217 | 5.316 | 3.342 | 3.102 | −16.61 |
| hsa-miR-562 | −16.61 | −16.61 | −16.61 | −16.61 | −16.61 |
| hsa-miR-563-3p | −16.61 | −16.61 | −16.61 | −16.61 | −16.61 |
| hsa-miR-563-5p | −16.61 | −16.61 | −16.61 | −16.61 | −16.61 |
| hsa-miR-564 | −16.61 | −16.61 | −16.61 | −16.61 | −16.61 |
| hsa-miR-567-3p | −16.61 | −16.61 | −16.61 | −16.61 | −16.61 |
| hsa-miR-567-5p | −16.61 | −16.61 | −16.61 | −16.61 | −16.61 |
| hsa-miR-568 | −16.61 | −16.61 | −16.61 | −16.61 | −16.61 |
| hsa-miR-5680 | −16.61 | −16.61 | −16.61 | −16.61 | −16.61 |
| hsa-miR-5681a | −16.61 | −16.61 | −16.61 | −16.61 | −16.61 |
| hsa-miR-5681b | −16.61 | −16.61 | −16.61 | −16.61 | −16.61 |
| hsa-miR-5682 | −16.61 | −16.61 | −16.61 | −16.61 | −16.61 |
| hsa-miR-5683 | 2.456 | −16.61 | −16.61 | −16.61 | −16.61 |
| hsa-miR-5684 | −16.61 | −16.61 | −16.61 | 2.313 | −16.61 |
| hsa-miR-5685 | −16.61 | −16.61 | −16.61 | −16.61 | −16.61 |
| hsa-miR-5687-3p | −16.61 | −16.61 | −16.61 | −16.61 | −16.61 |
| hsa-miR-5687-5p | −16.61 | −16.61 | −16.61 | −16.61 | −16.61 |
| hsa-miR-5688 | −16.61 | −16.61 | −16.61 | −16.61 | −16.61 |
| hsa-miR-5689 | −16.61 | −16.61 | −16.61 | −16.61 | −16.61 |
| hsa-miR-569 | −16.61 | −16.61 | −16.61 | −16.61 | −16.61 |
| hsa-miR-5690 | −16.61 | −16.61 | −16.61 | −16.61 | −16.61 |
| hsa-miR-5691 | −16.61 | −16.61 | −16.61 | −16.61 | −16.61 |
| hsa-miR-5692a | −16.61 | −16.61 | −16.61 | −16.61 | −16.61 |
| hsa-miR-5692b/5692c | −16.61 | −16.61 | −16.61 | −16.61 | −16.61 |
| hsa-miR-5693-3p | −16.61 | −16.61 | −16.61 | −16.61 | −16.61 |
| hsa-miR-5693-5p | −16.61 | −16.61 | −16.61 | −16.61 | −16.61 |
| hsa-miR-5694 | −16.61 | −16.61 | −16.61 | −16.61 | −16.61 |
| hsa-miR-5695-3p | 16.61 | −16.61 | −16.61 | 2.761 | −16.61 |
| hsa-miR-5695-5p | −16.61 | −16.61 | −16.61 | −16.61 | −16.61 |
| hsa-miR-5696 | −16.61 | −16.61 | 0.534 | −16.61 | −16.61 |
| hsa-miR-5697-3p | −16.61 | −16.61 | −16.61 | −16.61 | −16.61 |
| hsa-miR-5697-5p | −16.61 | −16.61 | 2.049 | −16.61 | 2.95 |
| hsa-miR-5698 | 2.202 | −16.61 | −16.61 | −16.61 | −16.61 |
| hsa-miR-5699-3p | 4.291 | 4.058 | −16.61 | −16.61 | −16.61 |
| hsa-miR-5699-5p | 6.173 | 2.857 | 1.634 | −16.61 | −16.61 |
| hsa-miR-570-3p | 3.967 | 4.342 | 3.61 | 3.498 | 3.868 |
| hsa-miR-5700 | −16.61 | −16.61 | −16.61 | −16.61 | −16.61 |
| hsa-miR-5701 | −16.61 | −16.61 | 1.049 | −16.61 | −16.61 |
| hsa-miR-5702-3p | −16.61 | −16.61 | −16.61 | −16.61 | −16.61 |
| hsa-miR-5702-5p | −16.61 | −16.61 | −16.61 | −16.61 | −16.61 |
| hsa-miR-5703 | −16.61 | −16.61 | −16.61 | −16.61 | −16.61 |
| hsa-miR-5704 | −16.61 | −16.61 | −16.61 | −16.61 | −16.61 |
| hsa-miR-5705 | −16.61 | −16.61 | −16.61 | −16.61 | −16.61 |
| hsa-miR-5706 | −16.61 | −16.61 | −16.61 | 1.024 | −16.61 |
| hsa-miR-5707 | −16.61 | −16.61 | −16.61 | −16.61 | −16.61 |
| hsa-miR-5708 | −16.61 | −16.61 | −16.61 | −16.61 | −16.61 |
| hsa-miR-571 | −16.61 | −16.61 | −16.61 | −16.61 | −16.61 |
| hsa-miR-572 | −16.61 | −16.61 | −16.61 | −16.61 | −16.61 |
| hsa-miR-573 | −16.61 | −16.61 | −16.61 | −16.61 | −16.61 |
| hsa-miR-5739 | −16.61 | −16.61 | −16.61 | −16.61 | −16.61 |
| hsa-miR-574-3p | 9.48 | 8.295 | 7.412 | 7.446 | 7.248 |
| hsa-miR-574-5p | 7.932 | 8.526 | 6.569 | 6.868 | 10.682 |
| hsa-miR-575 | −16.61 | −16.61 | −16.61 | −16.61 | −16.61 |
| hsa-miR-576-3p | −16.61 | 2.857 | 2.815 | 3.609 | 3.588 |
| hsa-miR-576-5p | 5.118 | 4.207 | 5.003 | 4.831 | 4.028 |
| hsa-miR-577-3p | −16.61 | −0.465 | −16.61 | −16.61 | −16.61 |
| hsa-miR-577-5p | 3.35 | 2.442 | 0.312 | −16.61 | −16.61 |
| hsa-miR-578-3p | −16.61 | −16.61 | −16.61 | −16.61 | −16.61 |
| hsa-miR-578-5p | −16.61 | −16.61 | −16.61 | −16.61 | −16.61 |
| hsa-miR-5787 | −16.61 | −16.61 | −16.61 | −16.61 | −16.61 |
| hsa-miR-579-3p | 3.306 | −16.61 | 0.727 | 0.176 | 2.365 |
| hsa-miR-579-5p | −16.61 | −0.465 | 0.049 | −16.61 | 3.481 |
| hsa-miR-580-3p | −16.61 | 3.29 | 3.956 | 3.468 | −16.61 |
| hsa-miR-580-5p | 0.69 | −16.61 | −16.61 | −16.61 | −16.61 |
| hsa-miR-581-3p | −16.61 | −16.61 | −16.61 | −16.61 | −16.61 |
| hsa-miR-581-5p | 1.626 | −16.61 | −16.61 | −16.61 | 3.365 |
| hsa-miR-582-3p | 5.904 | 5.417 | 4.738 | 5.313 | 3.588 |
| hsa-miR-582-5p | 4.056 | 1.12 | 1.428 | 2.102 | 3.24 |
| hsa-miR-583 | −16.61 | −16.61 | −16.61 | −16.61 | −16.61 |
| hsa-miR-584-3p | −16.61 | −16.61 | −16.61 | −16.61 | −16.61 |
| hsa-miR-584-5p | 3.863 | 3.489 | −16.61 | 3.313 | 3.481 |
| hsa-miR-585-3p | 4.821 | 4.089 | 2.187 | 0.176 | −16.61 |
| hsa-miR-585-5p | −16.61 | 1.705 | −16.61 | −16.61 | −16.61 |
| hsa-miR-586 | −16.61 | −16.61 | −16.61 | −16.61 | −16.61 |
| hsa-miR-587 | −16.61 | −16.61 | −16.61 | −16.61 | −16.61 |
| hsa-miR-588-3p | −16.61 | −16.61 | −16.61 | −16.61 | −16.61 |
| hsa-miR-588-5p | −16.61 | −16.61 | −16.61 | −16.61 | −16.61 |
| hsa-miR-589-3p | 3.892 | 4.12 | 3.877 | 3.102 | −16.61 |
| hsa-miR-589-5p | 5.732 | 6.792 | 6.588 | 6.828 | 7.775 |
| hsa-miR-590-3p | 8.366 | 8.383 | 8.911 | 8.65 | 9.084 |
| hsa-miR-590-5p | 5.322 | 5.754 | 6.211 | 5.767 | 5.365 |
| hsa-miR-591 | −16.61 | −16.61 | −16.61 | −16.61 | −16.61 |
| hsa-miR-592 | 5.247 | 5.149 | 1.049 | −16.61 | −16.61 |
| hsa-miR-593-3p | −16.61 | −16.61 | −16.61 | −16.61 | −16.61 |
| hsa-miR-593-5p | −16.61 | −16.61 | −16.61 | −16.61 | −16.61 |
| hsa-miR-595 | −16.61 | −16.61 | −16.61 | −16.61 | −16.61 |
| hsa-miR-596 | −16.61 | −16.61 | −16.61 | −16.61 | −16.61 |
| hsa-miR-597-3p | −16.61 | 0.535 | −16.61 | −16.61 | −16.61 |
| hsa-miR-597-5p | 0.739 | −16.61 | 0.534 | 1.761 | −16.61 |
| hsa-miR-598-3p | 11.391 | 9.512 | 9.035 | 8.603 | 9.555 |
| hsa-miR-598-5p | −16.61 | −16.61 | −16.61 | −0.146 | −16.61 |
| hsa-miR-599 | −16.61 | −16.61 | −16.61 | −16.61 | −16.61 |
| hsa-miR-600-3p | −16.61 | −16.61 | −16.61 | −16.61 | −16.61 |
| hsa-miR-600-5p | −16.61 | −16.61 | 3.049 | −16.61 | 3.687 |
| hsa-miR-601 | −16.61 | −16.61 | −16.61 | −16.61 | −16.61 |
| hsa-miR-602-3p | −16.61 | −16.61 | −16.61 | −16.61 | −16.61 |
| hsa-miR-602-5p | −16.61 | −16.61 | −16.61 | −16.61 | −16.61 |
| hsa-miR-603 | −16.61 | 0.12 | −16.61 | −16.61 | −16.61 |
| hsa-miR-604 | −16.61 | −16.61 | −16.61 | −16.61 | −16.61 |
| hsa-miR-605-3p | −16.61 | −16.61 | −16.61 | −16.61 | −16.61 |
| hsa-miR-605-5p | −16.61 | −16.61 | −16.61 | −16.61 | −16.61 |
| hsa-miR-606-3p | −16.61 | −16.61 | −16.61 | −16.61 | −16.61 |
| hsa-miR-606-5p | −16.61 | −16.61 | −16.61 | −16.61 | −16.61 |
| hsa-miR-6068 | −16.61 | −16.61 | −16.61 | −16.61 | −16.61 |
| hsa-miR-6069 | −16.61 | −16.61 | −16.61 | −16.61 | −16.61 |
| hsa-miR-607 | 16.61 | −16.61 | −16.61 | −16.61 | −16.61 |
| hsa-miR-6070 | −16.61 | −16.61 | −16.61 | −16.61 | −16.61 |
| hsa-miR-6071 | −16.61 | −16.61 | −16.61 | −16.61 | −16.61 |
| hsa-miR-6072 | −16.61 | −16.61 | −16.61 | −16.61 | −16.61 |
| hsa-miR-6073 | −16.61 | −16.61 | −16.61 | −16.61 | −16.61 |
| hsa-miR-6074 | −16.61 | −16.61 | −16.61 | −16.61 | −16.61 |
| hsa-miR-6075 | −16.61 | −16.61 | −16.61 | −16.61 | −16.61 |
| hsa-miR-6076 | −16.61 | −16.61 | −16.61 | −16.61 | −16.61 |
| hsa-miR-6077 | −16.61 | −16.61 | −16.61 | −16.61 | −16.61 |
| hsa-miR-6078 | −16.61 | −16.61 | −16.61 | −16.61 | −16.61 |
| hsa-miR-6079 | −16.61 | −16.61 | −16.61 | −16.61 | −16.61 |
| hsa-miR-608 | −16.61 | −16.61 | −16.61 | −16.61 | −16.61 |
| hsa-miR-6080 | −16.61 | −16.61 | −16.61 | −16.61 | −16.61 |
| hsa-miR-6081 | −16.61 | −16.61 | −16.61 | −16.61 | −16.61 |
| hsa-miR-6082 | −16.61 | −16.61 | −16.61 | −16.61 | −16.61 |
| hsa-miR-6083 | −16.61 | −16.61 | −16.61 | −16.61 | −16.61 |
| hsa-miR-6084 | −16.61 | −16.61 | −16.61 | −16.61 | −16.61 |
| hsa-miR-6085 | −16.61 | −16.61 | −16.61 | −16.61 | −16.61 |
| hsa-miR-6086 | −16.61 | −16.61 | −16.61 | −16.61 | −16.61 |
| hsa-miR-6088 | −16.61 | −16.61 | −16.61 | −16.61 | −16.61 |
| hsa-miR-6089 | −16.61 | −16.61 | −16.61 | −16.61 | −16.61 |
| hsa-miR-609 | −16.61 | −16.61 | −16.61 | −16.61 | −16.61 |
| hsa-miR-6090 | −16.61 | −16.61 | −16.61 | −16.61 | −16.61 |
| hsa-miR-610 | −16.61 | −16.61 | −16.61 | −16.61 | −16.61 |
| hsa-miR-611 | −16.61 | −16.61 | −16.61 | −16.61 | −16.61 |
| hsa-miR-612 | −16.61 | −16.61 | −16.61 | −16.61 | −16.61 |
| hsa-miR-6124 | −16.61 | −16.61 | −16.61 | −16.61 | −16.61 |
| hsa-miR-6125 | −16.61 | −16.61 | −16.61 | −16.61 | −16.61 |
| hsa-miR-6126 | −16.61 | −16.61 | −16.61 | −16.61 | −16.61 |
| hsa-miR-6127 | −16.61 | −16.61 | −16.61 | −16.61 | −16.61 |
| hsa-miR-6128 | −16.61 | −16.61 | −16.61 | −16.61 | −16.61 |
| hsa-miR-6129 | −16.61 | −16.61 | −16.61 | −16.61 | −16.61 |
| hsa-miR-613 | −16.61 | −16.61 | −16.61 | −16.61 | −16.61 |
| hsa-miR-6130 | −16.61 | −16.61 | −16.61 | −16.61 | −16.61 |
| hsa-miR-6131 | −16.61 | −16.61 | −16.61 | −16.61 | −16.61 |
| hsa-miR-6132-3p | −16.61 | 0.535 | −16.61 | −16.61 | −16.61 |
| hsa-miR-6132-5p | −16.61 | −16.61 | −16.61 | −16.61 | −16.61 |
| hsa-miR-6133 | −16.61 | −16.61 | −16.61 | −16.61 | −16.61 |
| hsa-miR-6134 | −16.61 | −16.61 | −16.61 | −16.61 | −16.61 |
| hsa-miR-614 | −16.61 | −16.61 | −16.61 | −16.61 | −16.61 |
| hsa-miR-615-3p | −16.61 | 4.557 | −16.61 | 1.024 | −16.61 |
| hsa-miR-615-5p | −16.61 | −16.61 | −16.61 | −16.61 | −16.61 |
| hsa-miR-616-3p | 3.751 | 1.705 | 1.634 | 2.176 | 4.95 |
| hsa-miR-616-5p | 3.302 | 4.368 | −16.61 | −16.61 | −16.61 |
| hsa-miR-6165 | −16.61 | −16.61 | −16.61 | −16.61 | −16.61 |
| hsa-miR-617 | −16.61 | −16.61 | −16.61 | −16.61 | −16.61 |
| hsa-miR-618-3p | −16.61 | −16.61 | −16.61 | −16.61 | −16.61 |
| hsa-miR-618-5p | −16.61 | −16.61 | 2.771 | 3.526 | −16.61 |
| hsa-miR-619-3p | −16.61 | −16.61 | −16.61 | −16.61 | −16.61 |
| hsa-miR-619-5p | −16.61 | −16.61 | 1.534 | −16.61 | −16.61 |
| hsa-miR-620-3p | −16.61 | −16.61 | −16.61 | −16.61 | −16.61 |
| hsa-miR-620-5p | −16.61 | −16.61 | −16.61 | −16.61 | −16.61 |
| hsa-miR-621 | −16.61 | −16.61 | −16.61 | −16.61 | −16.61 |
| hsa-miR-622 | −16.61 | −16.61 | −16.61 | −16.61 | −16.61 |
| hsa-miR-623 | −16.61 | −16.61 | −16.61 | −16.61 | −16.61 |
| hsa-miR-624-3p | −16.61 | −16.61 | −16.61 | −16.61 | −16.61 |
| hsa-miR-624-5p | 2.555 | 0.535 | 2.049 | 2.498 | 4.424 |
| hsa-miR-625-3p | 8.139 | 8.63 | 9.433 | 9.474 | 3.868 |
| hsa-miR-625-5p | 6.9 | 7.342 | 7.83 | 8.089 | 11.088 |
| hsa-miR-626 | −16.61 | −16.61 | −16.61 | −16.61 | −16.61 |
| hsa-miR-627-3p | −16.61 | −16.61 | 1.534 | 1.176 | −16.61 |
| hsa-miR-627-5p | 4.741 | 4.838 | 4.312 | 5.22 | 7.129 |
| hsa-miR-628-3p | 7.564 | 7.641 | 6.17 | 6.991 | 8.244 |
| hsa-miR-628-5p | 8.105 | 7.423 | 6.102 | 6.957 | 6.102 |
| hsa-miR-629-3p | 2.943 | 1.12 | 3.049 | 2.377 | 3.687 |
| hsa-miR-629-5p | 8.003 | 10.617 | 9.345 | 9.901 | 10.922 |
| hsa-miR-630 | −16.61 | −16.61 | −16.61 | −16.61 | −16.61 |
| hsa-miR-631 | −16.61 | −16.61 | −16.61 | −16.61 | −16.61 |
| hsa-miR-632 | −16.61 | −16.61 | −16.61 | −16.61 | −16.61 |
| hsa-miR-633 | −16.61 | −16.61 | −16.61 | −16.61 | −16.61 |
| hsa-miR-634 | −16.61 | −16.61 | −16.61 | −16.61 | −16.61 |
| hsa-miR-635 | −16.61 | −16.61 | −16.61 | −16.61 | −16.61 |
| hsa-miR-636-3p | −16.61 | −16.61 | −16.61 | −16.61 | −16.61 |
| hsa-miR-636-5p | −16.61 | −16.61 | −16.61 | −16.61 | −16.61 |
| hsa-miR-637 | −16.61 | −16.61 | −16.61 | −16.61 | −16.61 |
| hsa-miR-638 | −16.61 | −16.61 | −16.61 | −16.61 | −16.61 |
| hsa-miR-639 | −16.61 | −16.61 | −16.61 | −16.61 | −16.61 |
| hsa-miR-640 | −16.61 | −16.61 | −16.61 | −16.61 | −16.61 |
| hsa-miR-641 | 4.462 | 3.664 | 1.727 | −16.61 | 4.868 |
| hsa-miR-642a-3p/ | 4.751 | 4.892 | 4.956 | 4.887 | 6.651 |
| 642b-3p | | | | | |
| hsa-miR-642a-5p | 7.394 | 5.393 | 4.927 | 4.865 | 5.24 |
| hsa-miR-642b-5p | −16.61 | −16.61 | −16.61 | −16.61 | −16.61 |
| hsa-miR-643-3p | 2.83 | 1.342 | 2.585 | 2.808 | −16.61 |
| hsa-miR-643-5p | −16.61 | −16.61 | −16.61 | 1.176 | −16.61 |
| hsa-miR-644a | −16.61 | −16.61 | −16.61 | −16.61 | −16.61 |
| hsa-miR-645 | −16.61 | −16.61 | −16.61 | −16.61 | −16.61 |
| hsa-miR-646 | −16.61 | −16.61 | −16.61 | −16.61 | −16.61 |
| hsa-miR-647 | −16.61 | −16.61 | −16.61 | −16.61 | −16.61 |
| hsa-miR-648 | 16.61 | −16.61 | −16.61 | −16.61 | −16.61 |
| hsa-miR-649 | −16.61 | −16.61 | −16.61 | −16.61 | −16.61 |
| hsa-miR-6499-3p | −16.61 | −16.61 | −16.61 | −16.61 | −16.61 |
| hsa-miR-6499-5p | −16.61 | −16.61 | −16.61 | −16.61 | −16.61 |
| hsa-miR-650 | −16.61 | −16.61 | −16.61 | −16.61 | −16.61 |
| hsa-miR-6500-3p | −16.61 | −16.61 | −16.61 | −16.61 | −16.61 |
| hsa-miR-6500-5p | −16.61 | −16.61 | −16.61 | −16.61 | −16.61 |
| hsa-miR-6501-3p | −16.61 | −16.61 | −16.61 | −16.61 | −16.61 |
| hsa-miR-6501-5p | −16.61 | −16.61 | −16.61 | 2.176 | −16.61 |
| hsa-miR-6502-3p | −16.61 | −16.61 | −16.61 | −16.61 | −16.61 |
| hsa-miR-6502-5p | −16.61 | −16.61 | 1.187 | 1.313 | −16.61 |
| hsa-miR-6503-3p | 6.731 | 7.715 | 9.598 | 8.728 | 4.825 |
| hsa-miR-6503-5p | 2.662 | −16.61 | 4.836 | 3.736 | 6.803 |
| hsa-miR-6504-3p | −16.61 | −16.61 | −16.61 | −16.61 | −16.61 |
| hsa-miR-6504-5p | −16.61 | −16.61 | −16.61 | −16.61 | −16.61 |
| hsa-miR-6505-3p | −16.61 | −16.61 | −16.61 | −16.61 | −16.61 |
| hsa-miR-6505-5p | 4.245 | 3.058 | −16.61 | 1.176 | −16.61 |
| hsa-miR-6506-3p | −16.61 | −16.61 | −16.61 | −16.61 | −16.61 |
| hsa-miR-6506-5p | −16.61 | −16.61 | −16.61 | −16.61 | −16.61 |
| hsa-miR-6507-3p | −16.61 | −16.61 | −16.61 | −16.61 | −16.61 |
| hsa-miR-6507-5p | 3.945 | 0.857 | −16.61 | 2.808 | −16.61 |
| hsa-miR-6508-3p | −16.61 | −16.61 | −16.61 | −16.61 | −16.61 |
| hsa-miR-6508-5p | −16.61 | −16.61 | −16.61 | −16.61 | −16.61 |
| hsa-miR-6509-3p | −16.61 | −16.61 | −16.61 | −16.61 | −16.61 |
| hsa-miR-6509-5p | −16.61 | −16.61 | 0.727 | −16.61 | −16.61 |
| hsa-miR-651-3p | −16.61 | −16.61 | −16.61 | −16.61 | −16.61 |
| hsa-miR-651-5p | 5.626 | 5.953 | 5.804 | 6.034 | 6.78 |
| hsa-miR-6510-3p | −16.61 | −16.61 | −16.61 | −16.61 | −16.61 |
| hsa-miR-6510-5p | −16.61 | −16.61 | −16.61 | −16.61 | −16.61 |
| hsa-miR-6511a-3p | 3.696 | 1.342 | 0.534 | 2.439 | 3.481 |
| hsa-miR-6511a-5p | −16.61 | −16.61 | −16.61 | −16.61 | −16.61 |
| hsa-miR-6511b-3p | 4.116 | −16.61 | −0.273 | −16.61 | −16.61 |
| hsa-miR-6511b-5p | −16.61 | −16.61 | −16.61 | −16.61 | −16.61 |
| hsa-miR-6512-3p | −16.61 | −16.61 | −16.61 | −16.61 | −16.61 |
| hsa-miR-6512-5p | −16.61 | −16.61 | −16.61 | −16.61 | −16.61 |
| hsa-miR-6513-3p | −16.61 | 0.12 | 2.815 | 2.246 | 2.588 |
| hsa-miR-6513-5p | −16.61 | 3.892 | 2.585 | −16.61 | 2.102 |
| hsa-miR-6514-3p | −16.61 | −16.61 | −16.61 | −16.61 | −16.61 |
| hsa-miR-6514-5p | 3.86 | 2.442 | 2.482 | 3.246 | 3.24 |
| hsa-miR-6515-3p | −16.61 | −16.61 | −16.61 | −16.61 | −16.61 |
| hsa-miR-6515-5p | −16.61 | −16.61 | −16.61 | −16.61 | 4.481 |
| hsa-miR-6516-3p | 3.157 | −16.61 | −16.61 | 1.024 | −16.61 |
| hsa-miR-6516-5p | 2.784 | −16.61 | 1.634 | −16.61 | 3.78 |
| hsa-miR-652-3p | 5.034 | 5.221 | 5.761 | 5.362 | 10.568 |
| hsa-miR-652-5p | 3.45 | 0.535 | 2.482 | 2.808 | 7 |
| hsa-miR-6529-3p | −16.61 | −16.61 | −16.61 | −16.61 | −16.61 |
| hsa-miR-6529-5p | −16.61 | 3.927 | 0.897 | −16.61 | −16.61 |
| hsa-miR-653-3p | 5.529 | 4.557 | 1.534 | 1.854 | 4.102 |
| hsa-miR-653-5p | 4.697 | 5.705 | 2.815 | 2.941 | −16.61 |
| hsa-miR-654-3p | 4.277 | 5.405 | 1.727 | 2.024 | −16.61 |
| hsa-miR-654-5p | −16.61 | −16.61 | −16.61 | −16.61 | −16.61 |
| hsa-miR-655-3p | −16.61 | 1.994 | −16.61 | −16.61 | −16.61 |
| hsa-miR-655-5p | −16.61 | −16.61 | −16.61 | −16.61 | −16.61 |
| hsa-miR-656-3p | −16.61 | −16.61 | −16.61 | −16.61 | −16.61 |
| hsa-miR-656-5p | −16.61 | −16.61 | −16.61 | −16.61 | −16.61 |
| hsa-miR-657 | −16.61 | −16.61 | −16.61 | −16.61 | −16.61 |
| hsa-miR-658 | −16.61 | −16.61 | −16.61 | −16.61 | −16.61 |
| hsa-miR-659-3p | −16.61 | −16.61 | −16.61 | −16.61 | −16.61 |
| hsa-miR-659-5p | 3.838 | −16.61 | −16.61 | 1.661 | −16.61 |
| hsa-miR-660-3p | 2.802 | 0.857 | 0.049 | 2.246 | 3.24 |
| hsa-miR-660-5p | 10.852 | 10.858 | 11.746 | 11.709 | 12.538 |
| hsa-miR-661 | −16.61 | −16.61 | −16.61 | −16.61 | −16.61 |
| hsa-miR-662 | −16.61 | −16.61 | −16.61 | −16.61 | −16.61 |
| hsa-miR-663a-3p | −16.61 | 1.857 | 1.815 | −16.61 | −16.61 |
| hsa-miR-663a-5p | −16.61 | −16.61 | −16.61 | 1.313 | −16.61 |
| hsa-miR-663b | −16.61 | −16.61 | −16.61 | −16.61 | −16.61 |
| hsa-miR-664a-3p | 1.969 | 0.12 | −16.61 | 2.176 | 4.028 |
| hsa-miR-664a-5p | 6.474 | 7.164 | 5.342 | 6.246 | 5.638 |
| hsa-miR-664b-3p | 1.51 | 3.664 | 0.727 | 0.854 | 2.78 |
| hsa-miR-664b-5p | −16.61 | 5.263 | 2.815 | 3.687 | 4.24 |
| hsa-miR-665-3p | −16.61 | 1.342 | −16.61 | −16.61 | −16.61 |
| hsa-miR-665-5p | −16.61 | −16.61 | −16.61 | −16.61 | −16.61 |
| hsa-miR-668-3p | −16.61 | −16.61 | −16.61 | −16.61 | −16.61 |
| hsa-miR-668-5p | −16.61 | −16.61 | −16.61 | −16.61 | −16.61 |
| hsa-miR-670-3p | 5.517 | 3.342 | −16.61 | −16.61 | −16.61 |
| hsa-miR-670-5p | 4.83 | −16.61 | −16.61 | −16.61 | −16.61 |
| hsa-miR-671-3p | 4.946 | 2.994 | 3.681 | 3.941 | 3.687 |
| hsa-miR-671-5p | 5.483 | 6.883 | 5.97 | 6.034 | 9.331 |
| hsa-miR-6715a-3p | 2.696 | −16.61 | −16.61 | −16.61 | −16.61 |
| hsa-miR-6715b-3p | −16.61 | −16.61 | −16.61 | −16.61 | −16.61 |
| hsa-miR-6715b-5p | −16.61 | −16.61 | −16.61 | −16.61 | −16.61 |
| hsa-miR-6716-3p | 3.735 | −16.61 | −16.61 | −16.61 | −16.61 |
| hsa-miR-6716-5p | −16.61 | −16.61 | −16.61 | −16.61 | −16.61 |
| hsa-miR-6717-5p | −16.61 | −16.61 | −16.61 | −16.61 | −16.61 |
| hsa-miR-6718-3p | −16.61 | −16.61 | 2.897 | 2.609 | −16.61 |
| hsa-miR-6718-5p | −16.61 | −16.61 | 3.428 | 3.176 | 4.173 |
| hsa-miR-6719-3p | −16.61 | −16.61 | −16.61 | −16.61 | −16.61 |
| hsa-miR-6720-3p | −16.61 | −16.61 | −16.61 | −16.61 | −16.61 |
| hsa-miR-6720-5p | −16.61 | −16.61 | −16.61 | −16.61 | −16.61 |
| hsa-miR-6721-5p | 1.861 | −16.61 | 1.187 | −16.61 | −16.61 |
| hsa-miR-6722-3p | −16.61 | −16.61 | −16.61 | −16.61 | −16.61 |
| hsa-miR-6722-5p | −16.61 | −16.61 | −16.61 | −16.61 | −16.61 |
| hsa-miR-6724-5p | −16.61 | 2.235 | −16.61 | 3.102 | 4.481 |
| hsa-miR-6726-3p | −16.61 | 0.535 | −16.61 | −16.61 | −16.61 |
| hsa-miR-6726-5p | −16.61 | −16.61 | −16.61 | −16.61 | −16.61 |
| hsa-miR-6727-3p | −16.61 | −16.61 | −16.61 | −16.61 | −16.61 |
| hsa-miR-6727-5p | 3.283 | −16.61 | −16.61 | −16.61 | −16.61 |
| hsa-miR-6728-3p | −16.61 | −16.61 | −16.61 | −16.61 | −16.61 |
| hsa-miR-6728-5p | −16.61 | −16.61 | −16.61 | −16.61 | −16.61 |
| hsa-miR-6729-3p | −16.61 | −16.61 | −16.61 | −16.61 | −16.61 |
| hsa-miR-6729-5p | −16.61 | −16.61 | −16.61 | 0.439 | −16.61 |
| hsa-miR-6730-3p | −16.61 | −16.61 | −16.61 | −16.61 | −16.61 |
| hsa-miR-6730-5p | −16.61 | −16.61 | −16.61 | −16.61 | 3.24 |
| hsa-miR-6731-3p | −16.61 | −16.61 | −16.61 | −16.61 | −16.61 |
| hsa-miR-6731-5p | −16.61 | −16.61 | 1.312 | 4.102 | 4.588 |
| hsa-miR-6732-3p | −16.61 | −16.61 | −16.61 | −16.61 | −16.61 |
| hsa-miR-6732-5p | −16.61 | −16.61 | −16.61 | −16.61 | −16.61 |
| hsa-miR-6733-3p | −16.61 | −16.61 | −16.61 | −16.61 | −16.61 |
| hsa-miR-6733-5p | 4.555 | 1.705 | −16.61 | −16.61 | 3.481 |
| hsa-miR-6734-3p | −16.61 | −16.61 | −16.61 | −16.61 | −16.61 |
| hsa-miR-6734-5p | −16.61 | 3.705 | 1.534 | 3.211 | 4.535 |
| hsa-miR-6735-3p | −16.61 | −16.61 | −16.61 | −16.61 | −16.61 |
| hsa-miR-6735-5p | −16.61 | −16.61 | 2.187 | −16.61 | −16.61 |
| hsa-miR-6736-3p | −16.61 | −16.61 | −16.61 | −16.61 | −16.61 |
| hsa-miR-6736-5p | −16.61 | −16.61 | −16.61 | 1.024 | −16.61 |
| hsa-miR-6737-3p | −16.61 | −16.61 | −16.61 | −16.61 | −16.61 |
| hsa-miR-6737-5p | −16.61 | −16.61 | −16.61 | −16.61 | −16.61 |
| hsa-miR-6738-3p | −16.61 | −16.61 | −16.61 | −16.61 | −16.61 |
| hsa-miR-6738-5p | −16.61 | −16.61 | −16.61 | −16.61 | −16.61 |
| hsa-miR-6739-3p | −16.61 | 1.12 | 1.312 | −16.61 | −16.61 |
| hsa-miR-6739-5p | −16.61 | −16.61 | −16.61 | −16.61 | −16.61 |
| hsa-miR-6740-3p | −16.61 | −16.61 | −16.61 | −16.61 | −16.61 |
| hsa-miR-6740-5p | −16.61 | −16.61 | 2.534 | 2.246 | 4.78 |
| hsa-miR-6741-3p | −16.61 | −16.61 | −16.61 | −16.61 | −16.61 |
| hsa-miR-6741-5p | −16.61 | −16.61 | 0.312 | −16.61 | −16.61 |
| hsa-miR-6742-3p | −16.61 | −16.61 | −0.273 | 2.102 | −16.61 |
| hsa-miR-6742-5p | −16.61 | −16.61 | 0.534 | −16.61 | −16.61 |
| hsa-miR-6743-3p | −16.61 | −16.61 | −16.61 | −16.61 | −16.61 |
| hsa-miR-6743-5p | −16.61 | −16.61 | −16.61 | −16.61 | −16.61 |
| hsa-miR-6744-3p | −16.61 | −16.61 | −16.61 | −16.61 | −16.61 |
| hsa-miR-6744-5p | −16.61 | −16.61 | −16.61 | −16.61 | −16.61 |
| hsa-miR-6745-3p/ | −16.61 | −16.61 | −16.61 | −16.61 | −16.61 |
| 6745-5p | | | | | |
| hsa-miR-6746-3p | 3.54 | −16.61 | −16.61 | −16.61 | −16.61 |
| hsa-miR-6746-5p | −16.61 | −16.61 | −16.61 | −16.61 | −16.61 |
| hsa-miR-6747-3p | −16.61 | 1.705 | −16.61 | −16.61 | −16.61 |
| hsa-miR-6747-5p | −16.61 | −16.61 | −16.61 | −16.61 | −16.61 |
| hsa-miR-6748-3p | −0.155 | −16.61 | 0.727 | −16.61 | −16.61 |
| hsa-miR-6748-5p | −16.61 | −16.61 | −16.61 | −16.61 | −16.61 |
| hsa-miR-6749-3p | −16.61 | −16.61 | −16.61 | −16.61 | −16.61 |
| hsa-miR-6749-5p | −16.61 | −16.61 | −16.61 | −16.61 | −16.61 |
| hsa-miR-675-3p | 0.626 | 4.342 | 1.815 | 0.854 | −16.61 |
| hsa-miR-675-5p | 3.085 | 4.764 | 1.534 | 1.854 | −16.61 |
| hsa-miR-6750-3p | −16.61 | −16.61 | −16.61 | −16.61 | −16.61 |
| hsa-miR-6750-5p | −16.61 | −16.61 | −16.61 | 1.176 | −16.61 |
| hsa-miR-6751-3p | −16.61 | −16.61 | −16.61 | −16.61 | −16.61 |
| hsa-miR-6751-5p | 16.61 | −16.61 | −16.61 | −16.61 | −16.61 |
| hsa-miR-6752-3p | −16.61 | −16.61 | −16.61 | −16.61 | −16.61 |
| hsa-miR-6752-5p | −16.61 | −16.61 | −16.61 | −16.61 | −16.61 |
| hsa-miR-6753-3p | −16.61 | −16.61 | −16.61 | −16.61 | −16.61 |
| hsa-miR-6753-5p | −16.61 | −16.61 | −16.61 | −16.61 | −16.61 |
| hsa-miR-6754-3p | −16.61 | −16.61 | −0.273 | −16.61 | −16.61 |
| hsa-miR-6754-5p | −16.61 | −16.61 | −16.61 | 3.102 | −16.61 |
| hsa-miR-6755-3p | −16.61 | −16.61 | −16.61 | 1.661 | −16.61 |
| hsa-miR-6755-5p | −16.61 | −16.61 | 1.049 | 2.102 | −16.61 |
| hsa-miR-6756-3p | −16.61 | −16.61 | −16.61 | −16.61 | −16.61 |
| hsa-miR-6756-5p | −16.61 | −16.61 | −16.61 | −16.61 | −16.61 |
| hsa-miR-6757-3p | −16.61 | −16.61 | −16.61 | −16.61 | −16.61 |
| hsa-miR-6757-5p | −16.61 | −16.61 | −16.61 | −16.61 | −16.61 |
| hsa-miR-6758-3p | −16.61 | −16.61 | −16.61 | −16.61 | −16.61 |
| hsa-miR-6758-5p | −16.61 | −16.61 | −16.61 | −16.61 | 4.588 |
| hsa-miR-6759-3p | −16.61 | −16.61 | −16.61 | −16.61 | −16.61 |
| hsa-miR-6759-5p | −16.61 | −16.61 | −16.61 | −16.61 | 4.028 |
| hsa-miR-676-3p | 4.003 | 5.612 | 2.428 | 3.176 | 4.687 |
| hsa-miR-676-5p | −16.61 | 3.235 | −16.61 | −16.61 | −16.61 |
| hsa-miR-6760-3p | −16.61 | −16.61 | −16.61 | −16.61 | −16.61 |
| hsa-miR-6760-5p | −16.61 | −16.61 | −16.61 | −16.61 | −16.61 |
| hsa-miR-6761-3p | −16.61 | −16.61 | −16.61 | −16.61 | −16.61 |
| hsa-miR-6761-5p | 2.81 | −16.61 | 3.085 | 2.439 | 3.78 |
| hsa-miR-6762-3p | −16.61 | −16.61 | −16.61 | −16.61 | −16.61 |
| hsa-miR-6762-5p | −16.61 | −16.61 | −16.61 | −16.61 | −16.61 |
| hsa-miR-6763-3p | −16.61 | −16.61 | −16.61 | −16.61 | −16.61 |
| hsa-miR-6763-5p | 3.027 | 2.927 | 2.312 | −16.61 | −16.61 |
| hsa-miR-6764-3p | −16.61 | 2.783 | −16.61 | −16.61 | −16.61 |
| hsa-miR-6764-5p | 3.348 | 0.535 | 1.727 | 3.313 | 4.424 |
| hsa-miR-6765-3p | −16.61 | −16.61 | −16.61 | −16.61 | −16.61 |
| hsa-miR-6765-5p | −16.61 | −16.61 | −16.61 | −16.61 | −16.61 |
| hsa-miR-6766-3p | −16.61 | −16.61 | −16.61 | −16.61 | −16.61 |
| hsa-miR-6766-5p | −16.61 | −16.61 | −16.61 | −16.61 | −16.61 |
| hsa-miR-6767-3p | −16.61 | −16.61 | −16.61 | −16.61 | −16.61 |
| hsa-miR-6767-5p | −16.61 | 4.207 | −16.61 | −16.61 | −16.61 |
| hsa-miR-6768-3p | −16.61 | −16.61 | −16.61 | −16.61 | −16.61 |
| hsa-miR-6768-5p | −16.61 | −16.61 | −16.61 | −16.61 | −16.61 |
| hsa-miR-6769a-3p | −16.61 | −16.61 | −16.61 | −16.61 | −16.61 |
| hsa-miR-6769a-5p | −16.61 | −16.61 | −16.61 | −16.61 | 4.535 |
| hsa-miR-6769b-3p | −16.61 | −16.61 | −16.61 | −16.61 | −16.61 |
| hsa-miR-6769b-5p | −16.61 | −16.61 | −16.61 | −16.61 | −16.61 |
| hsa-miR-6770-3p | 2.202 | −16.61 | 1.897 | −16.61 | −16.61 |
| hsa-miR-6770-5p | −16.61 | −16.61 | −16.61 | −16.61 | −16.61 |
| hsa-miR-6771-3p | −16.61 | −16.61 | −16.61 | −16.61 | −16.61 |
| hsa-miR-6771-5p | −16.61 | −16.61 | −16.61 | −16.61 | −16.61 |
| hsa-miR-6772-3p | −16.61 | −16.61 | −16.61 | −16.61 | −16.61 |
| hsa-miR-6772-5p | −16.61 | −16.61 | −16.61 | −16.61 | −16.61 |
| hsa-miR-6773-3p | −16.61 | −16.61 | −16.61 | −16.61 | −16.61 |
| hsa-miR-6773-5p | −16.61 | −16.61 | −16.61 | −16.61 | −16.61 |
| hsa-miR-6774-3p | −16.61 | −16.61 | 0.534 | −16.61 | 3.78 |
| hsa-miR-6774-5p | −16.61 | −16.61 | −16.61 | −16.61 | −16.61 |
| hsa-miR-6775-3p | −16.61 | −16.61 | −16.61 | −16.61 | −16.61 |
| hsa-miR-6775-5p | −16.61 | −16.61 | −16.61 | −16.61 | −16.61 |
| hsa-miR-6776-3p | −16.61 | −16.61 | −16.61 | −16.61 | −16.61 |
| hsa-miR-6776-5p | 16.61 | −16.61 | −16.61 | −16.61 | −16.61 |
| hsa-miR-6777-3p | −16.61 | 0.12 | −16.61 | −16.61 | −16.61 |
| hsa-miR-6777-5p | −16.61 | −16.61 | −16.61 | −16.61 | −16.61 |
| hsa-miR-6778-3p | −16.61 | −16.61 | −16.61 | −16.61 | −16.61 |
| hsa-miR-6778-5p | 16.61 | −16.61 | −16.61 | −16.61 | −16.61 |
| hsa-miR-6779-3p | −16.61 | −16.61 | −16.61 | −16.61 | −16.61 |
| hsa-miR-6779-5p | 16.61 | −16.61 | −16.61 | −16.61 | −16.61 |
| hsa-miR-6780a-3p | −16.61 | −16.61 | −16.61 | −16.61 | −16.61 |
| hsa-miR-6780a-5p | −16.61 | −16.61 | −16.61 | −16.61 | −16.61 |
| hsa-miR-6780b-3p | 0.943 | −16.61 | −16.61 | −16.61 | −16.61 |
| hsa-miR-6780b-5p | −16.61 | −16.61 | −16.61 | −16.61 | −16.61 |
| hsa-miR-6781-3p | −16.61 | −16.61 | −16.61 | −16.61 | −16.61 |
| hsa-miR-6781-5p | −16.61 | −16.61 | −16.61 | −16.61 | −16.61 |
| hsa-miR-6782-3p | −16.61 | −16.61 | −16.61 | −16.61 | −16.61 |
| hsa-miR-6782-5p | −16.61 | −16.61 | −16.61 | −16.61 | −16.61 |
| hsa-miR-6783-3p | 2.135 | −16.61 | −16.61 | −16.61 | −16.61 |
| hsa-miR-6783-5p | −16.61 | −16.61 | −16.61 | −16.61 | −16.61 |
| hsa-miR-6784-3p | −16.61 | −16.61 | −16.61 | −16.61 | −16.61 |
| hsa-miR-6784-5p | −16.61 | −16.61 | −16.61 | −16.61 | −16.61 |
| hsa-miR-6785-3p | 2.684 | −16.61 | −16.61 | −16.61 | −16.61 |
| hsa-miR-6785-5p | −16.61 | −16.61 | −16.61 | −16.61 | −16.61 |
| hsa-miR-6786-3p | −16.61 | −16.61 | 2.049 | −16.61 | −16.61 |
| hsa-miR-6786-5p | −16.61 | −16.61 | −16.61 | −16.61 | −16.61 |
| hsa-miR-6787-3p | −16.61 | −16.61 | −16.61 | −16.61 | −16.61 |
| hsa-miR-6787-5p | −16.61 | −16.61 | −16.61 | −16.61 | −16.61 |
| hsa-miR-6788-3p | −16.61 | −16.61 | −16.61 | −16.61 | −16.61 |
| hsa-miR-6788-5p | −16.61 | −16.61 | −16.61 | −16.61 | −16.61 |
| hsa-miR-6789-3p | −16.61 | −16.61 | −0.273 | −16.61 | −16.61 |
| hsa-miR-6789-5p | −16.61 | −16.61 | −16.61 | −16.61 | −16.61 |
| hsa-miR-6790-3p | −16.61 | −16.61 | −16.61 | −16.61 | −16.61 |
| hsa-miR-6790-5p | −16.61 | −16.61 | −16.61 | −16.61 | −16.61 |
| hsa-miR-6791-3p | −16.61 | −16.61 | −16.61 | −16.61 | −16.61 |
| hsa-miR-6791-5p | −16.61 | −16.61 | −16.61 | −16.61 | −16.61 |
| hsa-miR-6792-3p | −16.61 | −16.61 | −16.61 | −16.61 | −16.61 |
| hsa-miR-6792-5p | −16.61 | −16.61 | −16.61 | −16.61 | −16.61 |
| hsa-miR-6793-3p | −16.61 | −16.61 | −16.61 | −16.61 | −16.61 |
| hsa-miR-6793-5p | −16.61 | −16.61 | −16.61 | −16.61 | 4.588 |
| hsa-miR-6794-3p | −16.61 | −16.61 | −16.61 | −16.61 | −16.61 |
| hsa-miR-6794-5p | −16.61 | −16.61 | 0.049 | −16.61 | −16.61 |
| hsa-miR-6795-3p | −16.61 | −16.61 | −16.61 | −16.61 | −16.61 |
| hsa-miR-6795-5p | −16.61 | −16.61 | −16.61 | −16.61 | −16.61 |
| hsa-miR-6796-3p | −16.61 | −16.61 | −16.61 | −16.61 | −16.61 |
| hsa-miR-6796-5p | −16.61 | 3.442 | −16.61 | −16.61 | 3.365 |
| hsa-miR-6797-3p | −16.61 | −16.61 | −16.61 | −16.61 | −16.61 |
| hsa-miR-6797-5p | −16.61 | −16.61 | −16.61 | −16.61 | −16.61 |
| hsa-miR-6798-3p | −16.61 | −16.61 | −16.61 | −16.61 | −16.61 |
| hsa-miR-6798-5p | −16.61 | −16.61 | −16.61 | −16.61 | −16.61 |
| hsa-miR-6799-3p | −16.61 | −16.61 | −16.61 | −16.61 | −16.61 |
| hsa-miR-6799-5p | −16.61 | −16.61 | −16.61 | −16.61 | −16.61 |
| hsa-miR-6800-3p | 2.308 | −16.61 | −16.61 | −16.61 | −16.61 |
| hsa-miR-6800-5p | −16.61 | −16.61 | −16.61 | −16.61 | −16.61 |
| hsa-miR-6801-3p | −16.61 | −16.61 | −16.61 | −16.61 | −16.61 |
| hsa-miR-6801-5p | −16.61 | −16.61 | −16.61 | −16.61 | −16.61 |
| hsa-miR-6802-3p | −16.61 | 2.705 | −16.61 | −16.61 | −16.61 |
| hsa-miR-6802-5p | −16.61 | −16.61 | −16.61 | −16.61 | 2.78 |
| hsa-miR-6803-3p | 1.312 | −16.61 | 2.187 | −16.61 | 2.588 |
| hsa-miR-6803-5p | −16.61 | −16.61 | −16.61 | −16.61 | −16.61 |
| hsa-miR-6804-3p | −16.61 | 3.664 | −16.61 | −16.61 | −16.61 |
| hsa-miR-6804-5p | −16.61 | −16.61 | −16.61 | −16.61 | −16.61 |
| hsa-miR-6805-3p | −16.61 | −16.61 | −16.61 | −16.61 | −16.61 |
| hsa-miR-6805-5p | −16.61 | −16.61 | −16.61 | 2.176 | −16.61 |
| hsa-miR-6806-3p | 2.671 | −16.61 | −16.61 | −16.61 | −16.61 |
| hsa-miR-6806-5p | −16.61 | −16.61 | −16.61 | −16.61 | −16.61 |
| hsa-miR-6807-3p | −16.61 | −16.61 | −16.61 | −16.61 | −16.61 |
| hsa-miR-6807-5p | 3.547 | 2.857 | 2.534 | 2.712 | 3.868 |
| hsa-miR-6808-3p | 1.943 | −16.61 | −16.61 | −16.61 | 2.102 |
| hsa-miR-6808-5p | −16.61 | −16.61 | −16.61 | −16.61 | −16.61 |
| hsa-miR-6809-3p | −16.61 | −16.61 | −16.61 | −16.61 | −16.61 |
| hsa-miR-6809-5p | −16.61 | −16.61 | −16.61 | −16.61 | −16.61 |
| hsa-miR-6810-3p | −16.61 | −16.61 | −16.61 | −16.61 | −16.61 |
| hsa-miR-6810-5p | −16.61 | −16.61 | −16.61 | 1.941 | −16.61 |
| hsa-miR-6811-3p | −16.61 | −16.61 | −16.61 | −16.61 | −16.61 |
| hsa-miR-6811-5p | −16.61 | −16.61 | −16.61 | −16.61 | −16.61 |
| hsa-miR-6812-3p | −16.61 | −16.61 | −16.61 | −16.61 | −16.61 |
| hsa-miR-6812-5p | −16.61 | −16.61 | −16.61 | −16.61 | −16.61 |
| hsa-miR-6813-3p | −16.61 | −16.61 | −16.61 | −16.61 | −16.61 |
| hsa-miR-6813-5p | −16.61 | 3.892 | 2.187 | 2.246 | 2.78 |
| hsa-miR-6814-3p | −16.61 | −16.61 | −16.61 | −16.61 | −16.61 |
| hsa-miR-6814-5p | −16.61 | −16.61 | −16.61 | −16.61 | −16.61 |
| hsa-miR-6815-3p | 3.603 | −16.61 | −16.61 | −16.61 | −16.61 |
| hsa-miR-6815-5p | −16.61 | −16.61 | −16.61 | −16.61 | −16.61 |
| hsa-miR-6816-3p | −16.61 | −16.61 | −16.61 | −16.61 | −16.61 |
| hsa-miR-6816-5p | −16.61 | −16.61 | −16.61 | −16.61 | −16.61 |
| hsa-miR-6817-3p | −16.61 | −16.61 | −16.61 | −16.61 | −16.61 |
| hsa-miR-6817-5p | −16.61 | −16.61 | −16.61 | 1.554 | −16.61 |
| hsa-miR-6818-3p | −16.61 | −16.61 | −16.61 | −16.61 | −16.61 |
| hsa-miR-6818-5p | −16.61 | −16.61 | 0.727 | −16.61 | 4.304 |
| hsa-miR-6819-3p | −16.61 | −16.61 | −16.61 | −16.61 | −16.61 |
| hsa-miR-6819-5p | −16.61 | −16.61 | −16.61 | −16.61 | −16.61 |
| hsa-miR-6820-3p | −16.61 | −16.61 | −16.61 | −16.61 | 3.102 |
| hsa-miR-6820-5p | −16.61 | −16.61 | −16.61 | −16.61 | 2.102 |
| hsa-miR-6821-3p | −16.61 | −16.61 | −16.61 | −16.61 | −16.61 |
| hsa-miR-6821-5p | −16.61 | 6.316 | −16.61 | 4.82 | −16.61 |
| hsa-miR-6822-3p | −16.61 | −16.61 | −16.61 | −16.61 | −16.61 |
| hsa-miR-6822-5p | −16.61 | −16.61 | −16.61 | −16.61 | 2.95 |
| hsa-miR-6823-3p | −16.61 | −16.61 | −16.61 | −16.61 | −16.61 |
| hsa-miR-6823-5p | −16.61 | −16.61 | 1.975 | −16.61 | −16.61 |
| hsa-miR-6824-3p | −16.61 | −16.61 | −16.61 | −16.61 | −16.61 |
| hsa-miR-6824-5p | −16.61 | −16.61 | −16.61 | −16.61 | 3.102 |
| hsa-miR-6825-3p | 16.61 | −16.61 | −16.61 | −16.61 | −16.61 |
| hsa-miR-6825-5p | 16.61 | −16.61 | −16.61 | −16.61 | −16.61 |
| hsa-miR-6826-3p | −16.61 | −16.61 | −16.61 | −16.61 | −16.61 |
| hsa-miR-6826-5p | 16.61 | −16.61 | −16.61 | −16.61 | 2.78 |
| hsa-miR-6827-3p | −16.61 | −16.61 | −16.61 | −16.61 | −16.61 |
| hsa-miR-6827-5p | −16.61 | −16.61 | −16.61 | −16.61 | −16.61 |
| hsa-miR-6828-3p | −16.61 | −16.61 | −16.61 | −16.61 | −16.61 |
| hsa-miR-6828-5p | −16.61 | −16.61 | −16.61 | −16.61 | −16.61 |
| hsa-miR-6829-3p | −16.61 | −16.61 | −16.61 | −16.61 | −16.61 |
| hsa-miR-6829-5p | −16.61 | −16.61 | −16.61 | −16.61 | −16.61 |
| hsa-miR-6830-3p | −16.61 | −16.61 | −16.61 | −16.61 | −16.61 |
| hsa-miR-6830-5p | −16.61 | −16.61 | −16.61 | −16.61 | −16.61 |
| hsa-miR-6831-3p | 2.593 | −16.61 | −16.61 | −16.61 | −16.61 |
| hsa-miR-6831-5p | 2.441 | −16.61 | −16.61 | 2.609 | −16.61 |
| hsa-miR-6832-3p | −16.61 | −16.61 | −16.61 | −16.61 | −16.61 |
| hsa-miR-6832-5p | 5.375 | 1.705 | −16.61 | −16.61 | −16.61 |
| hsa-miR-6833-3p | −16.61 | −16.61 | −16.61 | −16.61 | −16.61 |
| hsa-miR-6833-5p | −16.61 | −16.61 | −16.61 | −16.61 | 5.335 |
| hsa-miR-6834-3p | −16.61 | −16.61 | −16.61 | −16.61 | −16.61 |
| hsa-miR-6834-5p | −16.61 | −16.61 | −16.61 | −16.61 | −16.61 |
| hsa-miR-6835-3p | 16.61 | −16.61 | −16.61 | −16.61 | −16.61 |
| hsa-miR-6835-5p | −16.61 | −16.61 | −16.61 | −16.61 | −16.61 |
| hsa-miR-6836-3p | −16.61 | −16.61 | −16.61 | −16.61 | −16.61 |
| hsa-miR-6836-5p | 16.61 | −16.61 | −16.61 | −16.61 | −16.61 |
| hsa-miR-6837-3p | −16.61 | −16.61 | 2.634 | 2.661 | −16.61 |
| hsa-miR-6837-5p | −16.61 | −16.61 | −16.61 | −16.61 | −16.61 |
| hsa-miR-6838-3p | −16.61 | −16.61 | −16.61 | −16.61 | −16.61 |
| hsa-miR-6838-5p | −16.61 | −16.61 | −16.61 | −16.61 | −16.61 |
| hsa-miR-6839-3p | −16.61 | −16.61 | −16.61 | −16.61 | −16.61 |
| hsa-miR-6839-5p | −16.61 | −16.61 | −16.61 | 2.377 | −16.61 |
| hsa-miR-6840-3p | −16.61 | −16.61 | 0.312 | −16.61 | −16.61 |
| hsa-miR-6840-5p | −16.61 | −16.61 | −16.61 | −16.61 | −16.61 |
| hsa-miR-6841-3p | −16.61 | −16.61 | −16.61 | −16.61 | −16.61 |
| hsa-miR-6841-5p | −16.61 | −16.61 | −16.61 | −16.61 | −16.61 |
| hsa-miR-6842-3p | 1.948 | −16.61 | 3.012 | 3.176 | −16.61 |
| hsa-miR-6842-5p | −16.61 | −16.61 | 1.975 | 1.661 | −16.61 |
| hsa-miR-6843-3p | −16.61 | −16.61 | −16.61 | −16.61 | −16.61 |
| hsa-miR-6844 | 3.283 | 1.705 | 2.251 | 2.761 | −16.61 |
| hsa-miR-6845-3p | −16.61 | −16.61 | −16.61 | −16.61 | −16.61 |
| hsa-miR-6845-5p | −16.61 | −16.61 | −16.61 | −16.61 | −16.61 |
| hsa-miR-6846-3p | −16.61 | −16.61 | −16.61 | −16.61 | −16.61 |
| hsa-miR-6846-5p | −16.61 | −16.61 | −16.61 | −16.61 | −16.61 |
| hsa-miR-6847-3p | −16.61 | −16.61 | −16.61 | −16.61 | −16.61 |
| hsa-miR-6847-5p | −16.61 | 1.342 | −16.61 | 3.139 | −16.61 |
| hsa-miR-6848-3p | −16.61 | −16.61 | −16.61 | −16.61 | −16.61 |
| hsa-miR-6848-5p | −16.61 | −16.61 | −16.61 | −16.61 | 2.588 |
| hsa-miR-6849-3p | −16.61 | −16.61 | −16.61 | −16.61 | −16.61 |
| hsa-miR-6849-5p | −16.61 | −16.61 | −16.61 | −16.61 | −16.61 |
| hsa-miR-6850-3p | −16.61 | −16.61 | −16.61 | −16.61 | −16.61 |
| hsa-miR-6850-5p | −16.61 | −16.61 | 1.815 | −16.61 | −16.61 |
| hsa-miR-6851-3p | −16.61 | −16.61 | −16.61 | −16.61 | −16.61 |
| hsa-miR-6851-5p | −16.61 | −0.465 | −16.61 | −16.61 | −16.61 |
| hsa-miR-6852-3p | −16.61 | −16.61 | −16.61 | −16.61 | −16.61 |
| hsa-miR-6852-5p | 1.715 | 3.961 | 1.897 | −16.61 | −16.61 |
| hsa-miR-6853-3p | −16.61 | −16.61 | −16.61 | −16.61 | −16.61 |
| hsa-miR-6853-5p | −16.61 | −16.61 | −16.61 | −16.61 | −16.61 |
| hsa-miR-6854-3p | −16.61 | −16.61 | −16.61 | −16.61 | −16.61 |
| hsa-miR-6854-5p | −16.61 | −16.61 | −16.61 | 2.102 | −16.61 |
| hsa-miR-6855-3p | −16.61 | −16.61 | −16.61 | −16.61 | −16.61 |
| hsa-miR-6855-5p | −16.61 | −16.61 | −16.61 | −16.61 | −16.61 |
| hsa-miR-6856-3p | −16.61 | −16.61 | −16.61 | −16.61 | −16.61 |
| hsa-miR-6856-5p | −16.61 | −16.61 | −16.61 | −16.61 | −16.61 |
| hsa-miR-6857-3p | −16.61 | −16.61 | −16.61 | −16.61 | −16.61 |
| hsa-miR-6857-5p | −16.61 | −16.61 | −16.61 | −16.61 | −16.61 |
| hsa-miR-6858-3p | −16.61 | −16.61 | −16.61 | −16.61 | −16.61 |
| hsa-miR-6858-5p | −16.61 | −16.61 | −16.61 | −16.61 | 5.102 |
| hsa-miR-6859-3p | −16.61 | −16.61 | 0.897 | −16.61 | −16.61 |
| hsa-miR-6859-5p | 3.416 | 0.535 | 1.534 | −16.61 | 2.588 |
| hsa-miR-6860 | −16.61 | −16.61 | −16.61 | −16.61 | −16.61 |
| hsa-miR-6861-3p | −16.61 | −16.61 | −16.61 | −16.61 | −16.61 |
| hsa-miR-6861-5p | −16.61 | −16.61 | −16.61 | −16.61 | −16.61 |
| hsa-miR-6862-3p | −16.61 | −16.61 | −16.61 | −16.61 | −16.61 |
| hsa-miR-6862-5p | −16.61 | −16.61 | −16.61 | −16.61 | −16.61 |
| hsa-miR-6863 | −16.61 | −16.61 | 1.727 | −16.61 | −16.61 |
| hsa-miR-6864-3p | −16.61 | −16.61 | −16.61 | −16.61 | −16.61 |
| hsa-miR-6864-5p | −16.61 | −16.61 | 1.634 | −16.61 | −16.61 |
| hsa-miR-6865-3p | −16.61 | −16.61 | −16.61 | −16.61 | −16.61 |
| hsa-miR-6865-5p | −16.61 | −16.61 | −16.61 | −16.61 | −16.61 |
| hsa-miR-6866-3p | −16.61 | −16.61 | 0.312 | −16.61 | −16.61 |
| hsa-miR-6866-5p | 1.703 | 1.12 | −16.61 | −16.61 | 4.424 |
| hsa-miR-6867-3p | −16.61 | −16.61 | −16.61 | −16.61 | −16.61 |
| hsa-miR-6867-5p | −16.61 | −16.61 | −16.61 | −16.61 | −16.61 |
| hsa-miR-6868-3p | −16.61 | −16.61 | −16.61 | −16.61 | −16.61 |
| hsa-miR-6868-5p | −16.61 | −16.61 | −16.61 | −16.61 | −16.61 |
| hsa-miR-6869-3p | −16.61 | −16.61 | −16.61 | −16.61 | −16.61 |
| hsa-miR-6869-5p | −16.61 | −16.61 | −16.61 | −16.61 | −16.61 |
| hsa-miR-6870-3p | −16.61 | −16.61 | −16.61 | −16.61 | −16.61 |
| hsa-miR-6870-5p | −16.61 | −16.61 | −16.61 | −16.61 | −16.61 |
| hsa-miR-6871-3p | −16.61 | −16.61 | 0.534 | −16.61 | −16.61 |
| hsa-miR-6871-5p | −16.61 | 1.12 | −16.61 | −16.61 | −16.61 |
| hsa-miR-6872-3p | −16.61 | −16.61 | −16.61 | −16.61 | −16.61 |
| hsa-miR-6872-5p | −16.61 | −16.61 | −16.61 | −16.61 | −16.61 |
| hsa-miR-6873-3p | 3.211 | −16.61 | −16.61 | −16.61 | −16.61 |
| hsa-miR-6873-5p | −16.61 | −16.61 | −16.61 | −16.61 | −16.61 |
| hsa-miR-6874-3p | 2.739 | −16.61 | 1.049 | 2.377 | −16.61 |
| hsa-miR-6874-5p | −16.61 | −16.61 | −16.61 | −16.61 | −16.61 |
| hsa-miR-6875-3p | −16.61 | −16.61 | −16.61 | −16.61 | −16.61 |
| hsa-miR-6875-5p | −16.61 | −16.61 | −16.61 | −16.61 | −16.61 |
| hsa-miR-6876-3p | −16.61 | −16.61 | −16.61 | −16.61 | −16.61 |
| hsa-miR-6876-5p | −16.61 | −16.61 | −16.61 | −16.61 | −16.61 |
| hsa-miR-6877-3p | −16.61 | −16.61 | −16.61 | −16.61 | −16.61 |
| hsa-miR-6877-5p | −16.61 | 1.535 | 1.727 | −16.61 | 4.687 |
| hsa-miR-6878-3p | −16.61 | −16.61 | −16.61 | −16.61 | −16.61 |
| hsa-miR-6878-5p | −16.61 | −16.61 | −16.61 | −16.61 | −16.61 |
| hsa-miR-6879-3p | −16.61 | −16.61 | −16.61 | −16.61 | −16.61 |
| hsa-miR-6879-5p | −16.61 | −16.61 | −16.61 | −16.61 | −16.61 |
| hsa-miR-6880-3p | −16.61 | −16.61 | −16.61 | −16.61 | −16.61 |
| hsa-miR-6880-5p | −16.61 | −16.61 | −16.61 | −16.61 | 4.304 |
| hsa-miR-6881-3p | −16.61 | −16.61 | −16.61 | −16.61 | −16.61 |
| hsa-miR-6881-5p | −16.61 | −16.61 | 1.727 | −16.61 | 4.424 |
| hsa-miR-6882-3p | −16.61 | −16.61 | −16.61 | −16.61 | −16.61 |
| hsa-miR-6882-5p | −16.61 | 0.857 | 2.119 | −1.146 | 3.102 |
| hsa-miR-6883-3p | −16.61 | −16.61 | −16.61 | −16.61 | −16.61 |
| hsa-miR-6883-5p | −16.61 | −16.61 | −16.61 | −16.61 | −16.61 |
| hsa-miR-6884-3p | −16.61 | −16.61 | −16.61 | −16.61 | −16.61 |
| hsa-miR-6884-5p | −16.61 | −16.61 | −16.61 | −16.61 | 3.481 |
| hsa-miR-6885-3p | −16.61 | −16.61 | −16.61 | −16.61 | −16.61 |
| hsa-miR-6885-5p | −16.61 | −16.61 | −16.61 | −16.61 | −16.61 |
| hsa-miR-6886-3p | −16.61 | −16.61 | −16.61 | −16.61 | −16.61 |
| hsa-miR-6886-5p | 2.736 | 1.12 | 1.634 | −16.61 | 5.304 |
| hsa-miR-6887-3p | −16.61 | −16.61 | −16.61 | −16.61 | −16.61 |
| hsa-miR-6887-5p | −16.61 | −16.61 | −16.61 | −16.61 | −16.61 |
| hsa-miR-6888-3p | −16.61 | −16.61 | −16.61 | −16.61 | −16.61 |
| hsa-miR-6888-5p | −16.61 | −16.61 | −16.61 | −16.61 | −16.61 |
| hsa-miR-6889-3p | −16.61 | −16.61 | −16.61 | −16.61 | −16.61 |
| hsa-miR-6889-5p | −16.61 | −16.61 | −16.61 | −16.61 | −16.61 |
| hsa-miR-6890-3p | −16.61 | −16.61 | −16.61 | −16.61 | −16.61 |
| hsa-miR-6890-5p | −16.61 | −16.61 | −16.61 | −16.61 | −16.61 |
| hsa-miR-6891-3p | −16.61 | −16.61 | −16.61 | −16.61 | −16.61 |
| hsa-miR-6891-5p | −16.61 | −16.61 | −16.61 | −16.61 | −16.61 |
| hsa-miR-6892-3p | −16.61 | −16.61 | −16.61 | −16.61 | −16.61 |
| hsa-miR-6892-5p | −16.61 | −16.61 | −16.61 | −16.61 | 3.588 |
| hsa-miR-6893-3p | −16.61 | −16.61 | −16.61 | −16.61 | −16.61 |
| hsa-miR-6893-5p | −16.61 | −16.61 | −16.61 | −16.61 | −16.61 |
| hsa-miR-6894-3p | −16.61 | −16.61 | −16.61 | −16.61 | −16.61 |
| hsa-miR-6894-5p | −16.61 | −16.61 | −16.61 | −16.61 | 3.102 |
| hsa-miR-6895-3p | −16.61 | −16.61 | −16.61 | −16.61 | −16.61 |
| hsa-miR-6895-5p | −16.61 | −16.61 | −16.61 | −16.61 | −16.61 |
| hsa-miR-7-1-3p | 5.182 | 4.058 | 3.508 | 3.554 | 5.481 |
| hsa-miR-7-2-3p | 5.046 | 1.857 | −16.61 | −16.61 | −16.61 |
| hsa-miR-7-5p | 14.52 | 13.221 | 10.578 | 10.551 | 10.503 |
| hsa-miR-708-3p | 7.524 | 6.579 | 7.714 | 6.991 | 7.084 |
| hsa-miR-708-5p | 9.343 | 9.271 | 10.032 | 9.456 | 10.78 |
| hsa-miR-7106-3p | −16.61 | −16.61 | −16.61 | −16.61 | −16.61 |
| hsa-miR-7106-5p | −16.61 | −16.61 | −16.61 | −16.61 | −16.61 |
| hsa-miR-7107-3p | −16.61 | −16.61 | −16.61 | −16.61 | −16.61 |
| hsa-miR-7107-5p | −16.61 | −16.61 | −16.61 | −16.61 | −16.61 |
| hsa-miR-7108-3p | −16.61 | −16.61 | −16.61 | −16.61 | −16.61 |
| hsa-miR-7108-5p | −16.61 | −16.61 | −16.61 | −16.61 | −16.61 |
| hsa-miR-7109-3p | −16.61 | −16.61 | −16.61 | −16.61 | −16.61 |
| hsa-miR-7109-5p | −16.61 | −16.61 | −16.61 | 1.439 | −16.61 |
| hsa-miR-711 | −16.61 | −16.61 | −16.61 | −16.61 | −16.61 |
| hsa-miR-7110-3p | −16.61 | −16.61 | −16.61 | −16.61 | −16.61 |
| hsa-miR-7110-5p | −16.61 | −16.61 | −16.61 | −16.61 | −16.61 |
| hsa-miR-7111-3p | −16.61 | −16.61 | −16.61 | −16.61 | −16.61 |
| hsa-miR-7111-5p | −16.61 | −16.61 | −16.61 | −16.61 | −16.61 |
| hsa-miR-7112-3p | −16.61 | −16.61 | −16.61 | −16.61 | −16.61 |
| hsa-miR-7112-5p | −16.61 | −16.61 | −16.61 | −16.61 | −16.61 |
| hsa-miR-7113-3p | 16.61 | −16.61 | −16.61 | −16.61 | −16.61 |
| hsa-miR-7113-5p | 4.17 | −16.61 | −16.61 | 2.313 | 3.95 |
| hsa-miR-7114-3p | −16.61 | −16.61 | −16.61 | −16.61 | −16.61 |
| hsa-miR-7114-5p | −16.61 | −16.61 | −16.61 | −16.61 | −16.61 |
| hsa-miR-7150 | −16.61 | −16.61 | −16.61 | −16.61 | −16.61 |
| hsa-miR-7151-3p | −16.61 | 3.783 | 3.342 | −16.61 | −16.61 |
| hsa-miR-7151-5p | −16.61 | −16.61 | −16.61 | −16.61 | −16.61 |
| hsa-miR-7152-3p | −16.61 | −16.61 | −16.61 | −16.61 | −16.61 |
| hsa-miR-7152-5p | −16.61 | −16.61 | −16.61 | −16.61 | −16.61 |
| hsa-miR-7153-3p | −16.61 | −16.61 | −16.61 | −16.61 | −16.61 |
| hsa-miR-7153-5p | −16.61 | −16.61 | −16.61 | −16.61 | −16.61 |
| hsa-miR-7154-3p | −16.61 | −16.61 | −16.61 | −16.61 | −16.61 |
| hsa-miR-7154-5p | −16.61 | −16.61 | −16.61 | −16.61 | −16.61 |
| hsa-miR-7155-3p | −16.61 | −16.61 | −16.61 | −16.61 | −16.61 |
| hsa-miR-7155-5p | −16.61 | −16.61 | −16.61 | −16.61 | −16.61 |
| hsa-miR-7156-3p | 4.052 | −16.61 | −16.61 | −16.61 | −16.61 |
| hsa-miR-7156-5p | 3.237 | 0.535 | −16.61 | 2.983 | −16.61 |
| hsa-miR-7157-3p | −16.61 | −16.61 | −16.61 | −16.61 | −16.61 |
| hsa-miR-7157-5p | −16.61 | −16.61 | −16.61 | −16.61 | −16.61 |
| hsa-miR-7158-3p | −16.61 | −16.61 | −16.61 | −16.61 | −16.61 |
| hsa-miR-7158-5p | −16.61 | −16.61 | −16.61 | −16.61 | −16.61 |
| hsa-miR-7159-3p | −16.61 | −16.61 | −16.61 | −16.61 | −16.61 |
| hsa-miR-7159-5p | −16.61 | −16.61 | −16.61 | −16.61 | −16.61 |
| hsa-miR-7160-3p | −16.61 | −16.61 | −16.61 | −16.61 | −16.61 |
| hsa-miR-7160-5p | −16.61 | −16.61 | −16.61 | −16.61 | −16.61 |
| hsa-miR-7161-3p | −16.61 | −16.61 | −16.61 | −16.61 | −16.61 |
| hsa-miR-7161-5p | −16.61 | −16.61 | −16.61 | −16.61 | −16.61 |
| hsa-miR-7162-3p | −16.61 | −16.61 | −16.61 | −16.61 | −16.61 |
| hsa-miR-7162-5p | −16.61 | −16.61 | −16.61 | −16.61 | −16.61 |
| hsa-miR-718 | −16.61 | −16.61 | −16.61 | −16.61 | −16.61 |
| hsa-miR-744-3p | 3.913 | 2.342 | 1.727 | −16.61 | 3.24 |
| hsa-miR-744-5p | 10.574 | 9.495 | 8.451 | 8.546 | 10.318 |
| hsa-miR-7515 | −16.61 | −16.61 | −16.61 | −16.61 | −16.61 |
| hsa-miR-758-3p | −16.61 | 1.535 | −16.61 | −16.61 | −16.61 |
| hsa-miR-758-5p | −16.61 | 1.705 | −16.61 | −16.61 | −16.61 |
| hsa-miR-759 | 16.61 | −16.61 | −16.61 | −16.61 | −16.61 |
| hsa-miR-760 | 7.868 | 7.582 | 4.622 | 5.699 | 5.453 |
| hsa-miR-761 | −16.61 | −16.61 | −16.61 | −16.61 | −16.61 |
| hsa-miR-762 | −16.61 | −16.61 | −16.61 | −16.61 | −16.61 |
| hsa-miR-764-3p | 2.699 | −16.61 | −16.61 | −16.61 | −16.61 |
| hsa-miR-764-5p | −16.61 | −16.61 | −16.61 | −16.61 | −16.61 |
| hsa-miR-765 | −16.61 | 5.164 | −16.61 | −16.61 | 5.825 |
| hsa-miR-766-3p | 2.935 | 4.892 | 4.658 | 3.246 | 5.066 |
| hsa-miR-766-5p | −16.61 | 2.994 | 3.085 | 3.024 | 5.847 |
| hsa-miR-767-3p | 2.47 | 0.12 | −16.61 | −16.61 | −16.61 |
| hsa-miR-767-5p | 7.548 | 6.792 | 3.482 | 4.82 | 3.78 |
| hsa-miR-769-3p | 6.305 | 5.674 | 5.508 | 5.338 | 5.453 |
| hsa-miR-769-5p | 8.905 | 9.233 | 9.412 | 8.948 | 8.847 |
| hsa-miR-770-3p | −16.61 | −16.61 | −16.61 | −16.61 | −16.61 |
| hsa-miR-770-5p | −16.61 | 0.857 | −16.61 | −16.61 | −16.61 |
| hsa-miR-7702-3p | −16.61 | −16.61 | −16.61 | −16.61 | −16.61 |
| hsa-miR-7702-5p | −16.61 | −16.61 | −16.61 | −16.61 | −16.61 |
| hsa-miR-7703 | −16.61 | −16.61 | −16.61 | −16.61 | −16.61 |
| hsa-miR-7704 | 4.652 | 5.43 | 5.04 | 5.401 | 7.594 |
| hsa-miR-7705 | −16.61 | −16.61 | 2.428 | 1.554 | −16.61 |
| hsa-miR-7706 | 6.073 | 6.042 | 4.153 | 4.313 | 5.424 |
| hsa-miR-7843-3p | −16.61 | −16.61 | −16.61 | −16.61 | −16.61 |
| hsa-miR-7843-5p | −16.61 | −16.61 | −16.61 | −16.61 | −16.61 |
| hsa-miR-7844-5p | −16.61 | −16.61 | −16.61 | −16.61 | −16.61 |
| hsa-miR-7845-3p | −16.61 | −16.61 | −16.61 | −16.61 | −16.61 |
| hsa-miR-7845-5p | −16.61 | −16.61 | −16.61 | −16.61 | 4.868 |
| hsa-miR-7846-3p | −16.61 | −16.61 | −16.61 | −16.61 | −16.61 |
| hsa-miR-7846-5p | −16.61 | −16.61 | −16.61 | −16.61 | −16.61 |
| hsa-miR-7847-3p | −16.61 | −16.61 | −16.61 | −16.61 | 3.102 |
| hsa-miR-7848-3p | −16.61 | −16.61 | −16.61 | −16.61 | −16.61 |
| hsa-miR-7849-3p | −16.61 | −16.61 | −16.61 | −16.61 | −16.61 |
| hsa-miR-7849-5p | −16.61 | −16.61 | −16.61 | −16.61 | −16.61 |
| hsa-miR-7850-5p | −16.61 | −16.61 | −16.61 | −16.61 | −16.61 |
| hsa-miR-7851-3p | −16.61 | 1.705 | 3.012 | −16.61 | 5.138 |
| hsa-miR-7852-3p | −16.61 | −16.61 | −16.61 | −16.61 | −16.61 |
| hsa-miR-7852-5p | −16.61 | −16.61 | −16.61 | −16.61 | −16.61 |
| hsa-miR-7853-5p | −16.61 | −16.61 | −16.61 | −16.61 | −16.61 |
| hsa-miR-7854-3p | −16.61 | −16.61 | 1.534 | 1.024 | 4.102 |
| hsa-miR-7854-5p | −16.61 | −16.61 | −16.61 | −16.61 | −16.61 |
| hsa-miR-7855-5p | −16.61 | −16.61 | −16.61 | −16.61 | −16.61 |
| hsa-miR-7856-5p | −16.61 | −16.61 | −16.61 | −16.61 | −16.61 |
| hsa-miR-7973 | −16.61 | −16.61 | −16.61 | −16.61 | −16.61 |
| hsa-miR-7974 | −16.61 | −16.61 | −16.61 | −16.61 | −16.61 |
| hsa-miR-7975 | −16.61 | −16.61 | −16.61 | −16.61 | −16.61 |
| hsa-miR-7976 | 2.748 | 1.994 | 2.251 | 2.808 | −16.61 |
| hsa-miR-7977 | −16.61 | −16.61 | −16.61 | −16.61 | −16.61 |
| hsa-miR-7978 | −16.61 | 0.12 | −16.61 | −16.61 | −16.61 |
| hsa-miR-802 | 4.016 | 1.342 | −16.61 | −16.61 | −16.61 |
| hsa-miR-8052 | −16.61 | −16.61 | −16.61 | −16.61 | −16.61 |
| hsa-miR-8053 | −16.61 | −16.61 | −16.61 | −16.61 | −16.61 |
| hsa-miR-8054 | −16.61 | −16.61 | −16.61 | −16.61 | −16.61 |
| hsa-miR-8055 | −16.61 | −16.61 | −16.61 | −16.61 | −16.61 |
| hsa-miR-8056 | −16.61 | −16.61 | −16.61 | −16.61 | −16.61 |
| hsa-miR-8057 | −16.61 | −16.61 | −16.61 | −16.61 | −16.61 |
| hsa-miR-8058 | −16.61 | −16.61 | −16.61 | −16.61 | −16.61 |
| hsa-miR-8059 | −16.61 | −16.61 | −16.61 | −16.61 | −16.61 |
| hsa-miR-8060 | −16.61 | −16.61 | −16.61 | −16.61 | −16.61 |
| hsa-miR-8061 | −16.61 | −16.61 | −16.61 | −16.61 | −16.61 |
| hsa-miR-8062 | −16.61 | −16.61 | −16.61 | −16.61 | −16.61 |
| hsa-miR-8063 | −16.61 | −16.61 | −16.61 | −16.61 | −16.61 |
| hsa-miR-8064 | −16.61 | −16.61 | 1.049 | −16.61 | −16.61 |
| hsa-miR-8065 | 16.61 | −16.61 | −16.61 | −16.61 | −16.61 |
| hsa-miR-8066 | −16.61 | −16.61 | −16.61 | −16.61 | −16.61 |
| hsa-miR-8067 | −16.61 | −16.61 | −16.61 | −16.61 | −16.61 |
| hsa-miR-8068 | −16.61 | −16.61 | −16.61 | −16.61 | −16.61 |
| hsa-miR-8069 | −16.61 | −16.61 | −16.61 | −16.61 | −16.61 |
| hsa-miR-8070 | −16.61 | −16.61 | −16.61 | −16.61 | −16.61 |
| hsa-miR-8071 | −16.61 | −16.61 | −16.61 | −16.61 | −16.61 |
| hsa-miR-8072 | −16.61 | −16.61 | −16.61 | −16.61 | −16.61 |
| hsa-miR-8073 | −16.61 | −16.61 | −16.61 | −16.61 | −16.61 |
| hsa-miR-8074 | −16.61 | −16.61 | −16.61 | −16.61 | −16.61 |
| hsa-miR-8075 | −16.61 | −16.61 | −16.61 | −16.61 | −16.61 |
| hsa-miR-8076 | −16.61 | −16.61 | −16.61 | −16.61 | −16.61 |
| hsa-miR-8077-3p | −16.61 | −16.61 | −16.61 | −16.61 | −16.61 |
| hsa-miR-8077-5p | −16.61 | −16.61 | −16.61 | −16.61 | −16.61 |
| hsa-miR-8078 | −16.61 | −16.61 | −16.61 | −16.61 | −16.61 |
| hsa-miR-8079 | 16.61 | −16.61 | −16.61 | −16.61 | −16.61 |
| hsa-miR-8080 | 16.61 | −16.61 | −16.61 | −16.61 | −16.61 |
| hsa-miR-8081 | −16.61 | −16.61 | −16.61 | −16.61 | −16.61 |
| hsa-miR-8082 | −16.61 | −16.61 | −16.61 | −16.61 | −16.61 |
| hsa-miR-8083 | −16.61 | −16.61 | −16.61 | −16.61 | −16.61 |
| hsa-miR-8084 | −16.61 | −16.61 | −16.61 | −16.61 | −16.61 |
| hsa-miR-8085 | −16.61 | −16.61 | −16.61 | −16.61 | −16.61 |
| hsa-miR-8086 | 16.61 | −16.61 | −16.61 | −16.61 | −16.61 |
| hsa-miR-8087 | −16.61 | −16.61 | −16.61 | −16.61 | −16.61 |
| hsa-miR-8088 | −16.61 | −16.61 | −16.61 | −16.61 | −16.61 |
| hsa-miR-8089 | −16.61 | −16.61 | −16.61 | −16.61 | −16.61 |
| hsa-miR-8485 | −16.61 | −16.61 | 3.917 | 4.229 | 4.424 |
| hsa-miR-873-3p | 4.351 | 4.207 | −16.61 | 1.941 | −16.61 |
| hsa-miR-873-5p | 6.638 | 5.612 | 2.187 | 1.313 | −16.61 |
| hsa-miR-874-3p | 6.697 | 5.961 | 3.994 | 3.661 | 2.588 |
| hsa-miR-874-5p | 5.212 | 4.149 | 3.342 | 2.661 | −16.61 |
| hsa-miR-875-3p | −16.61 | −16.61 | −16.61 | −16.61 | −16.61 |
| hsa-miR-875-5p | −16.61 | −16.61 | −16.61 | −16.61 | −16.61 |
| hsa-miR-876-3p | 1.958 | −16.61 | −16.61 | −16.61 | −16.61 |
| hsa-miR-876-5p | 3.646 | 1.342 | −16.61 | −16.61 | −16.61 |
| hsa-miR-877-3p | −16.61 | −16.61 | −16.61 | −16.61 | −16.61 |
| hsa-miR-877-5p | 7.355 | 6.801 | 4.585 | 5.139 | 9.432 |
| hsa-miR-885-3p | −16.61 | −16.61 | −16.61 | −16.61 | −16.61 |
| hsa-miR-885-5p | −1.986 | −16.61 | −16.61 | 0.439 | −16.61 |
| hsa-miR-887-3p | 8.533 | 6.848 | 3.012 | 4.102 | 4.424 |
| hsa-miR-887-5p | −16.61 | 2.857 | −16.61 | −16.61 | −16.61 |
| hsa-miR-888-3p | −16.61 | −16.61 | −16.61 | −16.61 | −16.61 |
| hsa-miR-888-5p | 4.831 | −16.61 | −16.61 | 2.102 | 3.481 |
| hsa-miR-889-3p | 5.132 | 4.91 | 1.049 | 2.661 | 2.95 |
| hsa-miR-889-5p | −16.61 | −16.61 | −16.61 | −16.61 | −16.61 |
| hsa-miR-890 | −16.61 | −16.61 | −16.61 | −16.61 | 3.481 |
| hsa-miR-891a-3p | −16.61 | −16.61 | −16.61 | −16.61 | −16.61 |
| hsa-miR-891a-5p | 6.524 | 4.875 | 3.704 | 3.063 | 3.24 |
| hsa-miR-891b | 3.809 | 1.535 | 1.049 | −16.61 | −16.61 |
| hsa-miR-892a | 4.533 | 1.857 | 0.897 | 1.313 | −16.61 |
| hsa-miR-892b | 1.671 | −16.61 | 0.049 | −16.61 | −16.61 |
| hsa-miR-892c-3p | −16.61 | −16.61 | −16.61 | −16.61 | −16.61 |
| hsa-miR-892c-5p | −16.61 | −16.61 | −16.61 | −16.61 | −16.61 |
| hsa-miR-9-3p | 10.731 | 7.633 | 5.334 | 7.29 | 7.474 |
| hsa-miR-9-5p | 14.756 | 11.619 | 8.284 | 9 | 5.91 |
| hsa-miR-920 | −16.61 | −16.61 | −16.61 | −16.61 | −16.61 |
| hsa-miR-921 | −16.61 | −16.61 | −16.61 | −16.61 | −16.61 |
| hsa-miR-922 | −16.61 | −16.61 | −16.61 | −16.61 | −16.61 |
| hsa-miR-924 | −16.61 | −16.61 | −16.61 | −16.61 | −16.61 |
| hsa-miR-92a-1-5p | −16.61 | −16.61 | 0.312 | −16.61 | 4.638 |
| hsa-miR-92a-2-5p | −16.61 | −16.61 | −16.61 | −16.61 | −16.61 |
| hsa-miR-92a-3p | 10.8 | 11.497 | 11.384 | 11.443 | 12.004 |
| hsa-miR-92b-3p | 8.168 | 6.157 | 2.482 | 3.211 | 3.481 |
| hsa-miR-92b-5p | 7.08 | 4.875 | 1.534 | 2.246 | −16.61 |
| hsa-miR-93-3p | 6.555 | 6.089 | 6.076 | 5.842 | 8.084 |
| hsa-miR-93-5p | 14.979 | 15.332 | 15.698 | 15.419 | 15.68 |
| hsa-miR-933 | −16.61 | −16.61 | −16.61 | −16.61 | −16.61 |
| hsa-miR-934 | −16.61 | 1.12 | −16.61 | −16.61 | −16.61 |
| hsa-miR-935 | 7.21 | 4.149 | 0.897 | −0.561 | −16.61 |
| hsa-miR-936-3p | −16.61 | −16.61 | −16.61 | −16.61 | −16.61 |
| hsa-miR-936-5p | −16.61 | −16.61 | −16.61 | −16.61 | −16.61 |
| hsa-miR-937-3p | −16.61 | −16.61 | 1.897 | −16.61 | 3.24 |
| hsa-miR-937-5p | −16.61 | −16.61 | −16.61 | −16.61 | −16.61 |
| hsa-miR-938-3p | −16.61 | −16.61 | −16.61 | −16.61 | −16.61 |
| hsa-miR-938-5p | −16.61 | −16.61 | −16.61 | −16.61 | −16.61 |
| hsa-miR-939-3p | −16.61 | −16.61 | −16.61 | −16.61 | −16.61 |
| hsa-miR-939-5p | 3.076 | 2.535 | −16.61 | −16.61 | −16.61 |
| hsa-miR-940-3p | 2.549 | 0.857 | −16.61 | −16.61 | −16.61 |
| hsa-miR-940-5p | −16.61 | −16.61 | −16.61 | −16.61 | −16.61 |
| hsa-miR-941 | 7.492 | 8.627 | 8.05 | 8.252 | 7.797 |
| hsa-miR-942-3p | −16.61 | −16.61 | 2.371 | −16.61 | 3.687 |
| hsa-miR-942-5p | 4.307 | 5.405 | 6.219 | 5.865 | 5.207 |
| hsa-miR-943-3p | 3.044 | −16.61 | 0.727 | 2.498 | −16.61 |
| hsa-miR-943-5p | 3.356 | −16.61 | −16.61 | −16.61 | −16.61 |
| hsa-miR-944-3p | −16.61 | −16.61 | −16.61 | −16.61 | −16.61 |
| hsa-miR-944-5p | −16.61 | −16.61 | −16.61 | −16.61 | −16.61 |
| hsa-miR-95-3p | 11.291 | 8.622 | 5.482 | 6.246 | 6.395 |
| hsa-miR-95-5p | 6.267 | 4.393 | −16.61 | −16.61 | −16.61 |
| hsa-miR-9500 | −16.61 | −16.61 | −16.61 | −16.61 | −16.61 |
| hsa-miR-96-3p | −16.61 | −16.61 | −16.61 | −16.61 | −16.61 |
| hsa-miR-96-5p | 2.184 | 3.535 | 0.727 | 0.854 | −16.61 |
| hsa-miR-9718 | −16.61 | −16.61 | −16.61 | −16.61 | −16.61 |
| hsa-miR-98-3p | −16.61 | −16.61 | −16.61 | −16.61 | −16.61 |
| hsa-miR-98-5p | 4.991 | 5.557 | 5.003 | 5.439 | 9.89 |
| hsa-miR-9851-3p | −16.61 | −16.61 | −16.61 | −16.61 | −16.61 |
| hsa-miR-9851-5p | −16.61 | −16.61 | −16.61 | −16.61 | −16.61 |
| hsa-miR-9898 | −16.61 | −16.61 | −16.61 | −16.61 | −16.61 |
| hsa-miR-9899 | −16.61 | −16.61 | −16.61 | −16.61 | −16.61 |
| hsa-miR-9900 | −16.61 | −16.61 | −16.61 | −16.61 | −16.61 |
| hsa-miR-9901 | −16.61 | −16.61 | 1.634 | −16.61 | −16.61 |
| hsa-miR-9902 | −16.61 | −16.61 | −16.61 | −16.61 | −16.61 |
| hsa-miR-9903 | 2.411 | −16.61 | −16.61 | 1.554 | −16.61 |
| hsa-miR-9983-3p | 3.613 | 4.744 | 1.534 | 3.313 | −16.61 |
| hsa-miR-9985 | −16.61 | −16.61 | −16.61 | −16.61 | −16.61 |
| hsa-miR-9986 | −16.61 | −16.61 | −16.61 | −16.61 | 3.588 |
| hsa-miR-99a-3p | 8.899 | 6.082 | 4.385 | 5.148 | 4.24 |
| hsa-miR-99a-5p | 11.259 | 9.442 | 9.183 | 9.002 | 10.249 |
| hsa-miR-99b-3p | 10.452 | 8.791 | 7.675 | 7.141 | 7.797 |
| hsa-miR-99b-5p | 13.887 | 11.618 | 11.455 | 11.202 | 10.39 |

TABLE 15
Biological pathways predicted to be affected by MGL-EV miR. The enrichment ratio, p-Value and
false discovery rate (FDR) are given. Results are ranked from highest to lowest significance.

| | | | | | Enrichment | | |
|---|---|---|---|---|---|---|---|
| geneSet | description | size | overlap | expect | Ratio | pValue | FDR |
| GO: 0070482 | response to oxygen levels | 337 | 55 | 21.61861 | 2.544104326 | 1.17E−10 | 5.69E−08 |
| GO: 0097193 | intrinsic apoptotic signaling pathway | 285 | 49 | 18.2828 | 2.680114497 | 1.96E−10 | 5.69E−08 |
| GO: 0007050 | cell cycle arrest | 240 | 44 | 15.39604 | 2.857877193 | 2.01E−10 | 5.69E−08 |
| GO: 0018209 | peptidyl-serine modification | 305 | 50 | 19.5658 | 2.555478861 | 7.11E−10 | 1.35E−07 |
| GO: 0010608 | posttranscriptional regulation of gene expression | 486 | 68 | 31.17699 | 2.18109595 | 7.93E−10 | 1.35E−07 |
| GO: 1902532 | negative regulation of intracellular signal transduction | 495 | 67 | 31.75434 | 2.1099479 | 4.25E−09 | 6.01E−07 |
| GO: 0009896 | positive regulation of catabolic process | 409 | 58 | 26.23742 | 2.210582937 | 8.83E−09 | 9.90E−07 |
| GO: 1903706 | regulation of hemopoiesis | 389 | 56 | 24.95442 | 2.244091463 | 9.32E−09 | 9.90E−07 |
| GO: 0006401 | RNA catabolic process | 341 | 51 | 21.87521 | 2.331406081 | 1.20E−08 | 1.04E−06 |
| GO: 0042326 | negative regulation of phosphorylation | 423 | 59 | 27.13553 | 2.174271494 | 1.22E−08 | 1.04E−06 |
| GO: 0033002 | muscle cell proliferation | 182 | 34 | 11.67533 | 2.912122614 | 1.42E−08 | 1.10E−06 |
| GO: 1903829 | positive regulation of cellular protein localization | 317 | 48 | 20.33561 | 2.360391831 | 2.18E−08 | 1.55E−06 |
| GO: 0071496 | cellular response to external stimulus | 330 | 49 | 21.16956 | 2.314644338 | 2.95E−08 | 1.93E−06 |
| GO: 1901342 | regulation of vasculature development | 313 | 47 | 20.07901 | 2.340753321 | 3.99E−08 | 2.42E−06 |
| GO: 0031647 | regulation of protein stability | 267 | 42 | 17.1281 | 2.452111177 | 5.59E−08 | 3.17E−06 |
| GO: 0030099 | myeloid cell differentiation | 379 | 53 | 24.31292 | 2.179911123 | 6.25E−08 | 3.32E−06 |
| GO: 0071900 | regulation of protein serine/threonine kinase activity | 497 | 63 | 31.88264 | 1.975997035 | 1.47E−07 | 7.35E−06 |
| GO: 0001525 | angiogenesis | 487 | 62 | 31.24114 | 1.984562844 | 1.58E−07 | 7.48E−06 |
| GO: 0009314 | response to radiation | 443 | 57 | 28.41853 | 2.005733634 | 3.55E−07 | 1.59E−05 |
| GO: 0042176 | regulation of protein catabolic process | 370 | 50 | 23.73557 | 2.106543385 | 4.29E−07 | 1.82E−05 |
| GO: 0010948 | negative regulation of cell cycle process | 278 | 41 | 17.83375 | 2.299011738 | 4.78E−07 | 1.85E−05 |
| GO: 0050769 | positive regulation of neurogenesis | 447 | 57 | 28.67513 | 1.987785235 | 4.79E−07 | 1.85E−05 |
| GO: 0097191 | extrinsic apoptotic signaling pathway | 220 | 35 | 14.11304 | 2.479976077 | 5.40E−07 | 2.00E−05 |
| GO: 0048872 | homeostasis of number of cells | 245 | 37 | 15.71679 | 2.35416971 | 9.60E−07 | 3.40E−05 |
| GO: 0035690 | cellular response to drug | 349 | 47 | 22.38841 | 2.099300256 | 1.05E−06 | 3.58E−05 |
| GO: 0031050 | dsRNA processing | 48 | 14 | 3.079209 | 4.546622807 | 1.12E−06 | 3.67E−05 |
| GO: 0010498 | proteasomal protein catabolic process | 449 | 56 | 28.80343 | 1.944212871 | 1.23E−06 | 3.88E−05 |
| GO: 0104004 | cellular response to environmental stimulus | 320 | 44 | 20.52806 | 2.143407895 | 1.32E−06 | 4.00E−05 |
| GO: 0034248 | regulation of cellular amide metabolic process | 385 | 50 | 24.69782 | 2.024470267 | 1.42E−06 | 4.02E−05 |
| GO: 2001233 | regulation of apoptotic signaling pathway | 385 | 50 | 24.69782 | 2.024470267 | 1.42E−06 | 4.02E−05 |
| GO: 0001101 | response to acid chemical | 332 | 45 | 21.29786 | 2.112888396 | 1.49E−06 | 4.09E−05 |
| GO: 1904951 | positive regulation of establishment of protein localization | 437 | 54 | 28.03363 | 1.926257979 | 2.54E−06 | 6.73E−05 |
| GO: 0198738 | cell-cell signaling by wnt | 460 | 56 | 29.50908 | 1.897720824 | 2.64E−06 | 6.79E−05 |
| GO: 0050673 | epithelial cell proliferation | 372 | 48 | 23.86387 | 2.011409168 | 2.77E−06 | 6.91E−05 |
| GO: 0016049 | cell growth | 473 | 57 | 30.34303 | 1.878520085 | 2.95E−06 | 7.00E−05 |
| GO: 0002521 | leukocyte differentiation | 496 | 59 | 31.81849 | 1.854267827 | 3.00E−06 | 7.00E−05 |
| GO: 0061614 | pri-miRNA transcription by RNA polymerase II | 45 | 13 | 2.886758 | 4.503321637 | 3.05E−06 | 7.00E−05 |
| GO: 0044843 | cell cycle G1/S phase transition | 247 | 36 | 15.84509 | 2.271996591 | 3.13E−06 | 7.00E−05 |
| GO: 0030705 | cytoskeleton-dependent intracellular transport | 170 | 28 | 10.90553 | 2.567504644 | 3.65E−06 | 7.95E−05 |
| GO: 0045926 | negative regulation of growth | 250 | 36 | 16.03754 | 2.244732632 | 4.15E−06 | 8.83E−05 |
| GO: 0031667 | response to nutrient levels | 483 | 57 | 30.98454 | 1.839627329 | 5.64E−06 | 1.17E−04 |
| GO: 0060537 | muscle tissue development | 371 | 47 | 23.79972 | 1.974813449 | 5.80E−06 | 1.17E−04 |
| GO: 0051052 | regulation of DNA metabolic process | 405 | 50 | 25.98082 | 1.924496426 | 6.11E−06 | 1.19E−04 |
| GO: 0061919 | process utilizing autophagic mechanism | 473 | 56 | 30.34303 | 1.845563592 | 6.18E−06 | 1.19E−04 |
| GO: 0071826 | ribonucleoprotein complex subunit organization | 245 | 35 | 15.71679 | 2.226917293 | 6.74E−06 | 1.25E−04 |
| GO: 0051348 | negative regulation of transferase activity | 266 | 37 | 17.06395 | 2.168314207 | 6.94E−06 | 1.25E−04 |
| GO: 0045930 | negative regulation of mitotic cell cycle | 256 | 36 | 16.42245 | 2.192121711 | 7.18E−06 | 1.25E−04 |
| GO: 0072593 | reactive oxygen species metabolic process | 256 | 36 | 16.42245 | 2.192121711 | 7.18E−06 | 1.25E−04 |
| GO: 0045787 | positive regulation of cell cycle | 374 | 47 | 23.99217 | 1.958972699 | 7.21E−06 | 1.25E−04 |
| GO: 0006979 | response to oxidative stress | 431 | 52 | 27.64873 | 1.880737575 | 7.76E−06 | 1.32E−04 |
| GO: 1905475 | regulation of protein localization to membrane | 177 | 28 | 11.35458 | 2.465964912 | 8.08E−06 | 1.35E−04 |
| GO: 0045862 | positive regulation of proteolysis | 343 | 44 | 22.00351 | 1.999680835 | 8.32E−06 | 1.36E−04 |
| GO: 0040029 | regulation of gene expression, epigenetic | 258 | 36 | 16.55075 | 2.175128519 | 8.57E−06 | 1.37E−04 |
| GO: 0071559 | response to transforming growth factor beta | 238 | 34 | 15.26774 | 2.226917293 | 9.09E−06 | 1.40E−04 |
| GO: 0051271 | negative regulation of cellular component movement | 301 | 40 | 19.3092 | 2.07155097 | 9.16E−06 | 1.40E−04 |
| GO: 0016569 | covalent chromatin modification | 468 | 55 | 30.02228 | 1.83197256 | 9.30E−06 | 1.40E−04 |
| GO: 0072331 | signal transduction by p53 class mediator | 218 | 32 | 13.98474 | 2.288208595 | 9.40E−06 | 1.40E−04 |
| GO: 0002009 | morphogenesis of an epithelium | 480 | 56 | 30.79209 | 1.818649123 | 9.59E−06 | 1.40E−04 |
| GO: 0007178 | transmembrane receptor protein serine/threonine kinase | 328 | 42 | 21.04126 | 1.996078306 | 1.39E−05 | 1.98E−04 |
| | signaling pathway | | | | | | |
| GO: 0016458 | gene silencing | 202 | 30 | 12.95834 | 2.315112038 | 1.40E−05 | 1.98E−04 |
| GO: 0061842 | microtubule organizing center localization | 25 | 9 | 1.603754 | 5.611831579 | 1.42E−05 | 1.98E−04 |
| GO: 1901987 | regulation of cell cycle phase transition | 386 | 47 | 24.76197 | 1.898071993 | 1.66E−05 | 2.28E−04 |
| GO: 1903311 | regulation of mRNA metabolic process | 266 | 36 | 17.06395 | 2.10971112 | 1.70E−05 | 2.29E−04 |
| GO: 0048545 | response to steroid hormone | 388 | 47 | 24.89027 | 1.888288117 | 1.90E−05 | 2.53E−04 |
| GO: 0040013 | negative regulation of locomotion | 314 | 40 | 20.14316 | 1.985786121 | 2.50E−05 | 3.17E−04 |
| GO: 0070997 | neuron death | 325 | 41 | 20.84881 | 1.966539271 | 2.50E−05 | 3.17E−04 |
| GO: 0097327 | response to antineoplastic agent | 94 | 18 | 6.030117 | 2.985016797 | 2.50E−05 | 3.17E−04 |
| GO: 0045927 | positive regulation of growth | 250 | 34 | 16.03754 | 2.120025263 | 2.61E−05 | 3.26E−04 |
| GO: 0034405 | response to fluid shear stress | 33 | 10 | 2.116956 | 4.723763955 | 2.68E−05 | 3.30E−04 |
| GO: 0090130 | tissue migration | 283 | 37 | 18.1545 | 2.038062116 | 2.82E−05 | 3.43E−04 |
| GO: 0044772 | mitotic cell cycle phase transition | 487 | 55 | 31.24114 | 1.760499298 | 2.91E−05 | 3.48E−04 |
| GO: 0018210 | peptidyl-threonine modification | 114 | 20 | 7.31312 | 2.734810711 | 3.44E−05 | 4.07E−04 |
| GO: 0002285 | lymphocyte activation involved in immune response | 172 | 26 | 11.03383 | 2.356389229 | 3.79E−05 | 4.41E−04 |
| GO: 0003170 | heart valve development | 56 | 13 | 3.59241 | 3.618740602 | 4.09E−05 | 4.70E−04 |
| GO: 0060759 | regulation of response to cytokine stimulus | 163 | 25 | 10.45648 | 2.390862125 | 4.15E−05 | 4.70E−04 |
| GO: 0051098 | regulation of binding | 367 | 44 | 23.54312 | 1.868911516 | 4.47E−05 | 5.00E−04 |
| GO: 1904019 | epithelial cell apoptotic process | 99 | 18 | 6.350868 | 2.834258373 | 5.15E−05 | 5.69E−04 |
| GO: 1903320 | regulation of protein modification by small protein | 217 | 30 | 13.92059 | 2.155081252 | 5.63E−05 | 6.12E−04 |
| | conjugation or removal | | | | | | |
| GO: 0022613 | ribonucleoprotein complex biogenesis | 440 | 50 | 28.22608 | 1.771411483 | 5.69E−05 | 6.12E−04 |
| GO: 2000027 | regulation of animal organ morphogenesis | 207 | 29 | 13.27909 | 2.183885075 | 5.83E−05 | 6.20E−04 |
| GO: 0032103 | positive regulation of response to external stimulus | 293 | 37 | 18.796 | 1.968503682 | 6.00E−05 | 6.30E−04 |
| GO: 0060485 | mesenchyme development | 262 | 34 | 16.80735 | 2.022924869 | 6.85E−05 | 7.10E−04 |
| GO: 1901654 | response to ketone | 189 | 27 | 12.12438 | 2.226917293 | 7.40E−05 | 7.58E−04 |
| GO: 0070646 | protein modification by small protein removal | 288 | 36 | 18.47525 | 1.948552632 | 9.27E−05 | 9.38E−04 |
| GO: 0016311 | dephosphorylation | 461 | 51 | 29.57323 | 1.724532481 | 9.59E−05 | 9.59E−04 |
| GO: 2001020 | regulation of response to DNA damage stimulus | 203 | 28 | 13.02249 | 2.150127042 | 1.02E−04 | 0.001012 |
| GO: 0031349 | positive regulation of defense response | 427 | 48 | 27.39213 | 1.752328362 | 1.04E−04 | 0.001015 |
| GO: 1901652 | response to peptide | 487 | 53 | 31.24114 | 1.696481141 | 1.08E−04 | 0.001032 |
| GO: 0001667 | ameboidal-type cell migration | 381 | 44 | 24.44122 | 1.800237602 | 1.08E−04 | 0.001032 |
| GO: 0031023 | microtubule organizing center organization | 134 | 21 | 8.596124 | 2.442961508 | 1.22E−04 | 0.001151 |
| GO: 0000075 | cell cycle checkpoint | 216 | 29 | 13.85644 | 2.092889864 | 1.26E−04 | 0.001177 |
| GO: 0007369 | gastrulation | 175 | 25 | 11.22628 | 2.226917293 | 1.35E−04 | 0.001246 |
| GO: 0046677 | response to antibiotic | 316 | 38 | 20.27146 | 1.874556962 | 1.36E−04 | 0.001246 |
| GO: 0003007 | heart morphogenesis | 250 | 32 | 16.03754 | 1.995317895 | 1.44E−04 | 0.001287 |
| GO: 0009612 | response to mechanical stimulus | 207 | 28 | 13.27909 | 2.108578693 | 1.44E−04 | 0.001287 |
| GO: 2000147 | positive regulation of cell motility | 493 | 53 | 31.62604 | 1.675834312 | 1.47E−04 | 0.001296 |
| GO: 0010821 | regulation of mitochondrion organization | 176 | 25 | 11.29043 | 2.214264354 | 1.48E−04 | 0.001296 |
| GO: 0033044 | regulation of chromosome organization | 329 | 39 | 21.10541 | 1.847867541 | 1.52E−04 | 0.001316 |
| GO: 0048732 | gland development | 434 | 48 | 27.84118 | 1.724065001 | 1.54E−04 | 0.00132 |
| GO: 2001057 | reactive nitrogen species metabolic process | 72 | 14 | 4.618813 | 3.031081871 | 1.64E−04 | 0.001397 |
| GO: 0048880 | sensory system development | 355 | 41 | 22.77331 | 1.800352854 | 1.83E−04 | 0.001535 |
| GO: 0006457 | protein folding | 210 | 28 | 13.47154 | 2.07845614 | 1.84E−04 | 0.001535 |
| GO: 0051090 | regulation of DNA-binding transcription factor activity | 404 | 45 | 25.91667 | 1.736334028 | 2.08E−04 | 0.001721 |
| GO: 0042110 | T cell activation | 452 | 49 | 28.99588 | 1.689895203 | 2.13E−04 | 0.001741 |
| GO: 0090287 | regulation of cellular response to growth factor stimulus | 256 | 32 | 16.42245 | 1.948552632 | 2.23E−04 | 0.001805 |
| GO: 0006260 | DNA replication | 268 | 33 | 17.19225 | 1.919469756 | 2.37E−04 | 0.001885 |
| GO: 0034976 | response to endoplasmic reticulum stress | 268 | 33 | 17.19225 | 1.919469756 | 2.37E−04 | 0.001885 |
| GO: 0048144 | fibroblast proliferation | 84 | 15 | 5.388615 | 2.783646617 | 2.62E−04 | 0.002059 |
| GO: 0009895 | negative regulation of catabolic process | 282 | 34 | 18.09035 | 1.879455021 | 2.87E−04 | 0.002235 |
| GO: 0043254 | regulation of protein complex assembly | 447 | 48 | 28.67513 | 1.673924408 | 3.07E−04 | 0.002371 |
| GO: 0006925 | inflammatory cell apoptotic process | 22 | 7 | 1.411304 | 4.959952153 | 3.18E−04 | 0.002433 |
| GO: 0003205 | cardiac chamber development | 164 | 23 | 10.52063 | 2.186181001 | 3.24E−04 | 0.002455 |
| GO: 0048193 | Golgi vesicle transport | 366 | 41 | 23.47897 | 1.746243888 | 3.49E−04 | 0.002626 |
| GO: 0010586 | miRNA metabolic process | 29 | 8 | 1.860355 | 4.300254083 | 3.56E−04 | 0.002653 |
| GO: 0050867 | positive regulation of cell activation | 310 | 36 | 19.88656 | 1.810268251 | 3.99E−04 | 0.002938 |
| GO: 0097305 | response to alcohol | 231 | 29 | 14.81869 | 1.956987924 | 4.01E−04 | 0.002938 |
| GO: 0045088 | regulation of innate immune response | 369 | 41 | 23.67142 | 1.732046784 | 4.13E−04 | 0.002994 |
| GO: 0071897 | DNA biosynthetic process | 188 | 25 | 12.06023 | 2.072928331 | 4.16E−04 | 0.002994 |
| GO: 0007059 | chromosome segregation | 312 | 36 | 20.01486 | 1.798663968 | 4.51E−04 | 0.003219 |
| GO: 0002694 | regulation of leukocyte activation | 481 | 50 | 30.85624 | 1.620417989 | 4.95E−04 | 0.003508 |
| GO: 0001655 | urogenital system development | 326 | 37 | 20.91296 | 1.769237972 | 5.19E−04 | 0.003647 |
| GO: 0072089 | stem cell proliferation | 109 | 17 | 6.99237 | 2.431221632 | 5.55E−04 | 0.003863 |
| GO: 0034341 | response to interferon-gamma | 192 | 25 | 12.31683 | 2.029742325 | 5.71E−04 | 0.003948 |
| GO: 0060560 | developmental growth involved in morphogenesis | 225 | 28 | 14.43379 | 1.939892398 | 5.77E−04 | 0.003955 |
| GO: 0031098 | stress-activated protein kinase signaling cascade | 305 | 35 | 19.5658 | 1.788835203 | 5.94E−04 | 0.004042 |
| GO: 0031346 | positive regulation of cell projection organization | 365 | 40 | 23.41482 | 1.708320115 | 6.36E−04 | 0.004291 |
| GO: 0002237 | response to molecule of bacterial origin | 330 | 37 | 21.16956 | 1.747792663 | 6.54E−04 | 0.004377 |
| GO: 0030522 | intracellular receptor signaling pathway | 284 | 33 | 18.21865 | 1.811330615 | 6.77E−04 | 0.004494 |
| GO: 0002181 | cytoplasmic translation | 92 | 15 | 5.901816 | 2.541590389 | 7.17E−04 | 0.004706 |
| GO: 0019058 | viral life cycle | 285 | 33 | 18.2828 | 1.804975069 | 7.20E−04 | 0.004706 |
| GO: 0061351 | neural precursor cell proliferation | 143 | 20 | 9.173476 | 2.180198749 | 8.01E−04 | 0.005199 |
| GO: 0001819 | positive regulation of cytokine production | 418 | 44 | 26.81477 | 1.640886427 | 8.16E−04 | 0.005253 |
| GO: 0022407 | regulation of cell-cell adhesion | 383 | 41 | 24.56952 | 1.668734369 | 8.73E−04 | 0.005549 |
| GO: 0001503 | ossification | 371 | 40 | 23.79972 | 1.680692297 | 8.75E−04 | 0.005549 |
| GO: 0051656 | establishment of organelle localization | 494 | 50 | 31.69019 | 1.57777541 | 9.01E−04 | 0.005675 |
| GO: 0033028 | myeloid cell apoptotic process | 33 | 8 | 2.116956 | 3.779011164 | 9.15E−04 | 0.005722 |
| GO: 0043491 | protein kinase B signaling | 232 | 28 | 14.88284 | 1.881361162 | 9.36E−04 | 0.005806 |
| GO: 0035966 | response to topologically incorrect protein | 188 | 24 | 12.06023 | 1.990011198 | 9.67E−04 | 0.005958 |
| GO: 0048638 | regulation of developmental growth | 314 | 35 | 20.14316 | 1.737562856 | 0.001003208 | 0.006135 |
| GO: 0030258 | lipid modification | 291 | 33 | 18.6677 | 1.767759088 | 0.001030993 | 0.00626 |
| GO: 0098727 | maintenance of cell number | 146 | 20 | 9.365926 | 2.135400144 | 0.001042788 | 0.006274 |
| GO: 0007568 | aging | 303 | 34 | 19.4375 | 1.749195762 | 0.001048125 | 0.006274 |
| GO: 0070265 | necrotic cell death | 50 | 10 | 3.207509 | 3.117684211 | 0.001100795 | 0.006543 |
| GO: 1904029 | regulation of cyclin-dependent protein kinase activity | 96 | 15 | 6.158417 | 2.435690789 | 0.001126722 | 0.006629 |
| GO: 0043276 | anoikis | 34 | 8 | 2.181106 | 3.667863777 | 0.001130767 | 0.006629 |
| GO: 0010975 | regulation of neuron projection development | 475 | 48 | 30.47133 | 1.57525097 | 0.001171257 | 0.006819 |
| GO: 0070972 | protein localization to endoplasmic reticulum | 137 | 19 | 8.788575 | 2.16189781 | 0.001186254 | 0.006859 |
| GO: 0046777 | protein autophosphorylation | 225 | 27 | 14.43379 | 1.870610526 | 0.001246876 | 0.007161 |
| GO: 0043620 | regulation of DNA-templated transcription in response to stress | 78 | 13 | 5.003714 | 2.598070175 | 0.001289692 | 0.007357 |
| GO: 0009615 | response to virus | 319 | 35 | 20.46391 | 1.710328329 | 0.001323288 | 0.007449 |
| GO: 0010721 | negative regulation of cell development | 319 | 35 | 20.46391 | 1.710328329 | 0.001323288 | 0.007449 |
| GO: 0045165 | cell fate commitment | 249 | 29 | 15.97339 | 1.815518918 | 0.001343911 | 0.007515 |
| GO: 0045785 | positive regulation of cell adhesion | 392 | 41 | 25.14687 | 1.63042159 | 0.001367564 | 0.007598 |
| GO: 0031109 | microtubule polymerization or depolymerization | 108 | 16 | 6.928219 | 2.309395712 | 0.001400916 | 0.007732 |
| GO: 0019216 | regulation of lipid metabolic process | 382 | 40 | 24.50537 | 1.632295398 | 0.001522536 | 0.008296 |
| GO: 0010657 | muscle cell apoptotic process | 70 | 12 | 4.490513 | 2.672300752 | 0.001522635 | 0.008296 |
| GO: 0061008 | hepaticobiliary system development | 140 | 19 | 8.981025 | 2.115571429 | 0.001538265 | 0.008328 |
| GO: 0031032 | actomyosin structure organization | 184 | 23 | 11.80363 | 1.948552632 | 0.001621489 | 0.008723 |
| GO: 0060249 | anatomical structure homeostasis | 408 | 42 | 26.17327 | 1.604690402 | 0.001633573 | 0.008733 |
| GO: 0048285 | organelle fission | 459 | 46 | 29.44493 | 1.562238275 | 0.001736488 | 0.009225 |
| GO: 0032386 | regulation of intracellular transport | 422 | 43 | 27.07138 | 1.588393614 | 0.001762436 | 0.009305 |
| GO: 1990778 | protein localization to cell periphery | 301 | 33 | 19.3092 | 1.709029551 | 0.001813997 | 0.009495 |
| GO: 0090150 | establishment of protein localization to membrane | 313 | 34 | 20.07901 | 1.693310913 | 0.001820901 | 0.009495 |
| GO: 0060627 | regulation of vesicle-mediated transport | 499 | 49 | 32.01094 | 1.530726717 | 0.00191335 | 0.009907 |
| GO: 0045023 | G0 to G1 transition | 45 | 9 | 2.886758 | 3.117684211 | 0.00193151 | 0.009907 |
| GO: 0000209 | protein polyubiquitination | 255 | 29 | 16.3583 | 1.772800826 | 0.001934817 | 0.009907 |
| GO: 0034504 | protein localization to nucleus | 267 | 30 | 17.1281 | 1.751507983 | 0.001968909 | 0.010021 |
| GO: 0048017 | inositol lipid-mediated signaling | 165 | 21 | 10.58478 | 1.983980861 | 0.00202733 | 0.010257 |
| GO: 0042113 | B cell activation | 233 | 27 | 14.94699 | 1.806383555 | 0.002081271 | 0.010468 |
| GO: 0018212 | peptidyl-tyrosine modification | 389 | 40 | 24.95442 | 1.602922473 | 0.002125522 | 0.010628 |
| GO: 0071216 | cellular response to biotic stimulus | 222 | 26 | 14.24134 | 1.825670934 | 0.002148582 | 0.01068 |
| GO: 0007162 | negative regulation of cell adhesion | 257 | 29 | 16.4866 | 1.75900471 | 0.002176051 | 0.010754 |
| GO: 0061564 | axon development | 490 | 48 | 31.43359 | 1.527029001 | 0.002223399 | 0.010924 |
| GO: 0007498 | mesoderm development | 124 | 17 | 7.954622 | 2.137122241 | 0.002381946 | 0.011636 |
| GO: 0007517 | muscle organ development | 380 | 39 | 24.37707 | 1.599864266 | 0.002481879 | 0.011958 |
| GO: 0051702 | interaction with symbiont | 74 | 12 | 4.747113 | 2.527852063 | 0.002482616 | 0.011958 |
| GO: 0007159 | leukocyte cell-cell adhesion | 319 | 34 | 20.46391 | 1.661461805 | 0.002489989 | 0.011958 |
| GO: 0046486 | glycerolipid metabolic process | 456 | 45 | 29.25248 | 1.538331025 | 0.002612368 | 0.012475 |
| GO: 0051235 | maintenance of location | 309 | 33 | 19.82241 | 1.664782831 | 0.002769297 | 0.01315 |
| GO: 0035265 | organ growth | 170 | 21 | 10.90553 | 1.925628483 | 0.00292098 | 0.013794 |
| GO: 0032886 | regulation of microtubule-based process | 204 | 24 | 13.08664 | 1.833931889 | 0.002939451 | 0.013804 |
| GO: 0018205 | peptidyl-lysine modification | 360 | 37 | 23.09406 | 1.602143275 | 0.003083734 | 0.014402 |
| GO: 0050920 | regulation of chemotaxis | 205 | 24 | 13.15079 | 1.824985879 | 0.003132918 | 0.014552 |
| GO: 0043112 | receptor metabolic process | 183 | 22 | 11.73948 | 1.874017831 | 0.003300295 | 0.015239 |
| GO: 0051493 | regulation of cytoskeleton organization | 500 | 48 | 32.07509 | 1.496488421 | 0.003316684 | 0.015239 |
| GO: 0071887 | leukocyte apoptotic process | 107 | 15 | 6.864069 | 2.185292671 | 0.003372458 | 0.015412 |
| GO: 0042063 | gliogenesis | 265 | 29 | 16.9998 | 1.705902681 | 0.00341614 | 0.015528 |
| GO: 0031099 | regeneration | 184 | 22 | 11.80363 | 1.863832952 | 0.003528281 | 0.015952 |
| GO: 0051193 | regulation of cofactor metabolic process | 78 | 12 | 5.003714 | 2.398218623 | 0.003886167 | 0.017477 |
| GO: 0035051 | cardiocyte differentiation | 141 | 18 | 9.045175 | 1.990011198 | 0.00396272 | 0.017728 |
| GO: 0033619 | membrane protein proteolysis | 59 | 10 | 3.784861 | 2.642105263 | 0.003996014 | 0.017783 |
| GO: 0002683 | negative regulation of immune system process | 416 | 41 | 26.68647 | 1.536358806 | 0.004043712 | 0.017902 |
| GO: 0072527 | pyrimidine-containing compound metabolic process | 99 | 14 | 6.350868 | 2.204423179 | 0.004191675 | 0.018461 |
| GO: 0042770 | signal transduction in response to DNA damage | 131 | 17 | 8.403673 | 2.022924869 | 0.004249209 | 0.018608 |
| GO: 0045444 | fat cell differentiation | 210 | 24 | 13.47154 | 1.781533835 | 0.004268932 | 0.018608 |
| GO: 1902850 | microtubule cytoskeleton organization involved in mitosis | 132 | 17 | 8.467824 | 2.007599681 | 0.00459441 | 0.019925 |
| GO: 0050878 | regulation of body fluid levels | 483 | 46 | 30.98454 | 1.484611529 | 0.004626333 | 0.019961 |
| GO: 0051653 | spindle localization | 42 | 8 | 2.694308 | 2.969223058 | 0.004666105 | 0.020031 |
| GO: 0007018 | microtubule-based movement | 271 | 29 | 17.3847 | 1.668133618 | 0.004700612 | 0.020078 |
| GO: 0048854 | brain morphogenesis | 34 | 7 | 2.181106 | 3.209380805 | 0.005107614 | 0.021707 |
| GO: 0022604 | regulation of cell morphogenesis | 473 | 45 | 30.34303 | 1.483042172 | 0.005156471 | 0.021775 |
| GO: 0034330 | cell junction organization | 285 | 30 | 18.2828 | 1.640886427 | 0.005174823 | 0.021775 |
| GO: 0090596 | sensory organ morphogenesis | 250 | 27 | 16.03754 | 1.683549474 | 0.005518465 | 0.023107 |
| GO: 0007163 | establishment or maintenance of cell polarity | 203 | 23 | 13.02249 | 1.766175784 | 0.005643892 | 0.023507 |
| GO: 0032606 | type I interferon production | 113 | 15 | 7.24897 | 2.069259432 | 0.005669325 | 0.023507 |
| GO: 0032200 | telomere organization | 146 | 18 | 9.365926 | 1.92186013 | 0.005726501 | 0.023617 |
| GO: 0019080 | viral gene expression | 62 | 10 | 3.977311 | 2.51426146 | 0.005751389 | 0.023617 |
| GO: 0030900 | forebrain development | 375 | 37 | 24.05632 | 1.538057544 | 0.006008775 | 0.024555 |
| GO: 0006471 | protein ADP-ribosylation | 35 | 7 | 2.245256 | 3.117684211 | 0.006038371 | 0.024558 |
| GO: 0032615 | interleukin-12 production | 53 | 9 | 3.399959 | 2.647090367 | 0.006107385 | 0.02472 |
| GO: 0010256 | endomembrane system organization | 401 | 39 | 25.72422 | 1.516080851 | 0.006188058 | 0.024928 |
| GO: 0070371 | ERK1 and ERK2 cascade | 326 | 33 | 20.91296 | 1.577969002 | 0.006290554 | 0.025222 |
| GO: 0000910 | cytokinesis | 159 | 19 | 10.19988 | 1.862767296 | 0.006454521 | 0.025575 |
| GO: 0060840 | artery development | 83 | 12 | 5.324465 | 2.253747622 | 0.006468961 | 0.025575 |
| GO: 0106027 | neuron projection organization | 83 | 12 | 5.324465 | 2.253747622 | 0.006468961 | 0.025575 |
| GO: 0061458 | reproductive system development | 428 | 41 | 27.45628 | 1.493283325 | 0.006570882 | 0.025858 |
| GO: 0008380 | RNA splicing | 417 | 40 | 26.75062 | 1.495292187 | 0.007047574 | 0.027606 |
| GO: 0071604 | transforming growth factor beta production | 36 | 7 | 2.309406 | 3.031081871 | 0.007089999 | 0.027644 |
| GO: 1902903 | regulation of supramolecular fiber organization | 329 | 33 | 21.10541 | 1.563580227 | 0.007195491 | 0.027899 |
| GO: 0050918 | positive chemotaxis | 64 | 10 | 4.105611 | 2.435690789 | 0.007221044 | 0.027899 |
| GO: 0043900 | regulation of multi-organism process | 367 | 36 | 23.54312 | 1.529109422 | 0.007294169 | 0.028054 |
| GO: 0006109 | regulation of carbohydrate metabolic process | 161 | 19 | 10.32818 | 1.839627329 | 0.007365876 | 0.028203 |
| GO: 0001763 | morphogenesis of a branching structure | 196 | 22 | 12.57344 | 1.74972073 | 0.007453964 | 0.028412 |
| GO: 0010038 | response to metal ion | 356 | 35 | 22.83746 | 1.532569486 | 0.007808379 | 0.029534 |
| GO: 0055081 | anion homeostasis | 55 | 9 | 3.52826 | 2.550832536 | 0.007817895 | 0.029534 |
| GO: 0062012 | regulation of small molecule metabolic process | 344 | 34 | 22.06766 | 1.540716034 | 0.008009678 | 0.030041 |
| GO: 0002764 | immune response-regulating signaling pathway | 485 | 45 | 31.11284 | 1.446348345 | 0.008022689 | 0.030041 |
| GO: 1903008 | organelle disassembly | 96 | 13 | 6.158417 | 2.110932018 | 0.008175986 | 0.030275 |
| GO: 0031589 | cell-substrate adhesion | 332 | 33 | 21.29786 | 1.54945149 | 0.008206348 | 0.030275 |
| GO: 0051258 | protein polymerization | 270 | 28 | 17.32055 | 1.616576998 | 0.008225119 | 0.030275 |
| GO: 0030865 | cortical cytoskeleton organization | 46 | 8 | 2.950908 | 2.711029748 | 0.008229954 | 0.030275 |
| GO: 0070741 | response to interleukin-6 | 37 | 7 | 2.373557 | 2.94916074 | 0.008271398 | 0.030275 |
| GO: 0009636 | response to toxic substance | 499 | 46 | 32.01094 | 1.437008754 | 0.008298985 | 0.030275 |
| GO: 0006970 | response to osmotic stress | 76 | 11 | 4.875414 | 2.256218837 | 0.008858511 | 0.032178 |
| GO: 0002200 | somatic diversification of immune receptors | 66 | 10 | 4.233912 | 2.361881978 | 0.008965277 | 0.032428 |
| GO: 0051961 | negative regulation of nervous system development | 297 | 30 | 19.0526 | 1.574587985 | 0.00915828 | 0.032985 |
| GO: 1901522 | positive regulation of transcription from RNA polymerase | 21 | 5 | 1.347154 | 3.711528822 | 0.009230006 | 0.033103 |
| | II promoter involved in cellular response to chemical | | | | | | |
| | stimulus | | | | | | |
| GO: 0055123 | digestive system development | 142 | 17 | 9.109325 | 1.866219422 | 0.009477527 | 0.033848 |
| GO: 0032984 | protein-containing complex disassembly | 323 | 32 | 20.72051 | 1.544363696 | 0.009568874 | 0.034032 |
| GO: 0048771 | tissue remodeling | 154 | 18 | 9.879128 | 1.82202324 | 0.009838289 | 0.034662 |
| GO: 0009755 | hormone-mediated signaling pathway | 237 | 25 | 15.20359 | 1.644348212 | 0.009844332 | 0.034662 |
| GO: 0009743 | response to carbohydrate | 225 | 24 | 14.43379 | 1.662764912 | 0.009904363 | 0.034662 |
| GO: 0044839 | cell cycle G2/M phase transition | 213 | 23 | 13.66399 | 1.683256733 | 0.009926976 | 0.034662 |
| GO: 0033627 | cell adhesion mediated by integrin | 67 | 10 | 4.298062 | 2.326630008 | 0.009949998 | 0.034662 |
| GO: 0043543 | protein acylation | 238 | 25 | 15.26774 | 1.637439186 | 0.010353431 | 0.03592 |
| GO: 0042692 | muscle cell differentiation | 338 | 33 | 21.68276 | 1.521946434 | 0.010582438 | 0.036565 |
| GO: 0032620 | interleukin-17 production | 30 | 6 | 1.924505 | 3.117684211 | 0.010792553 | 0.036991 |
| GO: 0046685 | response to arsenic-containing substance | 30 | 6 | 1.924505 | 3.117684211 | 0.010792553 | 0.036991 |
| GO: 0009791 | post-embryonic development | 89 | 12 | 5.709366 | 2.10180958 | 0.011176677 | 0.038153 |
| GO: 0031929 | TOR signaling | 111 | 14 | 7.12067 | 1.96610716 | 0.011430404 | 0.038863 |
| GO: 0007164 | establishment of tissue polarity | 79 | 11 | 5.067864 | 2.17053964 | 0.011751733 | 0.039327 |
| GO: 0032602 | chemokine production | 79 | 11 | 5.067864 | 2.17053964 | 0.011751733 | 0.039327 |
| GO: 0042116 | macrophage activation | 79 | 11 | 5.067864 | 2.17053964 | 0.011751733 | 0.039327 |
| GO: 0090559 | regulation of membrane permeability | 79 | 11 | 5.067864 | 2.17053964 | 0.011751733 | 0.039327 |
| GO: 0052547 | regulation of peptidase activity | 418 | 39 | 26.81477 | 1.45442206 | 0.011908575 | 0.039695 |
| GO: 0051051 | negative regulation of transport | 458 | 42 | 29.38078 | 1.429505861 | 0.012292541 | 0.040815 |
| GO: 0051302 | regulation of cell division | 158 | 18 | 10.13573 | 1.775896069 | 0.012635934 | 0.041792 |
| GO: 0002064 | epithelial cell development | 206 | 22 | 13.21494 | 1.664782831 | 0.012971139 | 0.042696 |
| GO: 0051648 | vesicle localization | 305 | 30 | 19.5658 | 1.533287317 | 0.013009692 | 0.042696 |
| GO: 0051188 | cofactor biosynthetic process | 280 | 28 | 17.96205 | 1.558842105 | 0.013079671 | 0.04276 |
| GO: 0006352 | DNA-templated transcription, initiation | 231 | 24 | 14.81869 | 1.619576213 | 0.013414555 | 0.043687 |
| GO: 0051701 | interaction with host | 207 | 22 | 13.27909 | 1.656740402 | 0.013666524 | 0.044338 |
| GO: 0097696 | STAT cascade | 148 | 17 | 9.494226 | 1.790561878 | 0.013984206 | 0.045196 |
| GO: 0071772 | response to BMP | 160 | 18 | 10.26403 | 1.753697368 | 0.014252427 | 0.045888 |
| GO: 0060348 | bone development | 208 | 22 | 13.34324 | 1.648775304 | 0.014391236 | 0.046161 |
| GO: 0002931 | response to ischemia | 41 | 7 | 2.630157 | 2.661437741 | 0.0144706 | 0.046241 |
| GO: 0048645 | animal organ formation | 61 | 9 | 3.913161 | 2.299930975 | 0.015161408 | 0.048267 |
| GO: 0006605 | protein targeting | 412 | 38 | 26.42987 | 1.43776699 | 0.015317031 | 0.04858 |
| GO: 0030856 | regulation of epithelial cell differentiation | 138 | 16 | 8.852725 | 1.807353166 | 0.015460586 | 0.048853 |
| GO: 0006338 | chromatin remodeling | 150 | 17 | 9.622527 | 1.766687719 | 0.015810973 | 0.049775 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Dhanapani et al., Cell Reports, 39, 110883, 2022.
El Harane et al., Eur. Heart J, 39: 1835-1847, 2018.
Hur, Experimental and Molecular Medicine, 54, 433-446, 2022.
International Patent Publication No. WO 02/016536
International Patent Publication No. WO 03/016496
International Patent Publication No. WO 98/30679
International Patent Publication No. WO 98/53058
International Patent Publication No. WO 98/53059
International Patent Publication No. WO 98/53060
International Patent Publication No. WO2022/235911
PCT Publication No. 2012/149484
Sudom et al., J. Biol Chem., 10; 293(32): 12634-12646, 2018.
U.S. patent application Ser. No. 12/715,136
U.S. Pat. No. 6,140,081
U.S. Pat. No. 6,453,242
U.S. Pat. No. 6,534,261
U.S. Pat. No. 6,617,152
U.S. Pat. No. 8,372,642
U.S. Patent Publication No. 2002/0076747
U.S. Patent Publication No. 2005/0064474
U.S. Patent Publication No. 2005/0260186
U.S. Patent Publication No. 2006/0104968
U.S. Patent Publication No. 2006/0188987
U.S. Patent Publication No. 2007/0218528
U.S. Patent Publication No. 2011/0301073
U.S. Patent Publication No. 2011/0301073
U.S. Patent Publication No. 2011/0301073.
U.S. Patent Publication No. 20200239844
Vlassov et al., Biochim Biophys Acta., 940-948, 2012.
Wunderlich et al., Journal of Biological Chemistry, Molecular Bases of Disease, 288:46 (P33027-33036), 2013.

1. A composition comprising a microglia-derived extracellular vesicle-enriched secretome, wherein the microglia are induced pluripotent stem cell (iPSC)-derived microglia.

2. (canceled)

3. The composition of claim 1, wherein the extracellular vesicle-enriched secretome comprises hsa-miR-4669, hsa-miR-4777-3p, hsa-miR-16-5p, hsa-miR-223-3p, hsa-miR-93-5p, hsa-miR-146a-5p, hsa-miR-142-3p, hsa-miR-191-5p, hsa-miR-142-5p, hsa-miR-21-5p, hsa-miR-103a-3p/107, hsa-miR-26b-5p, hsa-miR-122-5p, hsa-miR-125b-5p, hsa-miR-25-3p, hsa-miR-146b-5p, hsa-miR-101-3p, hsa-miR-29a-3p, hsa-miR-30e-5p, hsa-let-7a-5p/7c-5p, hsa-miR-342-3p, hsa-miR-148b-3p, hsa-miR-27a-3p/27b-3p, hsa-miR-224-5p, hsa-let-7f-5p, hsa-miR-125a-5p, and/or hsa-miR-26a-5p.

4. (canceled)

5. The composition of claim 1, wherein the iPSC-derived microglia are positive for TREM2, P2RY12, TMEM119, IBA-1, and/or CX3CR1.

6. (canceled)

7. The composition of claim 1, wherein the extracellular vesicle-enriched secretome is positive for CD9, CD63, CD81, beta-actin, Flottilin-1, GBA, LRRK2, and/or phosphor-LRRK2.

8-9. (canceled)

10. The composition of claim 1, wherein the iPSC-derived microglia and/or extracellular vesicle-enriched secretome comprise a GBA protein larger than 80 kDa.

11. (canceled)

12. The composition of claim 1, wherein the extracellular vesicle-enriched secretome and/or iPSC-derived microglia comprises a TREM2 C-terminal cleavage fragment.

13. The composition of claim 1, wherein the extracellular vesicle-enriched secretome has no or essentially no HLA-ABC, CD86, and/or CD142.

14. The composition of claim 1, wherein the composition comprises relative protein levels of about 35-60% CD9, less than 10% CD63, and/or about 30-60% CD81.

15-17. (canceled)

18. The composition of claim 1, wherein the composition comprises less than 10% CD63, greater than 30% CD9 and/or less than 60% CD81.

19. The composition of claim 1, wherein the composition comprises between 0.5 and 8% CD63, between 36 and 62% CD9, and between 34 and 60% CD81.

20. The composition of claim 1, wherein the extracellular vesicles-enriched secretome was obtained from microglia which have not been cryopreserved after differentiation from iPSCs.

21. (canceled)

22. The composition of claim 1, wherein the microglia were not stimulated with lipopolysaccharide (LPS), phosphatidylserine positive (PS+) neurons, TNF-alpha, IFN-gamma, IL-4, or IL-10.

23. The composition of claim 1, wherein the conditioned media was produced from a spent media which was previously diluted about 1:1 with phosphate-buffered saline (PBS) prior to isolation of the extracellular-enriched secretome.

24. The composition of claim 1, wherein the extracellular vesicle-enriched secretome was isolated from the conditioned media by ultracentrifugation, TFF and size exclusion chromatography (TFF-SEC), or phosphatidyl-serine (PS) affinity capture.

25-27.

28. The composition of any claim 1, wherein the extracellular vesicle-enriched secretome comprises a median D50 of 110-130 nm.

29. (canceled)

30. (canceled)

31. The composition of claim 1, wherein the extracellular vesicles are singlets, doublets, concentric multi-vesicular bodies, and/or non-concentric multi-vesicular bodies.

32. (canceled)

33. The composition of claim 1, wherein the composition comprises at least 2% CD63⁺CD9⁺ clusters or at least 2% CD63⁺CD81⁺ clusters.

34-37. (canceled)

38. The composition of claim 1, wherein the extracellular vesicle-enriched secretome increases endothelial cell migration as measured by HUVEC scratch wound healing assay as compared to mock-EV (MV) controls.

39. (canceled)

40. The composition of claim 1, wherein the extracellular vesicle-enriched secretome influences electrical activity of neurons and/or Ca²⁺ signaling.

41. (canceled)

42. The composition of claim 1, wherein the extracellular vesicle-enriched secretome increases dopaminergic neuron viability, decreases neurite outgrowth, increasing the clustering of neuron cells, decreases the number of cell body clusters, and/or increases cell body cluster area when contacted with dopaminergic neurons as compared to untreated controls.

43. The composition of claim 1, wherein the extracellular vesicle-enriched secretome decreases peak amplitude, increases the standard deviation for peak-to-peak timing, increased peak bottom intensity, decreased ratio of peak amplitude to peak bottom, decreased peak amplitude, and/or decreased rising slope when contacted with neurospheres as compared to untreated controls.

44-45. (canceled)

46. A composition comprising an extracellular vesicle-enriched secretome produced by induced pluripotent stem cell (iPSC)-derived microglia.

47-94. (canceled)

95. A method of making a composition comprising an extracellular vesicle-enriched secretome, the method comprising culturing induced pluripotent stem cell (iPSC)-derived microglia to produce a conditioned media and isolating the extracellular vesicle-enriched secretome therefrom.

96-153. (canceled)
